(19) United States
(12) Patent Application Publication
(10) Pub. No.: US 2026/0109712 A1
(43) Pub. Date: Apr. 23, 2026

(54) RIFAMYCINS FOR NONTUBERCULOUS MYCOBACTERIA

(71) Applicants: REGENTS OF THE UNIVERSITY OF MINNESOTA, MINNEAPOLIS, MN (US); HACKENSACK MERIDIAN HEALTH, INC., EDISON, NJ (US)

(72) Inventors: Courtney C. ALDRICH, Minneapolis, MN (US); Tian LAN, Minneapolis, MN (US); Thomas DICK, Edison, NJ (US); Uday GANAPATHY, Edison, NJ (US); Veronique DARTOIS, Edison, NJ (US); Matthew ZIMMERMAN, Edison, NJ (US); Martin GENGENBACHER, Edison, NJ (US)

(21) Appl. No.: 19/118,284

(22) PCT Filed: Oct. 5, 2023
(86) PCT No.: PCT/US2023/034573

§ 371 Date: Apr. 3, 2025

Related U.S. Application Data
(60) Provisional application No. 63/413,472, filed on Oct. 5, 2022.

(51) Int. Cl. C07D 498/22; A61K 31/438; A61K 31/444; A61K 31/4545; A61K 31/496; A61K 31/497; A61K 31/501; A61K 31/506; A61K 31/517; A61K 31/52; A61P 31/04; C07D 519/00

(52) U.S. Cl. CPC C07D 498/22; A61K 31/438; A61K 31/444; A61K 31/4545; A61K 31/496; A61K 31/497; A61K 31/501; A61K 31/506; A61K 31/517; A61K 31/52; A61P 31/04; C07D 519/00

(57) ABSTRACT

Disclosed herein are compounds of Formula I and methods for using the same for treating or preventing an infection caused by a Mycobacterium in a subject in need thereof. The method comprises administering to the subject the compound of Formula I, or a pharmaceutically acceptable salt thereof, or a pharmaceutical composition comprising the compound of Formula I.

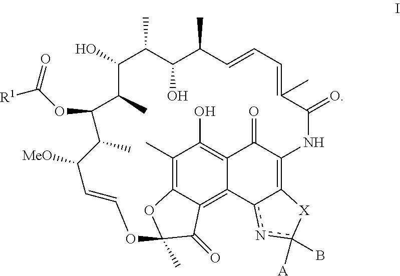

### CROSS-REFERENCE TO RELATED APPLICATIONS

This application claims priority to U.S. Provisional Application No. 63/413,472 filed on Oct. 5, 2022, the contents of which are incorporated by reference in their entireties.

### STATEMENT REGARDING FEDERALLY SPONSORED RESEARCH

This invention was made with government support under AI132374, AI142731, and AI177342 awarded by the National Institutes of Health. The government has certain rights in the invention.

### BACKGROUND OF THE INVENTION

Nontuberculous mycobacteria (NTM) can cause pulmonary and extrapulmonary infections. First isolated in 1952, Mycobacterium abscessus (M. abscessus) is recognized as the second most common pathogenic NTM and the most common agent of pulmonary infections caused by rapid-growing mycobacteria. Current treatment outcomes of mycobacteria, such as M. abscessus infections, remain unstable and varied between subspecies, with the subspecies abscessus being the deadliest with the treatment success usually below 50% and among 20-40%. The poor treatment outcome of mycobacteria infections is largely related to the intrinsically resistance of the pathogen to most of the existing antibiotics.

As such, there exists a need for developing novel therapeutic agents that can be used as potent and safe antibiotics against mycobacteria, in particular nontuberculous mycobacteria.

### SUMMARY OF THE INVENTION

Disclosed herein are compounds and methods for treating or preventing bacterial infections, such as mycobacterial infections. One aspect of the technology is a compound of formula I, or a pharmaceutically acceptable salt thereof:

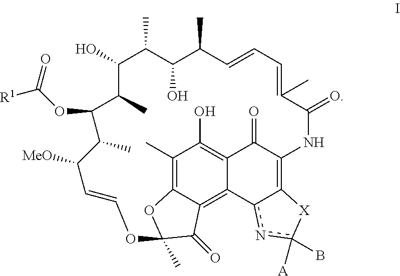

For the compound of formula I, R¹ is selected from the group consisting of optionally substituted alkyl, optionally substituted alkenyl, optionally substituted alkynyl, optionally substituted cycloalkyl, optionally substituted aryl, optionally substituted heteroaryl, optionally substituted heterocycloalkyl, —NR⁴R⁵, and —NHSO₂R⁶; R⁴ and R⁵ are independently selected from the group consisting of hydrogen, optionally substituted alkyl, optionally substituted haloalkyl, optionally substituted alkenyl, optionally substituted alkynyl, optionally substituted cycloalkyl, optionally substituted aryl, optionally substituted heteroaryl, optionally substituted heterocycloalkyl, or R⁴ and R⁵, together with the nitrogen atom to which they are attached, form an optionally substituted 4-8 membered heterocycloalkyl containing one or more heteroatoms selected from the group consisting of N, O, and S(O)₀₋₂; R⁶ is selected from the group consisting of optionally substituted alkyl, optionally substituted aryl, and optionally substituted heteroaryl;
is formula (a), formula (b), or formula (c),

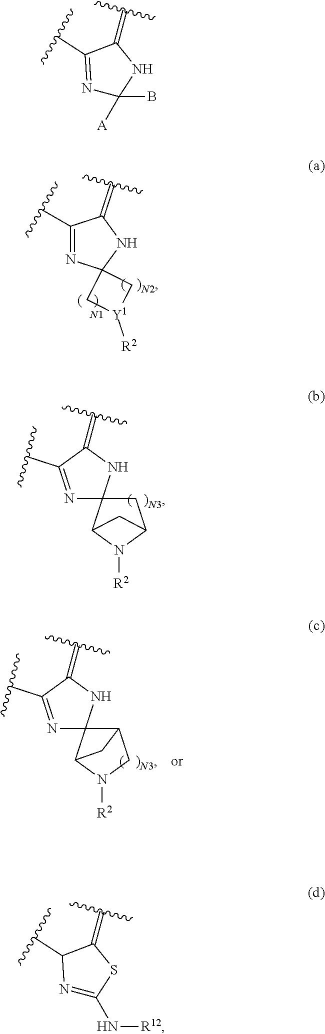

wherein N1 and N2 are independently an integer of 1-3; Y¹ is N, O, or CRⁱ; ² is selected from the group consisting of hydrogen, optionally substituted alkyl, optionally substituted haloalkyl, optionally substituted alkenyl, optionally substituted alkynyl, optionally substituted cycloalkyl, optionally substituted aryl, optionally substituted heteroaryl, optionally substituted heterocycloalkyl, and —(CH₂)ₙR³; Rⁱ is hydrogen, halo, optionally substituted alkyl, optionally substituted aryl, optionally substituted heteroaryl, or Rⁱ and R², together with the carbon atom to which they are attached, form an optionally substituted 4-8 membered heterocycloalkyl; n is an integer of 1-6; R³ is selected from the group consisting of —OR⁷, —NR⁸R⁹, —C(O)OR¹⁰, —S(O)₂OR¹¹, and heteroaryl; R⁷, R⁸, R⁹, R¹⁰, and R¹¹ are independently selected from the group consisting of hydrogen, alkyl, and haloalkyl; R¹² is heteroaryl; and N3 is an integer of 1-2, and wherein R¹ is not methyl. Exemplary embodiments of the compounds are disclosed herein.

Another aspect of the technology provides for a method for treating or preventing an infection caused by a Mycobacterium in a subject in need thereof. The method comprises administering to the subject one or more of the compounds disclosed herein or a pharmaceutically acceptable salt thereof. Suitably, the Mycobacterium may selected from the group consisting of Mycobacterium abscessus, Mycobacterium simiae, Mycobacterium chelonae, Mycobacterium fortuitum, Mycobacterium avium complex, Mycobacterium avium subspecies hominisuis, Mycobacterium intracellulare, Mycobacterium chimaera, Mycobacterium kansasii, Mycobacterium szulgai, Mycobacterium xenopi, and combinations thereof. In some embodiments, the Mycobacterium is Mycobacterium abscessus which may be optionally selected from the group consisting of Mycobacterium abscessus subspecies abscessus, Mycobacterium abscessus subspecies bolletii, Mycobacterium abscessus subspecies massiliense, and combinations thereof.

Another aspect of the technology provides for an infection caused by a bacterium in a subject in need thereof. The method comprises administering to the subject one or more of the compounds disclosed herein or a pharmaceutically acceptable salt thereof. The compounds may be administered to a subject where the bacterium is resistant to rifampicin, rifabutin, or rifapentine. wherein the bacterium expresses an enzyme that catalyzes ADP-ribosylation of rifampicin, rifabutin, or rifapentine. The compounds may be administered to a subject where the bacterium expresses an enzyme that catalyzes ADP-ribosylation of rifampicin, rifabutin, or rifapentine. In some embodiments, the enzyme is a rifamycin ADP-ribosyltransferase. Suitably, the bacterium is a Mycobacterium, which may be optionally selected from any of the Mycobacterium disclosed herein.

Another aspect of the technology provides for a method for treating or preventing an infection in a subject in need of an antibacterial compound. The method comprises administering to the subject one or more of the compounds disclosed herein or a pharmaceutically acceptable salt thereof. The administered compound may have less inductive effect on a cytochrome P450 enzyme than any one or more of rifampicin, rifabutin, and rifapentine. The cytochrome P450 enzyme may be P450 3A4. In some embodiments, the subject is undergoing treatment with a compound metabolized by the cytochrome P450 enzyme or is in need of treatment with the compound metabolized by the cytochrome P450 enzyme. In some embodiments, the subject is undergoing treatment with a compound that is sensitive to the co-administration of an inducer of the cytochrome P450 enzyme or is in need of treatment with the compound that is sensitive to the co-administration of an inducer of the cytochrome P450 enzyme. Suitably, the subject is infected with a Mycobacterium, which may be optionally selected from any of the Mycobacterium disclosed herein.

Pharmaceutical compositions comprising one or more compounds disclosed herein for use with any of the methods disclosed herein are also provided.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 illustrates that Rifabutin is inactivated in M. abscessus. Arr of M. abscessus catalyzes the formation of ADP-ribosyl-oxocarbenium intermediate from NAD⁺ and the consecutive ADP-ribosylation on C23-OH.

FIGS. 2A-2C demonstrate that homology-model-guided rifamycin design depicts C-25 modification blocking interaction with Arr_{Mab}. FIG. 2A shows the structure of the archetypical compound 25-O-benzoyl rifabutin (5a), where the C25 acetyl group of rifabutin is replaced by a benzoyl group. FIG. 2B shows the overall structure of Arr_{Mab} homology model complexed with the ADP-ribosyl-oxocarbenium intermediate (right molecule) and 5a (left molecule). FIG. 2C shows proposed binding modes of rifabutin (left panel) and 5a (right panel) to the Arr_{Mab} homology model. The distance between C23-OH and oxocarbenium-C1′ is measured and shown as a dashed line.

FIG. 3A demonstrates that the active site structure of 5a (center molecule) bound to RNAP_{Mtb}. Residues involving in important hydrogen bonds are shown as sticks. H-bonds are shown as dashed lines. FIG. 3B illustrates C-25 benzoate (C25-OBz) of 5a (left) and F439 (right) forms a π-stacking interaction. FIG. 3C shows that C-25 benzoate of 5a (bottom left molecule) is positioned in a cleft formed by F439 and R173. The cleft is demonstrated in darker shades towards the right of 5a.

FIG. 4 demonstrates in vitro characterization of ADP-ribosylation of rifamycins using overexpressed Arr_{Mab}. All the peaks were identified by MS (Table 7). The extension time difference of the peaks is labelled. Rifabutin was fully converted into the ADP-ribosyl adduct upon incubation in a 40-minute time course. By contrast, no Arr_{Mab}-catalyzed transformation was observed for the synthetic compound 5a under the same incubation condition.

FIG. 5 illustrates that analog 5j exhibited bactericidal in vivo efficacy at 10 mg/kg. Animals infected with M. abscessus underwent drug treatment for 10 consecutive days. Drugs were administered once daily to groups of 6 mice per study group. At 11 days postinfection, organ homogenates were plated on agar to determine the bacterial load. Results were analyzed using one-way analysis of variance (ANOVA) multicomparison and Dunnett's posttest. *, P<0.05; **, P<0.01; ***, P<0.001. D1: Day-1. D11: Day-11. CLR: clarithromycin. RBT: rifabutin.

FIG. 6 illustrates binding mode of rifampicin (center molecule) in RNAP_{Mtb} (PDB: 5UHB). The binding pocket is shown in lighter shades around the molecule. The space accommodating C25-OAc is contoured in dashed lines.

FIG. 7 illustrates proposed binding modes of rifabutin (molecule without the six-membered ring on top) and 5a (molecule with the six-membered ring on top) in the RNAP_{Mab} homology model. High overlap was observed for the two molecules.

FIG. 8 illustrates comparison of co-crystal structures of 5a-RNAP_{Mtb} and rifampicin-RNAP_{Mtb} (PDB: 5UHB). Residues with direct interactions with the ligands are labeled. The three-dimensional structure reveals that 5a adopts a highly similar binding mode to rifampicin in the same binding site.

FIG. 9 shows proposed binding modes and binding affinities of analogs 5b-5g in the RNAP_{Mab} homology model. High overlap was observed for these molecules.

FIGS. 10A-10G demonstrate in vitro characterization of ADP-ribosylation of rifamycins and synthetic compounds using overexpressed Arr_{Mab}. All the peaks were further confirmed by MS (Table 6). The extension time difference of the peaks is labelled. Controls rifampicin (FIG. 10A) and rifabutin (FIG. 10B) were fully ADP-ribosylated upon 40-minute incubations. No ADP-ribosylated adducts were observed for the synthetic compounds 5a (FIG. 10C), 5b (FIG. 10D) and 5m (FIG. 10E) bearing bulky C-25 substituents. Synthetic compounds 5k (FIG. 10F) and 5l (FIG. 10G) with small alkyl substituents on C-25 failed to block ADP-ribosylation and transformed into the respective ADP-ribosylated adducts upon incubation in a 40-minute time course.

FIG. 11 shows the ¹H NMR and ¹³C NMR data of compound 5a.

FIG. 12 shows the ¹H NMR and ¹³C NMR data of compound 5b.

FIG. 13 shows the ¹H NMR and ¹³C NMR data of compound 5c.

FIG. 14 shows the ¹H NMR and ¹³C NMR data of compound 5d.

FIG. 15 shows the ¹H NMR and ¹³C NMR data of compound 5e.

FIG. 16 shows the ¹H NMR and ¹³C NMR data of compound 5f.

FIG. 17 shows the ¹H NMR and ¹³C NMR data of compound 5g.

FIG. 18 shows the ¹H NMR and ¹³C NMR data of compound 5h.

FIG. 19 shows the ¹H NMR and ¹³C NMR data of compound 5i.

FIG. 20 shows the ¹H NMR and ¹³C NMR data of compound 5j.

FIG. 21 shows the ¹H NMR and ¹³C NMR data of compound 5k.

FIG. 22 shows the ¹H NMR and ¹³C NMR data of compound 5l.

FIG. 23 shows the ¹H NMR and ¹³C NMR data of compound 5m.

FIG. 24 shows the ¹H NMR and ¹³C NMR data of compound 5n.

FIG. 25 shows the ¹H NMR and ¹³C NMR data of compound 5o.

FIG. 26 shows that under the conditions of using anhydride along or with substoichiometric amount of DMAP for acylation from compound 3, the starting material had almost no conversion (Case 1 and Case 2). In certain cases, the relatively low yield was due to extremely slow conversion and, once heated up, the massive side reactions (Case 3 and Case 4).

FIG. 27 shows the cLog P-plasma unbound fraction plot.

FIG. 28 shows the growth of M. abscessus Bamboo in caseum surrogate. The surrogate matrix was generated as described previously from cultured THP-1 cells (ATCC TIB-202) (mBio, 2023, 14, e0059823). M. abscessus exponential cultures grown in Middlebrook 7H9 broth (Sigma Aldrich) (OD₆₀₀ 0.6-0.9) were spun down and resuspended in water to an OD₆₀₀ of 7, 0.7, and 0.07. As described for the M. tuberculosis caseum surrogate assay (mnBio, 2023, 14, e0059823), the bacterial suspensions (at three different dilutions resulting in ˜10⁹, 10⁸, and 10⁷ starting CFU/mL, represented by circle, square, and triangle indicators respectively) were added to the caseum surrogate in the ratio 2:1 (vol/wt), briefly homogenized with 1.4-mm zirconia beads, divided evenly into nine 1.5-mL microcentrifuge tubes, and incubated as standing cultures at 37° C. At the indicated time points, tubes were removed and used for CFU enumeration by plating on Middlebrook 7H11 agar (Sigma Aldrich). Separate tubes were used at each time point. To determine the kill curves, cultures with a starting CFU/mL of 10⁸ were used (resulting in the middle curve with square indicators). Arrows indicate the time points when drugs were added and the end of the treatment. The experiment was repeated three times independently, yielding similar results. A representative example is shown. Dots and error bars represent means and standard deviations of three technical replicates, respectively.

FIGS. 29A-29M show the Dose-response kill curves against M. abscessus Bamboo in caseum surrogate. M. abscessus cultures were set up as described in the legend of FIG. 28. Bacterial cell suspensions were added to yield a starting CFU of 10⁸/mL (FIG. 28, middle growth curve with square indicators). At day 5, after the cultures entered stationary phase (FIG. 28, first arrow), 50 μL mixtures (cultures in caseum surrogate) were exposed to drugs (1 μL in DMSO. Amikacin, clarithromycin, clofazimine, imipenem, rifabutin and tigecycline were purchased from Sigma Aldrich, moxifloxacin and linezolid from Sequoia Research Products, and cefoxitin and bedaquiline from MedChemExpress. The rifabutin analogs were synthesized as described (doi: 10.1002/anie.202211498).) in the range of 0.125-512 μM (128 μM for clofazimine and rifabutin analogs 5a, 5m, 5n) for 5 days (or 10 days for bedaquiline as described in Table 16), after which CFU was enumerated. Addition of 2% of the vehicle DMSO did not affect viable counts. The shaded areas indicate drug concentration windows achieved in caseum in vivo. (Antimicrob. Agents Chemother., 2021, 65, e0050621; Antimicrob. Agents Chemother., 2022, 66, e0221221; PLoS Med., 2019, 16, e1002773; ACS Infect. Dis., 2016, 2, 251-267). The experiment was repeated twice independently, yielding similar results. A representative example is shown. Dots and error bars represent means and standard deviations of three technical replicates, respectively. Horizontal dotted lines indicate the cutoff for 1 log reduction in CFU compared to 10-day drug-free control culture. The cMBC₉₀ values shown in Table 16 are the drug concentrations that reduce CFU by 90% relative to the CFU of drug-free controls at day 10. Since the cultures were in the stationary phase on day 5 when drug treatment started, the CFUs of the drug-free cultures at day 10 were similar to the CFUs of the drug-free cultures at day 5 (FIG. 28).

FIG. 30 shows that more than 150 compounds have been synthesized. Their MIC data against Mycobacterium abscessus has been evaluated.

FIG. 31 shows that 88 compounds meet activity criteria (MIC<100 nM against Mycobacterium abscessus); Comprehensive SAR has been developed for rational design of active compounds.

FIG. 32 shows the strong correlation between fᵤ and clog P, in which clog P and log fᵤ form a linear correlation, with r²=0.66.

FIG. 33 shows that UMN22 and UMN34 demonstrate significantly better efficacy vs RFB. Both show ˜2 log¹⁰ reduction in lung CFU burden vs RFB and untreated (UNRX) groups.

### DETAILED DESCRIPTION OF THE INVENTION

The present technology provides for rifabutin analogs and methods of using the same for treating an infection caused by a nontuberculous Mycobacterium. The disclosed compounds are derivatized on the ansa-chain, especially on C25, of rifabutin, to increase the potency against M. abscessus by directly addressing the issue of rifamycin inactivation via ADP-ribosylation. The rifabutin analogs include, but are not limited to, C25 carbamates, sulfonamides, and carboxylic esters. The disclosed compounds are demonstrated to restore the intrinsic low nanomolar antimycobacterial activity of rifamycin antibiotics and have significant in vitro and in vivo antibacterial efficacy. Moreover, the disclosed compounds demonstrate substantially lower induction of CYP than rifampicin or rifabutin.

One aspect of the technology is a compound of Formula I, or a pharmaceutically acceptable salt thereof:

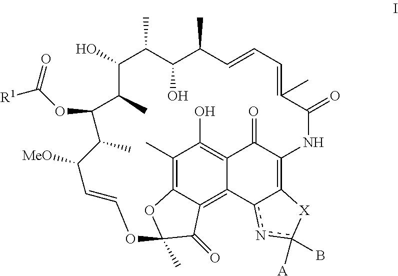

wherein:
R¹ is selected from the group consisting of optionally substituted alkyl, optionally substituted alkenyl, optionally substituted alkynyl, optionally substituted cycloalkyl, optionally substituted aryl, optionally substituted heteroaryl, optionally substituted heterocycloalkyl, —NR⁴R⁵, and —NHSO₂R⁶;
R⁴ and R⁵ are independently selected from the group consisting of hydrogen, optionally substituted alkyl, optionally substituted haloalkyl, optionally substituted alkenyl, optionally substituted alkynyl, optionally substituted cycloalkyl, optionally substituted aryl, optionally substituted heteroaryl, optionally substituted heterocycloalkyl, or
R⁴ and R⁵, together with the nitrogen atom to which they are attached, form an optionally substituted 4-8 membered heterocycloalkyl containing one or more heteroatoms selected from the group consisting of N, O, and S(O)₀₋₂;
R⁶ is selected from the group consisting of optionally substituted alkyl, optionally substituted aryl and optionally substituted heteroaryl;

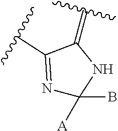

is formula (a), formula (b), or formula (c),

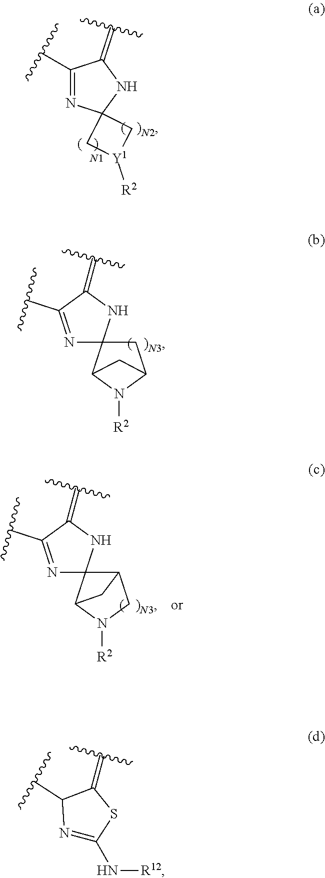

wherein
   N1 and N2 are independently an integer of 1-3;
   Y¹ is N, O, or CRⁱ;
   R² is selected from the group consisting of hydrogen, optionally substituted alkyl, optionally substituted haloalkyl, optionally substituted alkenyl, optionally substituted alkyny, optionally substituted cycloalkyl, optionally substituted aryl, optionally substituted heteroaryl, optionally substituted heterocycloalkyl, and —(CH₂)ₙR³;
   Rⁱ is hydrogen, halo, optionally substituted alkyl, optionally substituted aryl, optionally substituted heteroaryl, or Rⁱ and R², together with the carbon atom to which they are attached, form an optionally substituted 4-8 membered heterocycloalkyl;
   n is an integer of 1-6;
   R³ is selected from the group consisting of —OR⁷, —NR⁸R⁹, —C(O)OR¹⁰, —S(O)₂OR¹¹, and heteroaryl;
   R⁷, R⁸, R⁹, R¹⁰, and R¹¹ are independently selected from the group consisting of hydrogen, alkyl, and haloalkyl;
   R¹² is heteroaryl; and
   N3 is an integer of 1-2, and
wherein the compound is not rifabutin.

In some embodiments, R¹ is not methyl, ethyl, or propyl or a substituted methyl, ethyl, or propyl.

In some embodiments, R¹ is not aan alkyl substituted with a carbonyl containing group, such as a carboxylic acid (—COOH), ester (—COOR), or amide (—CONRR′).

The term “alkyl” includes a straight-chain or branched alkyl radical in all of its isomeric forms, such as a straight or branched group of 1-2, 1-10, or 6 carbon atoms, referred to herein as C₁-C₁₂ alkyl, C₁-C₁₀ alkyl, and C₁-C₆ alkyl, respectively.

The term “aryl” refers to a carbocyclic aromatic group. Representative aryl groups include phenyl, naphthyl, anthracenyl, and the like. The term “aryl” also includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. In some embodiments, the aryl group is a 6-10 membered ring structure (i.e. C₆-C₁₀ aryl).

The term “heteroaryl” refers to an aromatic ring structure containing a specified number of ring atoms in which at least one of the ring atoms is a heteroatom (i.e. oxygen, nitrogen, or S(O)₀₋₂), with the remaining ring atoms being independently selected from the group consisting of C(O)₀₋₁, oxygen, nitrogen, and sulfur. A 5- to 6-membered heteroaryl is an aromatic ring system which has five or six ring atoms with at least one of the ring atoms being N, O or S(O)₀₋₂. Similarly, a 5- to 10-membered heteroaryl is an aromatic ring system which has five to ten ring atoms with at least one of the ring atoms being N, O or S(O)₀₋₂. A heteroaryl may contain two or more fused rings.

Examples of heteroaryl substituents include six membered ring substituents such as pyridinyl, pyrazinyl, pyrimidinyl, and pyridazinyl; five membered ring substituents such as triazolyl, imidazolyl, furanyl, thiophenyl, pyrazolyl, oxazolyl, isoxazolyl, thiazolyl, 1,2,3-, 1,2,4-, 1,2,5-, or 1,3,4-oxadiazolyl and isothiazolyl; 6/5-membered fused ring substituents such as benzothiofuranyl, isobenzothiofuranyl, benzisoxazolyl, benzoxazolyl, purinyl, and anthranilyl; and 6/6-membered fused rings such as quinolinyl, isoquinolinyl, cinnolinyl, quinazolinyl, and 1,4-benzoxazinyl. In a group that has a heteroaryl substituent, the ring atom of the heteroaryl substituent that is bound to the group may be the at least one heteroatom, or it may be a ring carbon atom, where the ring carbon atom may be in the same ring as the at least one heteroatom or where the ring carbon atom may be in a different ring from the at least one heteroatom. Similarly, if the heteroaryl substituent is in turn substituted with a group or substituent, the group or substituent may be bound to the at least one heteroatom, or it may be bound to a ring carbon atom, where the ring carbon atom may be in the same ring as the at least one heteroatom or where the ring carbon atom may be in a different ring from the at least one heteroatom. Examples of 2-fused-ring heteroaryls include indolizinyl, pyranopyrrolyl, 4H-quinolizinyl, purinyl, naphthyridinyl, pyridopyridinyl (including pyrido[3,4-b]-pyridinyl, pyrido[3,2-b]-pyridinyl, or pyrido[4,3-5]-pyridinyl), pyrrolopyridinyl, pyrazolopyridinyl and imidazothiazolyl and pteridinyl.

Other examples of fused-ring heteroaryls include benzo-fused heteroaryls such as indolyl, isoindolyl, indoleninyl, isoindazolyl, benzazinyl (including quinolinyl or isoquinolinyl), phthalazinyl, quinoxalinyl, benzodiazinyl (including cinnolinyl or quinazolinyl), benzopyranyl, benzothiopyranyl, benzoxazolyl, indoxazinyl, anthranilyl, benzodioxolyl, benzodioxanyl, benzoxadiazolyl, benzofuranyl, isobenzofuranyl, benzothienyl, isobenzothienyl, benzothiazolyl, benzothiadiazolyl, benzimidazolyl, benzotriazolyl, benzoxazinyl, benzisoxazinyl.

The term “optionally substituted” refers to a group (e.g. alkyl, aryl, and heteroaryl) that is unsubstituted or substituted with one or more substituents independently selected from the group consisting of halo, azide, alkyl, alkenyl, alkynyl, alkylaryl, cycloalkyl, heterocycloalkyl, hydroxyl, alkoxy, amino, nitro, amido, —C(O)H, —C(O)-alkyl, —C(O)O-alkyl, carboxyl, alkylthio, sulfonamido, —S(O)-alkyl, aryl, heteroaryl, haloalkyl, cyano, carboxylic ester, and hydroxyalkyl.

The term “halo” refers to a halogen atom or halogen radical (e.g., —F, —Cl, —Br, or —I).

The term “haloalkyl” refers to an alkyl group that is substituted with at least one halogen. For example, —CH₂F, —CHF₂, —CF₃, —CH₂CF₃, —CF₂CF₃, and the like.

The term “azide” refers to the radical —N═N⁺═N⁻ (i.e. —N₃).

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C₂-C₁₂ alkenyl, C₂-C₁₀ alkenyl, and C₂-C₆ alkenyl, respectively.

The term “alkynyl” as used herein refers to an unsaturated straight or branched hydrocarbon having at least one carbon-carbon triple bond, such as a straight or branched group of 2-12, 2-10, or 2-6 carbon atoms, referred to herein as C2-C₁₂ alkynyl, C₂-C₁₀ alkynyl, and C₂-C₆ alkynyl, respectively.

The term “alkylaryl” refers to an alkyl substituted with an aryl. In some embodiments, the alkylaryl group is benzyl.

The term “alkylheteroaryl” refers to an alkyl substituted with a heteroaryl.

The term “cycloalkyl” refers to a carbocyclic substituent obtained by removing a hydrogen atom from a saturated carbocyclic molecule and having the specified number of carbon atoms. In one embodiment, a cycloalkyl substituent has three to seven carbon atoms (i.e., C₃-C₇ cycloalkyl). Examples of cycloalkyl include cyclopropyl, cyclobutyl, cyclopentyl, cyclohexyl and cycloheptyl. The term “cycloalkyl” includes mono-, bi- and tricyclic saturated carbocycles, as well as bridged and fused ring carbocycles, as well as spiro-fused ring systems.

The term “heterocycloalkyl” refers to a monovalent saturated cyclic, bicyclic, or bridged cyclic hydrocarbon group of 3-12, 3-8, 4-8, or 4-6 carbons in which at least one carbon of the cycloalkane is replaced with a heteroatom such as, for example, N, O, and/or S(O)ₙ, wherein n is an integer of 0-2. “Four to seven membered heterocycloalkyl” refers to a heterocycloalkyl containing from four to seven atoms, including one or more heteroatoms, in the cyclic moiety of the heterocycloalkyl. Examples of single-ring heterocycloalkyls include azetidinyl, oxetanyl, thietanyl, dihydrofuranyl, tetrahydrofuranyl, dihydrothiophenyl, tetrahydrothiophenyl, pyrrolinyl, pyrrolidinyl, imidazolinyl, imidazolidinyl, pyrazolinyl, pyrazolidinyl, thiazolinyl, isothiazolinyl, thiazolidinyl, isothiazolidinyl, dihydropyranyl, piperidinyl, morpholinyl, piperazinyl, azepinyl, oxepinyl, and diazepinyl. In some embodiments, the heterocycloalkyl described herein may be fused with a cycloalkyl, an aryl, or a heteroaryl, as described herein.

The term “hydroxyl” refers to the substituent of “—OH”.

The term “hydroxyalkyl” refers to an alkyl substituted with a hydroxyl group.

The term “cyano” refers to the substituent of “—CN”.

The terms “alkoxy” or “alkoxyl” refer to an alkyl group, as defined above, having an oxygen radical attached thereto. Representative alkoxy groups include methoxy, ethoxy, tert-butoxy and the like.

The terms “amine” and “amino” refer to both unsubstituted and substituted amines (e.g., mono-substituted amines or di-substituted amines), wherein substituents may include, for example, alkyl, cycloalkyl, heterocyclyl, alkenyl, aryl, and amino.

The term “nitro” refers to the substituent of “—NO₂”.

The term “amido” as used herein refers to the radical —C(O)NRR′ or —NR—C(O)R′, where R and R′ may be independently hydrogen, alkyl, aryl, arylalkyl, cycloalkyl, formyl, haloalkyl, heteroaryl, heterocyclyl, or amino.

The term “carboxy” or “carboxyl” as used herein refers to the radical —COOH.

The term “carboxylic ester” as used herein refers to the radical —C(O)OR or —OC(O)R, wherein R is a non-hydrogen group including, but are not limited to, alkyl, aryl, arylalkyl, cycloalkyl, haloalkyl, heteroaryl, or heterocyclyl.

The term “alkylthio” refers to the radical —S-alkyl.

The term “sulfonamido” as used herein refers to the radical —S(O)₂NRR′ or —NR—S(O)₂R′, where R and R′ may be the same or different. R and R′, for example, may be independently hydrogen, alkyl, aryl, arylalkyl, cycloalkyl, formyl, haloalkyl, heteroaryl, or heterocyclyl.

The term “pyridyl” as used herein refers to the radical

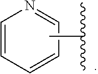

The term “pyrimidyl” as used herein refers to the radical

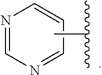

The term “thiazolyl” as used herein refers to the radical

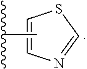

The term “pharmaceutically acceptable salt” refers to salts of the compounds disclosed herein that are substantially non-toxic to living organisms. Typical pharmaceutically acceptable salts include those salts prepared by reaction of the compounds as disclosed herein with an organic/inorganic acid or base. Such salts are known as acid addition and base addition salts. It will be appreciated by the skilled reader that all the compounds as disclosed herein are capable of forming salts and that the salt forms of pharmaceuticals are commonly used, often because they are more readily crystallized and purified than are the free acids or bases.

R¹ may be an aryl optionally substituted with one or more alkyl, alkoxy optionally substituted with halo or hydroxyl, hydroxyl, hydroxyalkyl, halo, haloalkyl, haloalkoxy, cyano, aryl, —C(O)OZ¹, —C(O)NZ²Z³, —S(O)Z⁴, —S(O)₂Z⁵, —S(O)₂NZ⁶Z⁷, or amido, wherein Z¹, Z², Z³, Z⁴, Z⁵, Z⁶, and Z⁷ are independently hydrogen, haloalkyl, alkyl, or —C(O)-alkyl.

R¹ may be a heteroaryl optionally substituted with one or more hydroxyl, halo, optionally substituted amino, amido, haloalkyl, or carboxylic ester.

R¹ may be —NR⁴R⁵ and R⁴ is hydrogen and R⁵ is an alkyl optionally substituted with one or more alkynyl or heteroaryl, said heteroaryl optionally substituted with one or more aryl optionally substituted with one or more aryl; or one or more alkyl optionally substituted with one or more aryl or carboxylic ester.

R¹ may be —NR⁴R⁵ and R⁴ is hydrogen and R⁵ is a heterocycloalkyl optionally substituted with one or more alkylaryl, alkoxy, amino, aryl, —C(O)OZ⁸, or alkyl optionally substituted with one or more haloalkyl or hydroxyl.

R¹ may be —NR⁴R⁵ and R⁴ is hydrogen and R⁵ is an aryl optionally substituted with one or more aryl, alkoxy, alkyl, —C(O)OZ⁸, —C(O)NZ⁹Z¹⁰, —S(O)₂Z¹¹, or —S(O)₂NZ¹²Z¹³.

R¹ may be —NR⁴R⁵ and R⁴ is hydrogen and R⁵ is a heteroaryl optionally substituted with one or more alkyl, halo, haloalkyl, amino, hydroxyl, —OC(O)Z⁸, or aryl optionally substituted with aryl.

In the foregoing, Z⁸, Z⁹, Z¹⁰, Z¹¹, Z¹², and Z¹³ may be independently hydrogen, alkyl, or —C(O)-alkyl.

R¹ may be R⁴ and R⁵, together with the nitrogen atom to which they are attached, form a 4-8 membered heterocycloalkyl containing one or more heteroatoms selected from the group consisting of N, O, and S(O)₀₋₂, said heterocycloalkyl is optionally substituted with one or more aryl, amino, haloalkyl, hydroxyalkyl, alkoxy, carboxylic ester, or alkyl.

R¹ may be —NHSO₂R⁶ and R⁶ is heteroaryl or aryl optionally substituted with one or more halo, alkoxy, or haloalkyl.

R¹ may be alkyl.

R¹ may be alkynyl.

R¹ may by cycloalkyl.

In some embodiments, R¹ in the compound of Formula I is an optionally substituted aryl or an optionally substituted heteroaryl.

In some embodiments, the compound of Formula I has a formula of I(a):

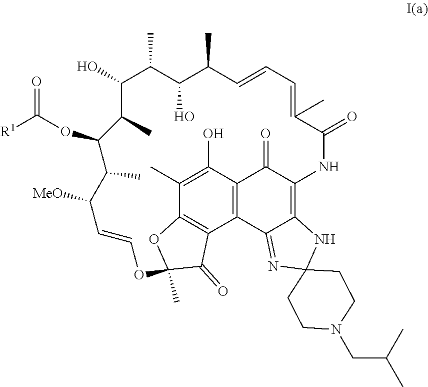

wherein R¹ is selected from the group consisting of alkyl. —NR⁴R⁵, —NHSO₂R⁶, cycloalkyl, heteroaryl optionally substituted with one or more hydroxyl, halo, optionally substituted amino, amido, haloalkyl, or carboxylic ester, and aryl optionally substituted with one or more alkyl, alkoxy optionally substituted with halo or hydroxyl, hydroxyl, hydroxyalkyl, halo, haloalkyl, haloalkoxy, cyano, aryl, —C(O)OZ¹, —C(O)NZ²Z³, S(O)Z⁴, S(O)₂Z⁵, —S(O)₂NZ⁶Z⁷, or amido, wherein Z¹, Z², Z³, Z⁴, Z⁵, Z⁶, and Z⁷ are independently hydrogen, alkyl, or —C(O)-alkyl.

In some embodiments, R¹ in the compound of Formula I is

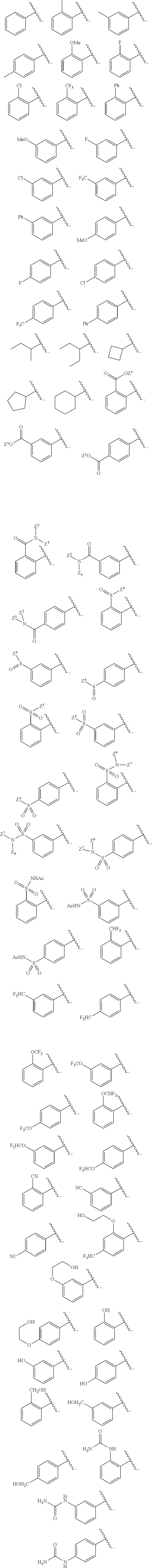

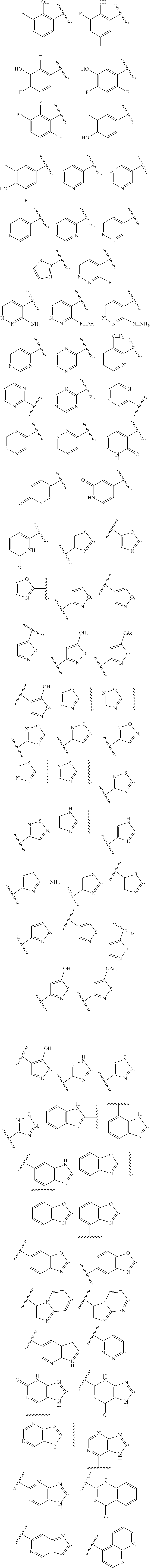

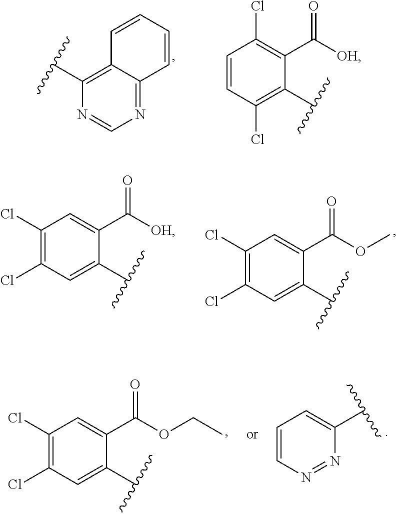 [SMILES: *c1ncnc2ccccc12 ; *c1c(Cl)ccc(Cl)c1C(=O)O ; *c1cc(Cl)c(Cl)cc1C(=O)O ; *c1cc(Cl)c(Cl)cc1C(=O)OC ; *c1cc(Cl)c(Cl)cc1C(=O)OCC ; *c1cccnn1]

In some embodiments, the compound of Formula I is

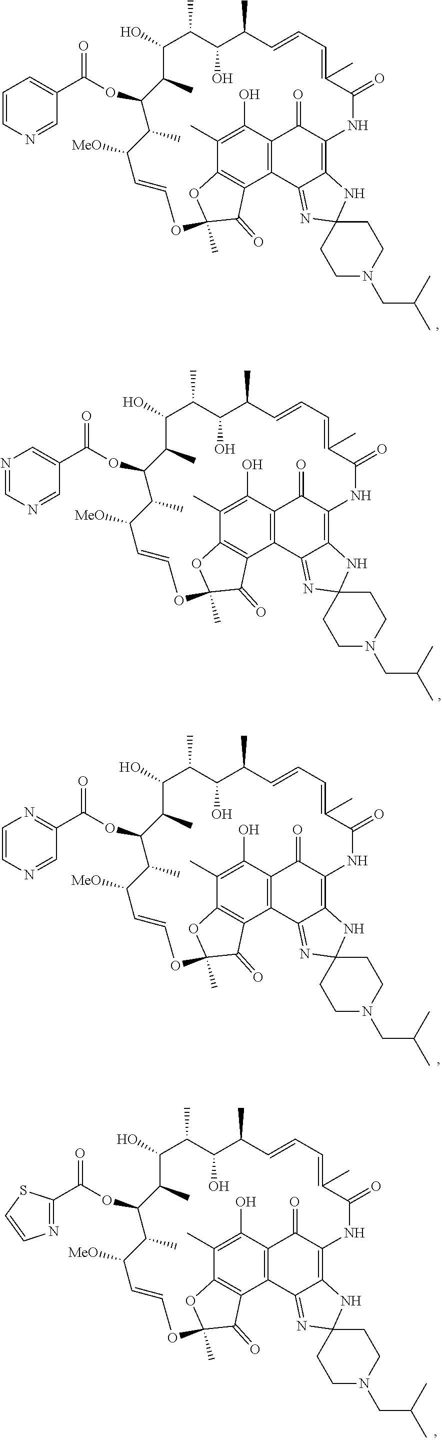

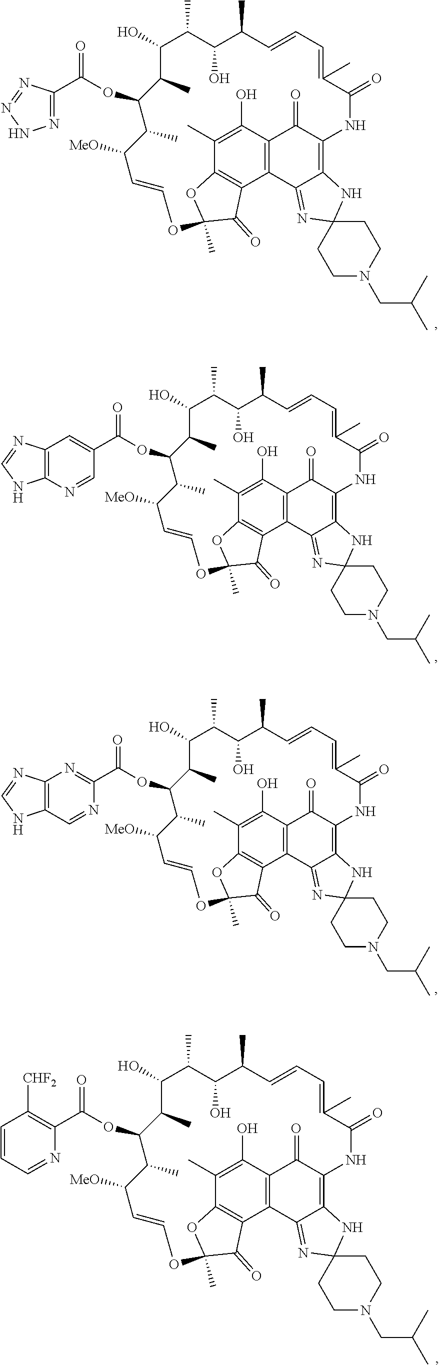

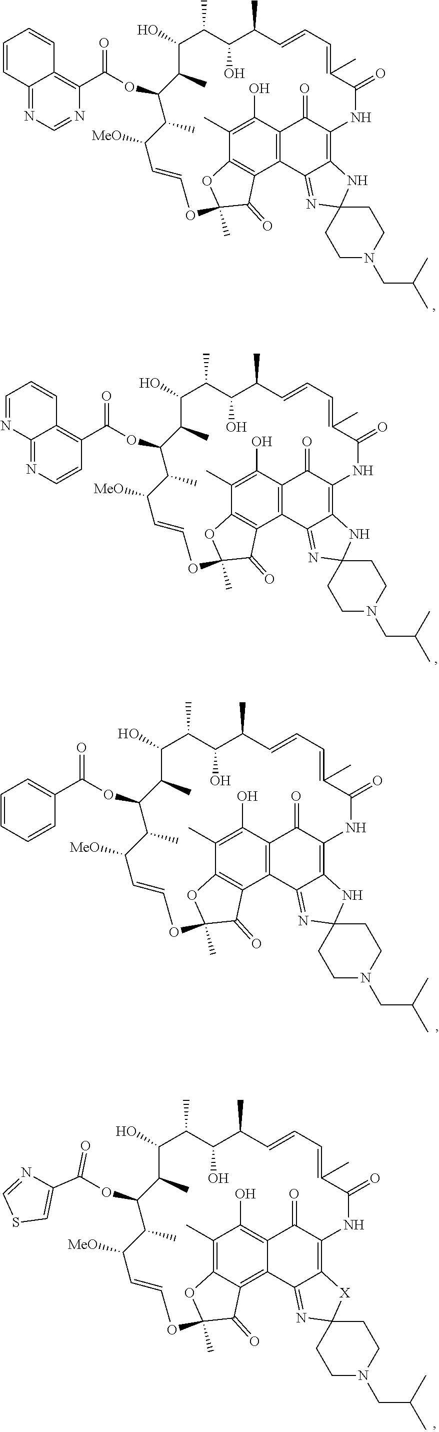

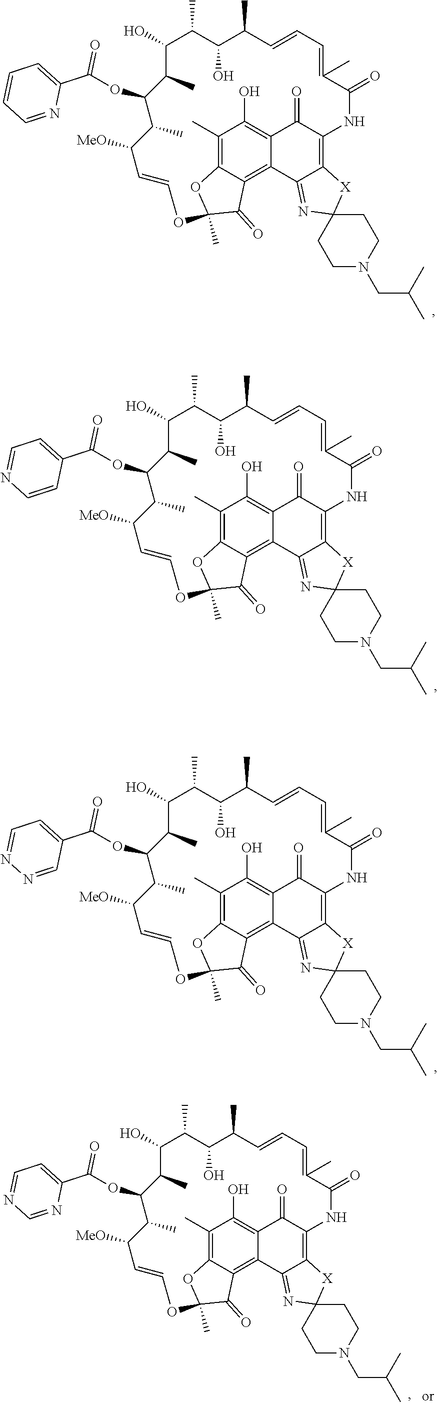

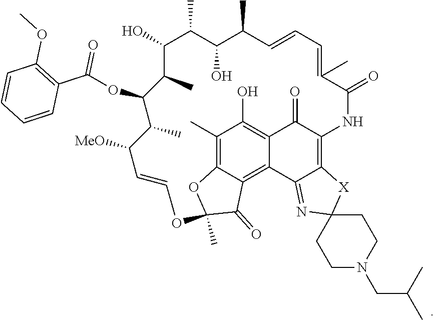

In some embodiments, R¹ in the compound of Formula I is —NR⁴R⁵. In some such embodiments, R⁴ and R⁵ are independently selected from the group consisting of hydrogen, alkylheteroaryl optionally substituted with aryl that is optionally substituted with aryl, heterocycloalkyl optionally substituted with one or more alkylaryl, alkoxy, amino, aryl, —C(O)OZ⁸, or alkyl optionally substituted with one or more haloalkyl or hydroxyl, aryl optionally substituted with one or more aryl, alkoxy, alkyl, —C(O)OZ⁸, —C(O)NZ⁹Z¹⁰, —S(O)₂Z¹¹, or —S(O)₂NZ¹²Z³, and heteroaryl optionally substituted with one or more aryl, halo, haloalkyl, —OC(O)Z⁸, amino, or hydroxyl, or R⁴ and R⁵, together with the nitrogen atom to which they are attached, form a 4-8 membered heterocycloalkyl containing one or more heteroatoms selected from the group consisting of N, O, and S(O)₀₋₂, wherein the heterocycloalkyl is optionally substituted with one or more alkyl, haloalkyl, hydroxyalkyl, aryl, alkoxy, carboxylic ester, or amino, wherein Z⁸, Z⁹, Z¹⁰. Z¹¹, Z¹², and Z¹³ are independently hydrogen, alkyl, or —C(O)-alkyl.

In some embodiments, R¹ in the compound of Formula I is

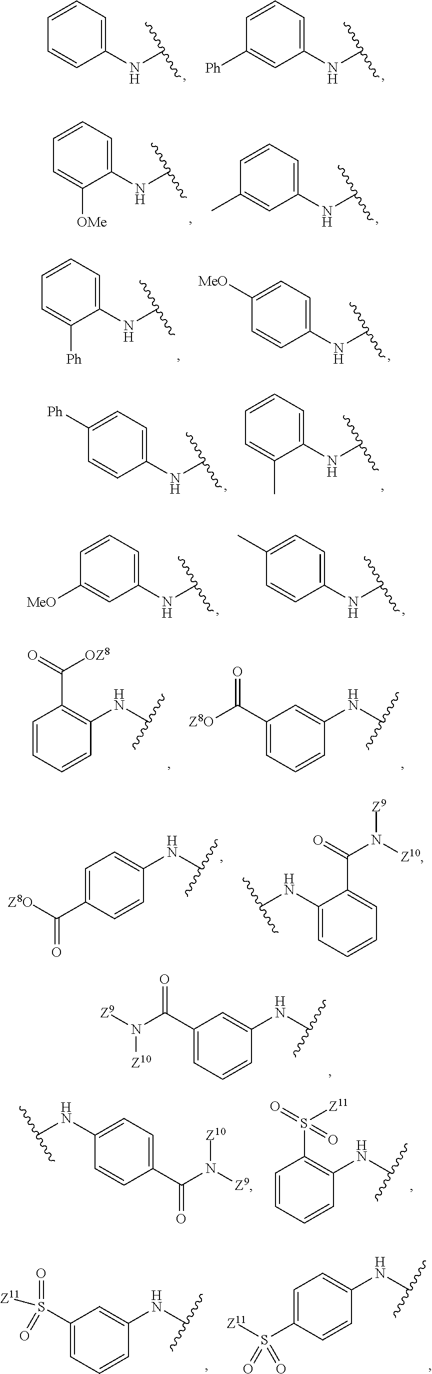

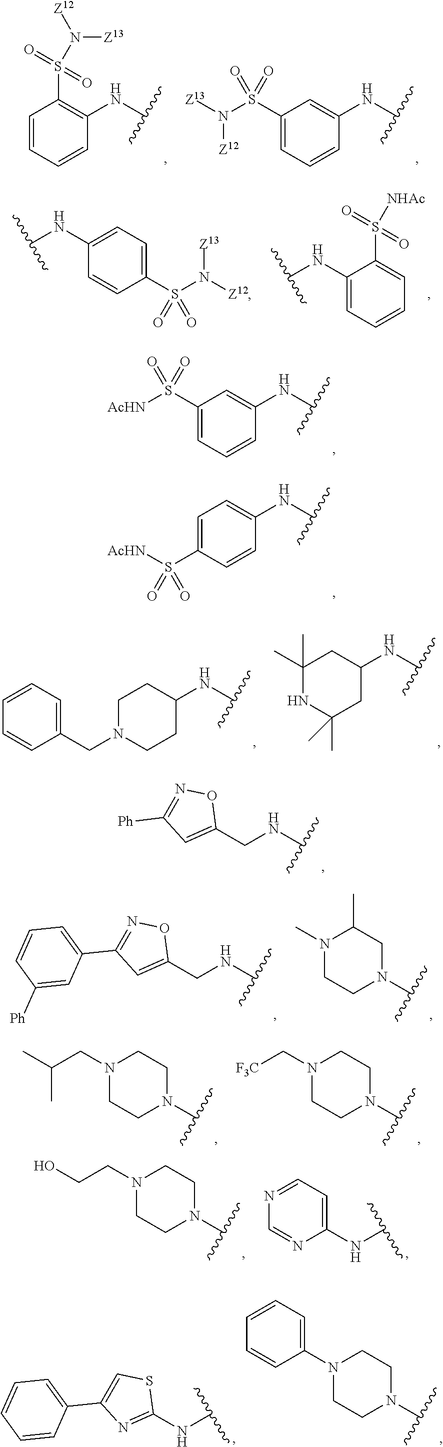

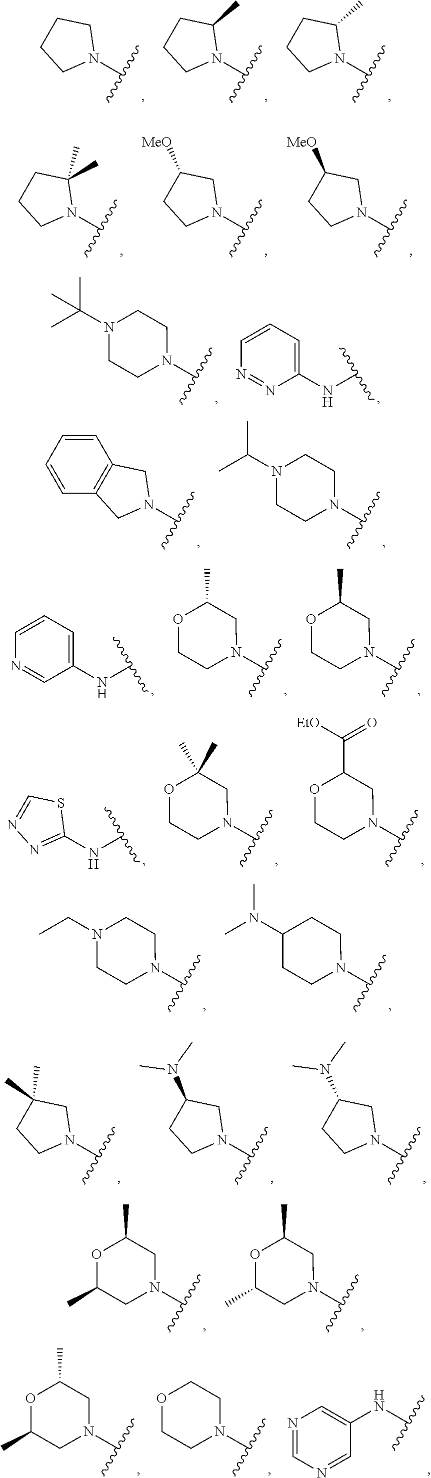

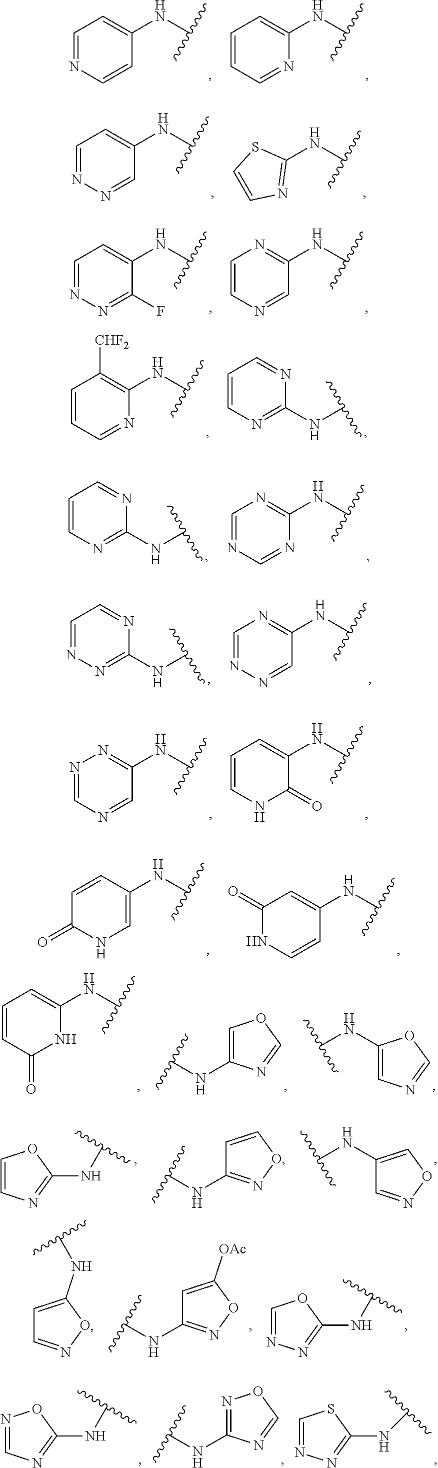

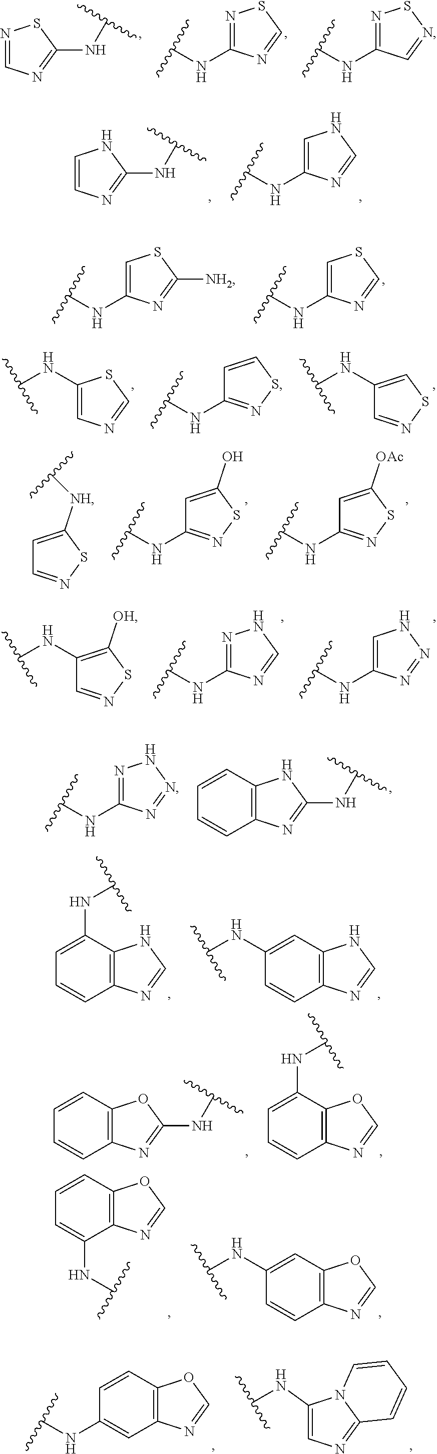

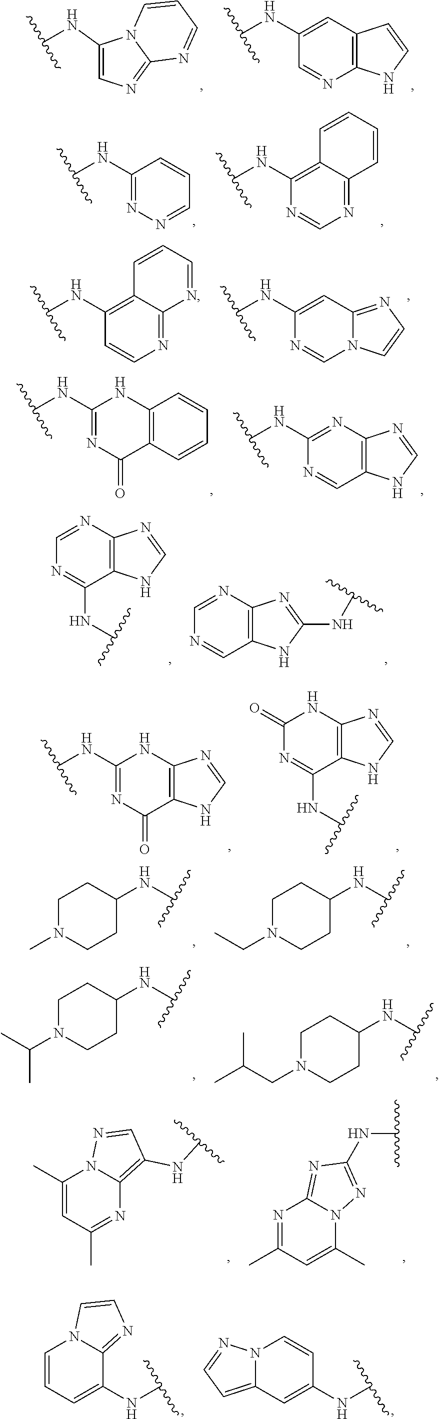

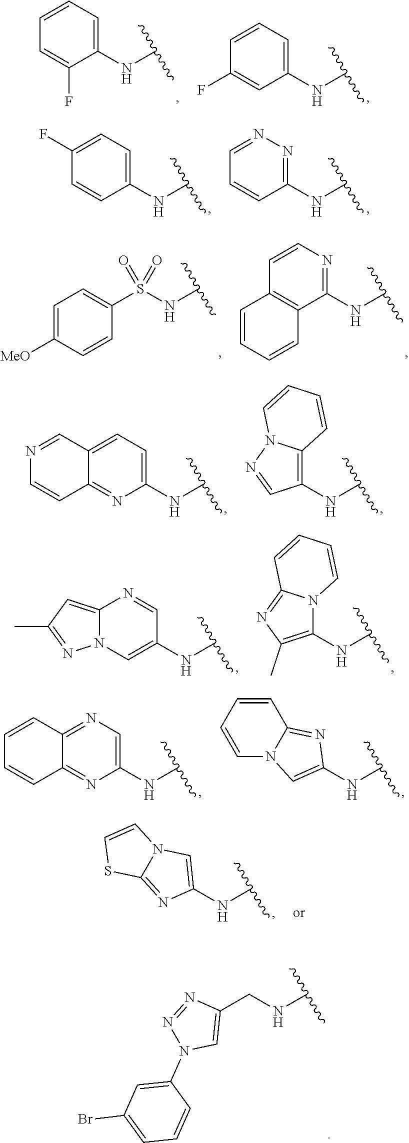

In some embodiments, the compound of Formula I is

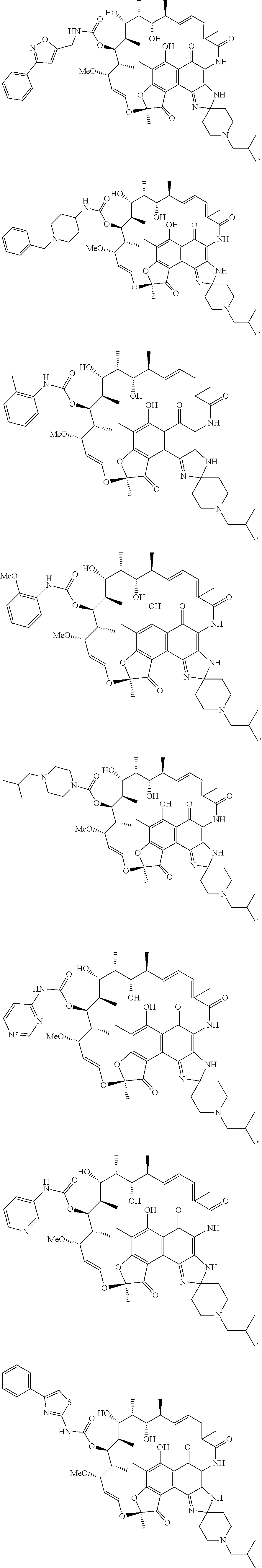

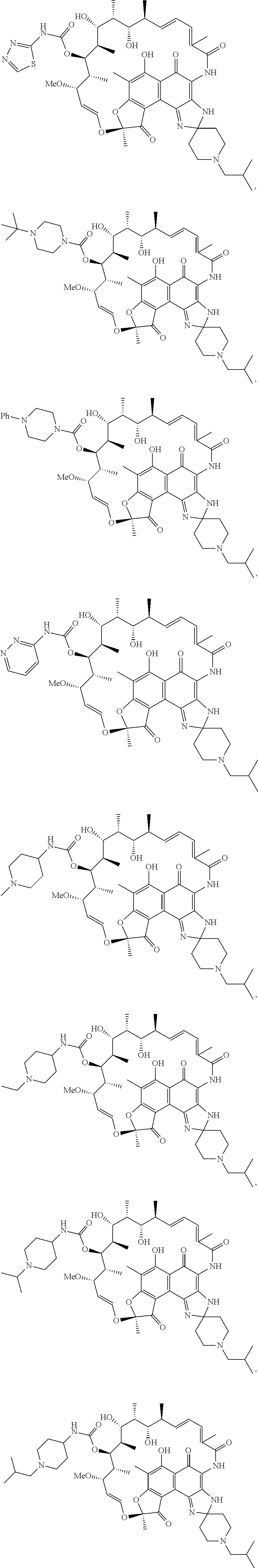

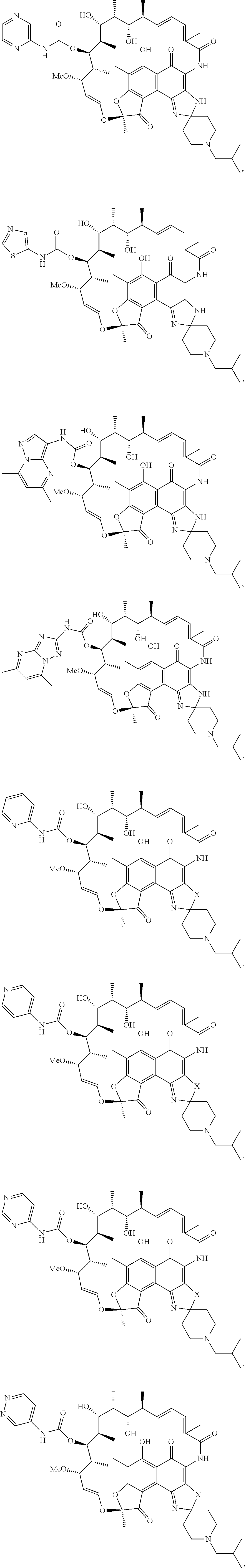

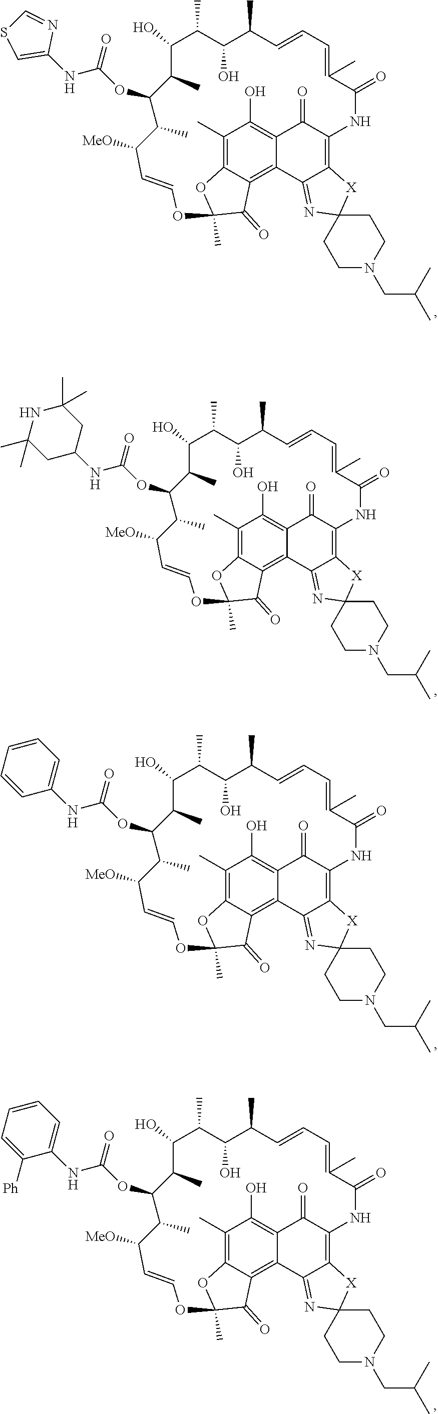

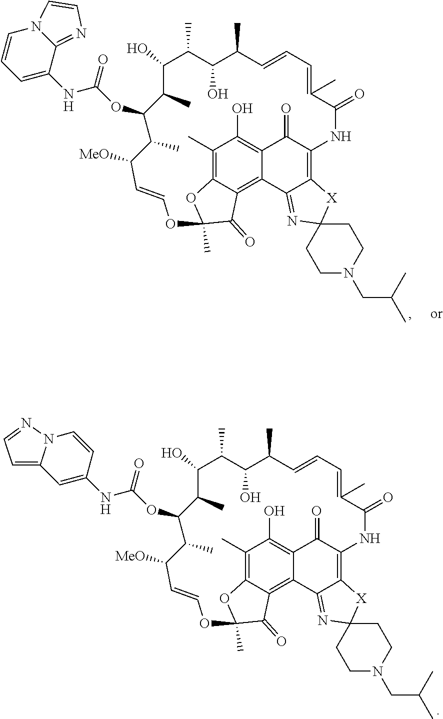

In some embodiments, R¹ in the compound of Formula I is NHS(O)₂R⁶; and R⁶ is heteroaryl or aryl optionally substituted with one or more halo, alkoxy, or haloalkyl.

In some such embodiments, the compound of Formula I is

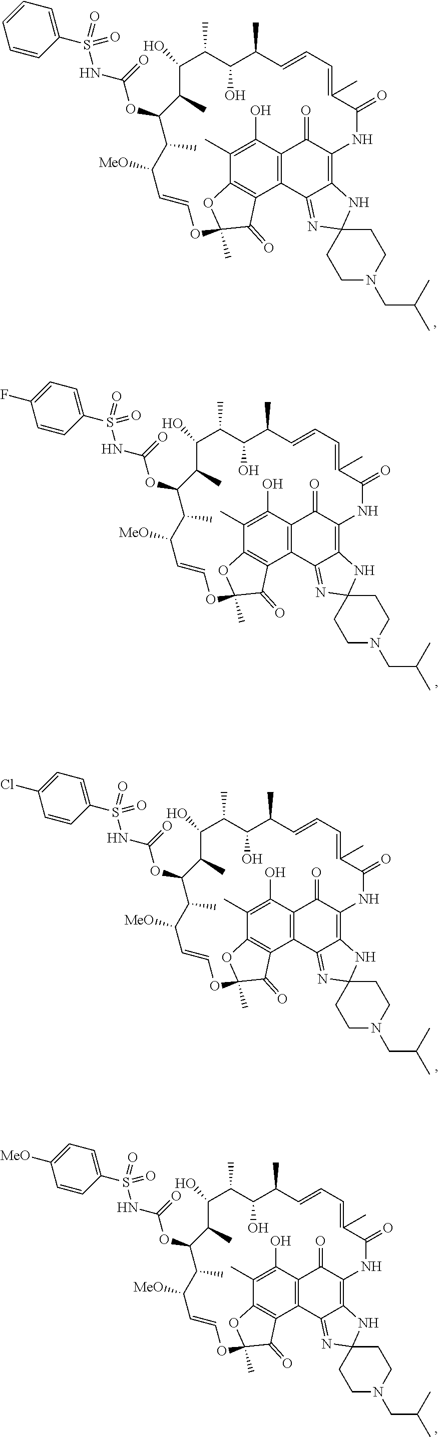

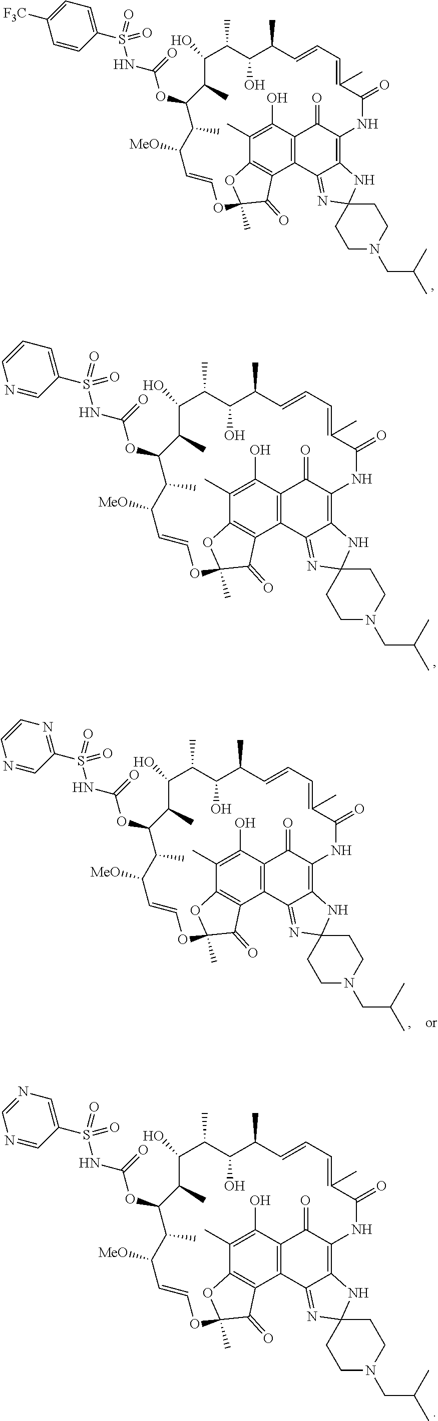

In some embodiments, formula (b) is

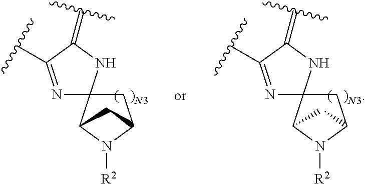

In some embodiments, formula (c) is or

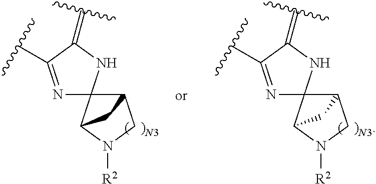

In some embodiments, R¹ in the compound of Formula I is an optionally substituted aryl or an optionally, substituted heteroaryl,

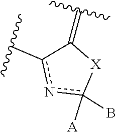

is formula (a) and N1 and N2 are 2,
Y¹ is N and
R² is selected from the group consisting of hydrogen an d alkyl optionally substituted with one or more hydroxyl, amino, carboxyl, or heteraryl.

In some such embodiments, R² is selected from the group consisting of

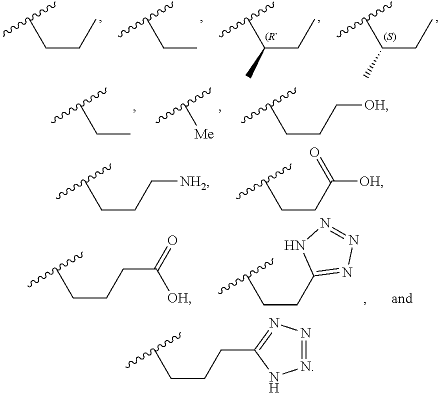

In some embodiments, R¹ in the compound of Formula I is an optionally substituted aryl or an optionally substituted heteroaryl, N1 and N2 are independently 1 or 3,N3 is 1, Y¹ is N, and R² is alkyl.

In some such embodiments,

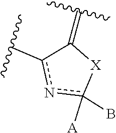

is selected from the group consisting of

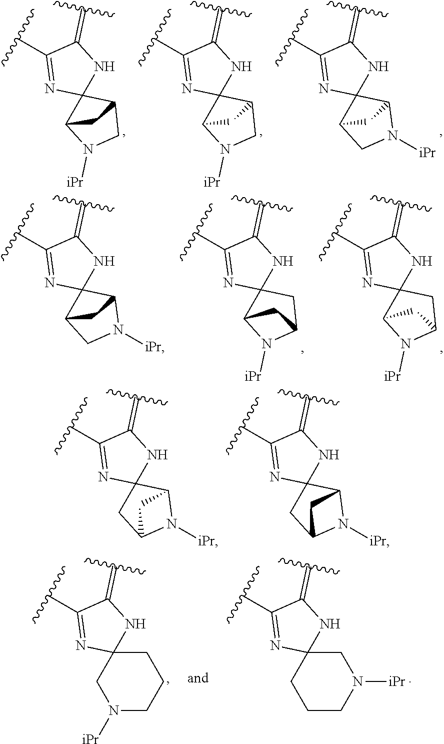

In some embodiments, R¹ in the compound of Formula I is selected from the group consisting of

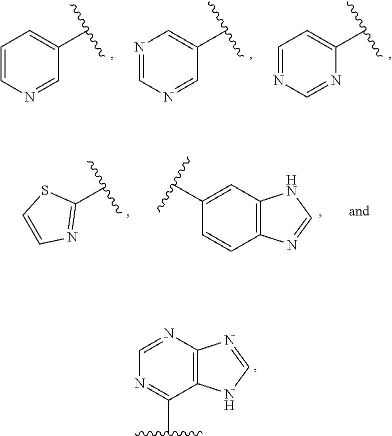 [SMILES: *c1cccnc1 ; *c1cncnc1 ; *c1ccncn1 ; *c1nccs1 ; *c1ccc2nc[nH]c2c1 ; *c1ncnc2nc[nH]c12]

and

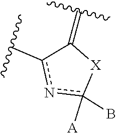

is selected from the group consisting of

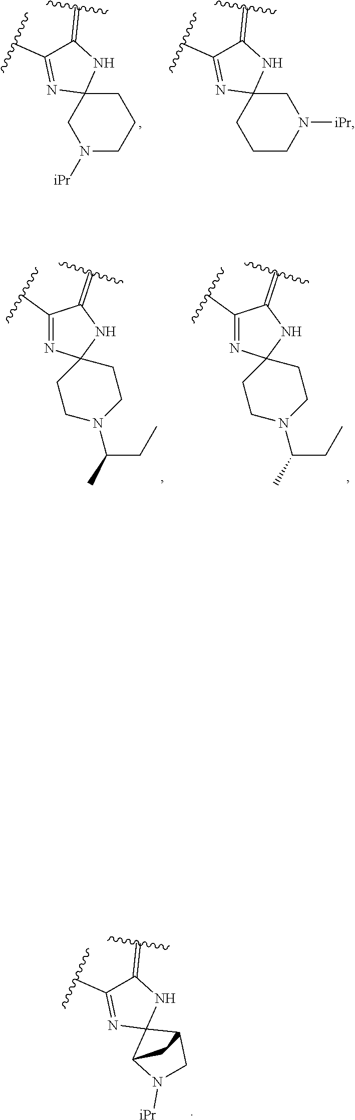

In some embodiments, the compound of Formula I is,

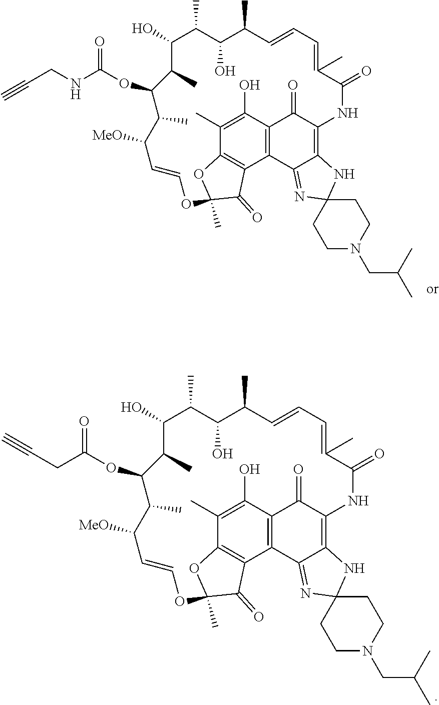

In some embodiments, the compound of Formula I is antibacterial. The term “antibacterial” refers to the property of a compound to prevent the growth or spread of bacteria. The antibacterial property of the compound as disclosed herein may be evaluated by its minimum inhibitory concentration that results in 90% growth inhibition (MIC) of a nontuberculous Mycobacterium, including, but are not limited to, Mycobacterium abscessus, Mycobacterium simiae, Mycobacterium chelonae, Mycobacterium fortuitum, Mycobacterium avium complex, Mycobacterium avium subspecies hominisuis, Mycobacterium intracellulare, Mycobacterium chimaera, Mycobacterium kansasii, Mycobacterium szulgai, Mycobacterium xenopi, and combinations thereof. In some embodiments, the Mycobacterium abscessus is selected from the group consisting of Mycobacterium abscessus subspecies abscessus. Mycobacterium abscessus subspecies bolletii, Mycobacterium abscessus subspecies massiliense, and combinations thereof.

In some embodiments, the compound as disclosed herein has an MIC less than 1200 nM. In some embodiments, the compound as disclosed herein has an MIC of from 1 nM to 1150 nM, from 1 nM to 1100 nM, from 1 nM to 600 nM, from 1 nM to 300 nM, from 1 nM to 200 nM, from 1 nM to 100 nM, from 1 nM to 75 nM, from 1 nM to 70 nM, from 1 nM to 65 nM, from 1 nM to 60 nM, from 1 nM to 55 nM, from 1 nM to 50 nM, from 1 nM to 45 nM, from 1 nM to 40 nM, from 1 nM to 35 nM, from 1 nM to 30 nM, or from 1 nM to 25 nM.

In some embodiments, the compound of Formula I has less inductive effect on a cytochrome P450 (CYP) enzyme than rifampicin, rifabutin, or rifapentine. Cytochrome P450 is a family of isozymes responsible for the biotransformation of several drugs. Cytochrome P450 represents a family of isozymes responsible for biotransformation of many drugs via oxidation. Drug metabolism via the cytochrome P450 system has emerged as an important determinant in the occurrence of several drug interactions that can result in drug toxicities, reduced pharmacological effect, and adverse drug reactions.

Inducers of CYP enzymes increase the amount of the CYP enzyme, increasing the rate of metabolism of a CYP substrate. The increased metabolism can effect an patient's response to a particular medication, for example, making it ineffective. Inducers of a CYP enzyme may be characterized as a strong inducer, i.e., a compound that causes ≥80% reduction in substrate exposure area under the curve (AUC), moderate inducer, i.e., a compound that causes ≥50% to <80% reduction in substrate exposure area under the curve (AUC), or weak inducer, i.e., a compound that causes <50% reduction in substrate exposure area under the curve (AUC). Rifampicin, rifabutin, and rifapentine are examples of CYP enzyme inducers that decrease plasma concentrations of coadministered CYP substrates. Rifampicin is a strong inducer of CYP3A4, CYP3A5, and CYP2C8 and moderate inducer of CYP1A2, CYP2B6, CYP2C9, and CYP2C19. Rifabutin is a moderate enhancer of CYP 3A4 or CYP3A5. Rifapentine is a strong inducer of CYP3A4.

In some embodiments, the compound as disclosed herein has CYP3A4 gene fold induction of less than 20 as determined by the assay described in Example 9. Suitably, the compound has a CYP3A4 gene fold induction of 0.5 and 20, 0.5 and 10, 0.5 and 5, 0.5 and 4, 0.5 and 3, 0.5 and 2, or 0.5 and 1.

Another aspect of the technology is a pharmaceutical composition comprising a therapeutically effective amount of the compound as disclosed herein, or a pharmaceutically acceptable salt thereof, and a pharmaceutically acceptable carrier, excipient, or diluent.

The phrase “therapeutically effective amount” refers to a dosage that provides the specific pharmacological response for which the compound is administered in a significant number of subject in need of such treatment. An effective amount of a drug that is administered to a particular subject in a particular instance will not always be effective in treating the conditions/diseases described herein, even though such dosage is deemed to be a therapeutically effective amount by those of skill in the art.

The phrase “pharmaceutically acceptable carrier, excipient, or diluent” refers to a carrier, an excipient, or a diluent that is useful in preparing a generally non-toxic pharmaceutical composition that is neither biologically nor otherwise undesirable. The pharmaceutical composition as disclosed herein may include a carrier, an excipient, or a diluent that is acceptable for veterinary use as well as human pharmaceutical use.

Examples of a “pharmaceutically acceptable carrier” may include proteins, carbohydrates, sugar, talc, magnesium stearate, cellulose, calcium carbonate, and/or starch-gelatin paste.

Examples of a “pharmaceutically acceptable excipient” include binding agents, filling agents, lubricating agents, suspending agents, sweeteners, flavoring agents, preservatives, buffers, wetting agents, disintegrants, and effervescent agents. Filling agents may include lactose monohydrate, lactose anhydrous, and various starches. Examples of binding agents include various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™). Suitable lubricants, including agents that act on the flowability of the powder to be compressed, may include colloidal silicon dioxide, such as Aerosil®200, talc, stearic acid, magnesium stearate, calcium stearate, and silica gel. Examples of sweeteners may include any natural or artificial sweetener, such as sucrose, xylitol, sodium saccharin, cyclamate, aspartame, and acsulfame. Examples of flavoring agents include Magnasweet® (trademark of MAFCO), bubble gum flavor, and fruit flavors, and the like. Examples of preservatives may include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride. Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present. Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of a “pharmaceutically acceptable diluent” may include pharmaceutically acceptable inert fillers, such as microcrystalline cellulose, lactose, dibasic calcium phosphate, saccharides, and mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Another aspect of the technology is a method for treating or preventing an infection caused by a Mycobacterium in a subject in need thereof. The method comprises administering to the subject the compound as disclosed herein, or a pharmaceutically acceptable salt thereof, or the pharmaceutical composition as disclosed herein.

In some embodiments, the nontuberculous Mycobacterium is selected from the group consisting of Mycobacterium abscessus, Mycobacterium simiae, Mycobacterium chelonae, Mycobacterium fortuitum, Mycobacterium avium, Mycobacterium intracellulare, Mycobacterium chimaera, Mycobacterium kansasii, Mycobacterium szulgai, Mycobacterium xenopi, and combinations thereof. In some embodiments, the Mycobacterium is Mycobacterium abscessus. In some embodiments, the Mycobacterium abscessus is selected from the group consisting of Mycobacterium abscessus subspecies abscessus, Mycobacterium abscessus subspecies bolletii, Mycobacterium abscessus subspecies massiliense, and combinations thereof. In some embodiments, the Mycobacterium is Mycobacterium avium. In some embodiments, the Mycobacteriumm avium may be Mycobacterium avium subspecies hominisuis.

In some embodiments, the compound as disclosed herein, or the pharmaceutically acceptable salt thereof, or the pharmaceutical composition as disclosed herein is administered orally or intravenously.

As used herein, the terms “treat,” “treating,” and “treatment” refer to eliminating, reducing, or ameliorating an infection, a disease, or a disorder, and/or symptoms associated therewith. Although not precluded, treating an infection, a disease, or a disorder does not require that the infection, disease, disorder, or symptoms associated therewith be completely eliminated.

As used herein, the term “prevent” or “preventing” refers to reducing the probability of developing or redeveloping an infection, a disease, or a disorder, or of a recurrence of a previously-controlled disease or condition, in a subject who does not have, but is at risk of or is susceptible to, redeveloping an infection, a disease, or a disorder or a recurrence of the infection, disease, or disorder.

The term “subject” refers to an animal, such as a mammal (e.g. human), who has been the object of treatment, observation or experiment. In some embodiments, the subject has an infection with any of the bacterium disclosed herein, such as a Mycobacterium. In some embodiments, the subject has an infection caused by a bacterium that is resistant to rifampicin, rifabutin, or rifapentine. In some embodiments, the subject has an infection caused by a bacterium that expresses an enzyme that catalyzes ADP-ribosylation of rifampicin, rifabutin, or rifapentine, such as a rifamycin ADP-ribosyltransferase. In some embodiments, the subject has an infection where the bacterium is within caseum in the subject. In some embodiments, the subject has caseous necrosis.

Another aspect of the technology is a method for treating or preventing an infection caused by a bacterium in a subject in need thereof. The method comprises administering to the subject the compound as disclosed herein, or a pharmaceutically acceptable salt thereof, or the pharmaceutical composition as disclosed herein. The disclosed compounds may be suitable for treating subjects for a bacterium that is resistant to rifampicin, rifabutin, or rifapentine. The rifamycin resistance in Al abscessus is caused by a group-transfer inactivation mechanism via a rifamycin ADP-ribosyltransferase (Arr). The disclosed compounds demonstrated the ability of overcome Arr-mediate resistance of bacteria that express a rifamycin ADP-ribosyltransferase. The bacterium may be, but is not limited to, a Mycobacterium, such as Mycobacterium abscessus, Mycobacterium simiae, Mycobacterium chelonae, Mycobacterium fortuitum, Mycobacterium avium complex, Mycobacterium avium subspecies hominisuis, Mycobacterium intracellulare, Mycobacterium chimaera, Mycobacterium kansasii, Mycobacterium szulgai, Mycobacterium xenopi, Mycobacterium obuense, Mycobacterium. phlei, Mycobacterium. gilvum, Mycobacterium. marinum, or Mycobacterium. scrofulaceun.

The methods of treatment disclosed herein may also be useful for treating or preventing infection by a bacterium that is not a Mycobacterium. Examples of bacterium that express rifamycin ADP-ribosyltransferase include, without limitation, Gordonia bronchialis, Gordonia terrae, Tsukamurella paurometabolum, Pseudomonas aeruginosa, Klebsiella pneumoniae, Acinetobacter baumannii, Streptomyces coelicolor, Clostridium bolteae, Klebsiella oxytoca.

Another aspect of the technology is a method for treating or preventing an infection in a subject in need of an antibacterial compound, the method comprising administering the subject the compound of claim 1 or a pharmaceutically acceptable salt thereof. The compound administered to the subject may be selected to reduce the likelihood of a drug-drug interaction when compared to rifampicin, rifabutin, or rifapentine. Suitably, the antibacterial compound administered to the subject has less inductive effect on a cytochrome P450 enzyme, e.g., CYP3A4, than rifampicin, rifabutin, or rifapentine. In some cases, the subject is undergoing treatment with another therapeutic agent or compound metabolized by the cytochrome P450 enzyme or sensitive to the co-administration of an inducer of the cytochrome P450 enzyme.

The compounds of Formula I, as described herein, may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” or “+” or “−” depending on the configuration of substituents around the stereogenic carbon atom and or the optical rotation observed. The compounds of Formula I may encompass various stereoisomers and mixtures thereof. Stereoisomers include enantiomers and diastereomers. Mixtures of enantiomers or diastereomers may be designated (±)″ in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise. Also contemplated herein are compositions comprising, consisting essentially of, or consisting of an enantiopure compound, which composition may comprise, consist essential of, or consist of at least about 50%, 60%, 70%, 80%, 90%, 95%, 96%, 97%, 98%, 99%, or 100% of a single enantiomer of a given compound (e.g., at least about 99% of an R enantiomer of a given compound).

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

Rifamycin antibiotics are a valuable class of antimicrobials for treating infections by mycobacteria and other persistent bacteria owing to their potent bactericidal activity against replicating and non-replicating pathogens. However, the clinical utility of rifamycins against Mycobacterium abscessus is seriously compromised by a novel resistance mechanism, namely, rifamycin inactivation by ADP-ribosylation. Using a structure-based approach, we rationally redesign rifamycins through strategic modification of the ansa-chain to block ADP-ribosylation while preserving on-target activity. Validated by a combination of biochemical, structural, and microbiological studies, the most potent analogs overcome ADP-ribosylation, restored their intrinsic low nanomolar activity and demonstrated significant in vivo antibacterial efficacy. Further optimization by tuning drug disposition properties afforded a preclinical candidate with remarkable potency and an outstanding pharmacokinetic profile. The disclosed compounds demonstrate a lower inductive effect on cytochrome p450 enzymes than rifampicin, rifabutin, or rifapentine.

### Example 1

Antimicrobial resistance (AMR) is a significant and ever-increasing burden to public health with roughly 2-5 million annual deaths attributed to AMR worldwide. Among the resistance mechanisms adopted by microorganisms, enzymatic inactivation of antibiotics is among the earliest identified, tracing back to 1940 when the first β-lactamase was reported to “destroy penicillin”. While microorganisms can degrade antibiotics by hydrolysis or redox transformation, modification of antibiotics via group transfer represents the most chemically diverse drug inactivation mechanism. Many antibiotics including aminoglycosides, macrolides, and lincosamides are prone to group-transfer inactivation via transformations such as acylation, phosphorylation, and glycosylation. Within this family of antibiotic-modifying chemistry, ADP-ribosylation of the rifamycins represents a particularly novel and noteworthy member.

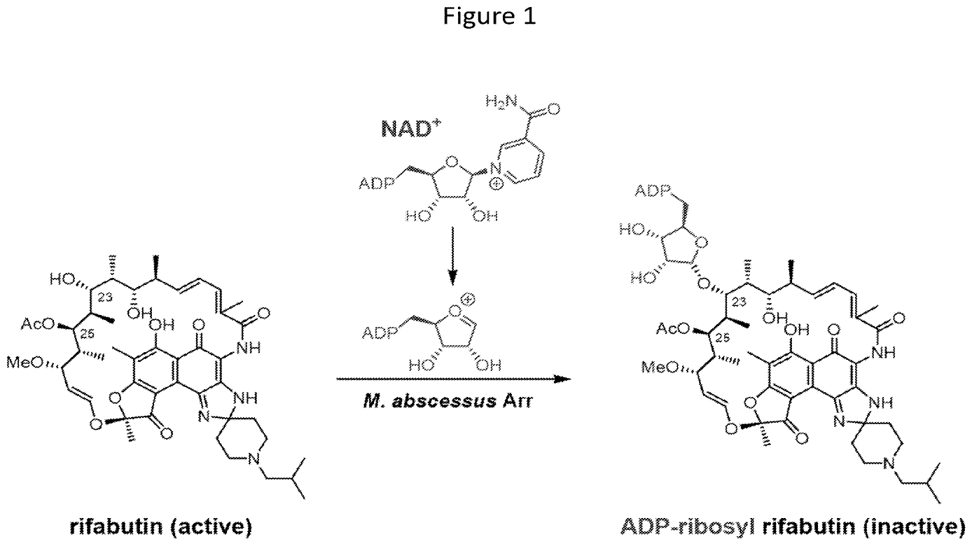

The natural product-derived rifamycin antibiotics function as inhibitors of bacterial transcription by allosteric binding to bacterial DNA-dependent RNA polymerases (RNAPs) preventing elongation of the nascent RNA and exhibit exceptional sterilizing activity against Mycobacterium tuberculosis and many other pathogens. With nanomolar antibacterial activity, rifamycins are specifically known for their clinical value in the treatment of persistent bacteria and mycobacteria infections due to their remarkable potency against bacterial persisters, often in biofilms and under dormant states, that are high tolerant to most antibiotics. However, rifamycin drugs are clinically ineffective against Mycobacterium abscessus, an emerging nontuberculous mycobacteria that causes an often fatal pulmonary infection with no reliable treatment options since M. abscessus is intrinsically drug resistant to virtually all antibacterial classes. The rifamycin resistance in M. abscessus is caused by a group-transfer inactivation mechanism via a rifamycin ADP-ribosyltransferase (Arr). With NAD⁺ as the donor, Arr catalyzes the formation of the ADP-ribosyl-oxocarbenium intermediate and transfers this intermediate regioselectively to C23-OH on the rifamycin polyketide ansa-chain. This, in turn, prevents binding to the bacterial RNAP and significantly reduces the rifamycin potency (FIG. 1). As a consequence of this, for example, the semi-synthetic rifamycin drug rifabutin, which exhibits low nanomolar activity against M. tuberculosis and many gram-positive pathogens, displays only modest micromolar activity against M. abscessus. Arr in M. abscessus and some other bacteria are so far the only known ADP-ribosyltransferases that target small molecules, while most bacterial ADP-ribosyltransferases are protein toxins that function through post-translational ADP-ribosylation of host proteins and are key virulence factors in Corynebacterium diphtheriae, Vibrio cholerae, Bordetalla pertussis, and Clostridrium botulinum. Therefore, rifamycin ADP-ribosylation represents an unprecedented and novel mechanism of antimicrobial resistance.

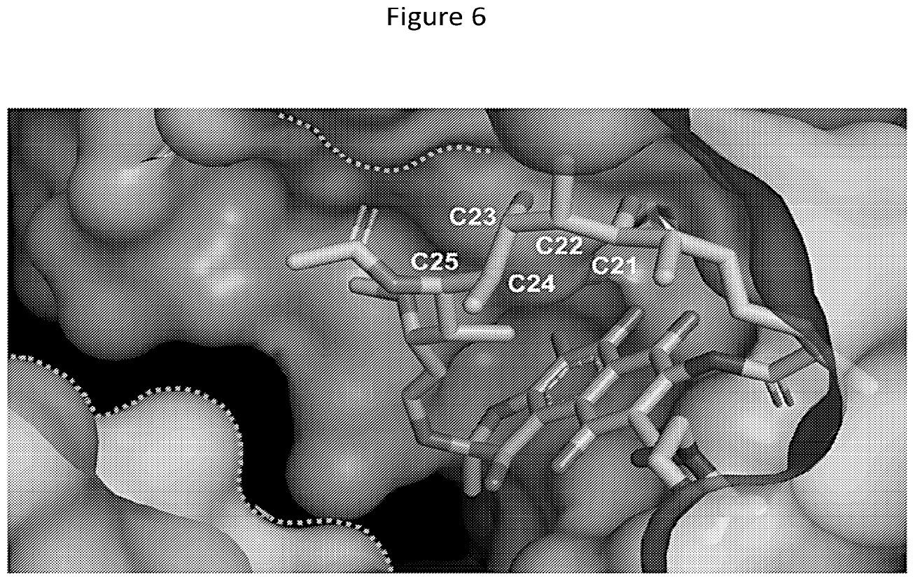

To determine possible modification sites on rifamycins, we investigated the rifamycin binding mode in mycobacterial RNAP using the 3D structure of M. tuberculosis RNAP (RNAP_{Mtb}) in complex with rifampicin (PDB: 5UHB, FIG. 6), which shares a 97% sequence identity to the M. abscessus RNAP (RNAP_{Mab}) rifamycin binding site. Around the inactivation position C23-OH, C21-OH and C22-CH₃ are closely enveloped by the binding pocket while C24-CH₃ and C25-OAc face larger spaces in the pocket.

### Results and Discussion

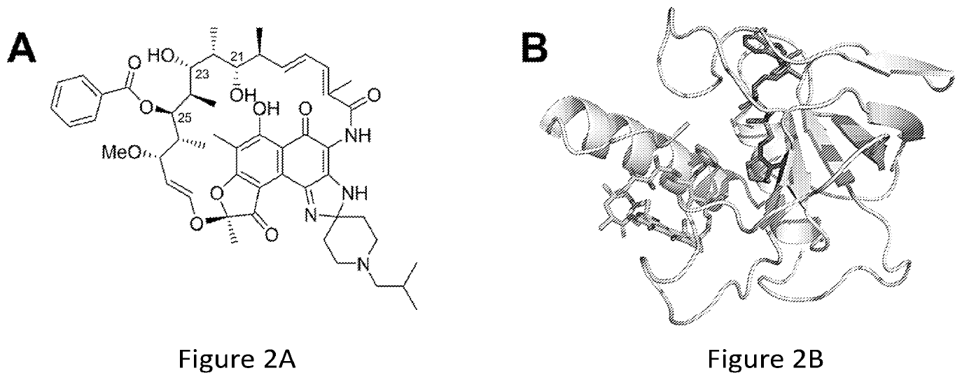

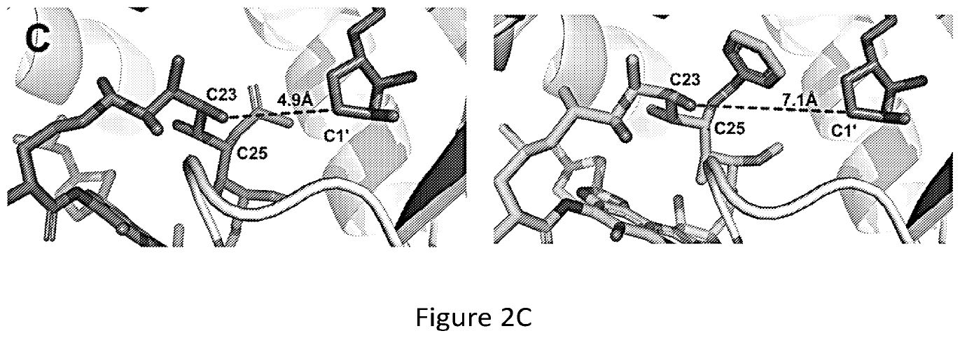

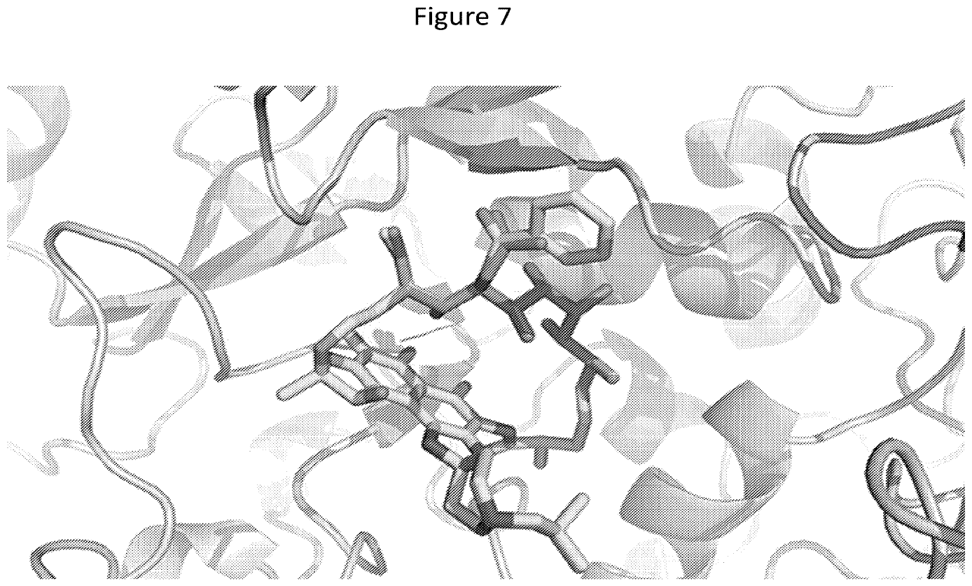

To explore whether C25 modification affects the binding of rifamycins against RNAP_{Mab}, molecular docking studies were conducted on a homology model of RNAP_{Mab} that uses the rifamycin-bounded RNAP_{Mtb} structure 5UHB as the template. The binding of an archetypical ligand 25-O-benzoyl rifabutin (5a, FIG. 2A), in which a bulkier benzoyl moiety replaced the C25 acetyl group, was investigated and demonstrated a highly consistent binding mode compared to unmodified rifabutin in the conserved rifamycin binding site (FIG. 7).

To determine whether C25 modification on rifamycins can disturb the interaction with M. abscessus Arr (Arr_{Mab}), a homology model of Arr_{Mab} was generated based on the Arr of M. smegmatis (64% sequence identity, PDB: 2HW2). The ADP-ribosyl-oxocarbenium intermediate was positioned in this model and the complex was further optimized using molecular dynamics simulations (FIG. 2B). A molecular docking analysis of 5a and rifabutin using this Arr_{Mab} model suggested that in contrast to rifabutin, 5a formed a catalytically incompetent complex due to the increase in the distance between C23-OH and NAD⁺-C1′ from 4.9 Å (rifabutin) to 7.1 Å (5a) (FIG. 2C). With these results, we hypothesized that rifamycin analogs with modifications on the C25 position could be well accommodated by RNAP but were unable to undergo ADP-ribosylation by Arr, thereby restoring their high potency against M. abscessus.

A series of C-25 substituted rifamycin analogs were synthesized containing a range of sterically variable alkyl, aryl, and heteroaryl esters (Scheme 1). Rifabutin was selected as the template since it is the most potent rifamycin against M. abscessus, has the lowest P450 induction potential and most favorable pharmacokinetic profile among clinically approved rifamycin antibiotics. Prior to initiating modifications to C25, the C21,23-diol was protected rendering an acetonide-containing rifabutin 2. Carefully controlled methanolysis of 2 afforded the deacetylated intermediate 3 using potassium carbonate to minimize competitive lactam opening due to the electron-withdrawing naphthoquinone core, which enhances the reactivity of the amide linkage. Acylation of the newly liberated C25-OH proved to be extremely challenging. The steric hindrance from the neighboring acetonide group prevented acylation with less-reactive reagents: under stronger acylation conditions, on the other hand, the nucleophilic spiroimidazopiperidine N-3 amine also reacted and lowered the regioselectivity. After extensive experimentation (Table 3), we found that the regioselective esterification on C25-OH could only be achieved using a large excess of acid anhydride or mixed anhydride, formed in situ from acids and pivaloyl chloride. The acetonide group was successfully cleaved using CSA in methanol to yield final rifabutin analogs. For the most sterically demanding substrate, the less hindered 25-O-desacetyl rifabutin 6^{[} was directly acylated using acid anhydrides to generate desired products after careful separation. The structures of all the final products are verified by 1D and 2D NMR. ¹H-¹H NOESY spectra were acquired for analogs 5e, 5g, and 5i (see Table 1) as representatives to ensure the configurations of the chiral centers, especially C25, are maintained throughout the synthesis.

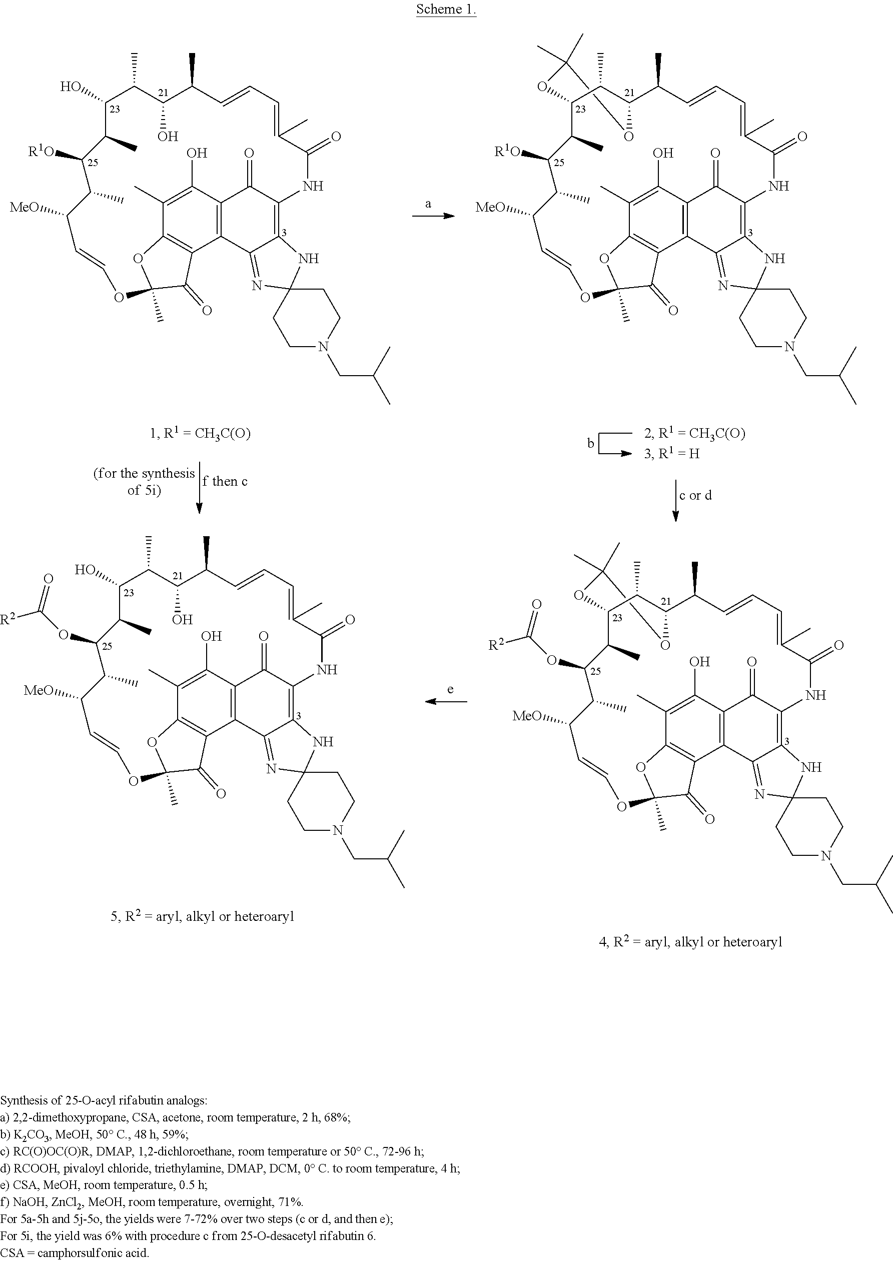

TABLE 3
Trials of C-25 esterification methods. Benzoic acid was used as the model
acid in all the entries except Entry 5, where benzoyl fluoride was used.

| Entry | condition | result |
|---|---|---|
| 1 | 2,4,6-trichlorobenzoyl chloride, acid, | starting material decomposed into |
| | Et₃N, DMAP, rt | inseparable byproducts, no desired product |
| | | detected |
| 2 | acid, CDI, rt | no conversion of the starting material |
| 3 | acid, Mukaiyama reagent | no conversion at rt; starting material |
| | | decomposition at 70° C. |
| 4 | acid, TsCl, N-methylimidazole | no conversion at rt; starting material |
| | | decomposition at 70° C. |
| 5 | acyl fluoride, Et₃N | no conversion of the starting material |
| 6 | acid, MSNT, N-methylimidazole | starting material decomposed into |
| | | inseparable byproducts, no desired product |
| | | detected |
| 7 | acid, EDC, DMAP, rt | C3—NH was first acylated. C25—OH, C3—NH- |
| | | diacylated product was generated upon |
| | | extended reaction time and isolated at 45% |
| | | yield. |

To reveal the impact of C-25 modifications on the potency of the synthetic rifabutin analogs, the minimum inhibitory concentration that results in 90% growth inhibition (MIC) of these analogs against M. abscessus were determined (Table 1). A wild-type (WT) M. abscessus ATCC 19977 and an isogenic arr-deleted (Δarr) M. abscessus strain were used in parallel to assess the resistance phenotype. Clarithromycin, rifampicin and rifabutin were included as controls. Compound 5a containing a 25-O-benzyol group had a MIC of 53 nM, which is 20 and 100 times lower than rifabutin and rifampicin, respectively. More importantly, 5a was equally potent against WT and Δarr M. abscessus strains, meaning that this compound was no longer inactivated by Arr. Compounds with ortho-, meta- and para-methyl benzoates (5b-5d) demonstrated that ortho-substitution was preferred. Further investigation on different ortho-substituted C-25 benzoates clearly delineated the impact of steric bulkiness on the activity: small groups (F, Cl and OMe 5e-5g) could be appended without compromising the activity, while bulkier groups (CF₃ and Ph 5h and 5i) caused 2- and 5-fold loss of potency, respectively, compared to 5a. Analog 5j with the small F on the meta position of C25-benzoate also retained the activity. On the other hand, analogs 5k and 5l with small alkyl groups display only modest micromolar MIC values against wild-type M. abscessus. Potent activity is only achieved against the isogenic Δarr deletion strain indicating 5k and 5l are inactivated by Arr. Furthermore, compounds 5a, 5b, and 5j were shown to maintain potent activity against a panel of drug-resistant M. abscessus clinical isolates (Table 4). Altogether, these results demonstrate C-25 modification of rifabutin as a viable strategy to significantly increase the potency and effectively block rifamycin inactivation by ADP-ribosylation.

TABLE 1
Activity of the synthetic rifabutin analogs against
WT or Δarr M. abscessus^{[a]}

| | R² (see | WT^{[b]} Mab | Δarr Mab | MIC |
|---|---|---|---|---|
| compound | Scheme 1) | MIC | MIC | ratio^{[c]} |
| clarithromycin | | 1450 | 1800 | 0.7 |
| rifampicin | — | 5300 | 260 | 20.4 |
| rifabutin | Me | 1150 | 31 | 39.3 |
| 5a | C₆H₅— | 53 | 60 | 0.9 |
| 5b | o-MeC₈H₄— | 43 | 64 | 0.7 |
| 5c | m-MeC₆H₄— | 75 | 65 | 1.2 |
| 5d | p-MeC₈H₄— | 200 | 110 | 1.8 |
| 5e | o-FC₆H₄— | 60 | 58 | 1.0 |
| 5f | o-ClC₆H₄— | 43 | 49 | 0.9 |
| 5g | o-OMeC₆H₄— | 30 | 30 | 1.0 |
| 5h | o-CF₃C₆H₄— | 100 | 120 | 0.8 |
| 5i | o-PhC₈H₄— | 260 | 140 | 1.8 |
| 5j | m-FC₈H₄— | 55 | 90 | 0.6 |
| 5k | 2-Me-butyryl | 1100 | 28 | 39.3 |
| 5l | 2-Et-butyryl | 1150 | 49 | 23.5 |
| 5m | 3-Pyridyl | 24 | 26 | 0.9 |
| 5n | 5-Pyrimidyl | 18 | 25 | 0.72 |
| 5o | 2-Thiazolyl | 17 | 17 | 1.0 |

^{[a]}All MIC values are given in nM.
^{[b]}M. abscessus ATCC 19977
^{[c]}MIC ratio = WT MIC/Δarr strain MIC

TABLE 4
Activity of the selected rifabutin analogs against M. abscessus
clinical isolates. Tested synthetic rifabutin analogs showed
equal potency against the clinical isolates^{[a]}

| compound | Mab 19977 MIC | Mab Bamboo MIC | Mab K21 MIC |
|---|---|---|---|
| nifabutin | 1150 | 1100 | 2300 |
| 5a | 53 | 65 | 98 |
| 5b | 43 | 54 | 66 |
| 5j | 55 | 68 | 102 |
| 5m | 25 | 23 | 33 |

^{[a]}All MIC values were determined as the concentrations that result in 90% inhibition of bacterial growth. All MIC values are given in nM.

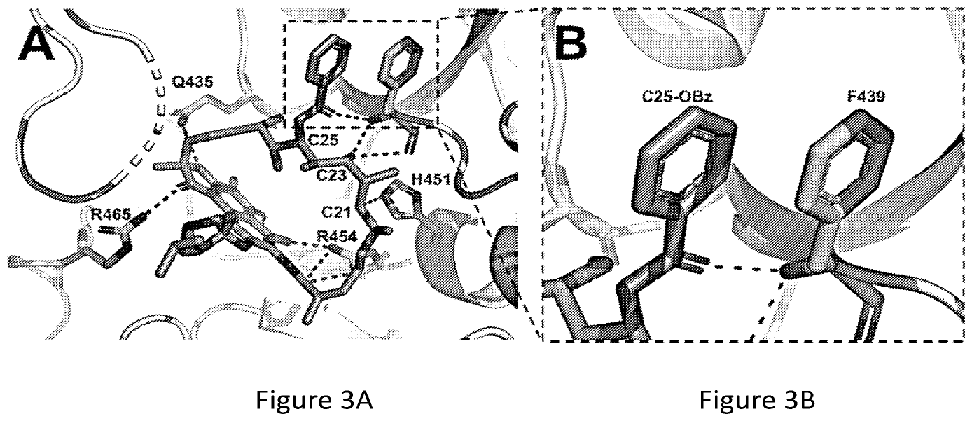

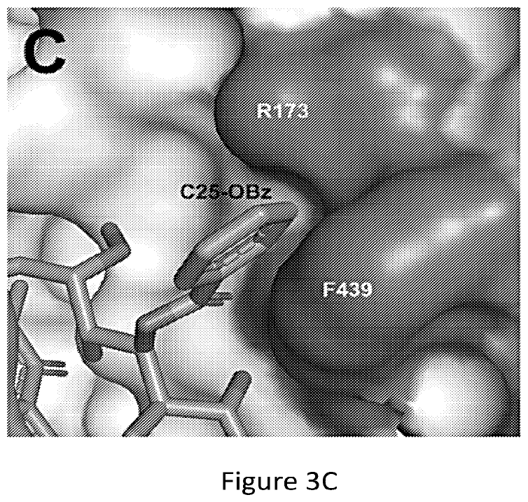

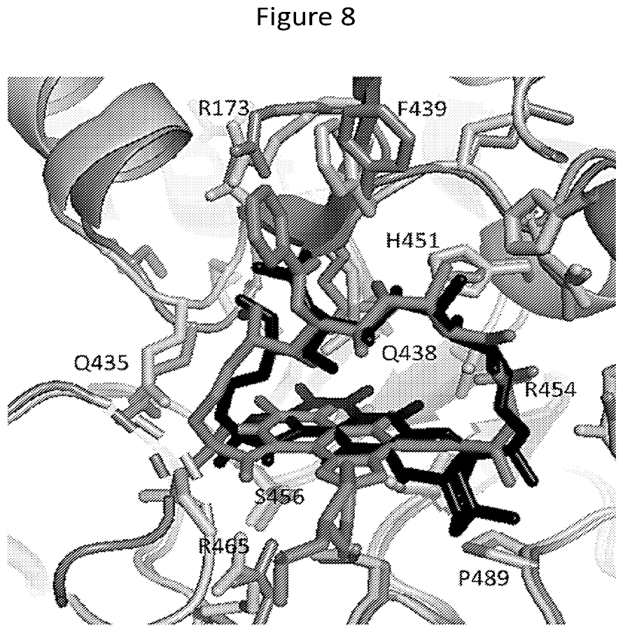

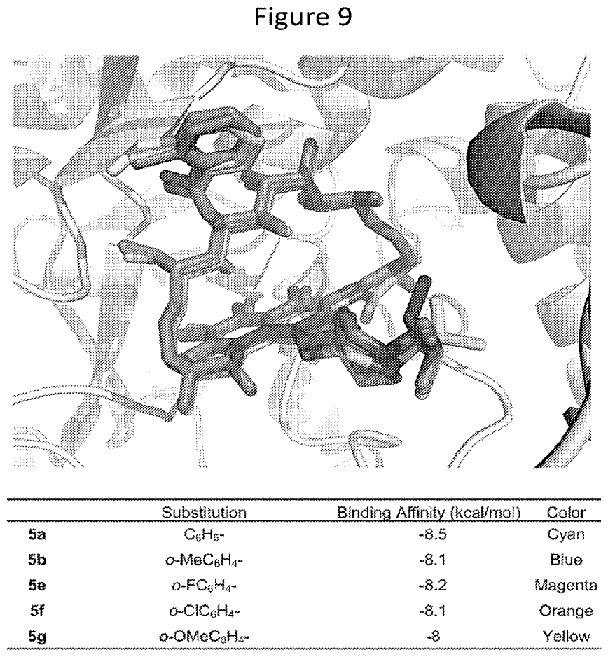

In the following activity screening using a rifamycin-resistant M. abscessus mutant RFB-R1 carrying an RNAP point mutation, analogs 5a, 5b, and 5j lost detectable activity (Table 5), suggesting their on-target activity through RNAP inhibition. To further understand how the C-25 modification on rifabutin affects binding to RNAP, the crystal structure of a representative compound 5a complexed with RNAP_{Mtb} was solved at a resolution of 3.9 Å (see Table 6 for the data-collection and refinement statistics). The three-dimensional structure reveals that 5a adopts a highly similar binding mode to rifampicin in the same binding site (FIG. 8) and preserves all the essential hydrogen bonds from C1-O, C8-OH, C21-OH and C23-OH (FIG. 3A). A noteworthy difference in the binding of 5a is that the C-25 benzoate forms a unique π-stacking interaction with Phe439, with the two phenyl rings demonstrating a “close parallel displaced” geometry (FIG. 3B). These results indicate that the molecular target RNAP can accommodate the bulky C-25 modification by forming novel ligand-target interactions. A small cleft formed by Phe439 and Arg173 was found to envelop the C-25 benzoate of 5a (FIG. 3C), which was not observed in the structure of the previous RNAP-rifampicin complex. This cleft may account for the lower potency of analogs with phenyl- and trifluoromethyl-substituted benzoates since these extremely large substituents cannot fit in this small cleft, thereby decreasing the overall binding affinity. Analogs 5b-5g were proposed to bind to RNAP with highly similar modes to 5a, evaluated by molecular docking studies using the RNAP_{Mab} homology model mentioned above (FIG. 9).

TABLE 5
Activity of the synthetic rifabutin analogs
against RFB-R1 M. abscessus^{[a]}

| compound | RFB-R1 M. abscessus MIC |
|---|---|
| clarithromycin | 2.1 |
| rifampicin | >100 |
| rifabutin | >100 |
| 5a | >100 |
| 5b | >100 |
| 5j | >100 |
| 5m | >100 |

^{[a]}All MIC values were determined as the concentrations that result in 90% inhibition of bacterial growth. All MIC values are given in μM.

TABLE 6
Data-collection and refinement statistics for crystal structure
of RNAP_{Mtb}(M. tuberculosis σ^{A} RPo)-5a complex.

| PDB code | 7U22 | |
|---|---|---|
| data collection | | |
| wavelength (Å) | 1.0000 | |
| space group | P2₁2₁2₁ | |
| cell dimensions | | |
| a.b.c (Å) | 149.482. 161.445. 196.603 | |
| α.β.γ (°) | 90. 90. 90 | |
| resolution (Å)^{a} | 48.30-3.87 | (3.97-3.87) |
| unique reflections | 44,566 | (4,248) |
| Rₘₑₐₛ | 0.328 | |
| Rₚᵢₘ | 0.106 | |
| I/σ(I) | 11.84 | (0.95) |
| completeness (%) | 98.96 | (96.0) |
| CC^{1/2} | (0.396) | |
| CC* | (0.753) | |
| redundance | 9.4 | (8.2) |
| refinement | | |
| resolution (Å) | 48.30-3.87 | (3.97-3.87) |
| number of atoms | 25,925 | |
| R_{work}/R_{free} | 0.24/0.2781 | (0.3301/0.3697) |
| total no. of reflections | 42,677 | (4,067) |
| reflections used for R_{free} | 1,991 | (187) |

| | |
|---|---|
| B factors (Å²) | |
| macromolecules | 190.45 |
| ligands | 137.27 |
| RMSDs | |
| bond lengths (Å) | 0.0066 |
| bond angles (°) | 1.125 |
| clashscore | 14.92 |
| Ramachandran plot, % | |
| favored | 94.21 |
| outliers | 0.59 |
| rotamer outliers, % | 0.63 |

^{a}Numbers in parentheses refer to highest-resolution shell.

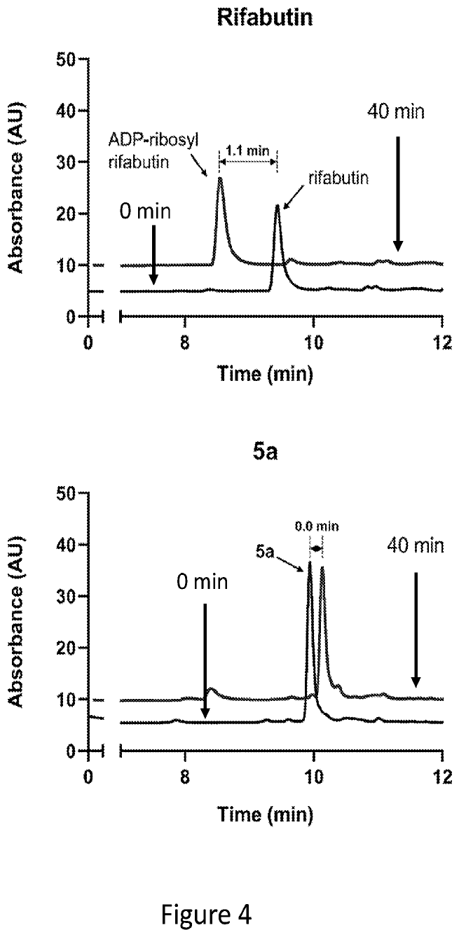

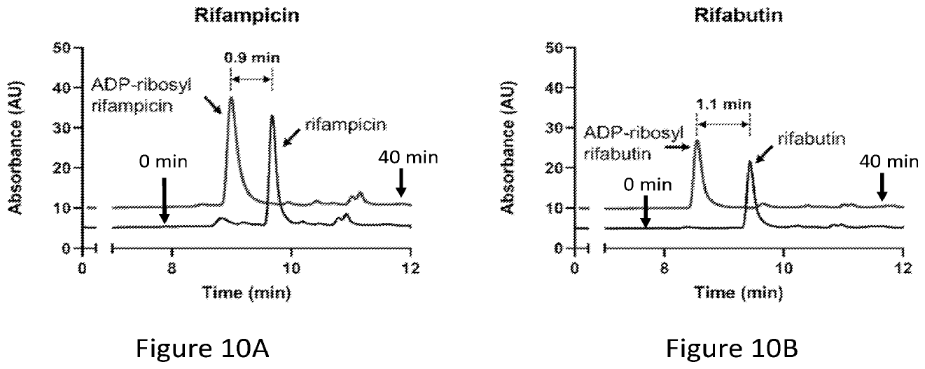

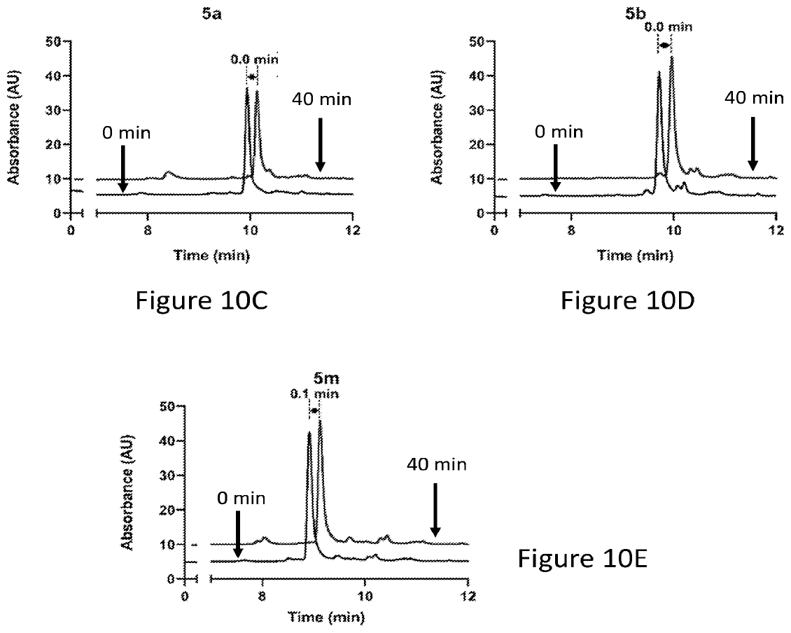

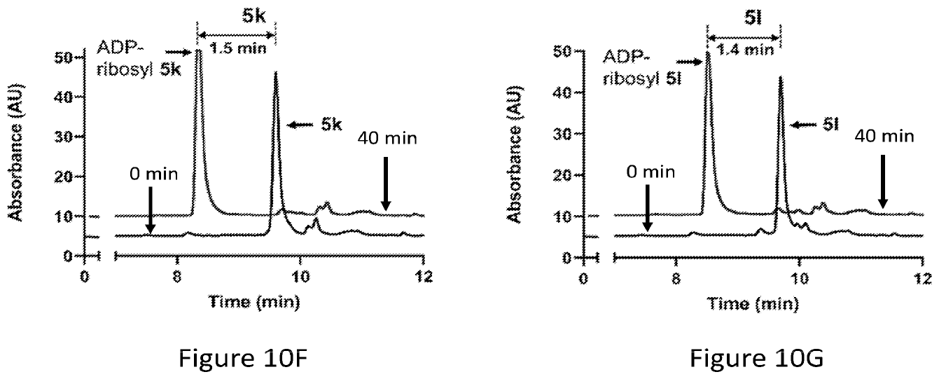

To biochemically validate the effect of C-25 modifications on ADP-ribosylation, we cloned, expressed, and purified recombinant Arr_{Mab} and developed a liquid chromatography mass spectrometry (LC-MS) enzymatic assay to quantify the rifamycins and ADP-ribosylated adducts. Upon incubation with Arr_{Mab} and NAD⁺, rifampicin and rifabutin were completely converted in 40 minutes (FIG. 4 and FIGS. 10A-10G) and the new peaks were confirmed as ADP-ribosyl adducts (m/z=694.90 for ADP-ribosyl rifabutin, see Table 7 for MS identifications for all the LC signals). By contrast, no transformation was observed for 5a and 5b which do not exhibit a loss of activity against WT M. abscessus with a functioning antibiotic-inactivating Arr (FIG. 4 and FIGS. 10A-10G). Meanwhile, the small-alkyl substituted analogs 5k and 5l, with considerable MIC shifts against WT and Δarr M. abscessus strains, were fully converted through ADP-ribosylation (FIGS. 10A-10G). These results further demonstrate that 25-O-benzoyl rifabutin compounds cannot be modified by Arr_{Mab}.

TABLE 7
MS validation of the peaks observed in LCMS-based in vitro validation
of ADP-ribosylation. For compounds converted to ADP-ribosyl
adducts upon incubation, the mass-to-charge ratio (m/z) of the expected
ADP-ribosyl adducts ([M + 2H]⁺⁺) and the observed
m/z of the post-incubation LC signals are labeled in red.

| | | expected ADP- | observed | observed |
|---|---|---|---|---|
| | MW | ribosyl | m/z | m/z |
| compound | (g/mol) | adduct m/z | (0 min) | (40 min) |
| rifampicin | 822.94 | 682.74 | 822.50 | 682.80 |
| rifabutin | 847.02 | 694.76 | 847.50 | 694.90 |
| 5a | 909.09 | 725.76 | 909.50 | 909.50 |
| 5b | 923.12 | 732.77 | 923.50 | 923.50 |
| 5k | 889.10 | 715.78 | 889.50 | 715.90 |
| 5l | 903.13 | 722.79 | 903.50 | 722.90 |
| 5m | 910.08 | 726.29 | 910.50 | 910.50 |

The promising in vitro activity of the rifabutin analogs led to the investigation of their in vivo PK properties. Candidates 5b and 5j were administered intravenously (i.v.) and orally (p.o.) to CD1 mice to assess their PK parameters and determine the optimal dosing regimen for future efficacy studies (Table 2). Both compounds exhibited an improved volume of distribution (V_{d}) and reduced clearance (CL) relative to rifabutin, resulting in a prolonged half-life (t_{1/2}) and greater in vivo drug exposure as measured by the area-under-the-curve (AUC) in the concentration-time curve. C25-desacetylation by esterases is a major metabolic pathway of rifamycin drugs. To assess whether candidates 5b and 5j were also prone to enzymatic ester hydrolysis, we evaluated the amount of 25-O-desacetyl rifabutin 6 in mouse plasma after intravenous and oral administration. Both 5b and 5j exhibited high metabolic stability to potential hydrolysis in either plasma or gut/liver, as less than 0.2% of 6 was detected for both candidates in both administration routes (Table 2). Hydrolysis of rifabutin C-25 acetate was not obvious either in this mouse model with only 0.4% of 6 detected in both administration routes.

TABLE 2
Important PK parameters of rifabutin and selected analogs^{[a]}

| compound | rifabutin | 5b | 5j | 5m |
|---|---|---|---|---|
| WT MIC (nM) | 1150 | 43 | 55 | 24 |
| V_{d} (L/kg) | 3.51 | 1.1 | 1.9 | 1.6 |
| t_{1/2} (h) | 4.0 | 10.3 | 11.7 | 6.8 |
| CL [mL/(kg × min)] | 10.3 | 1.5 | 2.7 | 3.0 |
| C-25 deacylation_{i.v.} (%) | 0.4 | n.d. | n.d. | 0.2 |
| C-25 deacylation_{p.o.} (%)^{[c]} | 0.4 | 0.2 | n.d. | 0.01 |
| PPB (% bound) | 96.24 | 99.99 | 99.98 | 97.95 |
| unbound fraction (%) | 3.76 | 0.01 | 0.02 | 2.05 |
| fAUC/MIC^{[a]} | 0.22 | 0.04 | 0.06 | 28.2 |

^{[a]}See Table S6 for the complete data and errors.
[b] fAUC/MIC was measured based on p.o. doses of 10 mg/kg
^{[c]}For both administrative routes, deacytation % = AUC₆/AUC_{compound} × 100%,
n.d.: undetectable.

TABLE 8
PK parameters of rifabutin and selected analogs with standard deviation.

| compound | rifabutin | | 5b | | 5j | | 5m | |
|---|---|---|---|---|---|---|---|---|
| WT MIC (nM) | 1150 | | 43 | | 55 | | 24 | |
| V_{d} (L/kg) (SD %^{[a]}) | 3.51 | (22.8) | 1.1 | (15.7) | 1.9 | (0.9) | 1.6 | (22.1) |
| t_{1/2} (h) | 4.0 | | 10.3 | | 11.7 | | 6.8 | |
| CL (mL/(kg × min)] (SD %) | 10.3 | (22.3) | 1.5 | (18.8) | 2.7 | (1.6) | 3.0 | (21.8) |
| C-25 deacylation_{i.v.} (%) | 0.4 | | n.d. | | n.d. | | 0.2 | |
| C-25 deacylation_{p.o.} (%)^{[c]} | 0.4 | | 0.2 | | n.d. | | 0.01 | |
| PPB (% bound) (SD %) | 96.24 | (0.3) | 99.99 | (0.0006) | 99.98 | (0.0017) | 97.95 | (0.15) |
| unbound fraction (%) (SD %) | 3.76 | (7.4) | 0.01 | (8.3) | 0.02 | (10.8) | 2.05 | (7.4) |

| | | | | |
|---|---|---|---|---|
| fAUC/MIC^{[b]} | 0.22 | 0.04 | 0.06 | 28.2 |

^{[a]}SD % = standard deviation/mean × 100%.
^{[b]}fAUC/MIC was measured based on p.o. doses of 10 mg/kg.
^{[c]}For both administrative routes, deacylation % = AUC₆/AUC_{compound} × 100%,
n.d.: undetectable.

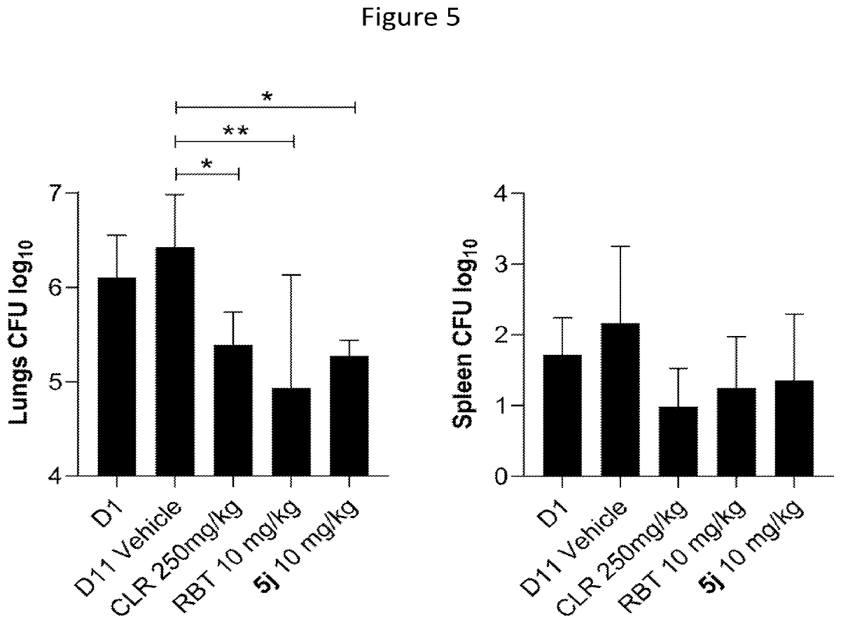

With the favorable PK profiles in hand, we next sought to characterize the in vivo efficacy of the candidate using an infected mouse model. Compound 5j, with the PK parameters validated, was selected as the candidate. 5j, clarithromycin, rifabutin, and vehicle were orally administered once daily to M. abscessus-infected mice for 10 consecutive days, and then the lung and spleen bacterial load was assessed. The efficacy of a drug was defined as a statistically significant reduction of colony-forming unit (CFU) in a study group relative to the vehicle control at the end of the experiment (FIG. 5). In this model, candidate 5j significantly reduced the bacterial load in lung by 10-fold and achieved in vivo bactericidal activity in a similar level to the positive control clarithromycin, which is a widely used anti-M. abscessus drug. The comparable efficacy of 5j and clarithromycin was also reflected in the spleen CFU reduction. However, 5j did not exhibit improved efficacy relative to rifabutin, as both compounds reduced lung and spleen CFU counts to a similar extent.

A further investigation of the pharmacokinetic/pharmacodynamic (PK/PD) profiles of the candidates led to plasma protein binding (PPB) as a likely limiting factor. Both 5b and 5j were highly protein bound as measured in a PPB assay and resulted in a significantly lower fraction of unbound drug in plasma than rifabutin (126- and 380-fold, Table 2). Given that the free concentration of antimycobacterial drugs correlates well with their in vivo efficacy, we selected the ratio of the area under the unbound drug concentration-time profile to MIC (fAUC/MIC) as the PK/PD index driving efficacy, and explored rifabutin analogs with higher fAUC/MIC in the following compound optimization.

In the characterization of the synthetic rifabutin analogs, it was discovered that switching C25-acetate to benzoates increased the lipophilicity of the molecule, which is reflected by a longer retention time on LC. We therefore suspected that the lipophilicity of the analogs contributed to their PPB. To further optimize the candidate aimed at reducing the lipophilicity of C-25 substituents while maintaining favorable potency, the phenyl group on 5a was replaced with bioisosteric heterocycles to generate analogs with 3-pyridyl (5m), 5-pyrimidyl (5n) and 2-thiazolyl (5o) groups. All the heterocyclic analogs were found to be even more potent with MICs as low as 17 nM and not susceptible to ADP-ribosylation (Table 1). In the PK characterization, the candidate compound 5m not only maintained a favorable Va, low clearance, and high stability against C-25 ester cleavage but also had an at least 100-fold increase in the unbound fraction in plasma. The drastically increased plasma free fraction of 5m, in combination with its low MIC, led to an astonishing fAUC/MIC value of 28.2, 128 times higher than rifabutin (Table 2). Further, 5m displays antibacterial activity against other Mycobacterium, including Mycobacterium simiae (M. simiae), Mycobacterium chelonae (M. chelonae), Mycobacterium fortuitum (M. fortuitium) and M. abscessus subspecies (abscessus, massilense, bolletii). See Table 9 for detailed data.

TABLE 9
Antibacterial activity of 5m against Mycobacterium simiae (M. simiae),
Mycobacterium chelonae (M. chelonae), Mycobacterium fortuitum (M. fortuitum)
and M. abscessus subspecies (abscessus, massilense, bolletii)

| | | | | M. abscessus subspecies | | |
|---|---|---|---|---|---|---|
| NTM | M. simiae | M. chelonae | M. fortuitum | abscessus | massilense | bolletii |
| MIC rifabutin (μM) | 10 | 0.58 | 0.6 | 1.5 | 0.3 | 1.2 |
| MIC 5m (μM) | 0.18 | 0.01 | 0.01 | 0.05 | 0.04 | 0.03 |
| fold | 56 | 58 | 60 | 30 | 8 | 40 |
| improvement | | | | | | |

In conclusion, we have rationally redesigned rifabutin to restore the low nanomolar antimycobacterial activity of rifamycin antibiotics and extend their clinical utility against intrinsically multidrug-resistant M. abscessus by evading a novel rifamycin resistance mechanism. Structure-based derivatization at the C-25 position of rifabutin afforded analogs that are more than a hundred-fold more potent than the widely used rifampicin and are no longer susceptible to the primary rifamycin resistance in M. abscessus through ADP-ribosylation. X-ray crystallography and molecular docking studies suggest that additional ligand-target interactions contribute to the favored on-target activity. The ability to overcome Arr-mediated resistance was validated using an in vitro biochemical assay to directly detect ADP-ribosylation, with results congruent with the observed microbiological activity using wild-type and Δarr M. abscessus strains. One representative compound also demonstrated strong in vivo efficacy comparable to the anti-M. abscessus drug clarithromycin. In a further stage of modification, three heterocyclic C-25 ester analogs were strategically designed based on compounds' free fraction in plasma, an important driving factor. Compound 5m emerged as an exemplary candidate with potent in vitro antibacterial activity, outstanding drug disposition and greatly improved in vivo PK properties.

### REFERENCES

[1] C. J. L. Murray, K. S. Ikuta, F. Sharara, L. Swetschinski, G. Robles Aguilar, A. Gray, C. Han, C. Bisignano, P. Rao, E. Wool, S. C. Johnson, A. J. Browne, M. G. Chipeta, F. Fell, S. Hackett, G. Haines-Woodhouse, B. H. Kashef Hamadani, E. A. P. Kumaran, B. McManigal, R. Agarwal, S. Akech, S. Albertson, J. Amuasi, J. Andrews, A. Aravkin, E. Ashley, F. Bailey, S. Baker, B. Basnyat, A. Bekker, R. Bender, A. Bethou, J. Bielicki, S. Boonkasidecha, J. Bukosia, C. Carvalheiro, C. Castañeda-Orjuela, V. Chansamouth, S. Chaurasia, S. Chiurchiù, F. Chowdhury, A. J. Cook, B. Cooper, T. R. Cressey, E. Criollo-Mora, M. Cunningham, S. Darboe, N. P. J. Day, M. De Luca, K. Dokova, A. Dramowski, S. J. Dunachie, T. Eckmanns, D. Eibach, A. Emami, N. Feasey, N. Fisher-Pearson, K. Forrest, D. Garrett, P. Gastmeier, A. Z. Giref, R. C. Greer, V. Gupta, S. Haller, A. Haselbeck, S. I. Hay, M. Holm, S. Hopkins, K. C. Iregbu, J. Jacobs, D. Jarovsky, F. Javanmardi, M. Khorana, N. Kissoon, E. Kobeissi, T. Kostyanev, F. Krapp, R. Krumkamp, A. Kumar, H. H. Kyu, C. Lim, D. Limmathurotsakul, M. J. Loftus, M. Lunn, J. Ma, N. Mturi, T. Munera-Huertas, P. Musicha, M. M. Mussi-Pinhata, T. Nakamura, R. Nanavati, S. Nangia, P. Newton, C. Ngoun, A. Novotney, D. Nwakanma, C. W. Obiero, A. Olivas-Martinez, P. Olliaro, E. Ooko, E. Ortiz-Brizuela, A. Y. Peleg, C. Perrone, N. Plakkal, A. Ponce-de-Leon, M. Raad, T. Ramdin, A. Riddell, T. Roberts, J. V. Robotham, A. Roca, K. E Rudd, N. Russell, J. Schnall, J. A. G. Scott, M. Shivamallappa, J. Sifuentes-Osornio, N. Steenkeste, A. J. Stewardson, T. Stoeva, N. Tasak, A. Thaiprakong, G. Thwaites, C. Turner, P. Turner, H R. van Doorn, S. Velaphi, A. Vongpradith, H. Vu, T. Walsh, S. Waner, T. Wangrangsimakul, T. Wozniak, P. Zheng, B. Sartorius, A. D Lopez, A. Stergachis, C. Moore, C. Dolecek, M. Naghavi, The Lancet 2022, 399, 629-655.
[2] E. P. Abraham, E. Chain, Nature 1940, 146, 837-837.
[3] G. D. Wright, Adv. Drug Delivery Rev. 2005, 57, 1451-1470.
[4] D. Awasthi, J. S. Freundlich, Trends Microbiol. 2017, 25, 756-767.
[5] D. M. Rothstein, Cold Spring Harbor Perspect. Med. 2016, 6, a027011.
[6] J. P. Sarathy, L. E. Via, D. Weiner, L. Blanc, H. Boshoff, E. A. Eugenin, C. E. Barry, 3rd, V. A. Dartois, Antimicrob. Agents Chemother. 2018, 62, e02266-17.
[7] U. S. Ganapathy, T. Lan, P. Krastel, M. Lindman, M. D. Zimmerman, H. Ho, J. P. Sarathy, J. C. Evans, V. Dartois, C. C. Aldrich, T. Dick, Antimicrob. Agents Chemother. 2021, 65, e00978-21.
[8] A. Rominski, A. Roditscheff, P. Selchow, E. C. Böttger, P. Sander, J Antimicrob. Chemother. 2017, 72, 376-384.
[9] D. Schäfle, P. Selchow, B. Borer, M. Meuli, A. Rominski, B. Schulthess, P. Sander, Antimicrob. Agents Chemother. 2021, 65, e02215-20.
[10] J. Baysarowich, K. Koteva, D. W. Hughes, L. Ejim, E. Griffiths, K. Zhang, M. Junop, G. D. Wright, Proc. Natl. Acad. Sci. U.S.A 2008, 105, 4886-4891.
[11] K. L. Chew, S. Octavia, J. Go, S. Ng, Y. E. Tang, P. Soh, J. Yong, R. Jureen, R. T. P. Lin, S. F. Yeoh, J. Teo, J. Antimicrob. Chemother. 2021, 76, 973-978.
[12] D. B. Aziz, J. L. Low, M.-L. Wu, M. Gengenbacher, J. W. P. Teo, V. Dartois, T. Dick, Antimicrob. Agents Chemother. 2017, 61, e00155-17.
[13] N. C. Simon, K. Aktories, J. T. Barbieri, Nat. Rev. Microbiol. 2014, 12, 599-611.
[14] T. A. Wencewicz, J. Mol. Biol. 2019, 431, 3370-3399.
[15] W. Kump, H. Bickel, Helv. Chim. Acta 1973, 56, 2323-2347.
[16] K. D. Combrink, D. A. Denton, S. Harran, Z. Ma, K. Chapo, D. Yan, E. Bonventre, E. D. Roche, T. B. Doyle, G. T. Robertson, A. S. Lynch, Bioorg. Med. Chem. Lett. 2007, 17, 522-526.
[17] K. D. Combrink, A. R. Ramos, S. Spring, S. Schmidl, K. Elizondo, P. Morin, B. De Jesus, F. P. Maurer, Bioorg. Med. Chem. Lett. 2019, 29, 2112-2115.
[18] L. Paulowski, K. S. H. Beckham, M. D. Johansen, L. Berneking, N. Van, Y. Degefu, S. Staack, F. V. Sotomayor, L. Asar, H. Rohde, B. B. Aldridge, M. Aepfelbacher, A. Parret, M. Wilmanns, L. Kremer, K. Combrink, F. P. Maurer, 2021, bioRxiv preprint DOI: 10.1101/2019.12.11.123456
[19] W. Wehrli, W. Zimmermann, W. Kump, W. Tosch, W. Vischer, O. Zak, J. Antibiot. 1987, 40, 1733-1739.
[20] Y. Crabol, E. Catherinot, N. Veziris, V. Jullien, O. Lortholary, J. Antimicrob. Chemother. 2016, 71, 1759-1771.
[21] C. A. Jamis-Dow, A. G. Katki, J. M. Collins, R. W. Klecker, Xenobiotica 1997, 27, 1015-1024.
[22] P. A. Aristoff, G. A. Garcia, P. D. Kirchhoff, H. D. Showalter, Tuberculosis 2010, 90, 94-118.
[23] T. Dick, S. J. Shin, W. J. Koh, V. Dartois, M. Gengenbacher, Antimicrob. Agents Chemother. 2020, 64, e01943-19.
[24] S. B. Lakshminarayana, T. B. Huat, P. C. Ho, U. H. Manjunatha, V. Dartois, T. Dick, S. P. Rao, J. Antimicrob. Chemother. 2015, 70, 857-867.
[25] R. Jayaram, S. Gaonkar, P. Kaur, B. L. Suresh, B. N. Mahesh, R. Jayashree, V. Nandi, S. Bharat, R. K. Shandil, E. Kantharaj, V. Balasubramanian, Antimicrob. Agents Chemother. 2003, 47, 2118-2124.
[26] The RNAP_{Mtb}-5a co-crystal structure has been deposited in the Protein Data Bank. PDB code: 7U22.

### Biological Procedures

### Ethical Approval Statements for the Animal Experiments

All experiments involving live mice were approved by the Institutional Animal Care and Use Committee of the Center for Discovery and Innovation, Hackensack Meridian Health. Accreditation number: A4278-01. IACUC number: 269.00 (in vivo pharmacokinetic studies); 287.00 (in vivo animal efficacy studies).

### Bacterial Strains, Culture Media and Compounds.

M. abscessus Bamboo was isolated from the sputum of a patient with amyotrophic lateral sclerosis and bronchiectasis and was provided by Wei Chang Huang, Taichung Veterans General Hospital, Taichung, Taiwan. M. abscessus Bamboo whole-genome sequencing showed that the strain belongs to M. abscessus subsp. Abscessus and harbors an inactive clarithromycin-sensitive erm(41) C28 sequevar^{[6-7]}. Mycobacterium abscessus subsp. Abscessus ATCC 19977, harboring the inducible clarithromycin resistance-conferring erm(41) T28 sequevar^{[8]}, was purchased from the American Type Culture Collection (ATCC). M. abscessus subsp. abscessus K21 was isolated from a patient and provided by Sung Jae Shin (Department of Microbiology, Yonsei University College of Medicine, Seoul, South Korea) and Won-Jung Koh (Division of Pulmonary and Critical Care Medicine, Samsung Medical Center, Seoul, South Korea). This strain harbors the inactive, clarithromycin-sensitive erm(41) C28 sequevar as determined previously^{[9]}.

Generation of Δarr_{Mab} in the M. abscessus ATCC 19977 genetic background by recombineering was described previously.^{[10]}

The selection and characterization of the rifamycin-resistant M. abscessus mutant RFB-R1 was described previously.^{[10]} RFB-R1 carries a rpoB (RNAP) c1339t nucleotide mutation, which corresponds to a H447Y missense mutation that was previously reported in M. tuberculosis.^{[11-12]}

For general bacteria culturing and MIC experiments, Middlebrook 7H9 broth (BD Difco) supplemented with 0.5% albumin, 0.2% glucose, 0.085% sodium chloride, 0.0003% catalase, 0.2% glycerol, and 0.05% Tween 80.

Clarithromycin was purchased from Sigma-Aldrich. Rifampicin was purchased from GoldBio. Rifabutin was purchased from Acros Organics. All drugs were prepared as 10 mM stocks in 100% DMSO.

MIC Assay in 96-well Plate Format.

MIC determination was carried out in 96-well plate format as previously described.^{[13-14]} 96-well plates were initially set up with 100 μL of 7H9 per well. For each compound, a ten-point two-fold dilution series starting at twice the desired highest concentration was dispensed onto the 96-well plates using a Tecan D300e Digital Dispenser, with the DMSO concentration normalized to 2%. M. abscessus culture grown to mid-log phase (OD₆₀₀=0.4-0.6) was diluted to OD₆₀₀=0.1 (1×10⁷ CFU/mL). 100 μL of the resulting bacteria suspension was dispensed onto the 96-well plates containing compounds to give a final volume of 200 μL per well with an initial OD₆₀₀=0.05 (5×10⁶ CFU/mL) and final DMSO concentration of 1%. Final compound concentration ranges were typically 50-0.098 μM, 6.25-0.012 μM, 0.006-3.13 μM, or 0.003-1.56 μM. Untreated control wells are included on each plate that contain bacteria suspension and 1% DMSO. Plates were sealed with parafilm, stored in boxes with wet paper towels and incubated at 37° C. with shaking (110 RPM). Plates were incubated for 3 days. To determine growth, OD₆₀₀ was measured using a Tecan Infinite M200 plate reader on Day 0 and Day 3. Two biological replicates were performed. Clarithromycin was included in each experiment as a positive control.

For each well on the 96-well plate, bacterial growth was calculated by subtracting the Day 0 OD₆₀₀ value from the Day 3 OD₆₀₀ value. For each compound series, the bacterial growth values for the untreated control wells were averaged to give the average drug-free bacterial growth. For compound-containing wells, percentage growth was calculated by dividing their growth values by the average drug-free bacterial growth for the compound series and multiplying by 100. For each compound series, we plotted percentage growth versus compound concentration. By visual inspection of the dose-response curve, we determined the MIC of a compound as the compound concentrations that would result in 90% growth inhibition.

### Pharmacokinetics Studies.

CD-1 female mice (22-25 g) were used in oral pharmacokinetic studies. Rifabutin, 5b, 5j, and 5m were administered as a single intravenous (IV) or oral (PO) dose gavage at 10 mg/kg in a solution formulation composed of 5% DMSO:95% (4% Cremophor EL). Aliquots of 50 μL of blood were taken by puncture of the lateral tail vein from each mouse (n=3 per route and dose) at 30 minutes, 1, 3, 5, 7, and 24 hours post-dose for oral dosing and at 1 minute, 15 minutes, 1, 3, 7, and 24 hours for IV dosing. Blood was captured in CB300 blood collection tubes containing K₂EDTA and stored on ice. Plasma was recovered after centrifugation and stored at −80° C. until analyzed by high pressure liquid chromatography coupled to tandem mass spectrometry (LC-MS/MS). Pharmacokinetic parameters were calculated using non-compartmental pharmacokinetic analysis.

### LC-MS/MS Analytical Methods for the Pharmacokinetic Studies

Neat 1 mg/mL DMSO stocks of rifabutin, C25-desacetyl rifabutin, 5b, 5j, and 5m were serial diluted in 50/50 Acetonitrile (ACN)/Milli-Q water to create neat standard solutions. Plasma standards were created by adding 10 μL of spiking solutions to 90 μL of drug free plasma (CD-1 K2EDTA Mouse, Bioreclamation IVT). 5 μL of control, standard, or study sample were added to 100 μL of ACN protein precipitation solvent containing 10 ng/mL of the internal standards Verapamil (Sigma Aldrich) and rifabutin-d7 (Toronto Research Chemical). Extracts were vortexed for 5 minutes and centrifuged at 4000 RPM for 5 minutes. 75 μL of supernatant was transferred for LC-MS/MS analysis and diluted with 75 μL of Milli-Q deionized water. Rifabutin was purchased from Carbosynth. C25-desacetyl rifabutin and rifabutin-d7 were purchased from Toronto Research Chemical. Verapamil was purchased from Sigma-Aldrich.

LC-MS/MS analysis was performed on a Sciex Applied Biosystems Qtrap 6500+ triple-quadrupole mass spectrometer coupled to a Shimadzu Nexera X2 UHPLC system to quantify each drug in plasma. Chromatography was performed on an Agilent SB-C8 (2.1×30 mm; particle size, 3.5 μm) using a reverse phase gradient. Milli-Q deionized water with 0.1% formic acid was used for the aqueous mobile phase and 0.1% formic acid in ACN for the organic mobile phase. Multiple-reaction monitoring of parent/daughter transitions in electrospray positive-ionization mode was used to quantify all analytes. The following MRM transitions were used for rifabutin (847.60/755.60), rifabutin-d7 (854.60/762.60), C25-desacetyl rifabutin (805.48/773.50), 5b (923.48/891.40), 5j (927.39/895.30), 5m (910.45/878.40) and Verapamil (455.40/165.00). Sample analysis was accepted if the concentrations of the quality control samples were within 20% of the nominal concentration. Data processing was performed using Analyst software (version 1.6.2; Applied Biosystems Sciex).

### Plasma Protein Binding Assays

DMSO stocks were spiked into plasma to a concentration of 10,000 ng/mL. 200 μL of spiked plasma was pipetted into the sample chamber of the rapid equilibrium dialysis (RED) cartridge. 350 μL of PBS was added to the adjacent cartridge. The plate containing the RED was sealed and incubated at 37° C. on the thermomixer at 300 RPM for 4 h. After incubation, 50 μL aliquots of plasma were removed and added to 50 μL of blank plasma in a deep well plate (1:1). Similarly, 50 μL aliquots of PBS were removed and added to 50 μL of blank plasma. This created an identical matrix between buffer and non-buffer samples. Samples were processed and quantified as specified in the LC-MS/MS analytical method.

### Calculation of Physicochemical Properties

For the physicochemical properties disclosed in the examples, clogP is calculated using ChemDraw 21.0.0.28. The rest of the physicochemical properties were calculated using “SwissADME” online service, http://wwxv.swissadme.ch/.

### Crystal Structure Determination

Crystals of M. tuberculosis RNAP (RNAPA_{Mtb}) M. tuberculosis σ^{A} RPo were prepared as described.^{[15]} Crystals were soaked overnight at 23° C. in cryoprotection solution (20 mM Tris-HCl, pH 8.2, 200 mM potassium chloride, 20 mM magnesium chloride, 7% (m/v) PEG-3350, 20% (v/v) (2R,3R)-(−)-2,3-butanediol, 1 mM CHAPSO) supplemented with 0.5 mM 5a and were flash-frozen in liquid nitrogen.

X-ray diffraction data were collected at the Stanford Synchrotron Radiation Lightsource (SSRL) beamline 12-2 and processed using HKL3000.^{[16]} The structure was solved by molecular replacement using the structure of M. tuberculosis σ^{A} RPo (PDB: 5UHA)^{[15]} as the search model. Iterative cycles of model building and refinement were performed using Coot^{[17]} and Phenix Refine^{[18]}. The final model was obtained by a refinement with secondary-structure restraints and individual and group B-factors. The atomic model and structure factors were deposited in the Protein Data Bank (PDB) with accession number 7U22.

### Arr_{Mab} Expression and Purification

Arr_{Mab} was codon optimized for E. Coli and cloned into pET-28b (+) expression vector. Clone was transformed in E. coli BL21 (DE3) cells. Single colony was picked and allowed to grow in 50 mL LB having desired antibiotic overnight at 37° C. shaking at 250 RPM. Overnight grown Primary cultures were transferred to 1 L LB broth with kanamycin 50 μg/mL and grown at 37° C. shaking at 250 RPM till OD (600 nm) reaches 0.6. Protein expressions were induced by adding 0.5 mM IPTG and reducing the temperature to 20° C. for 16 hours. Cells were harvested by centrifugation at 8000 g for 10 minutes and resuspended in lysis buffer (50 mM Tris pH 7.5, 150 mM NaCl, and 1.0 M sorbitol) containing complete protease inhibitor tablet, PMSF (5 mM) and hen egg white lysozyme (0.5 mg/mL). Cells were lysed by Avastin C3 ultra high-pressure liquidizer at 15000-20000 psi for 15 minutes. Supernatant was obtained after high-speed centrifugation and incubated with equilibrated Ni-NTA beads for 90 minutes at 4° C. on rotator. Both proteins were eluted with gradient of 50-200 mM imidazole.

### HPLC Analysis for In Vitro Validation of ADP-Ribosylation

Endpoint reactions were set up for rifampicin, rifabutin and rifabutin analogs in the presence of 5 μM enzyme in 50 mM HEPES buffer. Reactions were quenched using methanol and subjected to analysis on HPLC. Reverse-phase LC was performed on a Kinetex C8 column (100 mm×2.1 mm, 2.6 μm; Phenomenex, Torrance, CA) using LC 1200 Infinity Series, Agilent Technologies instrument with wavelength monitored at 260 nm (for rifampicin) and 277 nm (for rifabutin and rifabutin analogs). The elution gradient was carried out with binary solvent system consisting of 0.1% formic acid in H₂O (solvent A) and 0.1% formic acid in MeCN (solvent B). A linear gradient profile with the following proportions (v/v) of solvent B was applied (t (min), % B): (0, 5), (0.5, 5), (9, 100), (10.5, 100), (12, 10) with 5 min for re-equilibration to provide a total run time of 17 min. The flow rate was 0.3 mL/min and the column oven was maintained at 28° C. The injection volume was 10 μL.

Efficacy Evaluation in M. abscessus Mouse Infection Model

Eight-week-old female NOD·CB17-Prkdc^{scid}/NCrCrl (NOD SCID) mice (Charles River Laboratories) were infected intranasally with ˜10⁶ CFU of M. abscessus subsp. abscessus K21 as described previously.^{[9]} Acute infection was achieved within one day. Drugs or the vehicle control was administered once daily for 10 consecutive days by oral gavage, starting 1 day post-infection. Clarithromycin (250 mg/kg, Sandoz clinical tablets), rifabutin (10 mg/kg, Carbosynth) and 5j (10 mg/kg) were formulated in 0.5% carboxy-methyl-cellulose/0.5% tween 80 at 8 mL/kg dosing volume. All mice were euthanized 24 h after the last dose, and lungs and spleen were aseptically removed prior to homogenization. The bacterial load in these organs was determined by plating serial dilutions of the organ homogenates onto Middlebrook 7H11 agar (BD Difco) supplemented with 0.2% (v/v) glycerol and 10% (v/v) OADC. The agar plates were incubated for 5 days at 37° C. prior to counting of colonies.

### Computational Procedures

Sequence Retrieval and Homology Modeling of RNAP_{Mab} and Arr_{Mab}

The sequences of RNAP_{Mab} and Arr_{Mab} were retrieved from the UniProt database (entry B1MH62 & B1MH05). A protein-protein BLAST (Blastp) search was performed to find a suitable homologous sequence (template) with known 3D structure to this amino acid sequence.^{[19]} The sequences of the best template, RNAP_{Mtb} (PDB: 5UHB) and Arr_{Msm} (PDB: 2HW2), and the studied targets were then aligned using Clustal Omega.^{[20]} The initial homology models of RNAP_{Mab} and Arr_{Mab} based on the selected templates were generated with the automated homology modeling software, MODELLER (Version 9), in which the program is based on comparative structure modeling^{[21]} The catalytic domain of Arr_{Mab} was further aligned to the similar poly ADP-ribose polymerase (PARP) domains of Pseudomonas aeruginosa exotoxin (PDB: 1AER) and Gallus gallus poly ADP-ribose polymerase (PDB: 1A26) to predict the NAD binding site of Arr_{Mab}. The ADP-ribosyl-oxocarbenium intermediate was docked at the predicted binding site of Arr_{Mab} and the best binding mode was selected to build an Arr_{Mab} homology model with the ADP-ribosyl-oxocarbenium intermediate bound. Then, both the RNAP_{Mab} and Arr_{Mab}, models were further energy minimized with Discovery Studio 3.5 using the CHARMM force field. The quality and stability of the homology models were validated by checking the stereochemical parameters using PROCHECK, VERIFY3D, and ERRAT at SAVES server (http://nihserver.mbi.ucla.eduiSAVES).

### Rational for ADP-Ribosyl-Oxocarbeniun Intermediate in the Arr_{Mab} Model

The Arr_{Mab} homology model with the ADP-ribosyl-oxocarbenium intermediate was used to dock 5a and rifabutin after confirming that both compounds showed better binding affinity values when nicotinamide moiety of NAD⁺ was not present in the model. In this model, it was predicted that the hydroxyl groups at C23 position of rifabutin and 5a were more closely located to C1′ of the ribose ring, compared to NAD⁺ bound Arr_{Mab} homology model. This prediction also supports the proposed mechanism that the oxocarbenium transition state enables the hydroxyl group at position 23 of the antibiotic to attack C1′ of the ribose.^{[21]} Thus, the Arr_{Mab} homology model with the ADP-ribosyl-oxocarbenium intermediate was a better model to predict the interaction between rifabutin analogs and Arr_{Mab}.

### Ligand Generation

The 2D structures of rifabutin and synthetic rifabutin analogs were drawn in Chemdraw and their SMILES notation was obtained. The 3D structures were obtained and converted into SDF files after energy minimization with Discovery Studio 3.5.

### In Silico Molecular Docking Analysis

The molecular docking was carried out by Pyrx with Autodock Vina engine. The AutoDockTools package was employed to generate the docking input files in pdbqt. Both protein targets and ligands were opened in PyRx virtual screening tool as a starting protein structure in pdbqt format. Docking was carried out taking the rifampicin binding site residues for RNAP_{aI}b and for Arr_{Mab} inside a grid box with co-ordinates along X, Y, and Z-axis and dimensions conformed to 162.8 Å×163.38 Å×20.22 A and 20 Å×20 Å×20 Å, and 66.89 Å×66.62 Å×10.54 Å and 20 Å×20 Å×20 Å, respectively. Lamarckian Genetic Algorithm (LGA) was used as ligand conformation search process, and the other parameters were default settings. The quality of docking was validated by re-docking ligand to observe the precision of the docking condition. All the docking simulation using various ligands was performed with the exhaustiveness of 24. The best binding mode and affinity values were obtained in PyRx virtual screening GUI and log files.

### Example 2

### General Methods for Chemical Synthesis

Reagents and solvents were purchased from commercial sources (Fisher Scientific, MilliporeSigma, A2B Chem, Oakwood Chemical) and used as received unless otherwise noted. Rifabutin was purchased from WuXi AppTec (Tianjin). Reactions were monitored using Macherey-Nagel® ALUGRAM® SIL G/UV254 aluminum TLC plates. LC were visualized under visible light or UV fluorescence (254 nm). Flash chromatography was performed using Sorbtech® Silica Gel [porosity: 60 Å; particle size: 40-63 μm (230×400 mesh)]. Preparative TLC were performed on Silicyle® glass-backed TLC plates (thickness: 1000 μm; indicator: F-254). NMR Spectra were recorded on a Bruker 600-MHz Avance NEO. ¹H frequency is at 601 MHz. ¹³C frequency is at 151 MHz. Chemical shifts (δ) were reported in parts per million (ppm) relative to residual solvent peaks [CDCl₃ (¹H: 7.26, ¹³C: 77.2) or CD₂Cl₂ (¹H: 5.32, ¹³C: 53.8)]. Peak multiplicity was indicated as follows: s (singlet), d (doublet), t (triplet), q (quartet), m (multiplet) and br (broad). High resolution mass spectra (HRMS) were recorded on Bruker BioTOF II ESI/TOF-MS. Analytical HPLC analysis was performed on an Agilent® 1260 Infinity Quaternary LC system with a reversed-phase C18 column (Gemini-Nx 5 micron, 150×4.60 mm, Phenomenex).

### Experimental Procedures and Characterization Data

### Synthesis of 25-O-acyl rifabutin Analogs

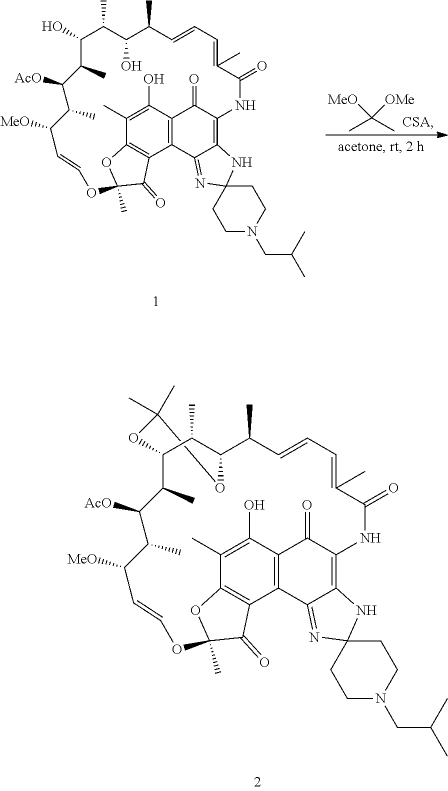

21,23-O-Isopropylidenerifabutin (2). To a solution of rifabutin 1 (5.01 g, 5.90 mmol, 1.00 equiv) and camphorsulfonic acid (1.66 g, 7.15 mmol, 1.20 equiv) in acetone (59.0 mL, dried with 4 Å molecular sieves) was added 2,2-dimethoxypropane (15.0 mL, 122 mmol, 20.7 equiv). The reaction mixture was stirred at 23° C. for 2 h; then NaHCO₃ (1.8 g, 21 mmol, 3.60 equiv) was added, and the mixture was further stirred for 30 min at room temperature. Then the reaction was partitioned between CH₂Cl₂ (80 mL) and H₂O (80 mL). The aqueous phase was back extracted with CH₂Cl₂ (3×50 mL). The combined organic layers were dried (Na₂SO₄) and concentrated under reduced pressure. Purification by silica gel flash chromatography (CH₂Cl₂:MeOH 50:1) on silica gel afforded the title compound (3.94 g, 75%) as a purple solid: R_{f}=0.13 (50:1 CH₂Cl₂:MeOH): ¹H NMR (601 MHz, CD₂Cl₂) δ 14.88 (s, 1H), 8.80 (s, 1H), 7.77 (s, 1H), 6.29 (dd, J=15.8, 10.8 Hz, 1H), 6.18-6.14 (m, 1H), 6.06 (dd, J=15.8, 7.1 Hz, 1H) 5.91 (dd, J=12.2, 1.1 Hz, 1H), 5.04 (dd, J=12.2, 6.6 Hz, 1H), 4.89 (dd, J=7.9, 1.5 Hz, 1H), 3.59 (dd, J=10.7, 3.4 Hz, 1H), 3.38 (ddd, J=6.6, 2.3, 1.1 Hz, 1H), 3.03 (dd, J=10.3, 5.2 Hz, 1H), 3.01-2.87 (m, 2H), 2.82 (s, 3H), 2.68-2.53 (m, 2H), 2.27 (s, 3H), 2.32-2.19 (m, 3H), 2.03 (s, 2H), 1.93 (s, 3H), 1.89-1.79 (m, 1H), 1.79-1.75 (m, 1H), 1.74 (s, 3H), 1.56-1.48 (m, 1H), 1.44 (pd, J=7.1, 2.2 Hz, 1H), 1.20 (s, 1H), 1.17 (s. 3H), 0.94 (d, J=6.6 Hz, 6H), 0.86 (s. 3H), 0.85 (d, J=6.6 Hz, 2H), 0.82 (d, J=6.8 Hz, 3H), 0.69 (d, J=7.2 Hz, 3H), 0.36 (d, J=7.1 Hz, 3H); ¹³C NMR (151 MHz, CD₂Cl₂) δ 192.6, 181.6, 172.4, 170.4, 169.0, 168.5, 155.4, 142.7, 141.2, 141.0, 132.5, 131.9, 125.8, 124.2, 115.5, 113.8, 112.1, 109.0, 106.3, 104.9, 100.2, 100.2, 95.4, 79.0, 77.3, 74.5, 71.3, 69.7, 66.8, 56.4, 51.9, 51.8, 41.5, 41.0, 36.6, 36.5, 35.9, 34.7, 32.0, 29.5, 26.2, 26.0, 24.0, 21.0, 21.0, 21.0, 20.4, 20.4, 17.9, 13.0, 9.9, 9.6, 7.8; MS (ESI): m/z [M+H]⁺ calcd for C₄₉H₆₆N₄O₁₁: 887.4801; found: 887.4786 (error 1.7 ppm).

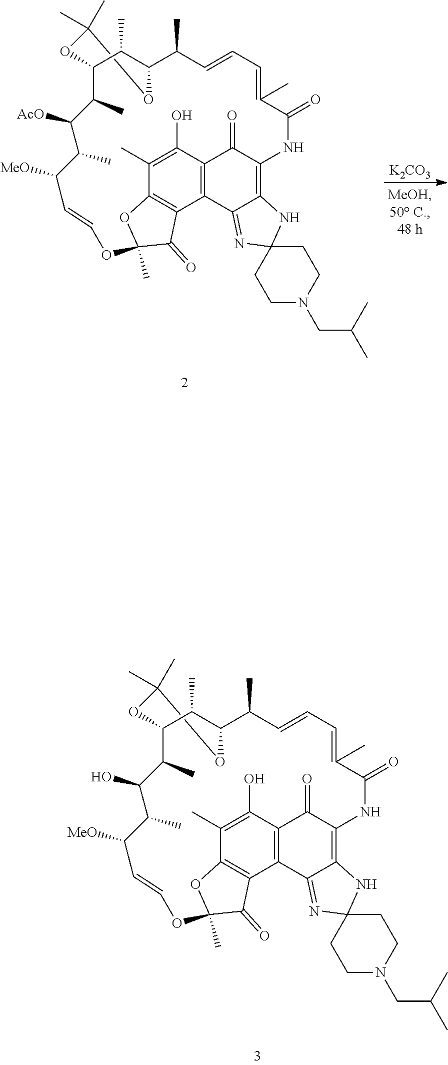 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@@H]2OC(C)(C)O[C@@H]([C@@H](C)[C@H](OC(C)=O)[C@@H]1C)[C@@H]2C)C4=O ; "K2CO3" ; CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@@H]2OC(C)(C)O[C@@H]([C@@H](C)[C@H](O)[C@@H]1C)[C@@H]2C)C4=O]

25-Desacetyl-21,23-O-isopropylidenerifabutin (3). A mixture of 2 (3.56 g, 4.01 mmol, 1.00 equiv) and K₂CO₃; (3.33 g, 24.1 mmol, 6.00 equiv) in MeOH (40 ml) was heated under 50° C. for 48 h. The reaction was cooled to room temperature and partitioned between CH₂Cl₂ (50 mL) and brine (50 mL). The organic layer was separated and the aqueous phase was extracted with CH₂Cl₂ (3×40 mL). The combined organic extracts were dried (Na₂SO₄) and concentrated under reduced pressure. Purification by flash chromatography (CH₂Cl₂:MeOH 60:1) on silica gel afforded the title compound (2.17 g, 64%, containing trace amount of the starting material) as a purple solid: R_{f}=0.19 (hexanes:EtOAc:MeOH:Et₃N 8:1:1:0.1); ¹H NMR (600 MHz, CD₂Cl₂) δ 14.93 (s, 1H), 8.72 (s, 1H), 7.81 (s, 1H), 6.28 (dd, J=15.8, 10.3 Hz, 1H), 6.16 (dd, J=11.4, 1.6 Hz, 1H), 6.15 (d, J=11.2 Hz, 1H), 5.96 (dd, J=15.8, 6.6 Hz, 1H), 4.95 (dd, J=12.2, 9.1 Hz, 1H), 3.60 (dd, J=9.1, 3.4 Hz, 1H), 3.52 (dd, J=10.4, 3.2 Hz, 1H), 3.44 (dt, J=8.8, 3.0 Hz, 1H), 3.15 (d, J=3.7 Hz, 1H), 3.14 (s, 3H), 3.11 (dd, J=9.1, 5.0 Hz, 1H), 2.99-2.86 (m, 1H), 2.67-2.50 (m, 2H), 2.25 (dd, J=7.4, 1.9 Hz, 2H), 2.23 (s, 3H), 2.03 (s, 3H), 2.09-1.87 (bs, 4H), 1.84 (dq, J=13.6, 6.7 Hz, 1H), 1.75 (s, 3H), 1.66-1.58 (m, 1H), 1.59-1.52 (m, 1H), 1.35 (ddd, J=9.5, 6.8, 2.7 Hz, 1H), 1.02 (s, 3H), 0.94 (d, J=6.6 Hz, 6H), 0.85 (d, J=7.0 Hz, 3H), 0.82 (d, J=6.8 Hz, 3H), 0.80 (s, 3H), 0.72 (d, J=6.8 Hz, 3H), 0.49 (d, J=7.1 Hz, 3H); ¹³C NMR (151 MHz, CD₂Cl₂) δ 191.3, 181.9, 171.6, 169.2, 168.5, 155.5, 143.0, 142.9, 140.6, 132.5, 132.2, 125.9, 124.5, 114.4, 112.2, 112.1, 108.9, 105.9, 105.2, 99.7, 95.4, 83.2, 76.0, 71.6, 71.1, 66.8, 56.4, 52.1, 51.8, 41.2, 40.1, 36.7, 35.8, 35.2, 35.1, 26.2, 25.7, 24.5, 21.0, 21.0, 20.0, 20.0, 17.8, 13.0, 13.0, 8.5, 7.8; MS (ESI): m/z [M+Na]⁺ calcd for C₄₉H₆₅N₄O₁₁Na: 867.4515; found: 867.4538 (error 2.6 ppm).

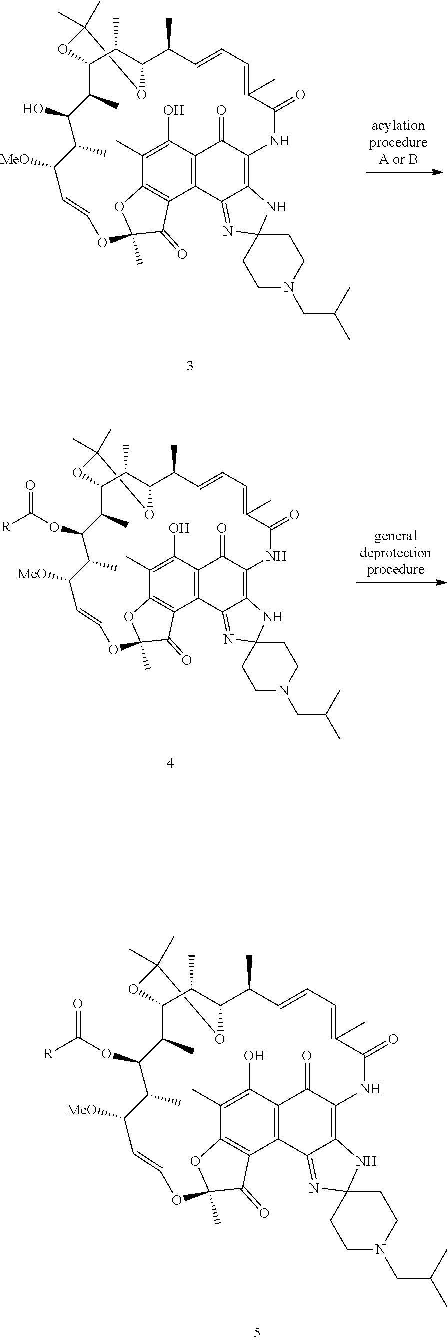

Acylation procedure A: To a solution of 3 (1.00 equiv) in 1,2-dichloroethane (0.1 M) at room temperature or 50° C., the acid anhydride (3.00-5.00 eqiv) prepared from the respective acid^{[1]} and DMAP (0.50-0.80 equiv) were added every 12 h until the majority of 3 was converted. For most cases a total amount of 10.0-20.0 equivalents anhydride and 2.00-3.00 equivalents DMAP are required in 2-3 days. Then the reaction mixture was poured into CH₂Cl₂ (10 mL) and saturated aqueous NaHCO₃ (10 mL) and the biphasic mixture was stirred at 23° C. for 16 h to quench any remaining anhydride. The organic layers were separated and the aqueous phase was extracted with CH₂Cl₂ (2×10 mL). The combined organic layers were dried (Na₂SO₄) and concentrated under reduced pressure. Purification by preparative silica gel TLC afforded the 25-O-acyl-21,23-isopropylidenerifabutin analogs 4, which were deprotected following the general procedure for deprotection.

Acylation procedure B. To a solution of the acid (4.00 equiv), triethylamine (5.00 equiv) and DMAP (2.00 equiv) in CH₂Cl₂ (0.4 M to the acid) at 0° C. was added pivaloyl chloride (4.00 equiv) and the reaction was allowed to warm to 23° C. over 1 h. Next, 3 (1.00 equiv) was added and the resulting solution was further stirred at 23° C. for 4 h. The reaction mixture was poured into CH₂Cl₂ (10 mL) and saturated NaHCO₃ aqueous solution (10 mL) and the biphasic mixture was stirred at 23° C. for 16 h to quench any remaining mixed anhydride. The organic layers were separated and the aqueous phase was extracted with CH₂Cl₂ (2×10 mL). The combined organic layers were dried (Na₂SO₄) and concentrated under reduced pressure. Purification by preparative silica gel TLC afforded the 25-O-acyl-21,23-isopropylidenerifabutin analogs 4, which were deprotected following the general procedure for deprotection.

General deprotection procedure. A solution of a 25-O-acyl-21,23-isopropylidene rifabutin analog (1.00 equiv), prepared using acylation procedure A or B, and camphorsulfonic acid (2.00 equiv) in methanol (0.1 M) was stirred at room temperature for 30 min; then the reaction was partitioned between CH2Cl₂ (10 mL) and saturated NaHCO₃ aqueous solution (10 mL). The aqueous phase was then back extracted with CH₂Cl₂ (2×10 mL). The combined organic layers were dried (Na₂SO₄) and concentrated under reduced pressure. The resulting crude was purified by preparative TLC (hexane:ethyl acetate:methanol:triethylamine=8:1:1:0.1 or hexane:ethyl acetate:methanol=7:2:1) to obtain 25-O-acyl rifabutin analogs 5 (see below for full characterizations).

### Synthesis of 25-O-carbamoyl and carbamoyl sulfonamide rifabutins (Carbamate and Sulfonamide Analogs)

### General Scheme

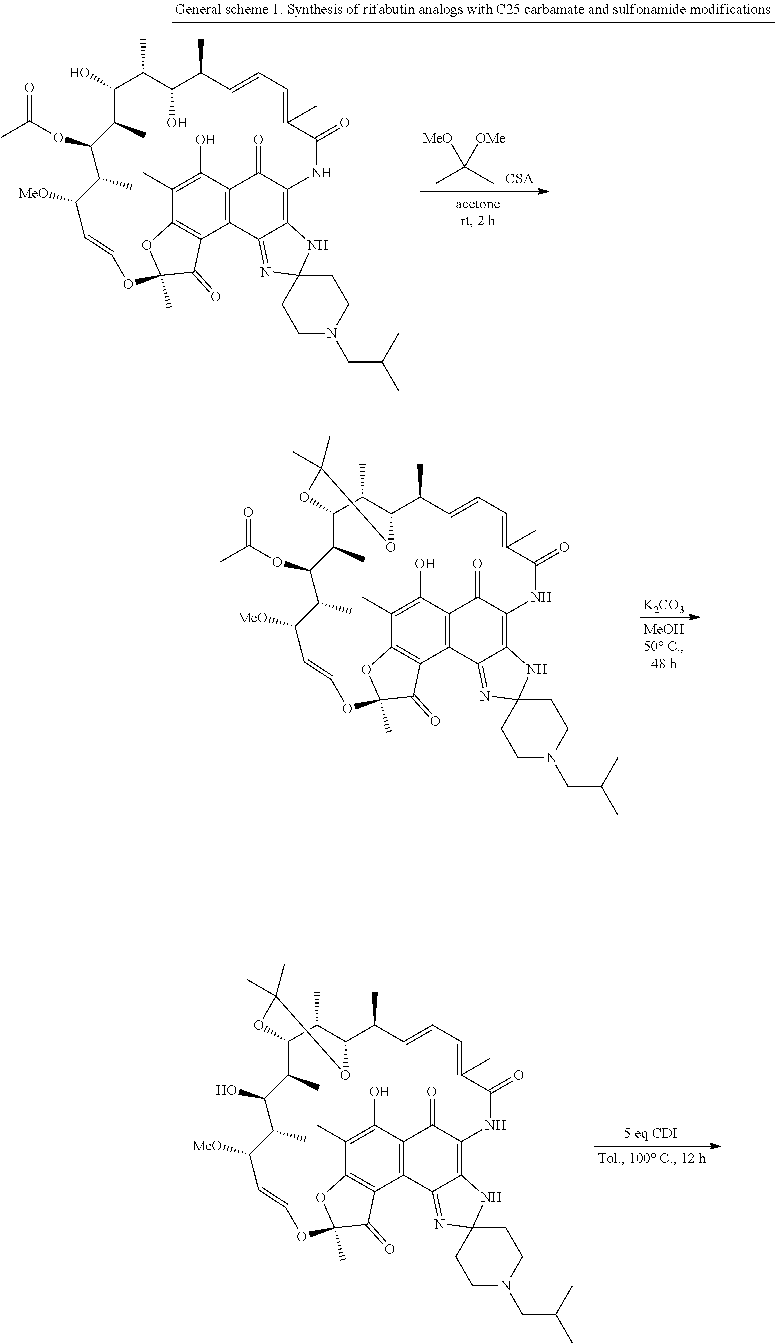

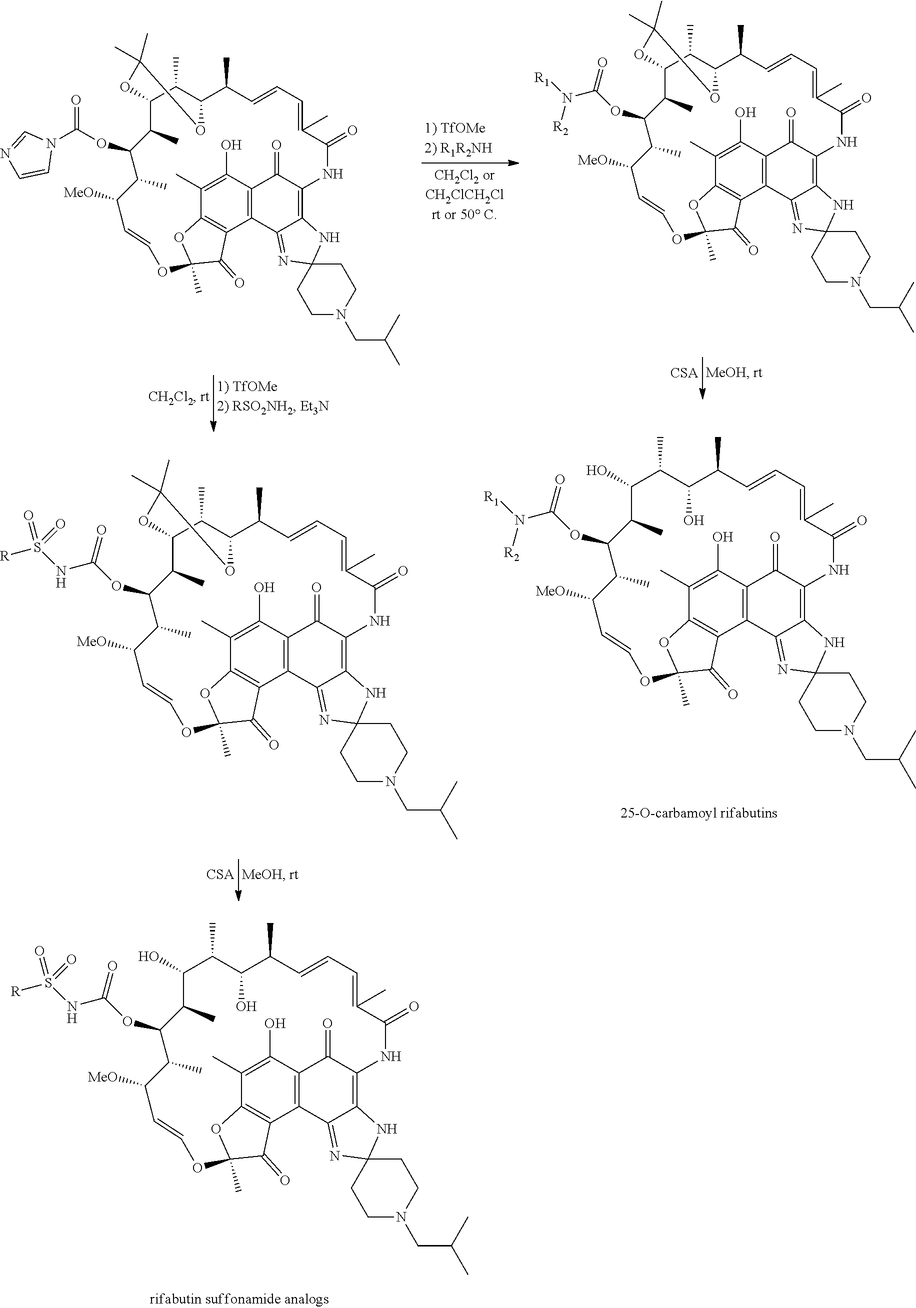

### Synthesis of 25-O-carbamoyl rifabutins (Carbamate Analogs)

Carbamate formation through acylimidazolidate intermediate LT-I-00F (carbamate formation procedure A)

### CDI Coupling Method I:

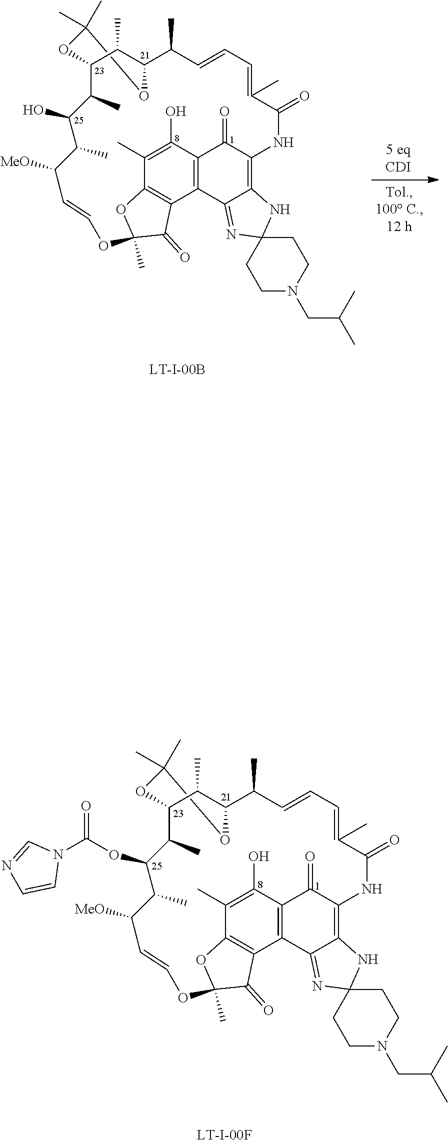 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@@H]2OC(C)(C)O[C@@H]([C@@H](C)[C@H](O)[C@@H]1C)[C@@H]2C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@@H]2OC(C)(C)O[C@@H]([C@@H](C)[C@H](OC(=O)n3ccnc3)[C@@H]1C)[C@@H]2C)C4=O]

To a solution of LT-I-00B (3.5 g, 4.14 mmol) in toluene (30 mL) was added CDI (3.36 g, 20.71 mmol. The mixture was stirred a: 100° C. for 12 hr. LC-MS showed reaction was consumed completely and desired compound was detected. The residue was concentrated and purified by flash silica gel chromatography (Fluent of 0˜6% Dichloromethane:Methanol gradient @100 mL/min) to afford LT-I-00F (1.8 g, 46% yield) as a purple solid.

### CDI Coupling Method II:

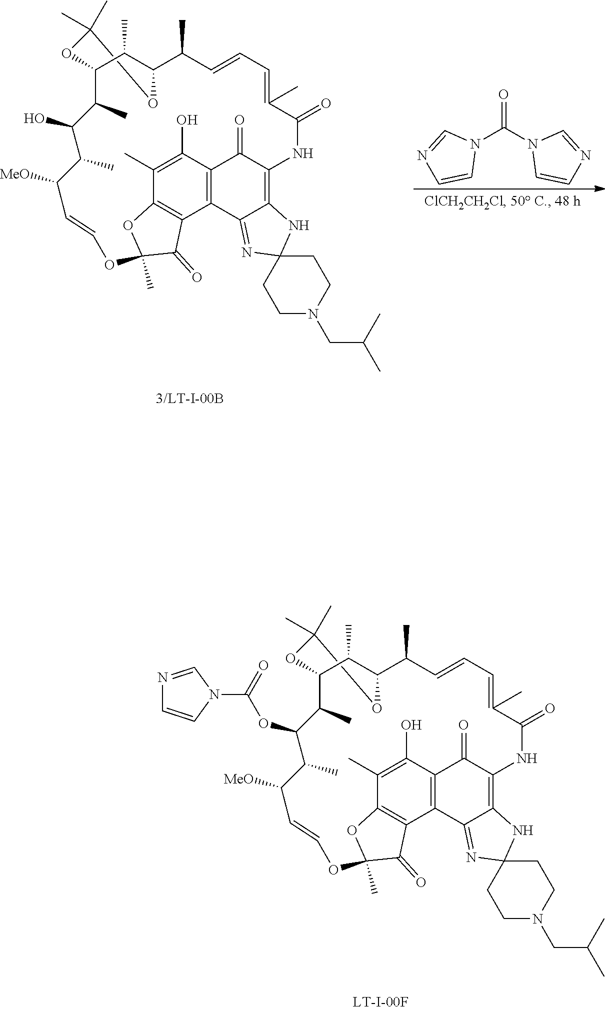 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@@H]2OC(C)(C)O[C@@H]([C@@H](C)[C@H](O)[C@@H]1C)[C@@H]2C)C4=O ; O=C(n1ccnc1)n1ccnc1 ; CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@@H]2OC(C)(C)O[C@@H]([C@@H](C)[C@H](OC(=O)n3ccnc3)[C@@H]1C)[C@@H]2C)C4=O]

A mixture of 3 (1.02 g, 1.21 mmol, 1.00 equiv.) and 1,1′-Carbonyldiimidazole (CDI) (0.59 g, 3.64 mmol, 3.00 equiv.) in 1,2-dichloroethane (8 mL) was heated under 50° C. for 24 h. Then CDI (0.20 g, 1.21 mmol, 1.00 equiv.) was added and the reaction was further stirred under 50° C. for 24 h. The reaction was then cooled to room temperature and partitioned between CH₂Cl₂ (20 mL) and water (20 mL). The organic layer was separated and the aqueous phase was extracted with CH₂Cl₂ (3×20 mL). The combined organic extracts were dried (Na₂SO₄) and concentrated under reduced pressure. Purification by flash chromatography (CH₂Cl₂:MeOH 50:1) on silica gel afforded the title compound (774.0 mg, 68%) as a purple solid: Rf=0.08 (hexanes:EtOAc:MeOH:Et₃N 8:1:1:0.1); ¹H NMR (600 MHz, CD₂Cl₂) δ 14.91 (s, 1H), 8.81 (s, 1H), 8.03 (s, 1H), 7.90 (s, 1H), 7.36 (t, J=1.5 Hz, 1H), 6.99 (dd, J=1.7, 0.9 Hz, 1H), 6.29 (dd, J=15.9, 10.7 Hz, 1H), 6.16 (d, J=10.7 Hz, 1H), 6.05 (dd, J=15.8, 7.0 Hz, 1H), 5.98 (dd, J=12.2, 0.9 Hz, 1H), 5.13 (dd, J=8.1, 1.6 Hz, 1H), 5.06 (dd, J=12.2, 7.3 Hz, 1H), 3.59 (dd, J=10.6, 3.4 Hz, 1H), 3.42-3.38 (m, 1H), 3.05 (dd, J=10.2, 5.3 Hz, 1H), 3.02-2.95 (m, 1H), 2.95-2.89 (m, 1H), 2.76 (s, 3H), 2.69-2.55 (m, 2H), 2.27 (s, 3H), 2.33-2.21 (m, 3H), 2.03 (s, 3H), 2.09-1.89 (m, 4H), 1.90-1.80 (m, 1H), 1.75 (s, 3H), 1.78-1.70 (m, 3H), 1.59-1.51 (m, 1H), 1.13 (s, 3H), 0.95 (d, J=6.6 Hz, 6H), 0.85 (d, J=4.3 Hz, 3H), 0.85 (d, J=4.3 Hz, 3H), 0.83 (d, J=7.2 Hz, 3H), 0.83 (d, J=6.6 Hz, 3H), 0.49 (d, J=7.1 Hz, 3H): ¹³C NMR (151 M Hz, CD₂Cl₂) δ 192.3, 181.7, 172.2, 169.0, 168.5, 155.5, 148.6, 142.8, 142.1, 140.7, 137.3, 132.4, 132.2, 130.6, 125.8, 124.3, 117.4, 114.1, 113.8, 112.1, 108.9, 106.2, 105.0, 100.3, 95.4, 79.7, 79.3, 76.8, 71.3, 66.8, 56.2, 51.9, 51.8, 41.0, 40.8, 36.7, 36.6, 35.8, 34.6, 26.2, 25.9, 23.8, 21.00, 20.98, 20.97, 20.3, 17.8, 13.0, 10.6, 9.8, 7.8; MS (ESI): m/z [M+H]⁺ calcd for C₅₁H₆₇N₆O₁₁: 939.4862; found: 939.4780 (error 8.7 ppm).

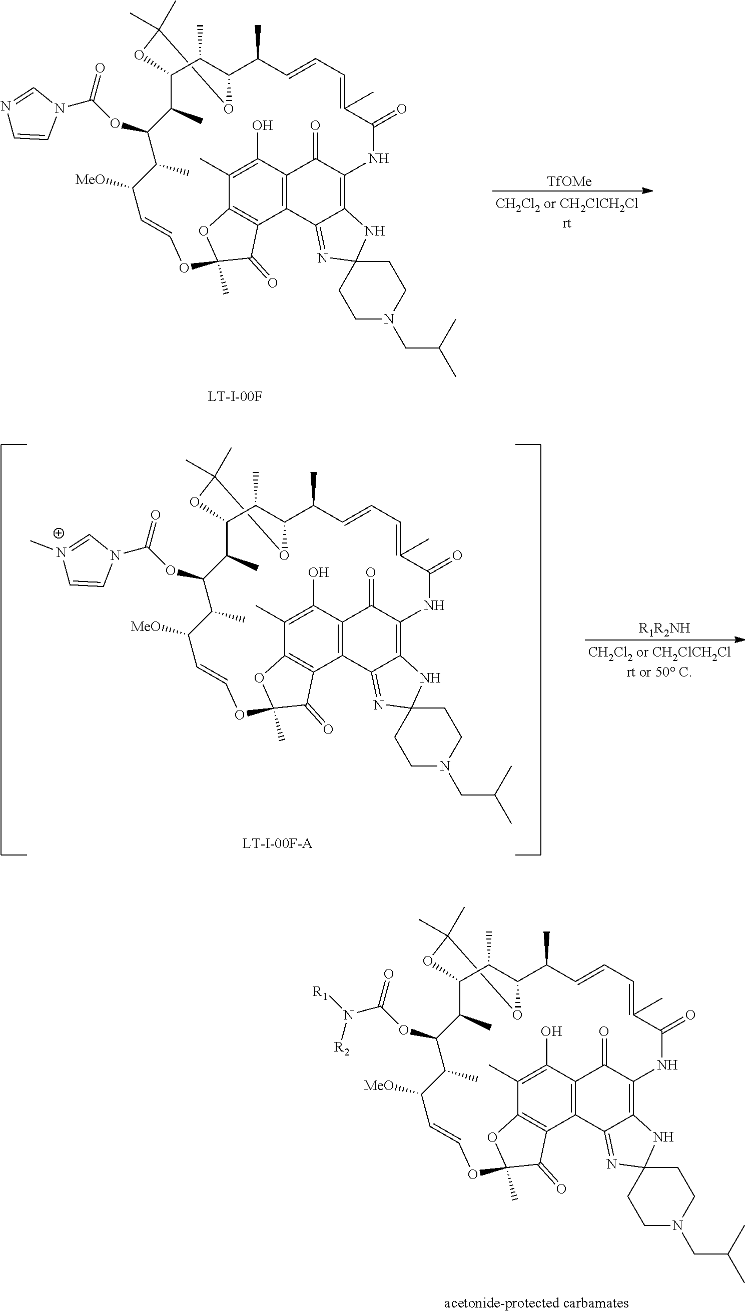

To a solution of LT-I-00F (1.00 equiv.) in dichloromethane or 1,2-dichloroethane (0.1 M), methyl trifluoromethanesulfonate (1.25-1.50 equiv.) was added. After the mixture was stirred for 10-15 min at room temperature, the activated intermediate LT-I-00F-A was concentrated under reduced pressure for further used. Alternatively, amines were added in the same pot and the reaction mixture was further stirred under room temperature until complete conversion of the starting material. Then the reaction mixture was partitioned between dichloromethane and H₂O. The aqueous phase was then back extracted with dichloromethane. The combined organic layers were concentrated under reduced pressure and the resulting crude was purified by preparative TLC (hexanes:ethyl acetate:methanol:triethylamine=8:1:1:0.1) or prep-HPLC to obtain the respective acetonide-protected carbamate analogs.

Direct synthesis of acetonide-protected carbamates (carbamate formation procedure B)

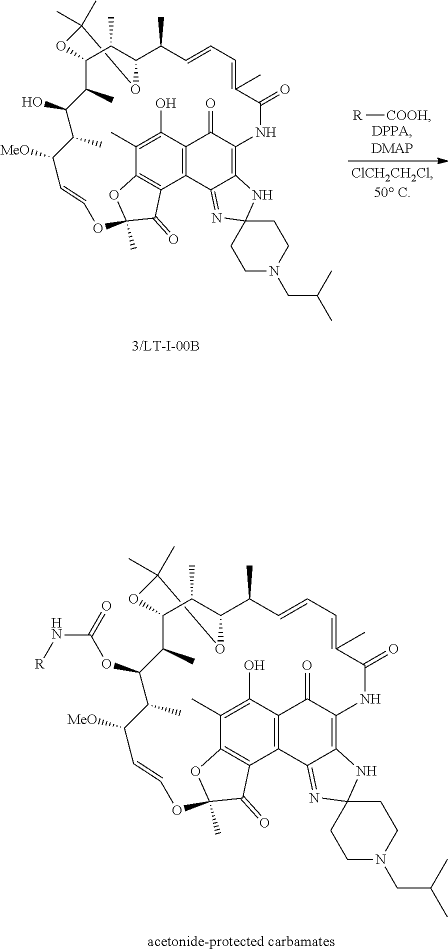

To a solution of the respective acid (5.00 equiv.) and DMAP (5.00 equiv.) in 1,2-dichloroethane (0.5 M to the acid), DPPA (5.00 equiv.) was added. After the mixture was stirred at 50° C. for 24 h, 3 (1.00 equiv.) was added and the mixture was further stirred at 50° C. until full consumption of 3. The reaction mixture was then partitioned between CH₂Cl₂ and H₂O. The aqueous phase was then back extracted twice with CH₂Cl₂. The combined organic layers were concentrated under reduced pressure and the resulting crude was purified by preparative TLC (hexanes:ethyl acetate:methanol:triethylamine=7:2:1:0.1) or flash chromatography (0-10% MeOH in CH₂Cl₂) to obtain the acetonide-protected 25-O-aminoacyl rifabutin intermediates.

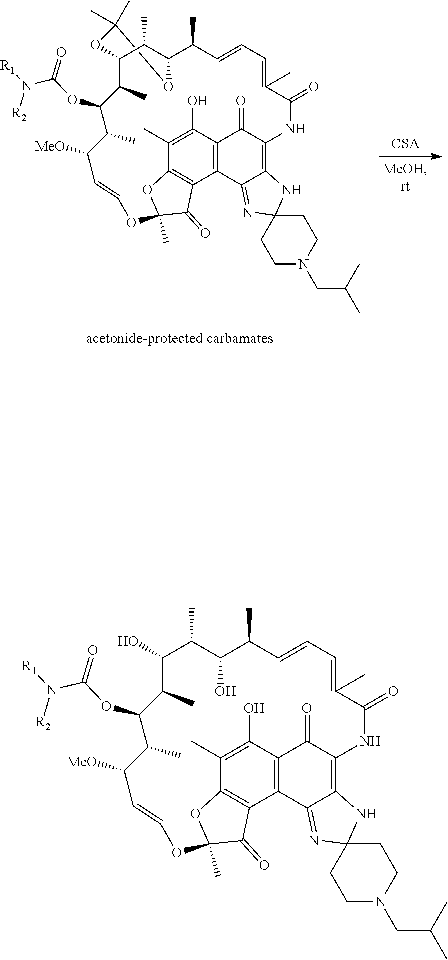

A solution of a 25-O-carbamoyl-21,23-isopropylidene rifabutin analog (1.00 equiv), and camphorsulfonic acid (2.00-5.00 equiv) in methanol (0.1 M) was stirred at room temperature for 30 min; then the reaction was partitioned between dichloromethane (10 mL) and saturated NaHCO3 aqueous solution (10 mL). The aqueous phase was then back extracted with dichloromethane (2×10 mL). The combined organic layers were dried (Na₂SO₄) and concentrated under reduced pressure. The resulting crude was purified by preparative TLC (hexane:ethyl acetate:methanol:triethylamine=8:1:1:0.1 or dichloromethane:methanol=15:1) or prep-HPLC to obtain 25-O-carbamoyl rifabutin analogs.

Note: for analogs with derivatization with basic heteroaromatic substituents, 5 equiv. CSA provided more efficient reactions; for the rest deprotection, 2 equiv. CSA was enough for full conversion within 30 minutes.

### Synthesis of 5i

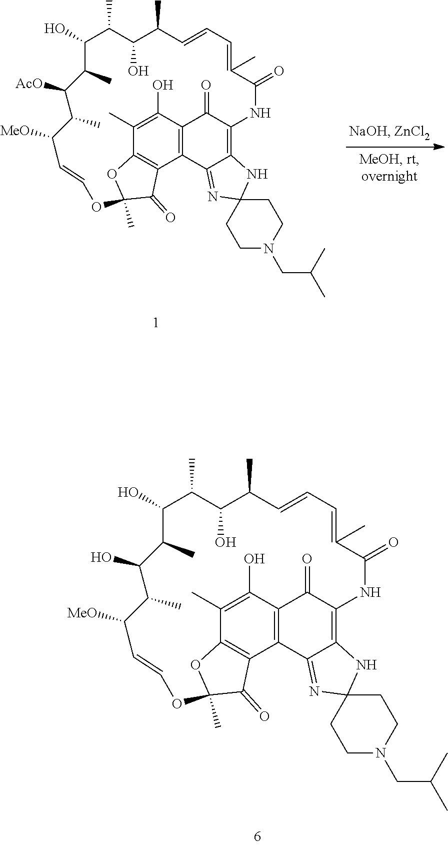 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(C)=O)[C@@H]1C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](O)[C@@H]1C)C4=O]

To a solution of NaOH (0.28 g, 6.00 equiv) and ZnCl₂ (0.16 g, 1.00 equiv) in MeOH (12 mL) was added 1 (1.00 g, 1.00 equiv), and the reaction mixture was stirred at room temperature overnight. Then the mixture was partitioned between CH₂Cl₂ (30 mL) and brine (30 mL). The aqueous phase was extracted with (CH₂Cl₂ (3×30 mL). The combined organic phases were dried (Na₂SO₄) and concentrated under reduced pressure. Purification by flash column chromatography (CH₂Cl₂:MeOH=9.1) afforded the titled compound (0.68 g, 71%); ¹H NMR (601 MHz, CDCl₃) δ 14.57 (s, 1H), 9.68 (s, 1H), 8.26 (s, 1H), 6.34 (d, J=12.8 Hz, 1H), 6.30-6.23 (m, 2H), 5.94-5.85 (m. 1H), 5.18 (dd, J=12.8, 10.1 Hz, 1H), 4.15 (s, 1H), 3.70 (d, J=9.8 Hz, 1H), 3.55 (ddd, J=10.3, 7.9, 2.4 Hz, 1H), 3.39 (dd, J=10.3, 4.2 Hz, 1H), 3.32 (dd, J=10.0, 2.1 Hz, 1H), 3.16 (s, 3H), 3.03-2.92 (m, 3H), 2.87 (dq, J=8.1, 2.6 Hz, 1H), 2.68 (br, 2H), 2.42 (dt, J=9.6, 6.6 Hz, 1H), 2.32 (s, 2H), 2.28 (s, 3H), 2.04 (s, 3H), 1.90-1.82 (m, 2H), 1.80-1.74 (m, 1H), 1.72 (s, 3H), 1.24 (s, 4H), 1.08 (d, J=7.0 Hz, 3H), 0.95 (d, J=6.5 Hz, 6H), 0.83 (d, J=7.0 Hz, 3H), 0.55 (d, J=6.8 Hz, 3H), −0.14 (d, J=7.0 Hz, 3H), ¹³C NMR (151 MHz, CDCl₃) δ 192.1, 180.5, 171.0, 168.3, 168.2, 155.0, 147.5, 141.6, 141.0, 132.9, 132.6, 124.9, 123.3, 114.7, 114.2, 111.5, 109.5, 108.1, 104.4, 94.8, 85.5, 76.8, 71.6, 70.8, 66.4, 56.1, 51.6, 51.6, 39.5, 38.9, 37.9, 36.1, 35.2, 32.8, 29.8, 25.9, 22.9, 21.0, 21.0, 20.2, 17.2, 12.2, 10.9, 8.4, 7.9; MS (ESI): m/z [M+Na]⁺ calcd for C₄₉H₆₅N₄O₁₁Na: 827.4202; found: 827.4198 (error 0.5 ppm).

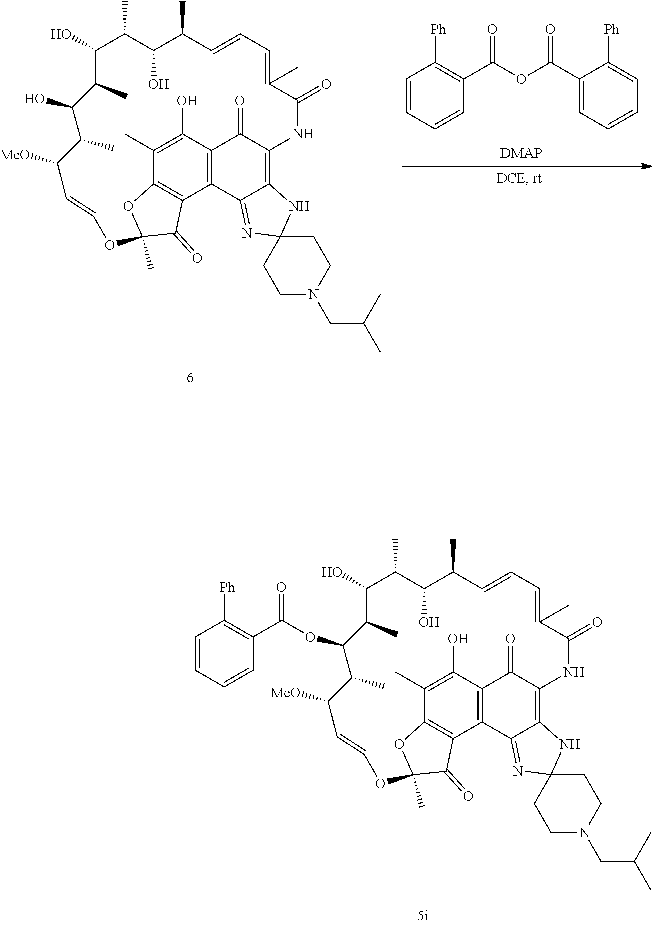 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](O)[C@@H]1C)C4=O ; O=C(OC(=O)c1ccccc1-c1ccccc1)c1ccccc1-c1ccccc1 ; CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)c2ccccc2-c2ccccc2)[C@@H]1C)C4=O]

Acylation procedure C. To a solution of 6 (1.00 equiv) in 1,2-dichloroethane (0.1 M) under room temperature, the acid anhydride (2.00 equiv) and DMAP (0.50 equiv) were added every 12 hours until completion of conversion. The reaction mixture was then concentrated to give a residue. The residue was purified by flash column chromatography (hexane:ethyl acetate:methanol=7:2:1) and then by Prep-TLC (hexane:ethyl acetate:methanol 7:2:1) to afford 25-O-acyl rifabutin analog 5i (see below for the full characterization).

### 25-O-benzoyl-25-O-desacetyrifabutin (5a)

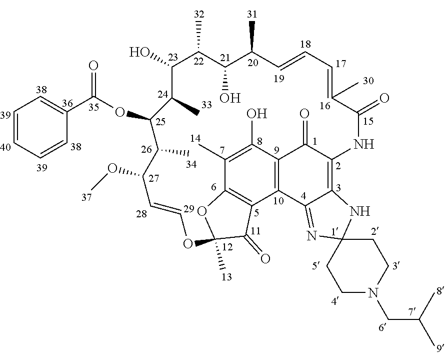 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)c2ccccc2)[C@@H]1C)C4=O]

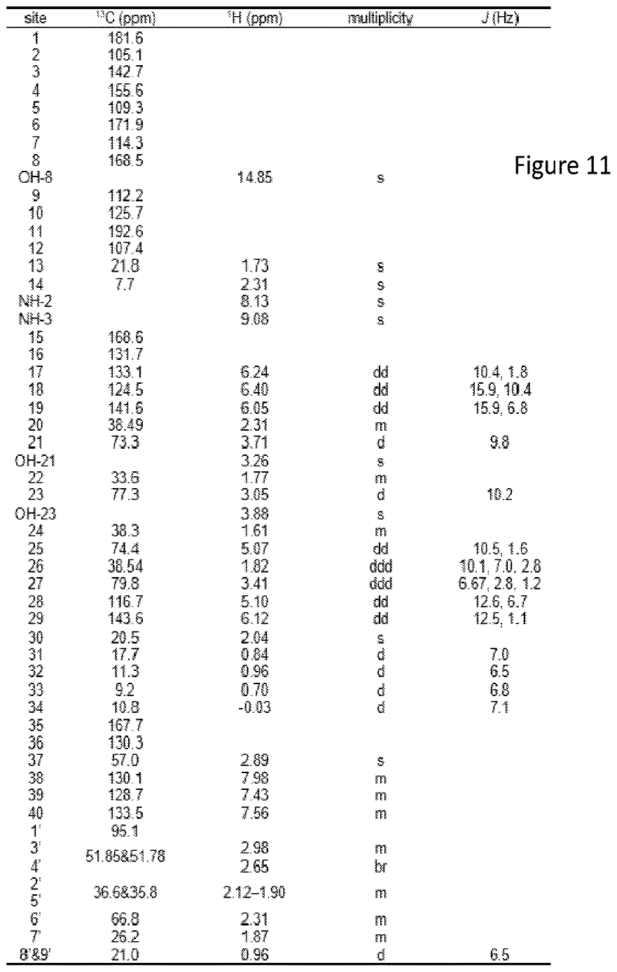

Prepared from 3 (50.0 mg) using acylation procedure A and the general deprotection procedure to afford the title compound (30.6 mg, 57% over two steps) as a purple solid. HRMS (ESI-TOF) m/z [M+H]⁺ calcd for C₅₁H₆₅N₄O₁₁ 909.4644, found 909.4665 (error 2.3 ppm). See FIG. 11 for ¹H NMR and ¹³C NMR data.

### 25-O-(2-methylbenzoyl)-25-O-desacetyrifabutin (5b)

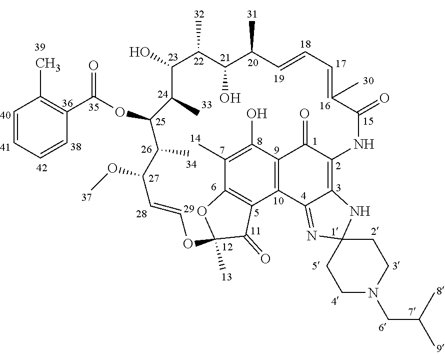 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)c2ccccc2C)[C@@H]1C)C4=O]

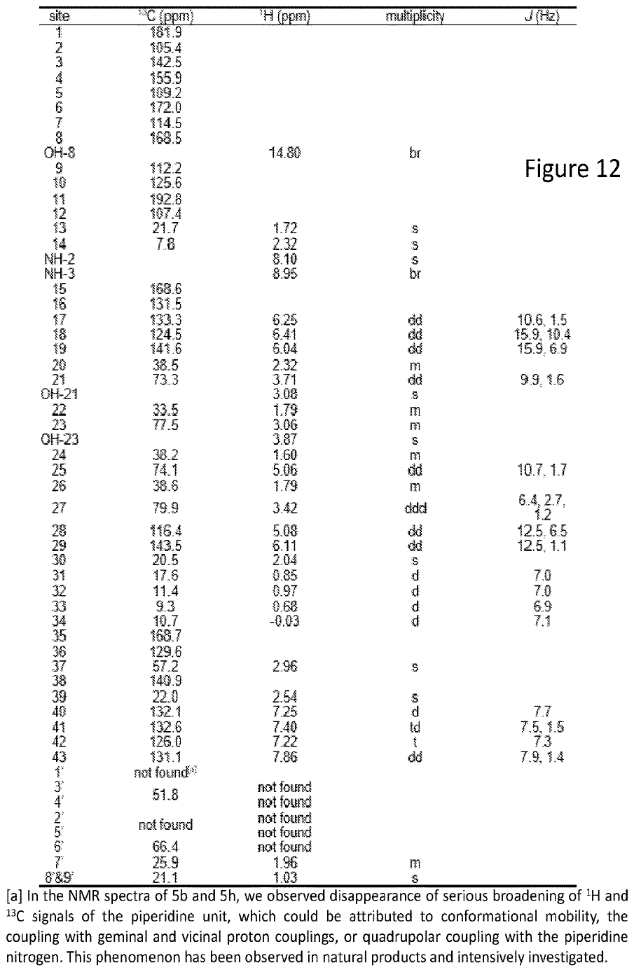

Prepared from 3 (30.0 mg) using acylation procedure A and the general deprotection procedure to afford 5b (3.9 mg, 12% over two steps) as a purple solid. HRMS (ESI-TOF) m/z [M+H]⁺ calcd for C₅₂H₆₇N₄O₁₁: 923.4801, found: 923.4829 (error 3.0 ppm). See FIG. 12 for ¹H NMR and ¹³C NMR data.

### 25-O-(3-methylbenzoyl)-25-O-desacetyrifabutin (5c)

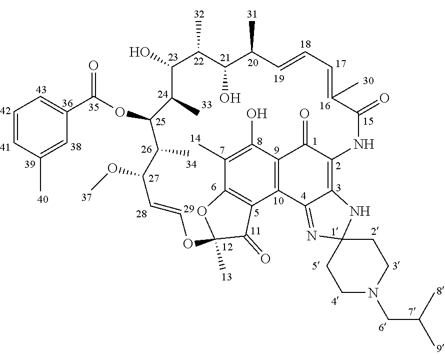 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)c2cccc(C)c2)[C@@H]1C)C4=O]

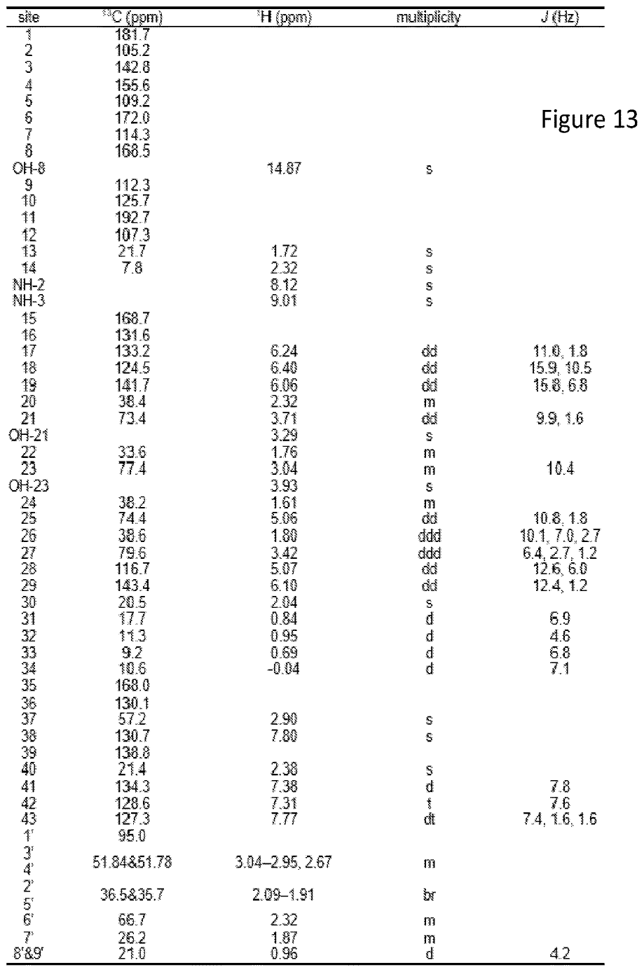

Prepared from 3 (25.0 mg) using acylation procedure A and the general deprotection procedure to afford 5c (2.5 mg, 9% over two steps) as a purple solid. HRMS (ESI-TOF) m/z [M+H]⁺ calcd for C₅₂H₆₇N₄O₁₁: 923.4801, found: 923.4815 (error 1.5 ppm). See FIG. 13 for ¹H NMR and ¹³C NMR data.

### 25-O-(4-methylbenzoyl)-25-O-desacetyrifabutin (5d)

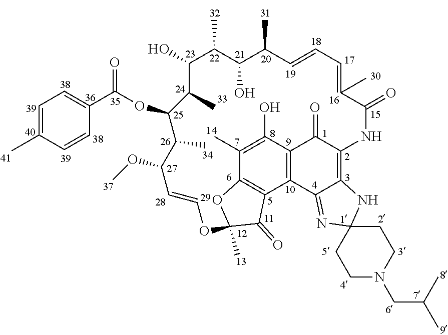 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)c2ccc(C)cc2)[C@@H]1C)C4=O]

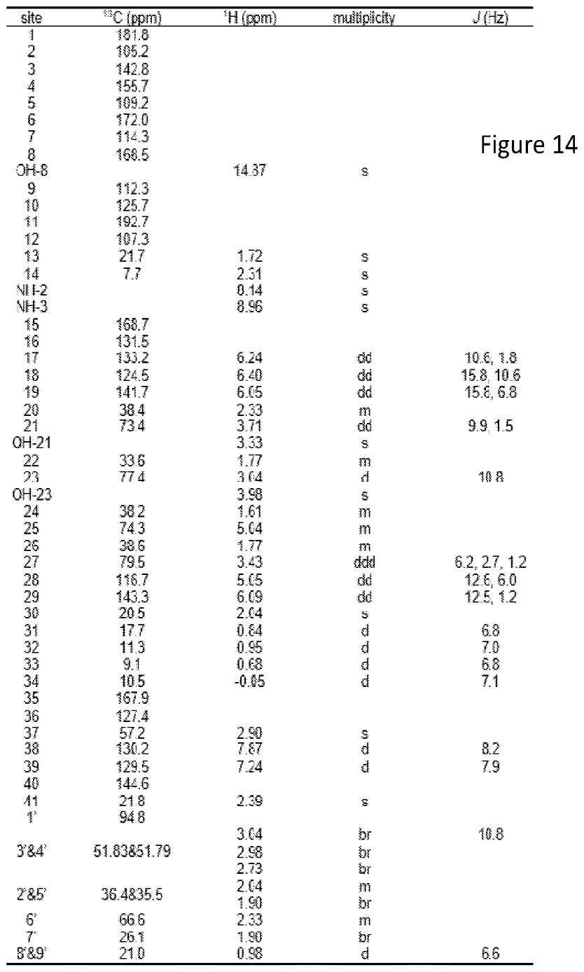

Prepared from 3 (18.9 mg) using acylation procedure A and the general deprotection procedure to afford 5d (8.1 mg, 39% over two steps) as a purple solid. HRMS (ESI-TOF) m/z [M+H)]⁺ calcd for C₅₂H₆₇N₄O₁₁: 923.4801, found: 923.4821 (error 2.2 ppm). See FIG. 14 for ¹H NMR and ¹³C NMR data.

### 25-O-(2-fluorobenzoyl)-25-O-desacetyrifabutin (5e)

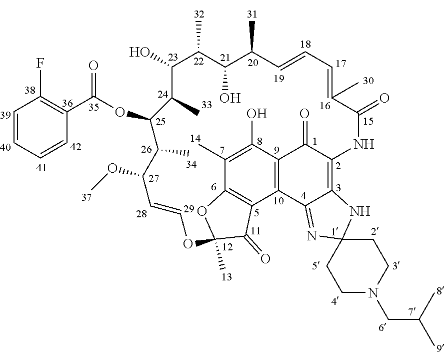 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)c2ccccc2F)[C@@H]1C)C4=O]

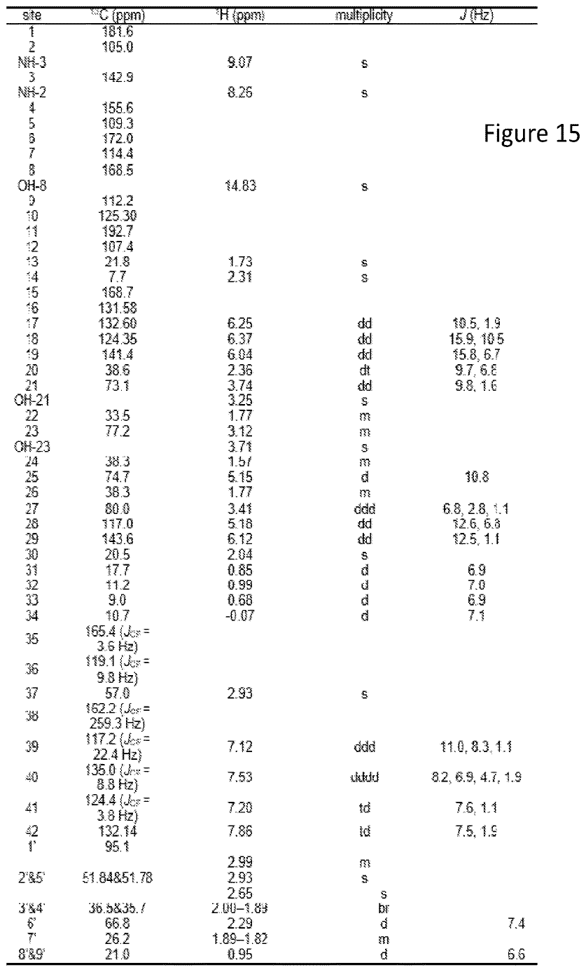

Prepared from 3 (30.0 mg) using acylation procedure A and the general deprotection procedure to afford 5e (17.5 mug, 53% over two steps) as a purple solid. HRMS (ESI-TOF) m/z [M+Na]⁻ calcd for C₅₁H₆₃FN₄O₁₁Na: 949.4370, found: 949.4341 (error 3.0 ppm). See FIG. 15 for ¹H NMR and ¹³C NMR data.

### 25-O-(2-chlorobenzoyl)-25-O-desacetyrifabutin (5f)

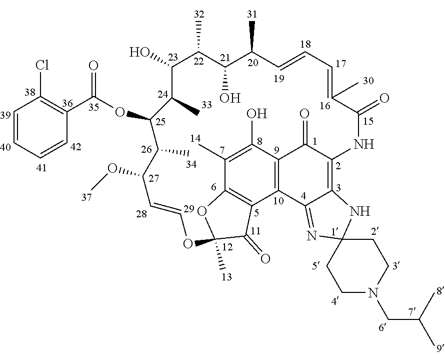 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)c2ccccc2Cl)[C@@H]1C)C4=O]

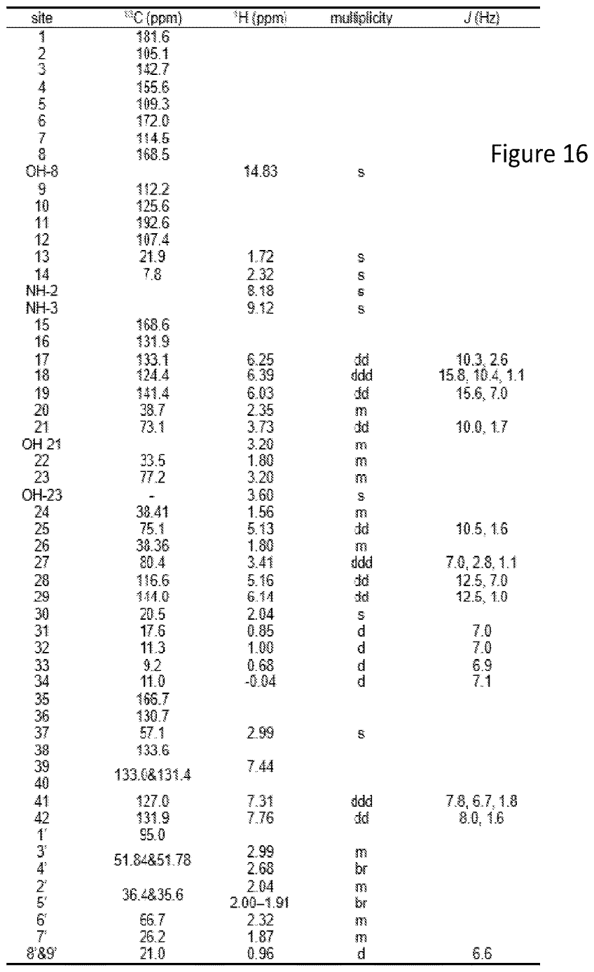

Prepared from 3 (25.0 mg) using acylation procedure A and the general deprotection procedure to afford 5f (19.0 mg, 68% over two steps) as a purple solid. HRMS (ESI-TOF) m/z [M+H]⁺ calcd for C₅₁H₆₄ClN₄O₁₁: 943.4255, found: 943.4245 (error 1.0 ppm). See FIG. 16 for ¹H NMR and ¹³C NMR data.

### 25-O-(2-methoxybenzoyl)-25-O-desacetyrifabutin (5g)

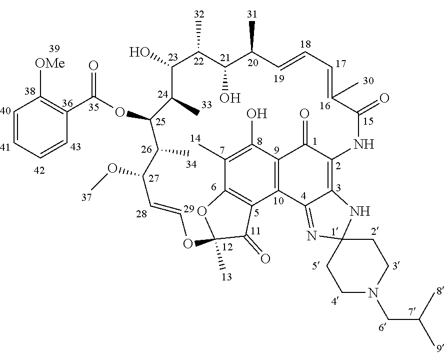 [SMILES: COc1ccccc1C(=O)O[C@H]1[C@H](C)[C@H](O)[C@H](C)[C@@H](O)[C@@H](C)/C=C/C=C(\C)C(=O)NC2=C3NC4(CCN(CC(C)C)CC4)N=C3c3c(c(O)c(C)c4c3C(=O)[C@@](C)(O/C=C/[C@H](OC)[C@H]1C)O4)C2=O]

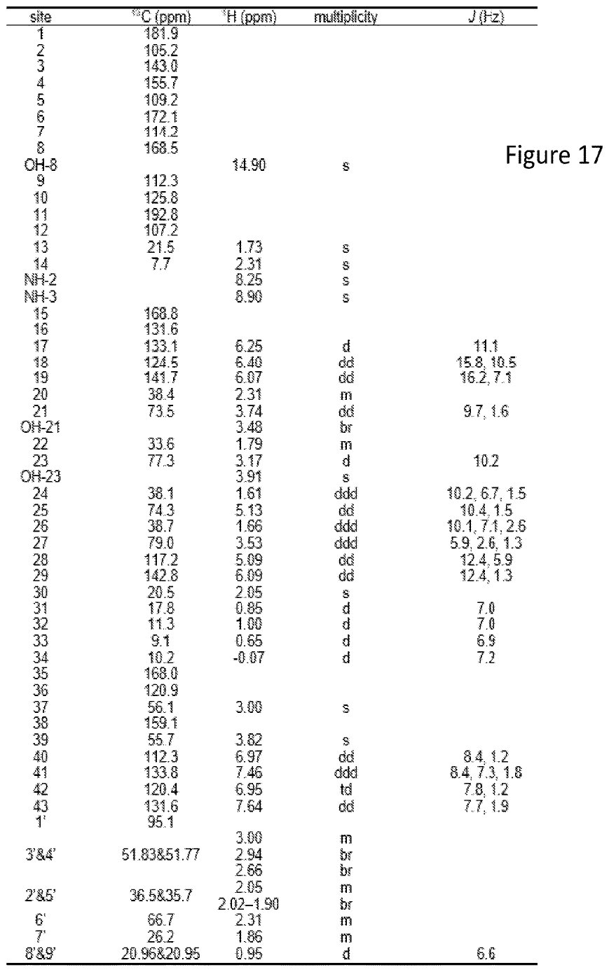

Prepared from 3 (600 mg) using acylation procedure A and the general deprotection procedure to afford 5g (116.2 mg, 26% over two steps) as a purple solid. HRMS (ESI-TOF) m/z [M+H]⁺ calcd for C₅₂H₆₇N₄O₁₂: 939.4750, found: 939.4777 (error 2.9 ppm). See FIG. 17 for ¹H NMR and ¹³C NMR data.

### 25-O-(2-trifluoromethylbenzoyl)-25-O-desacetyrifabutin (5h)

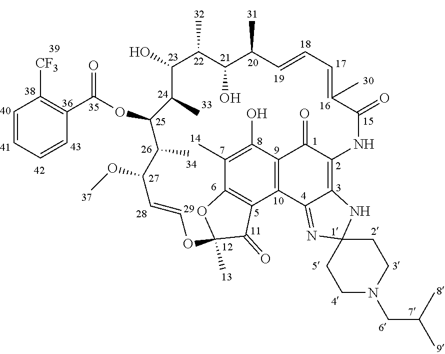 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)c2ccccc2C(F)(F)F)[C@@H]1C)C4=O]

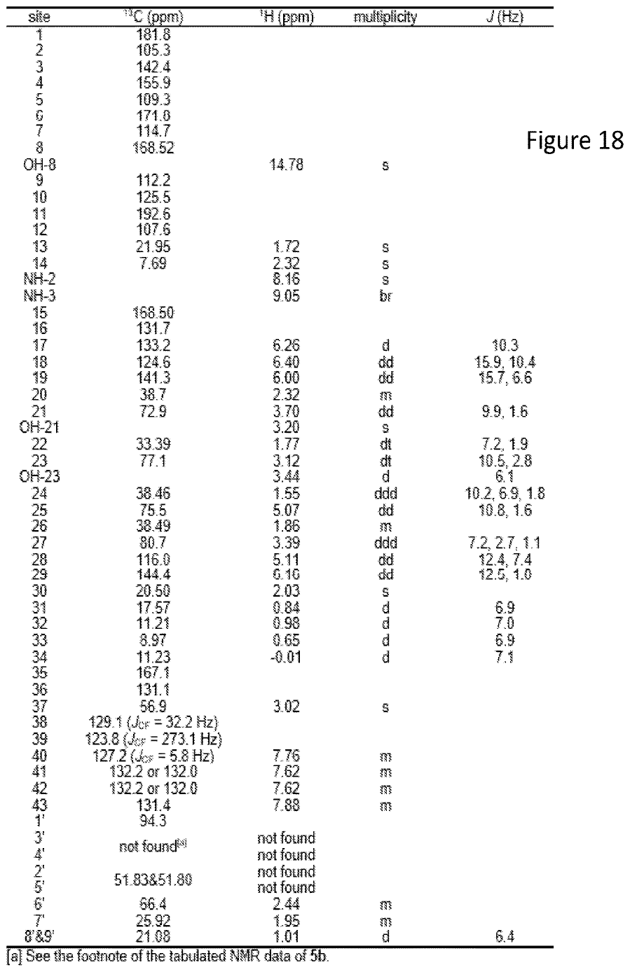

Prepared from 3 (30.0 mg) using acylation procedure A and the general deprotection procedure to afford 5h (10.4 mg, 30% over two steps) as a purple solid. HRMS (ESI-TOF) m/z [M+H]⁺ calcd for C₅₂H₆₄F₃N₄O₁₁: 977.4518, found: 977.4519 (error 0.1 ppm). See FIG. 18 for ¹H NMR and ¹³C NMR data.

### 25-O-(2-phenylbenzoyl)-25-O-desacetyrifabutin (5i)

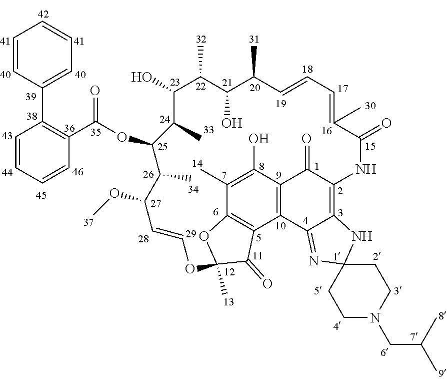 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)c2ccccc2-c2ccccc2)[C@@H]1C)C4=O]

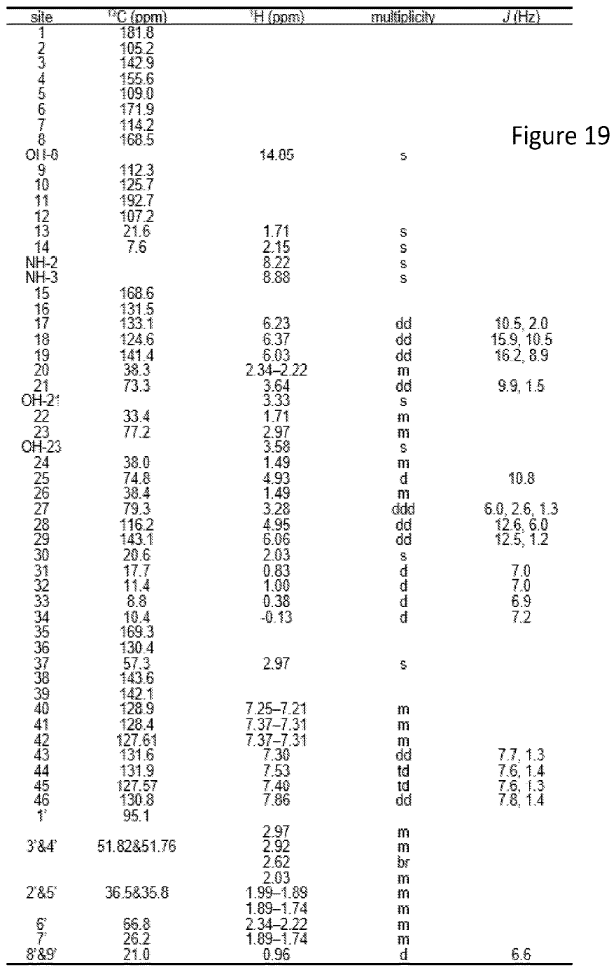

Prepared from 6 (415.5 mg) using acylation procedure C to afford 5i (29.8 mg, 6%) as a purple solid. HRMS (ESI-TOF) m/z [M+H]⁺ calcd for CH₅₇H₆₉N₄O₁₁: 985.4957, found: 985.4952 (error 0.5 ppm). See FIG. 19 for ¹H NMR and ¹³C NMR data.

### 25-O-(3-fluorobenzoyl)-25-O-desacetyrifabutin (5j)

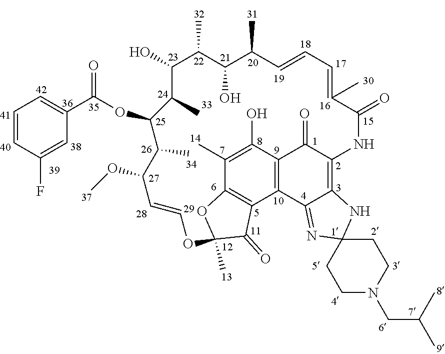 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)c2cccc(F)c2)[C@@H]1C)C4=O]

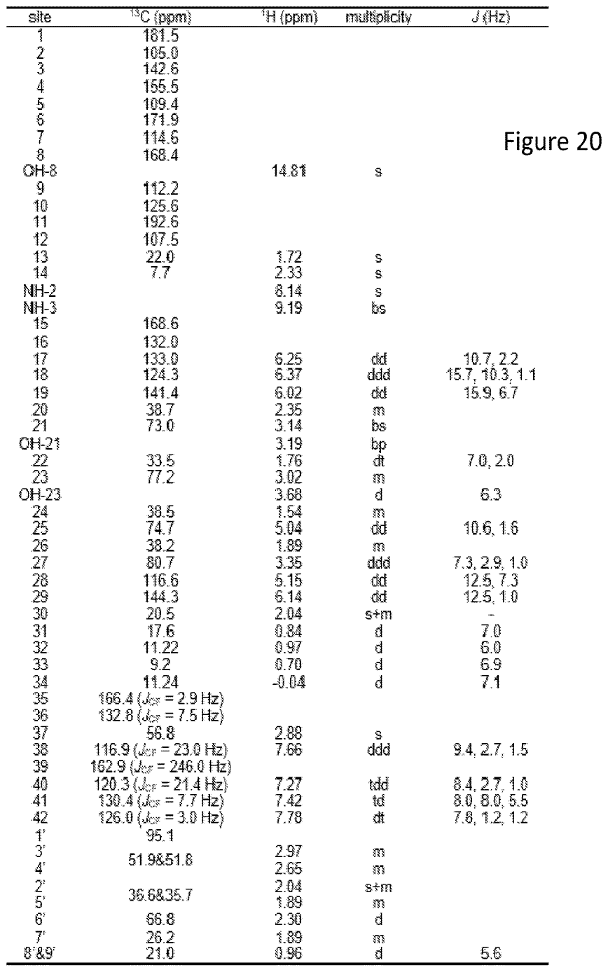

Prepared from 3 (20.0 mg) using acylation procedure A and the general deprotection procedure to afford 5j (12.0 mg, 55% over two steps) as a purple solid. HRMS (ESI-TOF) m/z [M+H]⁺ calcd for C₄₉H₆₉N₄O₁₁: 927.4550, found: 927.4556 (error 0.6 ppm). See FIG. 20 for ¹H NMR and ¹³C NMR data.

### 25-O-(2-methylbutyl)-25-O-desacetyrifabutin (5k)

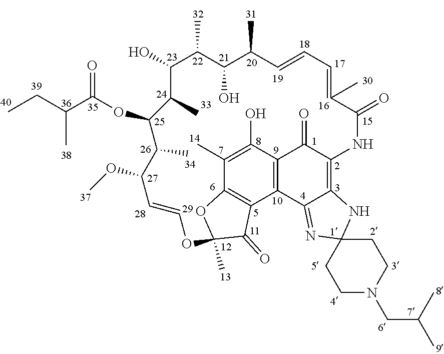 [SMILES: CCC(C)C(=O)O[C@H]1[C@H](C)[C@H](O)[C@H](C)[C@@H](O)[C@@H](C)/C=C/C=C(\C)C(=O)NC2=C3NC4(CCN(CC(C)C)CC4)N=C3c3c(c(O)c(C)c4c3C(=O)[C@@](C)(O/C=C/[C@H](OC)[C@H]1C)O4)C2=O]

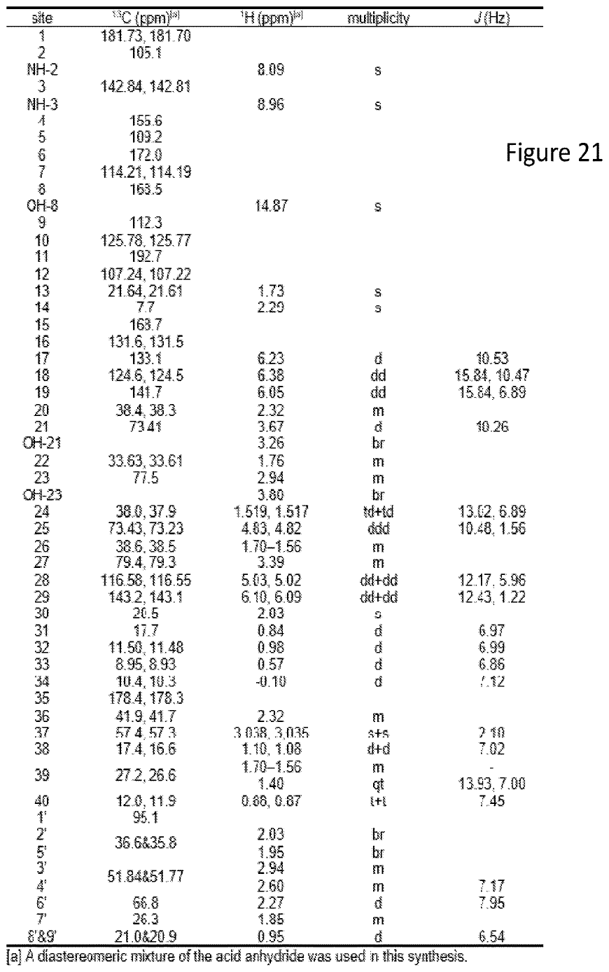

Prepared from 3 (32.8 mg) using acylation procedure A and the general deprotection procedure to afford 5k (22.2 mg, 64% over two steps) as a purple solid. HRMS (ESI-TOF) m/z [M+H]⁺ calcd for C₄₉H₆₉N₄O₁₁: 889.4957, found: 889.4995 (error 4.3 ppm). See FIG. 21 for ¹H NMR and ¹³C NMR data.

### 25-O-(2-ethylbutyl)-25-O-desacetyrifabutin (5l)

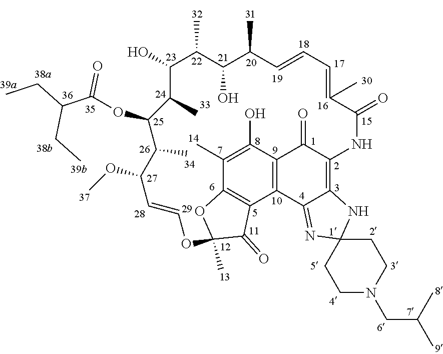 [SMILES: CCC(CC)C(=O)O[C@H]1[C@H](C)[C@H](O)[C@H](C)[C@@H](O)[C@@H](C)/C=C/C=C(\C)C(=O)NC2=C3NC4(CCN(CC(C)C)CC4)N=C3c3c(c(O)c(C)c4c3C(=O)[C@@](C)(O/C=C/[C@H](OC)[C@H]1C)O4)C2=O]

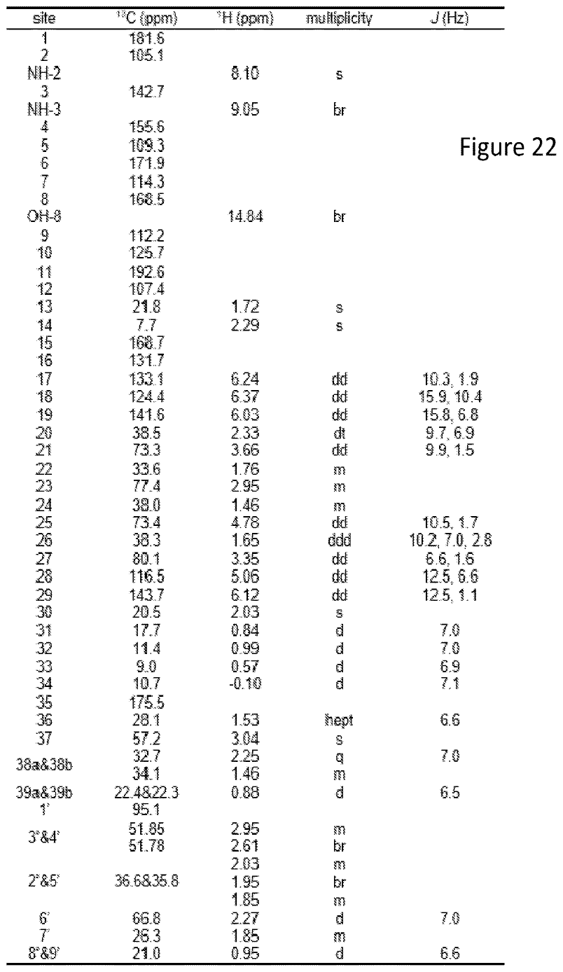

Prepared from 3 (20.9 mg) using acylation procedure A and the general deprotection procedure to deliver 5l (16.0 mg, 72% over two steps) as a purple solid. HRMS (ESI-TOF) m/z [M+H]⁺ calcd for C₅₀H₇₁N₄O₁₁: 903.5114, found: 903.5105 (error 1.0 ppm). See FIG. 22 for ¹H NMR and ¹³C NMR data.

### 25-O-(3-pyridinecarbonyl)-25-O-desacetyrifabutin (5m)

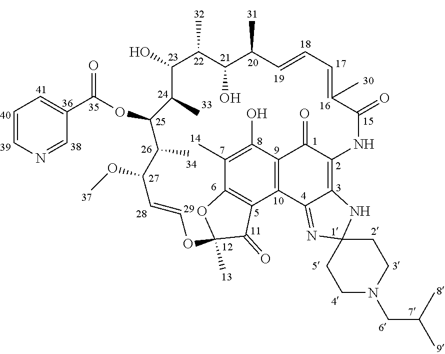 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)c2cccnc2)[C@@H]1C)C4=O]

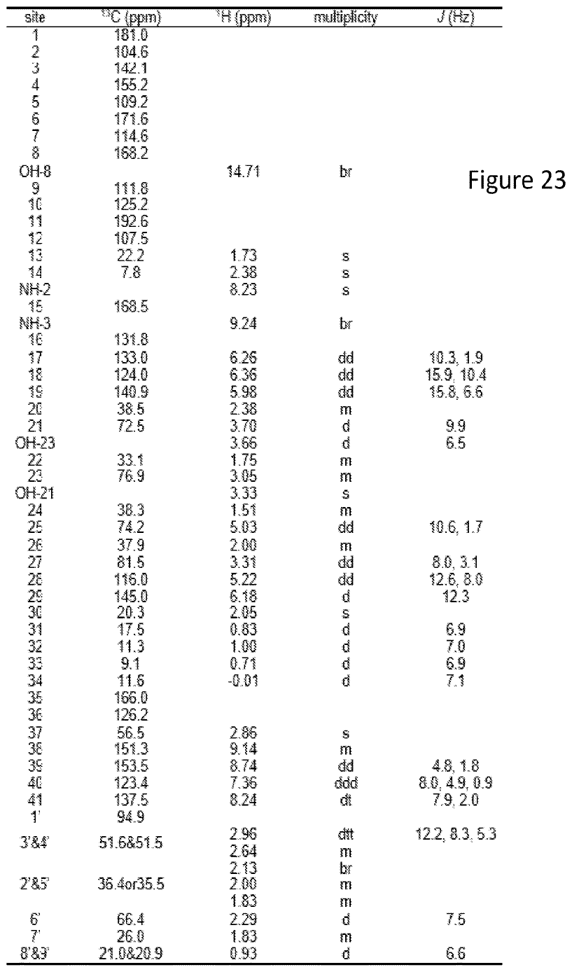

Prepared from 3 (59.7 mg) using acylation procedure A and the general deprotection procedure to afford 5m (29.3 mg, 46% over two steps) as a purple solid. HRMS (ESI-TOF) m/z [M+H]⁺ calcd for C₅₀H₆₄N₅O₁₁: 910.4597, found: 910.4606 (error 1.0 ppm). See FIG. 23 for ¹H NMR and ¹³C NMR data.

### 25-O-(5-pyrimidinecarbonyl)-25-O-desacetyrifabutin (5n)

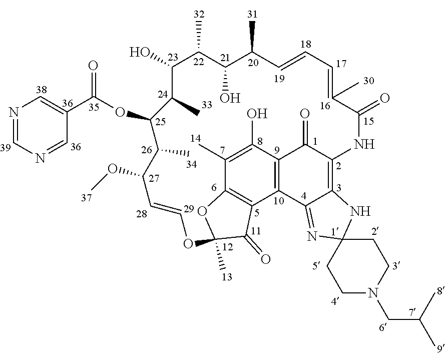 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)c2cncnc2)[C@@H]1C)C4=O]

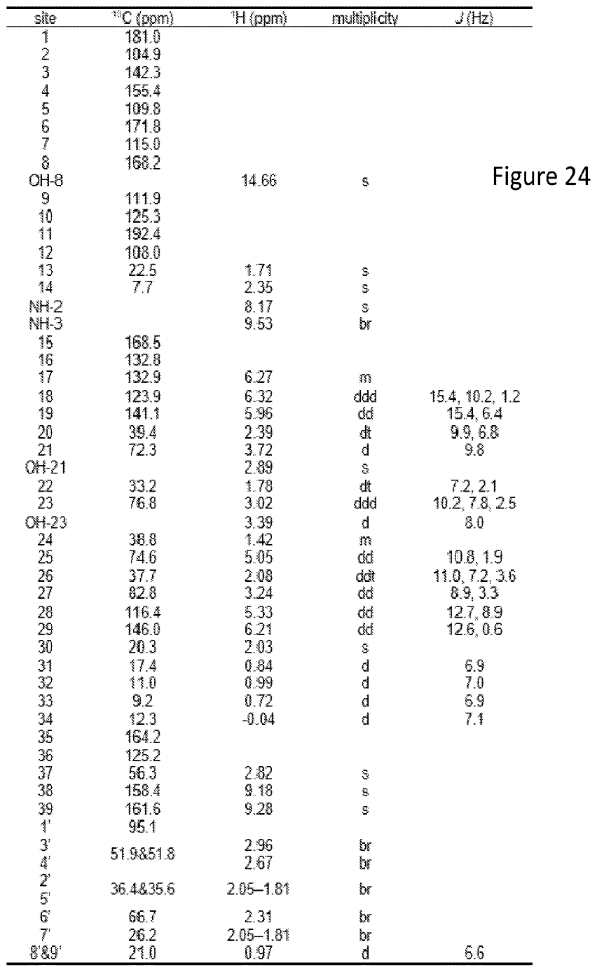

Prepared from 3 (40.0 mg) using acylation procedure B and the general deprotection procedure to afford 5n (29.3 mg, 56% over two steps) as a purple solid. HRMS (ESI-TOF) m/z [M+Na]⁻ calcd for C₄₉H₆₂N₆O₁₁Na: 933.4369, found: 933.4395 (error 2.8 ppm). See FIG. 24 for ¹H NMR and ¹³C NMR data.

### 25-O-(2-thiazolecarbonyl)-25-O-desacetyrifabutin (5o)

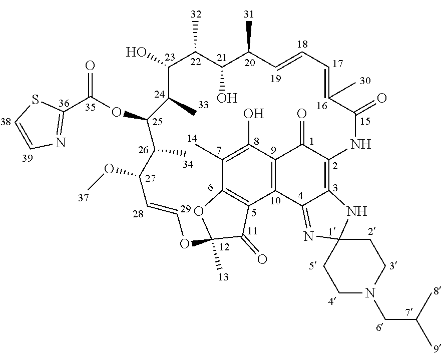 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)c2nccs2)[C@@H]1C)C4=O]

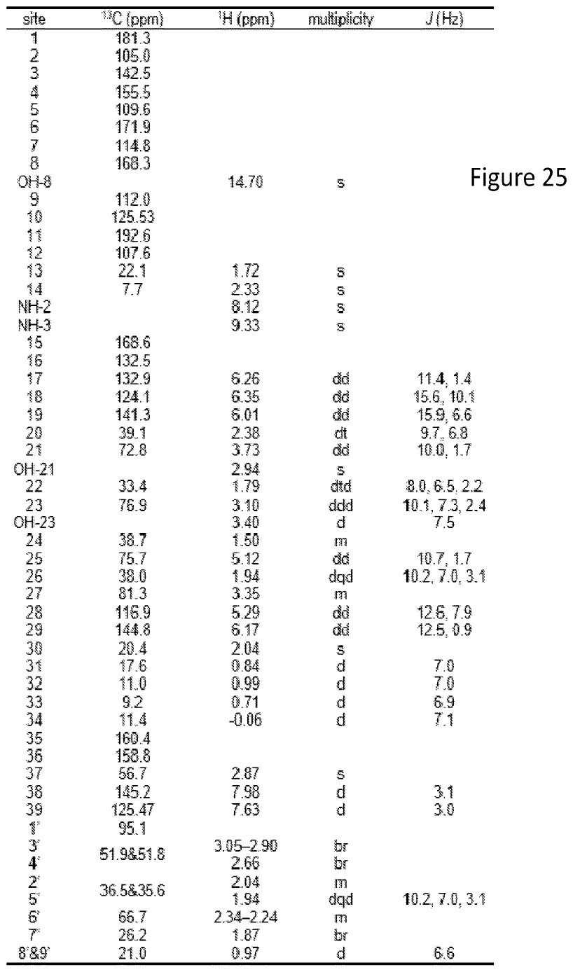

Prepared from 3 (40.0 mg) using acylation procedure B and the general deprotection procedure to afford 5o (29.3 mg, 51% over two steps) as a purple solid. HRMS (ESI-TOF) m/z [M+H]⁺ calcd for C₄₈H₆₂N₅O₁₁S: 916.4161, found: 916.4204 (error 4.7 ppm). See FIG. 25 for ¹H NMR and ¹³C NMR data.

### Notes on the Acylation Process of the Compound Synthesis

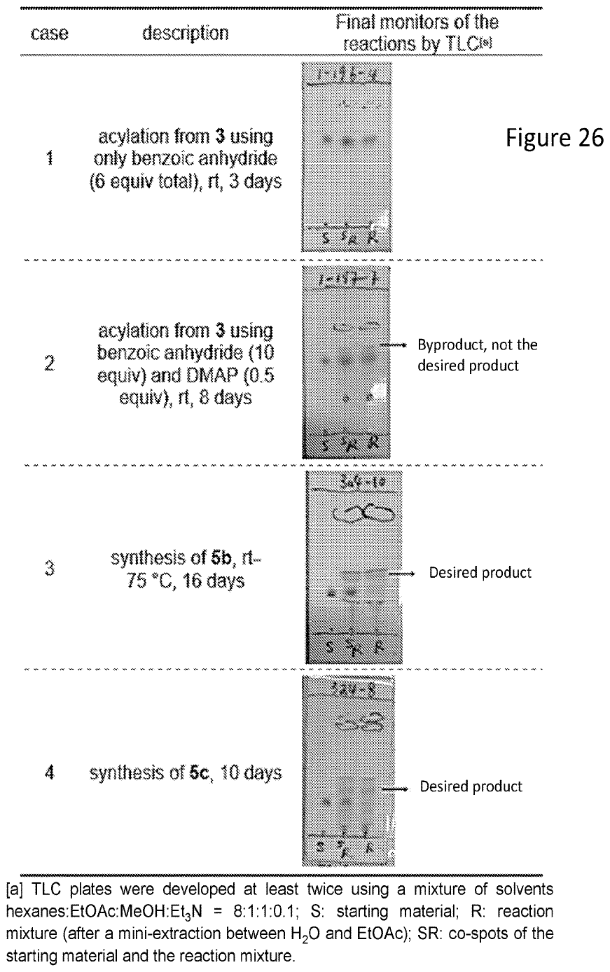

The acylation procedure reported above using excessive amounts of anhydrides and DMAP was the only method we found out that could afford the desired C25-OH acylation products. Under the conditions of using anhydride along or with substoichiometric amount of DMAP, the starting material had almost no conversion (see FIG. 26, Case 1 and Case 2). This method was proved effective for most of our synthesis with reasonable to high yield. In certain cases, the relatively low yield was due to extremely slow conversion and, once heated up, the massive side reactions (see FIG. 26, Case 3 and Case 4).

### 25-O-(3-methoxylbenzoyl)-25-O-desacetyrifabutin

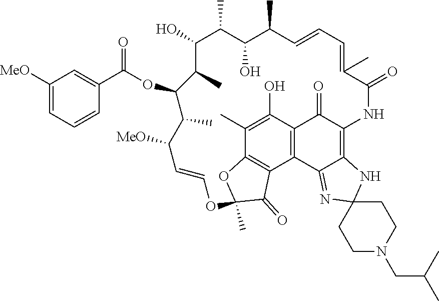 [SMILES: COc1cccc(C(=O)O[C@H]2[C@H](C)[C@H](O)[C@H](C)[C@@H](O)[C@@H](C)/C=C/C=C(\C)C(=O)NC3=C4NC5(CCN(CC(C)C)CC5)N=C4c4c(c(O)c(C)c5c4C(=O)[C@@](C)(O/C=C/[C@H](OC)[C@H]2C)O5)C3=O)c1]

Prepared from 3 (25.7 mg) using acylation procedure A and the general deprotection procedure to afford the title compound (15.0 mg, 52% over two steps) as a purple solid. ¹H NMR (601 MHz, CD₂Cl₂) δ 14.87 (s, 1H), 9.01 (s, 1H), 8.21 (s, 1H), 7.56 (dt, J: 7.6, 1.2 Hz, 1H), 7.50 (dd, J=2.7, 1.5 Hz, 1H), 7.34 (t, J=8.0 Hz, 1H), 7.10 (ddd, J=8.3, 2.7, 1.0 Hz, 1H), 6.39 (dd, J=15.8, 10.6 Hz, 1H), 6.24 (dd, J=10.4, 2.5 Hz, 1H), 6.10 (dd, J=12.5, 1.2 Hz, 1H), 6.05 (dd, J=15.9, 6.8 Hz, 1H), 5.09 (dd, J=12.5, 6.4 Hz, 1H), 5.07 (dd, J=9.2, 1.5 Hz, 1H), 3.90 (br, 1H), 3.82 (s, 3H), 3.72 (dd, J=9.9, 1.8 Hz, 1H), 3.42 (ddd, J=: 6.4, 2.7, 1.2 Hz, 1H), 3.31 (br, 11H), 3.05 (d, J=10.3 Hz, 1H), 3.03-2.98 (m, 1H), 2.97-2.92 (m, 1H), 2.91 (s, 3H), 2.67 (br, 2H), 2.36-2.32 (m, 1H), 2.31 (s, 3H), 2.30 (s, 2H), 2.04 (s, 3H), 2.14-1.90 (m, 4H), 1.88 (m, 1H), 1.80 (ddd, J=10.3, 7.1, 2.8 Hz, 1H), 1.78-1.74 (m, 1H), 1.73 (s, 3H), 1.60 (ddd, J=10.3, 6.9, 1.7 Hz, 1H), 0.96 (d, J=6.4 Hz, 9H), 0.84 (d, J=7.0 Hz, 3H), 0.69 (d, J=6.9 Hz, 3H), −0.05 (d, J=7.1 Hz, 3H); ¹³C NMR (151 MHz, CD₂Cl₂) δ 192.7, 181.7.172.0, 168.6, 168.5, 167.6, 160.0, 155.6, 143.4, 142.9, 141.5, 133.1, 131.7, 131.6, 129.8, 125.7, 124.5, 122.4, 119.5, 116.8, 115.0, 114.3, 112.2, 109.2, 107.3, 105.1, 95.1, 79.6, 77.3, 74.6, 73.3, 66.7, 57.1, 55.8, 51.8, 51.8, 38.5, 38.4, 38.2, 36.5, 35.7, 33.5, 26.2, 21.7, 21.0, 20.5, 17.7, 11.3, 10.6, 9.1, 7.7; HRMS (ESI-TOF) m/z [M+H]⁺ calcd for C₅₂H₆₇N₄O₁₂ 939.4750, found 939.4772 (error 2.3 ppm)

### 25-O-(4-methoxylbenzoyl)-25-O-desacetyrifabutin

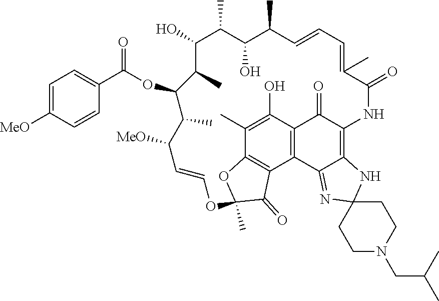 [SMILES: COc1ccc(C(=O)O[C@H]2[C@H](C)[C@H](O)[C@H](C)[C@@H](O)[C@@H](C)/C=C/C=C(\C)C(=O)NC3=C4NC5(CCN(CC(C)C)CC5)N=C4c4c(c(O)c(C)c5c4C(=O)[C@@](C)(O/C=C/[C@H](OC)[C@H]2C)O5)C3=O)cc1]

Prepared from 3 (65.2 mg) using acylation procedure A and the general deprotection procedure to afford the title compound (5.2 mg, 7% over two steps) as a purple solid. ¹H NMR (601 MHz, CD₂Cl₂) δ 14.89 (s, 1H), 8.99 (s, 1H), 8.11 (s, 1H), 7.97-7.91 (m, 2H), 6.91 (d, J=8.9 Hz, 1H), 6.40 (dd, J=15.9, 10.5 Hz, 1H), 6.24 (dd, J=10.8, 1.3 Hz, 1H), 6.09 (dd, J=12.4, 1.2 Hz, 1H), 6.06 (dd, J=15.8, 6.9 Hz, 1H), 5.04 (dd, J=12.5, 6.2 Hz, 1H), 5.01 (dd, J=10.5, 1.5 Hz, 1H), 4.07-4.03 (br, 1H), 3.84 (s, 3H), 3.70 (dd, J=9.9, 1.5 Hz, 1H), 3.43 (ddd, J=6.3, 2.7, 1.3 Hz, 1H), 3.33 (br, 1H), 3.06-2.99 (m, 1H), 3.04-2.92 (m, 2H), 2.90 (s, 3H), 2.71-2.60 (br, 2H), 2.31 (s, 3H), 2.35-2.26 (m, 3H), 2.04 (s, 3H), 2.08-1.90 (m, 4H), 1.90-1.82 (m, 1H), 1.82-1.73 (m, 3H), 1.72 (s, 3H), 1.61 (ddd, J=10.1, 6.7, 1.6 Hz, 1H), 0.96 (d, J=3.4 Hz, 5H), 0.95 (d, J=3.9 Hz, 5H), 0.83 (d, J=6.9 Hz, 3H), 0.67 (d, J=6.9 Hz, 3H), −0.04 (d, J=7.1 Hz, 3H); ¹³C NMR (151 MHz, CD₂Cl₂) δ 192.7, 181.7, 172.0, 168.7, 168.5, 167.7, 164.1, 155.7, 143.8, 142.8, 141.7, 133.2, 132.3, 131.5, 125.8, 124.5, 122.3, 116.7, 114.3, 114.0, 112.3, 109.2, 107.3, 105.2, 95.0, 79.5, 77.4, 74.2, 73.5, 66.7, 57.2, 55.9, 51.8, 51.8, 38.6, 38.3, 38.1, 36.6, 35.8, 33.6, 30.5, 26.2, 21.7, 21.0, 20.5, 17.7, 11.4, 10.6, 9.1, 7.7; HRMS (ESI-TOF) m/z [M+H]⁺ calcd for C₅₂H₆₇N₄O₁₂ 939.4750, found 939.4756 (error 0.6 ppm).

### 25-O-(4-pyrimidinecarbonyl)-25-O-desacetyrifabutin

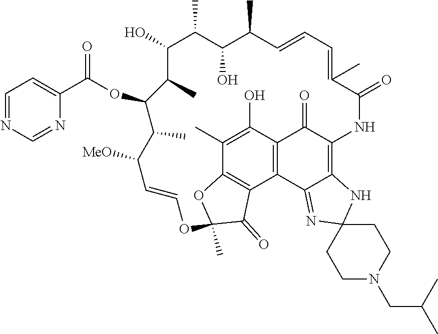 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)c2ccncn2)[C@@H]1C)C4=O]

Prepared from 3 (50.0 mg) using acylation procedure B and the general deprotection procedure to afford the title compound (36.2 mg, 67% over two steps) as a purple solid. ¹H NMR (601 M Hz, CD₂Cl₂) δ 14.68 (s, 1H), 9.45 (s, 1H), 9.31 (d, J=1.4 Hz, 1H), 8.91 (d, J=5.0 Hz, 1H), 8.18 (s, 1H), 7.89 (dd, J=5.0, 1.4 Hz, 1H), 6.33 (dd, J=15.4, 10.1 Hz, 1H), 6.26 (dd, J=10.3, 1.7 Hz, 1H), 6.19 (d, J=12.7 Hz, 1H), 5.99 (dd, J=15.7, 6.4 Hz, 1H), 5.34 (dd, J=12.7, 8.6 Hz, 1H), 5.12 (dd, J=10.7, 1.8 Hz, 1H), 3.73 (d, J=9.9 Hz, 1H) 3.39 (d, J=7.8 Hz, 1H), 3.29 (dd, J=8.5, 3.2 Hz, 1H), 3.07 (ddd, J=10.2, 7.6, 2.5 Hz, 1H), 3.00-2.90 (m, 3H), 2.82 (s, 3H), 2.65 (br, 2H) 2.42-2.36 (m, 1H), 2.34 (s, 3H), 2.29 (d, J=7.4 Hz, 2H), 2.11-1.94 (m, 4H), 2.04 (s, 3H), 1.95-1.88 (m, 1H), 1.88-1.81 (m, 1H), 1.79 (dtd, J=9.3, 6.7, 2.1 Hz, 1 Hz, 1.76-1.73 (m, 1H), 1.72 (s, 3H), 1.50-1.40 (m, 1H), 0.99 (d, J=7.0 Hz, 3H), 0.95 (d, J=6.6 Hz, 7H), 0.84 (d, J=7.0 Hz, 3H), 0.72 (d, J=6.9 Hz, 3H), −0.06 (d, J=7.1 Hz, 3H); ¹³C NMR (151 MHz, CD₂Cl₂) δ 192.5, 181.1, 171.8, 168.6, 168.3, 164.4, 159.5, 159.2, 155.8, 155.4, 145.5, 142.5, 141.1, 132.9, 132.8, 125.4, 124.0, 121.4, 116.8, 115.0, 112.0, 109.7, 107.8, 104.9, 95.2, 82.2, 76.8, 75.4, 72.5, 66.8, 56.4, 51.9, 51.8, 39.2, 38.8, 37.8, 36.5, 35.6, 33.3, 26.3, 22.3, 21.0, 20.3, 17.5, 11.9, 11.0, 9.1, 7.7; MS (ESI) m/z [M+H]³⁰ 911.4, [M−H]⁻ 909.3.

### 25-O-(2-pyrazinecarbonyl)-25-O-desacetyrifabutin

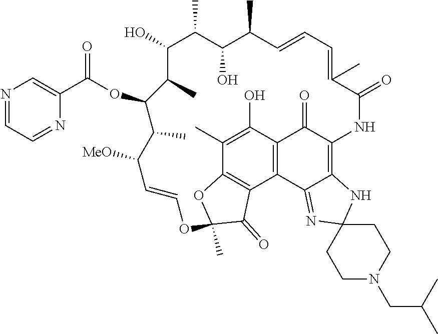 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)c2cnccn2)[C@@H]1C)C4=O]

Prepared from 3 (50.0 mg) using acylation procedure B and the general deprotection procedure to afford the title compound (36.1 mg, 67% over two steps) as a purple solid. ¹H NMR (601 MHz, CD₂Cl₂) δ 14.72 (s, 1H), 9.36 (s, 1H), 9.17 (d, J=1.5 Hz, 1H), 8.69 (d, J=2.5 Hz, 1H), 8.67 (dd, J=2.4, 1.5 Hz, 1H), 8.19 (s, 1H), 6.35 (dd, J=15.8, 10.3 Hz, 1H), 6.26 (dd, J=10.4, 1.7 Hz, 1H), 6.18 (dd, J=12.6, 0.8 Hz, 1H), 6.00 (dd, J=15.8, 6.5 Hz, 1H), 5.30 (dd, J=12.3, 7.8 Hz, 1H), 5.15 (dd, J=10.7, 1.8 Hz, 1H), 3.73 (d, J=9.9 Hz, 1H), 3.46 (d. J=7.4 Hz, 1H), 3.33 (dd, J=8.0, 2.9 Hz, 1H), 3.08 (ddd, J=10.0, 7.2, 2.4 Hz, 1H), 3.01 (s, 1H), 3.00-2.89 (m, 2H), 2.84 (s, 3H), 2.65 (br, 2H), 2.38 (dt, J=10.0, 7.0 Hz, 1H), 2.34 (s, 3H), 2.29 (d, J=7.4 Hz, 2H), 2.10-1.94 (m, 4H), 2.04 (s, 3H), 1.95-1.89 (m, 1H), 1.89-1.81 (m, 1H), 1.81-1.75 (m, 1H), 1.72 (s, 3H), 1.54-1.46 (m, 1H), 0.98 (d, J=7.0 Hz, 3H), 0.95 (d, J=6.5 Hz, 6H), 0.84 (d, J=7.0 Hz, 3H), 0.72 (d, J=6.9 Hz, 3H), −0.04 (d, J=7.1 Hz, 3H); ¹³C NMR (151 MHz, CD₂Cl₂) δ 192.6, 181.3, 171.9, 168.6, 168.3, 164.5, 155.4, 147.8, 146.7, 145.1, 144.8, 144.3, 142.6, 141.2, 132.9, 132.6, 125.5, 124.1, 116.7, 114.8, 112.0, 109.6, 107.7, 104.9, 95.2, 81.7, 76.9, 75.2, 72.7, 66.8, 56.6, 51.9, 51.8, 39.1, 38.7, 38.0, 36.6, 35.7, 33.3, 26.3, 22.2, 21.0, 20.4, 17.5, 11.7, 11.0, 9.2, 7.8; MS (ESI m/z [M+H]⁺ 911.4, [M−H]⁻ 909.4.

### 25-O-(6-carboxyl-2,5-dichlorobenzoyl)-25-O-desacetyrifabutin

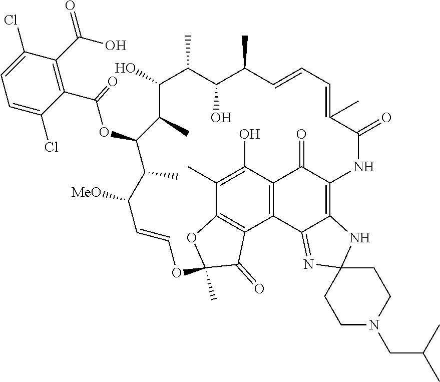 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)c2c(Cl)ccc(Cl)c2C(=O)O)[C@@H]1C)C4=O]

Prepared from 3 (100.0 mg) using acylation procedure A and the general deprotection procedure to afford the title compound (14.5 mg, 12% over two steps) as a purple solid. ¹H NMR (400 MHz, METHANOL-d₄) δ=7.52 (d, J=8.6 Hz, 1H), 7.41 (d, J=8.6 Hz, 1H), 6.70 (dd. J=11.0, 15.9 Hz, 1H) 6.45 (d, J=11.0 Hz, 1H), 6.25 (dd, J=7.2, 15.9 Hz, 1H), 6.11 (dd, J=1.3, 12.5 Hz, 1H), 5.69 (d, J=10.1 Hz, 1H), 5.10 (dd, J=4.9, 12.5 Hz, 1H), 4.03 (br d, J=10.3 Hz, 2H), 3.95 (br d, J=4.9 Hz, 2H), 3.85-3.72 (m, 3H), 3.30 (br d, J=7.0 Hz, 3H), 3.26 (s, 3H), 3.22 (s, 1H), 2.85-2.66 (m, 2H) 2.52-2.31 (m, 6H), 2.15 (s, 4H), 2.07-1.92 (m, 3H), 1.87-1.70 (m, 6H), 1.43-1.32 (m, 1H), 1.23 (d, J=1.8 Hz, 3H), 1.22 (d, J=1.9 Hz, 3H), 1.14 (d, J=7.0 Hz, 3H), 0.99 (d, J=6.9 Hz, 3H), 0.83-0.78 (m, 4H), −0.01 (d, J=7.2 Hz, 3H); MS (ESI) m/z [M+H]⁺ 1021.4.

### 25-O-(6-carboxyl-3,4-dichlorobenzoyl)-25-O-desacetyrifabutin (UMN99)

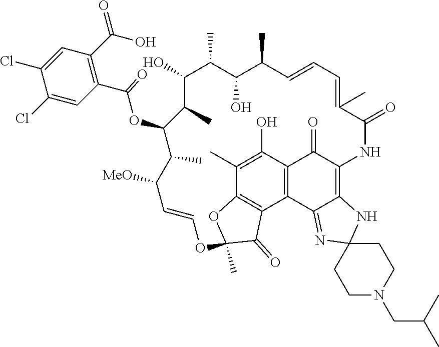 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)c2cc(Cl)c(Cl)cc2C(=O)O)[C@@H]1C)C4=O]

Prepared from 3 (150.0 mg) using acylation procedure A and the general deprotection procedure to afford the title compound (14.2 mg. 8% over two steps) as a purple solid. ¹H NMR (400 MHz, METHANOL-d₄) δ=7.94 (s, 1H), 7.64 (s, 1H), 6.69 (dd, J=11.0, 15.9 Hz, 1H), 6.45 (br d, J=11.0 Hz, 1H), 6.24 (dd, J=7.3, 15.6 Hz, 1H), 6.14 (dd, J=0.9, 12.4 Hz, 1H), 5.61 (d, J=10.4 Hz, 1H), 5.17 (dd, J=5.3, 12.5 Hz, 1H), 4.69 (br s, 4H), 4.04-3.88 (m, 3H), 3.83-3.70 (m, 2H), 3.66 (br d, J=5.1 Hz, 1H), 3.25 (br d, J=8.6 Hz, 2H), 3.15 (s, 3H), 2.82-2.67 (m, 2H), 2.52-2.34 (m, 6H), 2.16 (s, 3H), 2.08-1.90 (m, 3H), 1.86-1.75 (m, 6H), 1.53-1.43 (m, 1H), 1.24 (d, J=6.4 Hz, 6H), 1.10 (d, J=6.9 Hz, 3H), 0.98 (d, J=6.9 Hz, 3H), 0.83 (d, J=6.9 Hz, 3H), −0.01 (d·J=7.0 Hz, 3H); MS (ESI) m/z [M+H]⁺ 1021.4.

### Ester Versions of UMN99 on the Free Carboxylic Group can be Prepared by the Following Procedures:

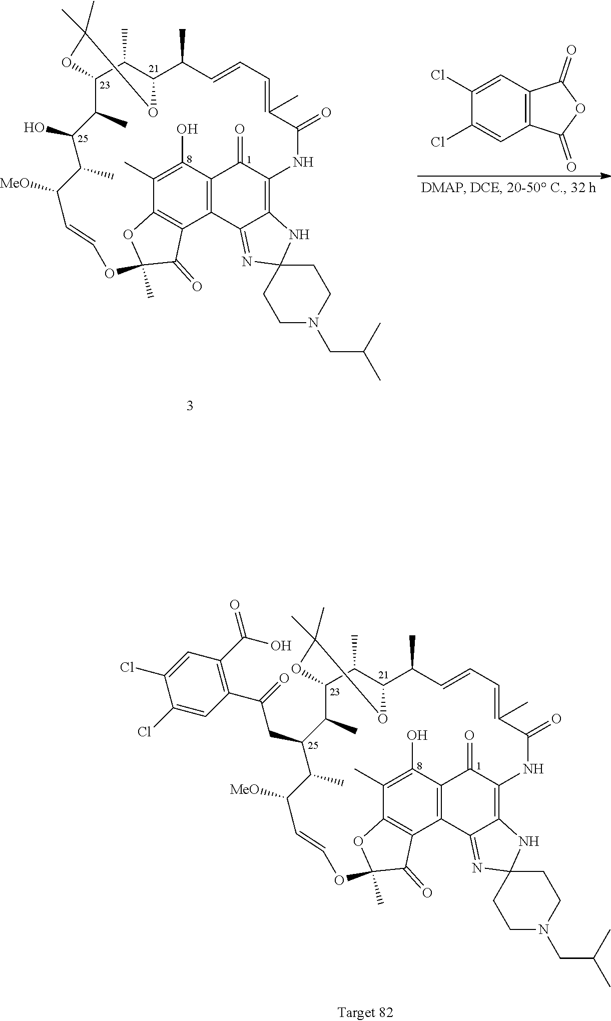 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@@H]2OC(C)(C)O[C@@H]([C@@H](C)[C@H](O)[C@@H]1C)[C@@H]2C)C4=O ; O=C1OC(=O)c2cc(Cl)c(Cl)cc21 ; CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@@H]2OC(C)(C)O[C@@H]([C@@H](C)[C@H](CC(=O)c3cc(Cl)c(Cl)cc3C(=O)O)[C@@H]1C)[C@@H]2C)C4=O]

To a solution of 3 (300 mg, 355.02 umol) in DCE (3 mL) was added 5,6-dichloroisobenzofuran-1,3-dione (385.20 mg, 1.78 mmol) and DMAP (34.70 mg, 284.01 umol) at 20° C. The mixture was stirred at 50° C. for 16 h. LC-MS showed ˜3% of Reactant 1 remained and desired compound was detected. Then 5,6-dichloroisobenzofuran-1,3-dione (385.20 mg, 1.78 mmol) and DMAP (34.70 mg, 284.01 umol) was added at 20° C., The mixture was stirred at 50° C. for 16 h. LC-MS showed Reactant 1 was consumed completely and desired compound was detected. The reaction mixture was diluted with DCM (10 ml) and a portion of saturated NaHCO₃ solution (30 ml) was added. The biphasic mixture was further stirred at room temperature for 16 h. Then the biphasic mixture was separated using a separatory funnel and the aqueous layer was back extracted with DCM (3*30 mL). The organic layers were then combined, dried (Na₂SO₄), concentrated under vacuum and separated to give a residue. The residue was purified by flash silica gel chromatography (ISCO®; 4g SepaFlash® Silica Flash Column, Eluent of 0˜6% Methanol/Dichloromethane gradient @75 mL/min)(Dichloromethane/Methanol=10, P1 Rf=0.46) to afford Target 83 (300 mg, 282.48 umol, 79.57% yield) as a purple solid.

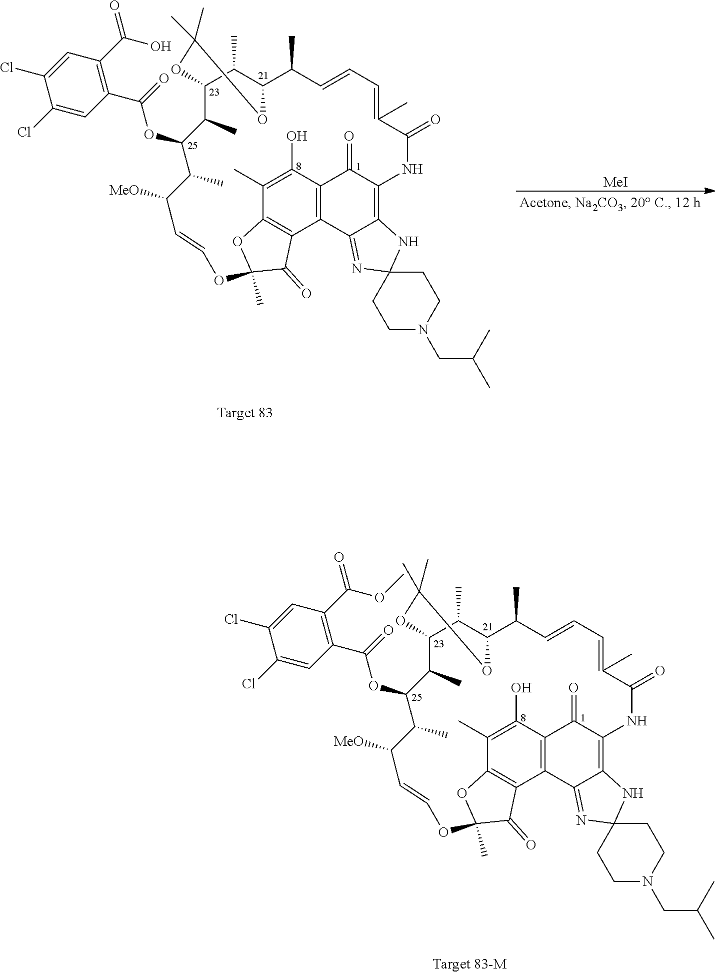 [SMILES: "MeI" ; CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@@H]2OC(C)(C)O[C@@H]([C@@H](C)[C@H](OC(=O)c3cc(Cl)c(Cl)cc3C(=O)O)[C@@H]1C)[C@@H]2C)C4=O ; COC(=O)c1cc(Cl)c(Cl)cc1C(=O)O[C@H]1[C@H](C)[C@@H]2OC(C)(C)O[C@H]([C@H]2C)[C@@H](C)/C=C/C=C(\C)C(=O)NC2=C3NC4(CCN(CC(C)C)CC4)N=C3c3c(c(O)c(C)c4c3C(=O)[C@@](C)(O/C=C/[C@H](OC)[C@H]1C)O4)C2=O]

To a solution of Target 83 (200 mg, 188.32 umol) in ACETONE (2 mL) was added Mel (53.46 mg, 376.64 umol, 23.45 uL) and Na₂CO₃ (39.92 mg, 376.64 umol). The mixture was stirred at 20° C. for 12 h. LC-MS showed reactant was consumed completely and desired mass was detected. The reaction mixture was concentrated under reduced pressure to remove solvent. The residue was purified by Prep-TLC (SiO₂, Dichloromethane/Methanol=10/1, P1 Rf=0.67) to afford Target 83-M (55 mg, 51.11 umol, 27.14% yield) as a purple solid.

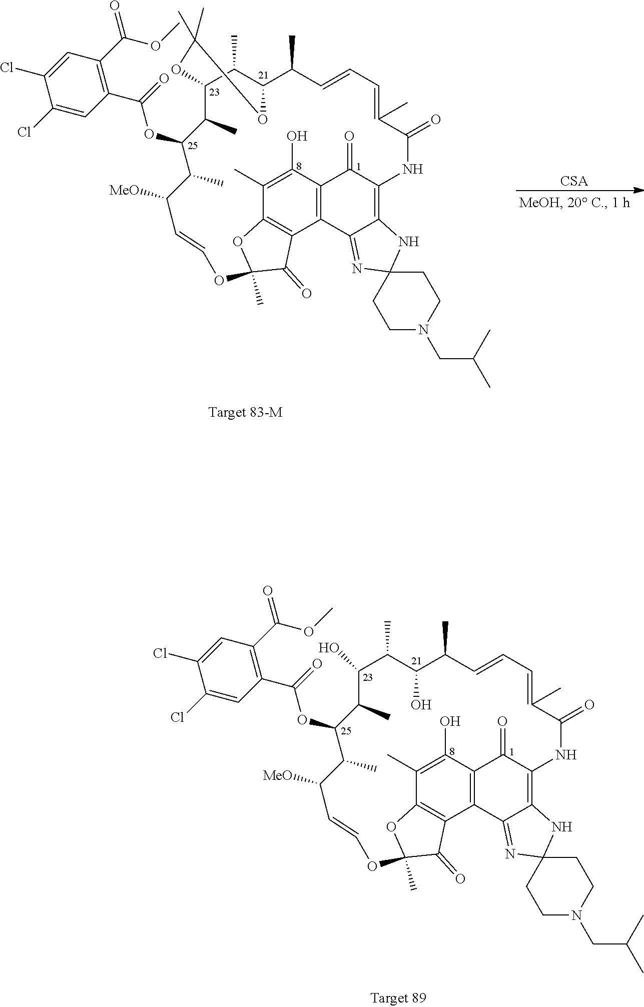 [SMILES: COC(=O)c1cc(Cl)c(Cl)cc1C(=O)O[C@H]1[C@H](C)[C@@H]2OC(C)(C)O[C@H]([C@H]2C)[C@@H](C)/C=C/C=C(\C)C(=O)NC2=C3NC4(CCN(CC(C)C)CC4)N=C3c3c(c(O)c(C)c4c3C(=O)[C@@](C)(O/C=C/[C@H](OC)[C@H]1C)O4)C2=O ; "CSA" ; COC(=O)c1cc(Cl)c(Cl)cc1C(=O)O[C@H]1[C@H](C)[C@H](O)[C@H](C)[C@@H](O)[C@@H](C)/C=C/C=C(\C)C(=O)NC2=C3NC4(CCN(CC(C)C)CC4)N=C3c3c(c(O)c(C)c4c3C(=O)[C@@](C)(O/C=C/[C@H](OC)[C@H]1C)O4)C2=O]

To a solution of Target 83-M (55 mg, 51.11 umol, 1 eq) in MeOH (1 mL) was added CSA (25.59 mg, 102.22 umol). The mixture was stirred at 20° C. for 1 h. LC-MS showed Reactant 1 was consumed completely and desired mass was detected. The NaHCO₃ (30 mg) was added after which the mixture was partitioned between DCM (3 mL) and water (3 mL). The aqueous phase was back extracted with DCM (3*5 mL). The organic layers were then combined, dried (Na₂SO₄) and concentrated under vacuum. The reaction mixture was concentrated under reduced pressure to remove solvent. The residue was purified by Prep-HPLC (neutral condition) column: Waters Xbridge BEH C18 100*30 mm*10 um; mobile phase: [water(NH₄HCO₃)-ACN]; B %: 25%-65%, 8 min) to afford Target 89 (14.6 mg, 13.37 umol, 26.16% yield, 94.88% purity) as a purple solid. ¹H NMR (400 MHz, CHLOROFORM-d) δ=14.69 (s, 1H), 9.14 (br s, 1H), 8.21 (s, 1H), 7.80 (s, 1H), 7.65 (s, 1H), 6.31-6.23 (m, 1H), 6.17 (d, J=12.4 Hz, 2H), 5.80 (dd, J=6.7, 15.7 Hz, 1H), 5.10 (dd, J=9.0, 12.7 Hz, 1H), 4.74-4.66 (m, 1H), 4.29-4.19 (m, 1H), 3.82 (s, 3H), 3.54-3.48 (m, 2H), 3.27 (d, J=6.0 Hz, 1H), 3.20 (dd, J=2.8, 9.0 Hz, 1H), 3.17-3.10 (m, 1H), 2.96 (s, 3H), 2.91-2.82 (m, 2H), 2.64-2.49 (m, 2H), 2.21 (s, 4H), 2.05-1.96 (m, 2H), 1.94 (s, 4H) 1.79-1.72 (m, 3H), 1.65 (br d, J=7.0 Hz, 2H), 1.61 (s, 3H), 1.33 (br dd, J=6.4, 14.4 Hz, 1H), 0.92 (d, J=7.0 Hz, 3H), 0.85 (d, J=6.5 Hz, 6H), 0.72 (d, J=6.9 Hz, 3H), 0.56 (d, J=6.9 Hz, 3H), 0.01 (d, J=7.0 Hz, 3H); MS (ESI) m/z [M+H]⁺ 1035.3.

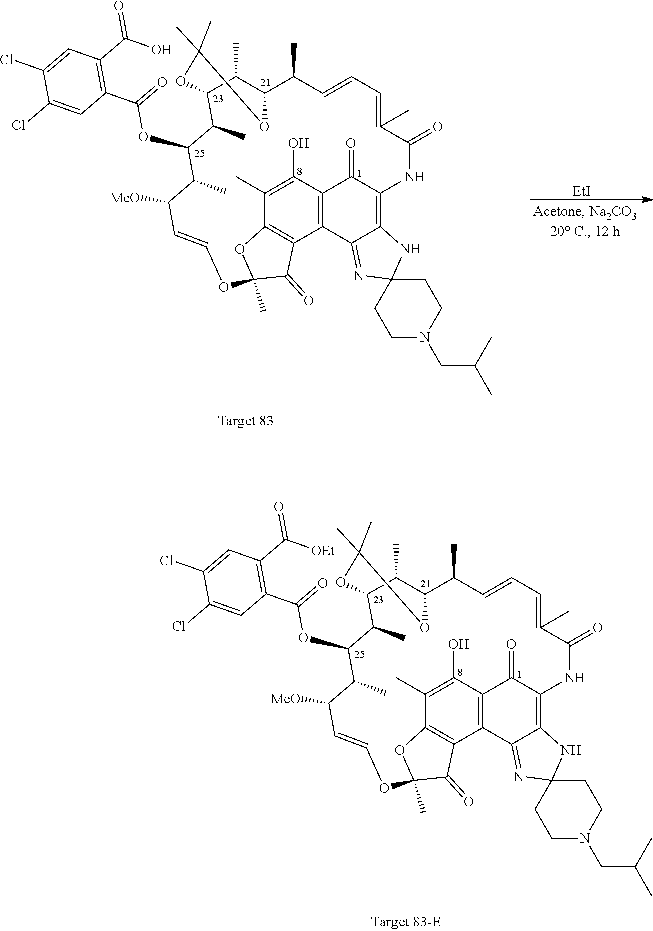 [SMILES: "EtI" ; CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@@H]2OC(C)(C)O[C@@H]([C@@H](C)[C@H](OC(=O)c3cc(Cl)c(Cl)cc3C(=O)O)[C@@H]1C)[C@@H]2C)C4=O ; CCOC(=O)c1cc(Cl)c(Cl)cc1C(=O)O[C@H]1[C@H](C)[C@@H]2OC(C)(C)O[C@H]([C@H]2C)[C@@H](C)/C=C/C=C(\C)C(=O)NC2=C3NC4(CCN(CC(C)C)CC4)N=C3c3c(c(O)c(C)c4c3C(=O)[C@@](C)(O/C=C/[C@H](OC)[C@H]1C)O4)C2=O]

To a solution of Target 83 (300 mg, 282.48 umol, 1 eq) in ACETONE (2 mL) was added EtI (88.11 mg, 564.95 umol, 45.19 uL, 2 eq) and Na₂CO₃ (59.88 mg, 564.95 umol, 2 eq). The mixture was stirred at 20° C. for 12 h. LC-MS showed Reactant 1 was consumed completely and desired mass was detected. The reaction mixture was concentrated under reduced pressure to remove solvent. The residue was purified by prep-TLC (SiO₂, Dichloromethane/Methanol=10/1, P1 Rf=0.67) to afford Target 83-E (100 mg, 91.74 umol, 32.48% yield) as a purple solid.

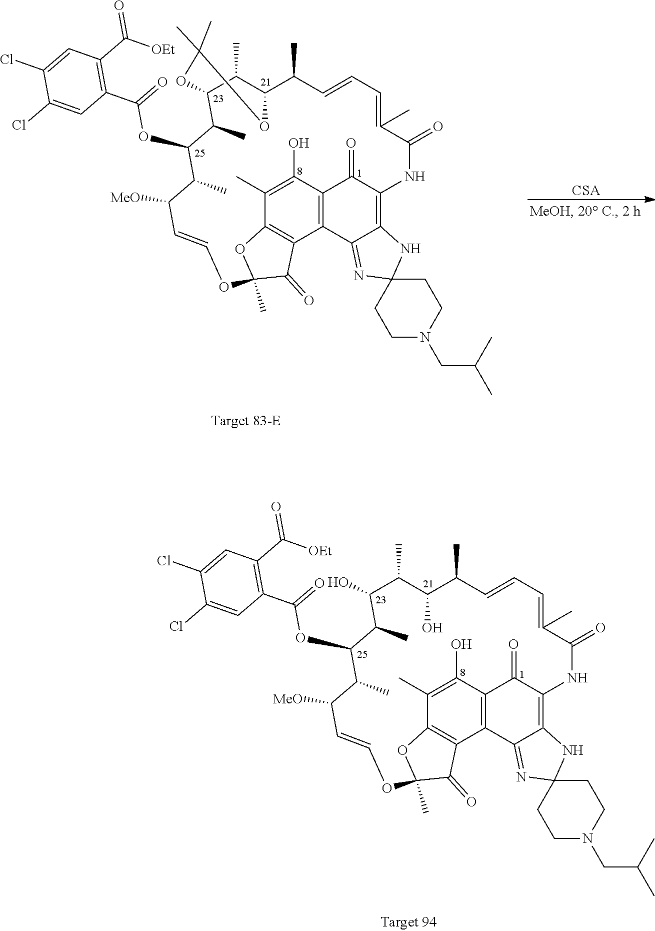 [SMILES: CCOC(=O)c1cc(Cl)c(Cl)cc1C(=O)O[C@H]1[C@H](C)[C@@H]2OC(C)(C)O[C@H]([C@H]2C)[C@@H](C)/C=C/C=C(\C)C(=O)NC2=C3NC4(CCN(CC(C)C)CC4)N=C3c3c(c(O)c(C)c4c3C(=O)[C@@](C)(O/C=C/[C@H](OC)[C@H]1C)O4)C2=O ; "CSA" ; CCOC(=O)c1cc(Cl)c(Cl)cc1C(=O)O[C@H]1[C@H](C)[C@H](O)[C@H](C)[C@@H](O)[C@@H](C)/C=C/C=C(\C)C(=O)NC2=C3NC4(CCN(CC(C)C)CC4)N=C3c3c(c(O)c(C)c4c3C(=O)[C@@](C)(O/C=C/[C@H](OC)[C@H]1C)O4)C2=O]

To a solution of Target 83-E (100 mg, 91.74 umol) in MeOH (1 mL) was added CSA (45.93 mg, 183.47 umol). The mixture was stirred at 20° C. for 1 h. The NaHCO₃; (30 mg) was added after which the mixture was partitioned between DCM (3 mL) and water (3 mL). The aqueous phase was back extracted with DCM (3*5 mL). The organic layers were combined, dried (Na₂SO₄) and concentrated under vacuum to give residue. The residue was purified by prep-HPLC (neutral condition) column: column: Waters Xbridge BEH C18 100*30 mm*10 um; mobile phase: [water (NH₄HCO₃)-ACN]; B %: 25%-65%, 8 min) to afford Target 94 (11.8 mg, 10.90 umol, 11.88% yield, 97% purity) as a purple solid. ¹H NMR (400 MHz, CHLOROFORM-d) δ=14.87 (br s, 1H), 8.77 (br s, 1H), 8.19 (s, 1H), 7.51-7.41 (m, 2H), 6.59-6.47 (m, 1H), 6.28 (br d, J=10.6 Hz, 1H), 6.19 (dd, J=7.0, 15.9 Hz, 1H), 6.06 (dd, J=1.2, 12.3 Hz, 1H), 5.50 (d, J=10.1 Hz, 1H), 5.09 (dd, J=4.7, 12.3 Hz, 1H), 4.42 (q, J=7.1 Hz, 2H), 3.84 (br d, J=9.6 Hz, 1H), 3.79-3.72 (m, 1H), 3.58 (s, 1H), 3.45-3.37 (m, 2H), 3.16 (s, 3H), 3.11-2.99 (m, 1H), 2.81-2.56 (m, 2H), 2.47-2.30 (m, 6H), 2.12 (s, 4H), 1.83 (s, 5H), 1.76-1.67 (m, 6H), 1.60-1.54 (m, 1H), 1.43 (t, J=7.1 Hz, 3H), 1.09 (d, J=7.0 Hz, 3H), 1.00 (br s, 5H), 0.92 (d, J=7.0 Hz, 3H), 0.69 (d, J=6.9 Hz, 3H), −0.01 (d, J=7.1 Hz, 3H); MS (ESI) m/z [M+H]⁺ 1049.7.

### Synthesis of 25-O-carbamoyl sulfonamide rifabutins (Sulfonamide Analogs)

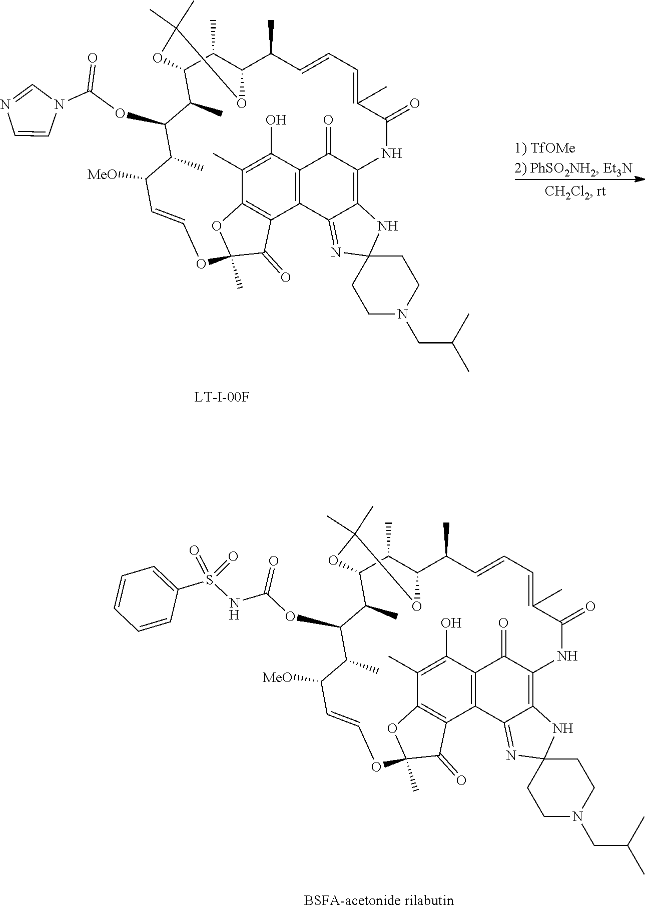

To a solution of LT-I-00F (40.0 mg, 0.043 mmol) in dichloromethane (0.5 mL), methyl trifluoromethanesulfonate (0.064 mmol, 7 μL) was added. Ater the mixture was stirred for 10 min at room temperature, benzenesulfonamide (33.5 mg, 0.21 mmol) and triethylamine (0.22 mmol, 30 μL) were added and the reaction mixture was further stirred under room temperature for 1 hr. Then the reaction mixture was partitioned between dichloromethane (5 mL) and H₂O (5 mL). The aqueous phase was then back extracted with dichloromethane (2×5 mL). The combined organic layers were concentrated under reduced pressure and the resulting crude was purified by preparative TLC (hexanes:ethyl acetate:methanol:triethylamine=7:2:1:0.1) to obtain BSFA-acetonide rifabutin (33.4 mg, 76%). MS (ESI): m/z [M+H]⁺ calcd for C₅₄H₆₉N₅O₁₃S: 1028.4685; found:1028.4637 (error 4.7 ppm). ¹H NMR (600 MHz, CDCl₃) δ 14.73 (s, 1H), 8.67 (s, 1H), 7.95-7.86 (m, 4H), 7.70 (s, 1H), 7.58-7.53 (m, 1H), 7.52-7.46 (m, 3H), 7.42 (t, J=7.7 Hz, 2H), 7.03 (t, J=1.1 Hz, 1H), 6.87 (t, J=1.3 Hz, 1H), 6.27 (dd, J=15.7, 10.8 Hz, 1H), 6.16 (dd, J=10.7, 1.6 Hz, 1H), 6.05 (dd, J=15.7, 7.0 Hz, 1H), 5.85-5.80 (m, 1H), 4.96 (dd, J=12.2, 6.6 Hz, 1H), 4.79 (dd, J=8.2, 1.7 Hz, 1H), 3.69 (s, 2H), 3.53 (dd, J=10.6, 3.2 Hz, 1H), 3.26 (s, 1H), 3.12 (h, J=11.4 Hz, 4H), 2.94 (dd, J=10.2, 5.1 Hz, 1H), 2.66 (s, 3H), 2.58 (s, 2H), 2.30 (s, 3H), 2.233 (dt, J=10.6, 6.9 Hz, 1H), 2.06-1.95 (m, 41H), 1.75 (s, 3H), 1.63 (t, J=8.2 Hz, 1H), 1.49-1.41 (m, 1H), 1.36-1.29 (m, 1H), 1.08 (s, 3H), 1.01 (d, J=6.5 Hz, 6H H), 0.81 (d, J=6.0 Hz, 6H), 0.75 (d, J=6.6 Hz, 3H), 0.55 (d, J=7.1 Hz, 3H), 0.22 (d, J=6.8 Hz, 3H). ¹³C NMR (151 MHz, CDCl₃) δ 192.8, 181.7, 172.1, 168.8, 168.2, 155.6, 142.4, 142.2, 141.2, 140.9, 140.8, 137.9, 132.9, 132.7, 132.6, 131.2, 129.2, 129.0, 128.7, 127.8, 126.5, 125.3, 123.9, 120.2, 114.8, 114.0, 111.7, 108.7, 106.1, 104.8, 100.0, 93.8, 70.9, 65.7, 56.1, 51.6, 51.3, 40.8, 40.6, 36.1, 35.2, 34.5, 34.4, 33.5, 25.8, 25.4, 23.8, 21.1, 21.1, 20.3, 20.3, 17.9, 12.9, 9.7, 9.6, 7.9.

### Synthesis or Carbamate 1

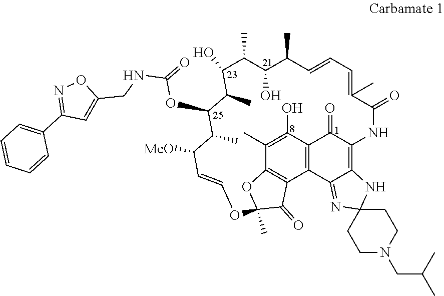 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)NCc2cc(-c3ccccc3)no2)[C@@H]1C)C4=O]

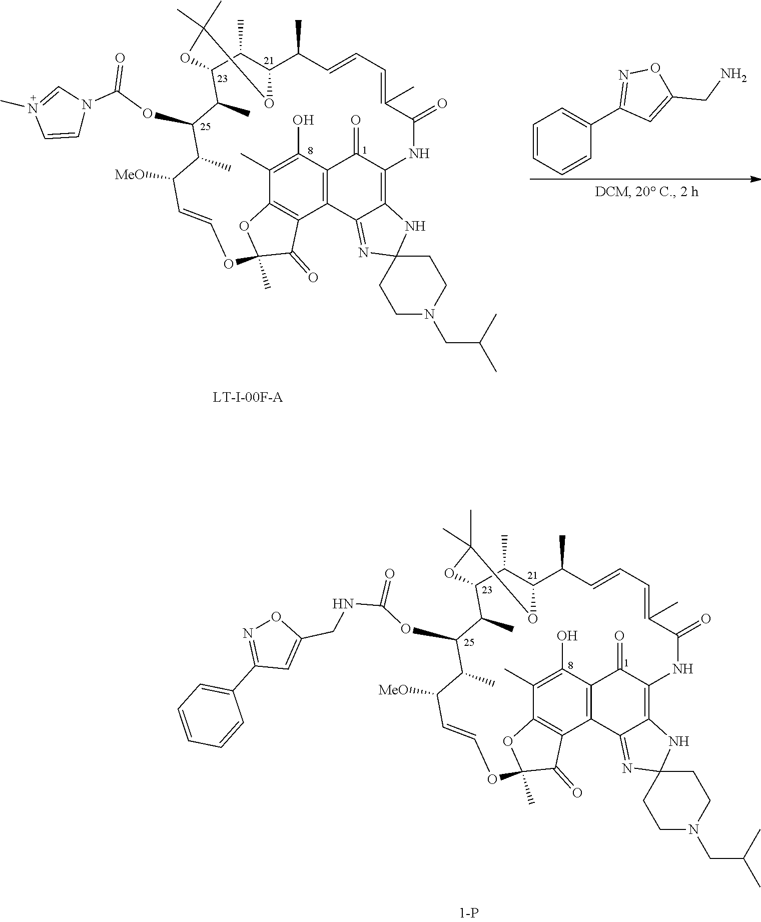

To a solution of LT-I-00F-A (200 mg, 209.61 umol) in DCM (0.5 ml) was added (3-phenylisoxazol-5-yl)methanamine (365.14 mg, 2.10 mmol). The mixture was stirred at 20° C. for 2 hr. LC-MS showed LT-I-00F-A was consumed completely and desired mass was detected. The reaction mixture was concentrated under reduced pressure to remove solvent. The residue was purified by prep-TLC (SiO₂, Petroleum ether/Ethyl acetate/Methanol=7/2/1, P1 Rf=0.31) to afford 1-P (70 mg, 66.97 umol, 31.95% yield) as a purple solid.

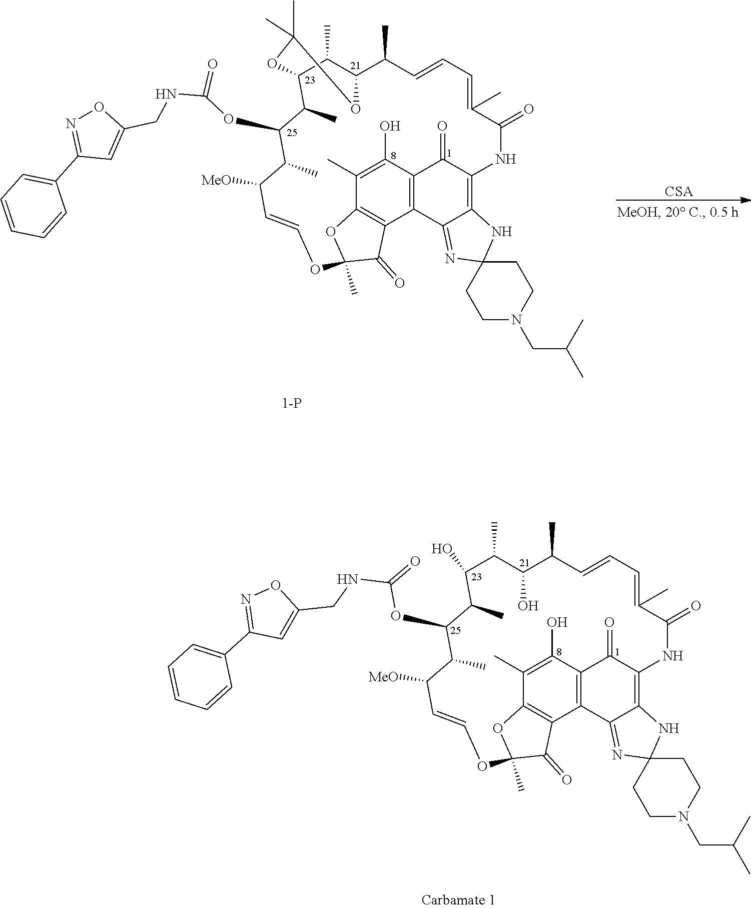 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@@H]2OC(C)(C)O[C@@H]([C@@H](C)[C@H](OC(=O)NCc3cc(-c5ccccc5)no3)[C@@H]1C)[C@@H]2C)C4=O ; "CSA" ; CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)NCc2cc(-c3ccccc3)no2)[C@@H]1C)C4=O]

To a solution of 1-P (70 mg, 66.97 umol) in MeOH (1 mL) was added CSA (33.53 mg, 133.94 umol). The mixture was stirred at 20° C. for 0.5 hr. LC-MS showed 1-P was consumed completely and desired mass was detected. The NaHCO₃ (30 mg) was added after which the mixture was partitioned between DCM (3 mL) and water (3 mL). The aqueous phase was back extracted with DCM (3*5 mL). The organic layers were then combined, dried (Na₂SO₄) and concentrated under vacuum. The reaction mixture was concentrated under reduced pressure to remove solvent. The residue was purified by prep-HPLC (neutral condition) column: Waters Xbridge BEH C18 100*30 mm*10 um; mobile phase: [water (NH₄HCO₃)-ACN]; B %: 70%-100%, 8 min to afford Carbamate 1 (11.4 mg, 10.74 umol, 16.04% yield, 94.74% purity) as a purple solid. ¹H NMR CHLOROFORM-d 400 MHz δ=14.81 (br s, 1H), 9.02 (br s, 1H), 8.16 (s, 1H), 7.80 (br d, J=3.6 Hz, 2H), 7.48-7.43 (m, 3H), 6.52 (s, 1H), 6.38 (br dd. J=10.1, 15.7 Hz, 1H), 6.28-6.14 (m, 2H), 5.98 (br dd, J=6.7, 15.4 Hz, 1H), 5.33-5.26 (m, 1H), 5.03 (br dd. J=7.4, 12.3 Hz, 1H), 4.62-4.48 (m, 2H), 4.40 (br dd, J=5.4, 16.2 Hz, 1H), 4.22 (br d, J=4.0 Hz, 1H), 3.70-3.56 (m, 2H), 3.35 (br d, J=5.6 Hz, 1H), 3.15-2.93 (m, 6H), 2.77-2.55 (m, 2H), 239-2.24 (m, 6H), 2.04 (s, 4H), 1.89-1.80 (m, 2H), 1.78-1.67 (m, 6H), 1.51-1.40 (m, 2H), 1.00 (br d, J=6.9 Hz, 3H), 0.94 (br d, J=5.9 Hz, 6H), 0.83 (br d, J=6.8 Hz, 3H), 0.58 (br d, J=6.6 Hz, 3H), −0.01 (d, J=7.1 Hz, 3H)

### Synthesis of Carbamate 2

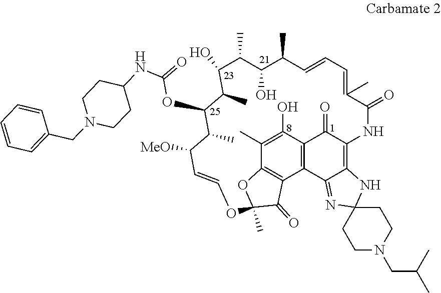 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)NC2CCN(Cc3ccccc3)CC2)[C@@H]1C)C4=O]

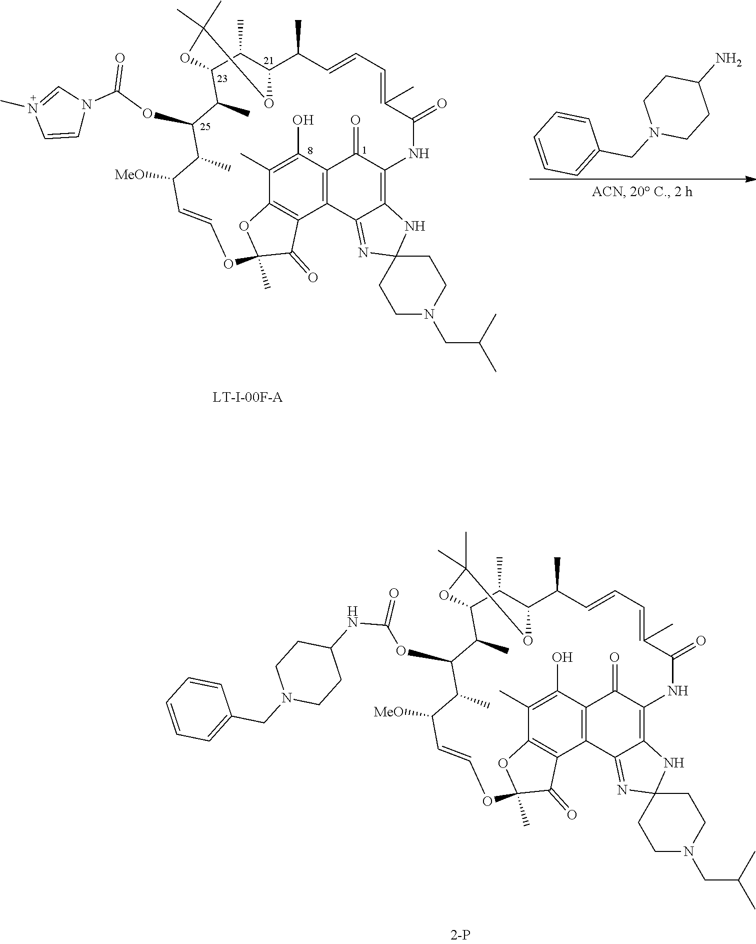

To a solution of LT-I-00F-A (200 mg, 212.74 umol) in ACN (2 mL) was added 1-benzylpiperidin-4-amine (404.81 mg, 2.13 mmol). The mixture was stirred at 20° C. for 2 hr. LC-MS showed LT-I-00F-A was consumed completely and desired mass was detected. The reaction mixture was concentrated under reduced pressure to remove solvent. The residue was purified by prep-TLC (SiO₂, Petroleum ether/Ethyl acetate/Methanol=7/2/1, P1 Rf=0.46) to afford 2-P (100 mg, 94.22 umol·44.29% yield) as a purple solid.

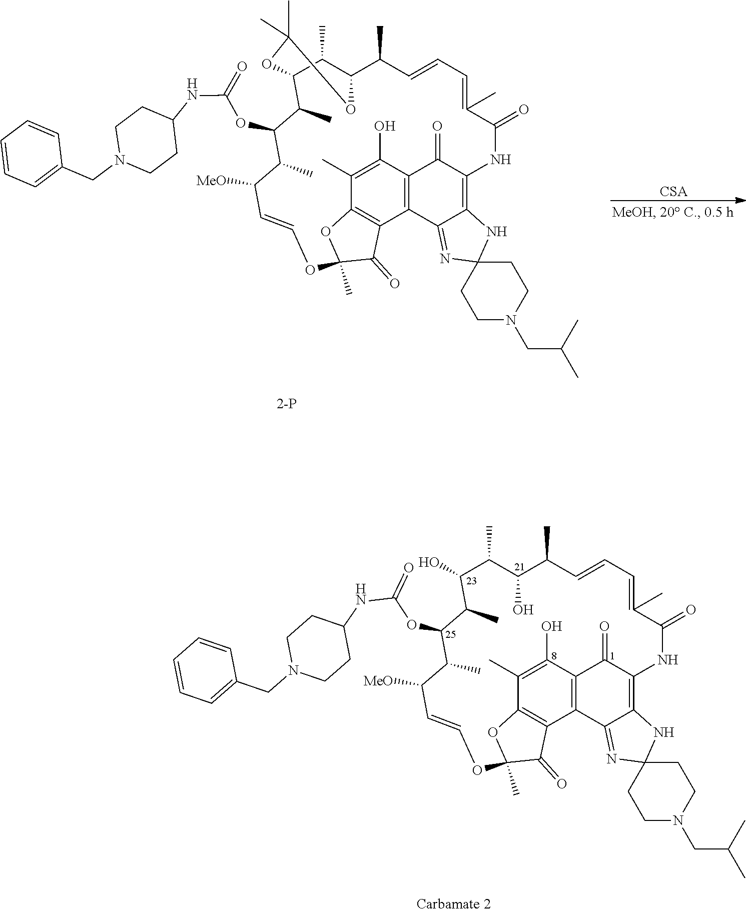 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@@H]2OC(C)(C)O[C@@H]([C@@H](C)[C@H](OC(=O)NC3CCN(Cc5ccccc5)CC3)[C@@H]1C)[C@@H]2C)C4=O ; "CSA" ; CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)NC2CCN(Cc3ccccc3)CC2)[C@@H]1C)C4=O]

To a solution of 2-P (100 mg, 94.22 umol) in MeOH (2 mL) was added CSA (47.17 mg, 188.45 umol). The mixture was stirred at 20° C. for 0.5 hr. LC-MS showed 2-P was consumed completely and ˜80% of desired mass was detected. The NaHCO₃ (30 mg) was added after which the mixture was partitioned between DCM (3 mL) and water (3 mL). The aqueous phase was back extracted with DCM (3*5 mL). The organic layers were combined, dried (Na₂SO₄) and concentrated to give a residue. The residue was purified by prep-HPLC (neutral condition column: column: Waters Xbridge BEH C18 100*30 mm*10 um; mobile phase: [water(NH₄HCO₄)-ACN]; B %: 75%-95% 8 min to afford Carbamate 2 (54.6 mg, 50.26 umol, 53.34% yield, 94% purity) as a purple solid. ¹H NMR CHLOROFORM-d 400 MHz δ=14.88 (s, 1H), 8.92 (br s, 1H), 8.21 (s, 1H), 7.37-7.33 (m, 2H), 7.32-7.26 (m, 3H), 6.42 (br dd, J=10.3, 15.6 Hz, 1H), 6.27 (br d, J=10.1 Hz, 1H), 6.16 (d, J=12.4 Hz, 1H), 6.07 (br dd, J=6.9, 15.7 Hz, 1H), 5.04 (dd, J=6.5, 12.4 Hz, 1H), 4.68 (br d, J=8.1 Hz, 1H), 4.61-4.50 (m, 2H), 3.80 (s, 1H), 3.67 (br d, J=9.8 Hz, 1H), 3.53-3.48 (m, 3H), 3.43 (br d, J=5.6 Hz, 1H), 3.11 (s, 4H), 2.99 (br d, J=4.1 Hz, 2H), 2.82 (br d, J=10.4 Hz, 2H), 2.64 (br dd, J=10.5, 12.0 Hz, 2H), 2.38-2.29 (m, 6H), 2.16-2.06 (m, 7H), 1.93-1.75 (m, 9H), 1.69-1.61 (m, 1H), 1.51-1.40 (m, 3H), 1.05 (d, J=7.0 Hz, 3H), 0.97 (d, J=6.5 Hz, 6H), 0.86 (d, J=6.9 Hz, 3H), 0.59 (br d, J=6.8 Hz, 3H), −0.01 (d, J=7.0 Hz, 3H).

### Synthesis of Carbamate 3

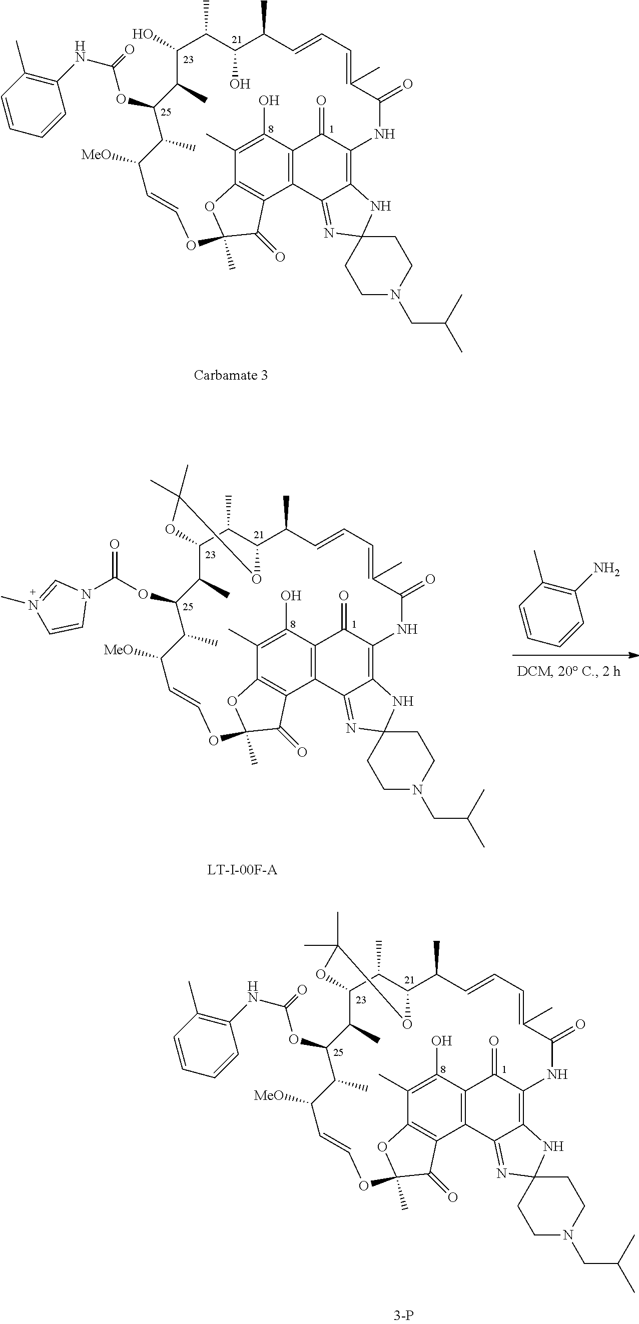

To a solution of LT-I-00F-A (200 mg, 212.74 umol) in DCM (2 mL) was added 2-methylaniline (227.96 mg, 2.13 mmol). The mixture was stirred at 20° C. for 2 hr. LC-MS showed LT-I-00F-A was consumed completely and desired mass was detected. The reaction mixture was concentrated and purified by prep-TLC (SiO₂, Petroleum ether/Ethyl acetate/Methanol=7/2/1, P1 Rf=0.46) to afford 3-P (90 mg, 34.60% yield, 80% purity) as a purple solid.

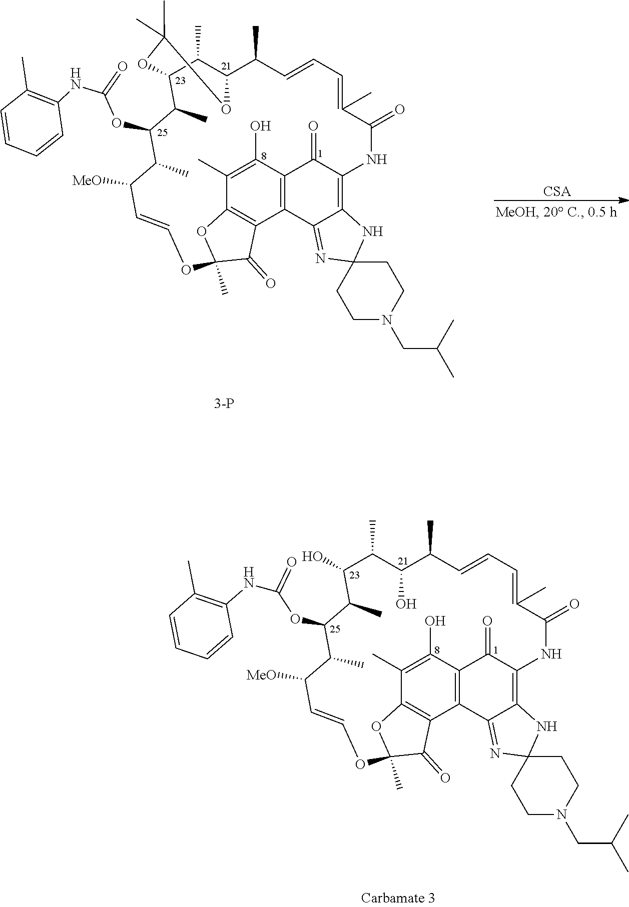 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@@H]2OC(C)(C)O[C@@H]([C@@H](C)[C@H](OC(=O)Nc3ccccc3C)[C@@H]1C)[C@@H]2C)C4=O ; "CSA" ; CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)Nc2ccccc2C)[C@@H]1C)C4=O]

To a solution of 3-P (90 mg, 92.01 umol) in MeOH (2 mL) was added CSA (46.06 mg, 184.02 umol). The mixture was stirred at 20° C. for 0.5 hr. LC-MS showed 3-P was consumed completely and desired mass was detected. The NaHCO₃ (30 mg) was added after which the mixture was partitioned between DCM (3 mL) and water (3 mL). The aqueous phase was back extracted with DCM (3*5 mL). The organic layers were combined, dried (Na₂SO₄) and concentrated to give a residue. The residue was purified by prep-HPLC (neutral condition column: column: Waters Xbridge BEH C18 100*30 mm*10 um: mobile phase: [water(NH₄HCO₃)-ACN]; B %: 75%-95%, 8 min to afford Carbamate 3 (53.2 mg, 56.09% yield, 91% purity) as a purple solid. ¹H NMR CHLOROFORM-d 400 MHz, δ=14.82 (s, 1H), 8.97 (br s, 1H), 8.18 (br s, 1H), 7.70 (br s, 1H), 7.19 (br t, J=7.8 Hz, 1H), 7.14 (d, J=7.4 Hz, 1H), 7.06-7.00 (m, 1H), 6.47-6.35 (m, 2H), 6.23 (br d, J=10.3 Hz, 11H), 6.16 (br d, J=12.4 Hz, 1H), 6.01 (br dd, J=6.9, 15.8 Hz, 1H), 5.05 (dd, J=6.9, 12.4 Hz, 1H), 4.62 (br d, J=9.8 Hz, 1H), 4.26 (br d, J=3.6 Hz, 1H), 3.71-3.61 (m, 2H), 3.42 (br d, J=6.0 Hz, 1H), 3.15 (br dd, J=3.7, 9.4 Hz, 1H), 3.09 (s, 3H), 2.95 (br d, J=5.1 Hz, 2H), 2.61 (br s, 2H), 2.36-2.25 (m, 6H), 2.21 (s, 3H), 2.15-1.91 (m, 7H), 1.80-1.71 (m, 6H), 1.56-1.46 (m, 1H), 1.01 (d, J=7.0 Hz, 3H), 0.92 (d, J=6.5 Hz, 6H), 0.83 (d, J=6.9 Hz, 3H), 0.61 (br d, J=6.6 Hz, 3H), −0.01 (br d, J=6.8 Hz, 3H.

### Synthesis of Carbamate 4

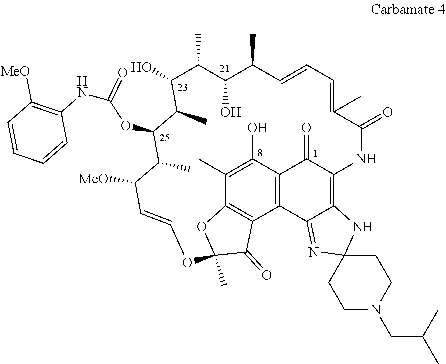 [SMILES: COc1ccccc1NC(=O)O[C@H]1[C@H](C)[C@H](O)[C@H](C)[C@@H](O)[C@@H](C)/C=C/C=C(\C)C(=O)NC2=C3NC4(CCN(CC(C)C)CC4)N=C3c3c(c(O)c(C)c4c3C(=O)[C@@](C)(O/C=C/[C@H](OC)[C@H]1C)O4)C2=O]

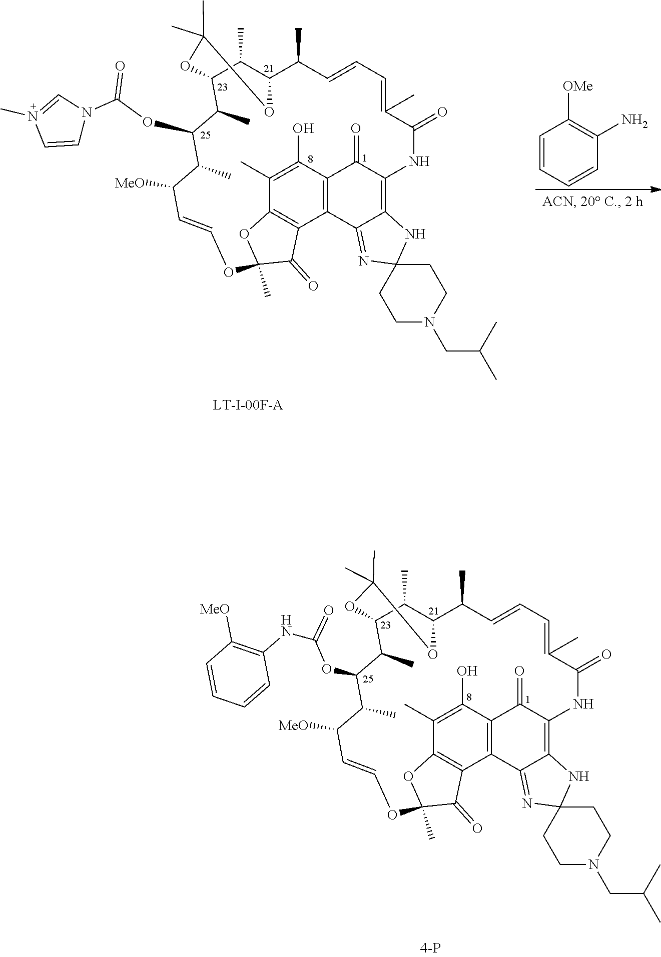

To a solution of LT-I-00F-A (200 mg, 212.74 umol) in DCM (2 mL) was added 2-methoxyaniline (262.00 mg, 2.13 mmol). The mixture was stirred at 20° C. for 2 hr. LC-MS showed LT-I-00F-A was consumed completely and desired mass was detected. The reaction mixture was concentrated and purified by prep-TLC (SiO₂, Petroleum ether/Ethyl acetate/Methanol=7/2/1, P1 Rf=0.46) to afford 4-P (90 mg, 34.04% yield, 80% purity) as a purple solid.

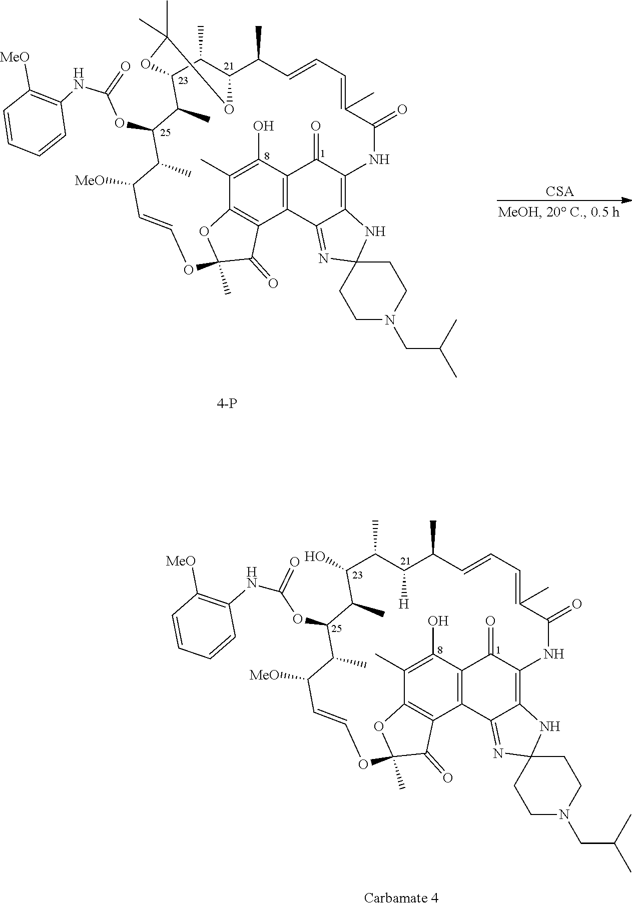

To a solution of 4-P (90 mg, 90.53 umol) in MeOH (2 mL) was added CSA (45.32 mg, 181.05 umol). The mixture was stirred at 20° C. for 0.5 hr. LC-MS showed 4-P was consumed completely and ˜85% of desired mass was detected. The NaHCO₃ (32 mg) was added after which the mixture was partitioned between DCM (3 mL) and water (3 mL). The aqueous phase was back extracted with DCM (3*5 mL). The organic layers were combined, dried (Na₂SO₄) and concentrated under vacuum. The reaction mixture was concentrated under reduced pressure to remove solvent. The residue was purified by prep-HPLC (neutral condition column: Waters Xbridge BEH C18 100*30 mm*10 um; mobile phase: [water (NH₄HCO₃-ACN]; B %: 75%-95%, 8 min and further purified by prep-HPLC (neutral condition) column: Phenonenex Luna C18 75*30 mm*3 um; mobile phase: [water (FA)-ACN]; B %: 30%-70%, 8 min) to afford Carbamate 4 (22.7 mg, 25.76% yield, 98% purity) as a purple solid. ¹H NMR CHLOROFORM-d 400 MHz δ=14.81 (s, 1H), 8.89 (br s, 1H), 8.15 (s, 1H), 8.04 (br d, J=5.5 Hz, 1H), 7.24 (br s, 1H), 7.08-6.92 (m, 2H), 6.86 (d, J=8.1 Hz, 1H), 6.47-6.35 (m, 1H), 6.25 (br d, J=10.1 Hz, 1H), 6.14 (d, J=12.5 Hz, 1H), 6.04 (dd, J=6.9, 15.7 Hz, 1H), 5.04 (dd, J=6.6, 12.5 Hz, 1H), 4.67 (br d, J=9.3 Hz, 1H), 4.33 (br s, 1H), 3.85 (s, 3H), 3.78-3.63 (m, 2H), 3.46 (br d, J=5.3 Hz, 1H), 3.17 (br d, J=10.1 Hz, 1H), 3.07 (s, 5H), 2.73 (br d, J=5.0 Hz, 2H), 2.42-2.29 (m, 6H), 2.15-1.83 (m, 8H), 1.78-1.70 (m, 5H), 1.58-1.49 (m, 1H), 1.01 (d, J=7.0 Hz, 3H), 0.96 (br d, J=6.5 Hz, 6H), 0.84 (d, J=6.8 Hz, 3H), 0.63 (d, J=6.8 Hz, 3H), −0.01 (d, J=7.1 Hz, 3H)

### Synthesis of Carbamate 5

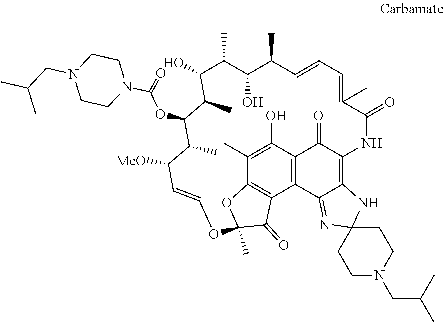 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)N2CCN(CC(C)C)CC2)[C@@H]1C)C4=O]

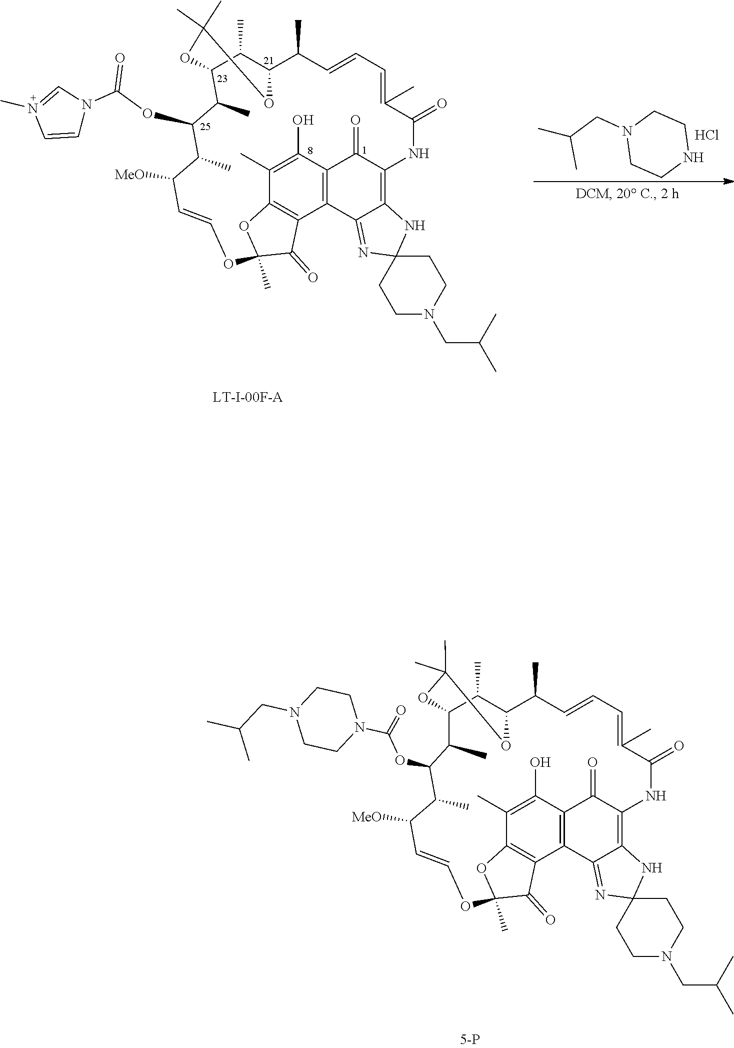

To a solution of LT-I-00F-A (250 mg, 262.02 umol) in DCM (2 mL) was added 1-isobutylpiperazine; dihydrochloride (169.13 mg, 786.06 umol). The mixture was stirred at 20° C. for 3 hr. LC-MS showed LT-I-00F-A was consumed completely and desired mass was detected. The reaction mixture was concentrated and purified by prep-TLC (SiO₂, Petroleum ether/Ethyl acetate/Methanol=7/2/1, P1 Rf=0.31) to afford 5-P (66 mg, 65.14 umol, 24.86% yield) as a purple solid.

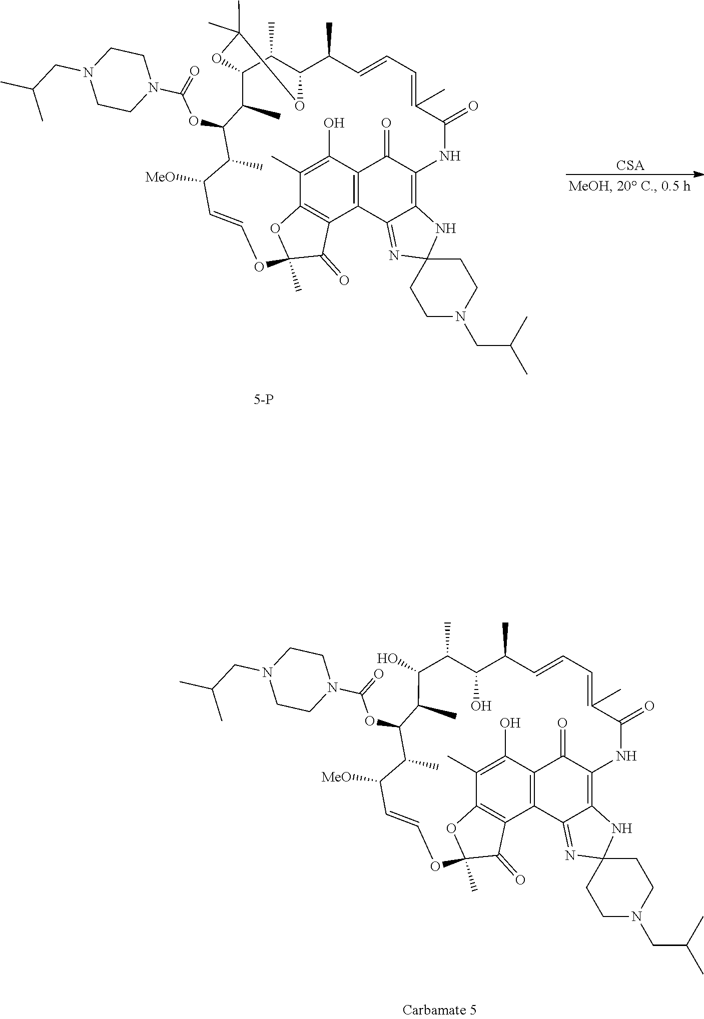

To a solution of 5-P (66 mg, 65.14 umol) in MeOH (1 mL) was added CSA 32.61 mg. 130.27 umol). The mixture was stirred at 20° C. for 0.5 hr. LC-MS showed 5-P was consumed completely and desired mass was detected. The NaHCO₃ 30 mg) was added after which the mixture was partitioned between DCM (3 mL) and water (3 mL). The aqueous phase was back extracted with DCM (3*5 mL). The organic layers were combined, dried (Na₂SO₄) and concentrated to give a residue. The residue was purified by prep-HPLC (neutral condition column: column: Waters Xbridge BEH C18 100*30 mm*10 um; mobile phase: [water (NH₄HCO₃)-ACN]; B %: 75%-95%, 10 min to afford Carbamate 5 (22.27 mg, 33.73% yield, 96% purity) as a purple solid. ¹H NMR CHLOROFORM-d 400 MHz δ=14.86 (s, 1H), 8.97 (br s, 1H), 8.12 (s, 1H), 6.45 (br dd, J=10.4, 15.8 Hz, 1H), 6.27 (br d, J=10.5 Hz, 1H), 6.19-6.05 (m, 2H), 5.05 (dd, J=6.3, 12.4 Hz, 1H), 4.76 (br d, J=3.6 Hz, 1H), 4.62 (br d, J=10.6 Hz, 1H), 3.82 (s, 1H), 3.69 (br d, J=9.8 Hz, 1H), 3.52-3.39 (m, 5H), 3.10 (s, 3H), 3.08-2.94 (m, 3H), 2.71-2.58 (m, 2H), 2.41-2.25 (m, 1H), 2.20-2.15 (m, 1H), 2.08 (br s, 5H), 1.97 (br d, J=15.3 Hz, 1H), 1.89-1.83 (m, 2H), 1.79 (s, 5H), 1.71-1.64 (m, 1H), 1.58-1.52 (m, 1H), 1.06 (br d, J=6.9 Hz, 3H), 0.96 (br d, J=6.3 Hz, 6H), 0.91 (d, J=6.4 Hz, 6H), 0.87 (br d, J=6.9 Hz, 3H), 0.60 (br d, J=6.6 Hz, 3H), −0.01 (br d, J=7.0 Hz, 31H)

### Synthesis of Carbamate 6

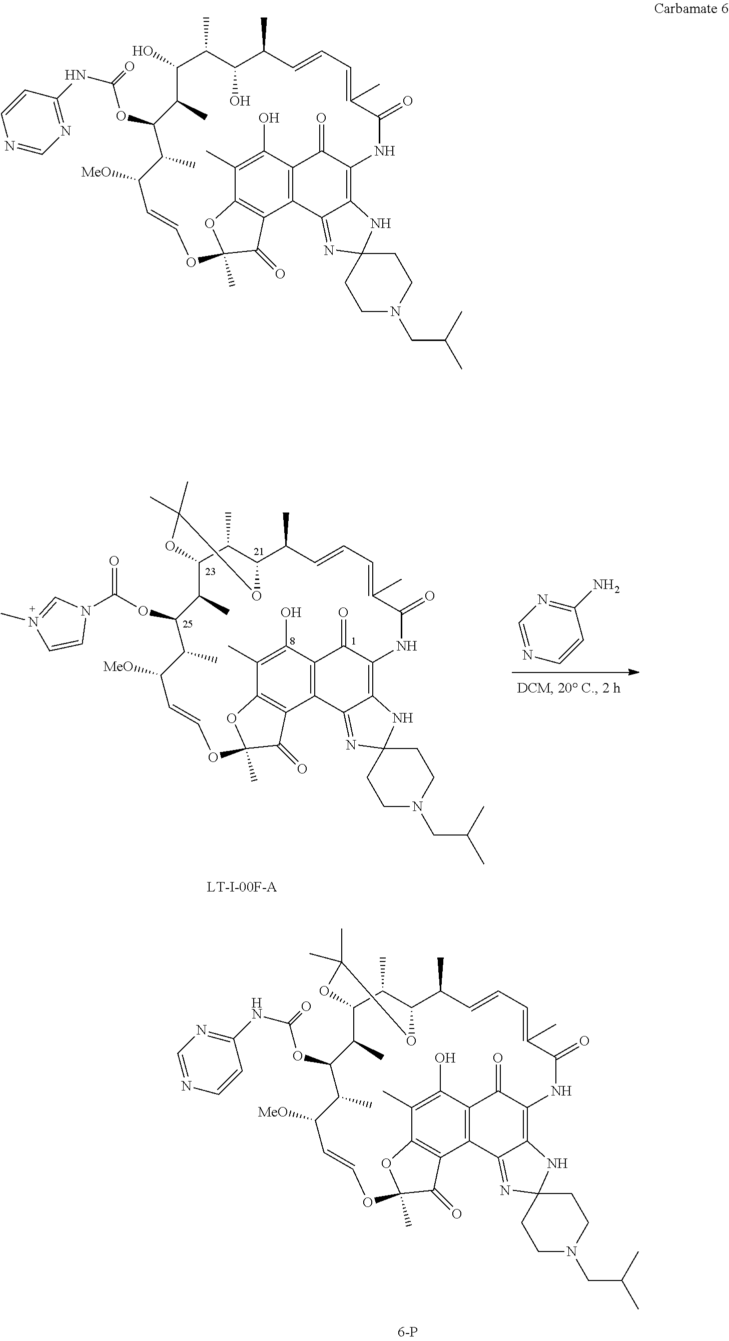

To a solution of LT-I-00F-A (250 mg, 262.02 umol) in DCM (2 mL) was added pyrimidin-4-amine (74.76 mg, 786.06 mmol). The mixture was stirred at 20° C. for 2 hr. LC-MS showed LT-I-00F-A was consumed completely and desired mass was detected. The reaction mixture was concentrated under reduced pressure to remove solvent. The residue was purified by prep-TLC (SiO₂, Petroleum ether/Ethyl acetate/Methanol=7/2/1, P1 Rf=−0.31) to afford 6-P (70 mg, 23.78% yield, 86%, purity) as a purple solid.

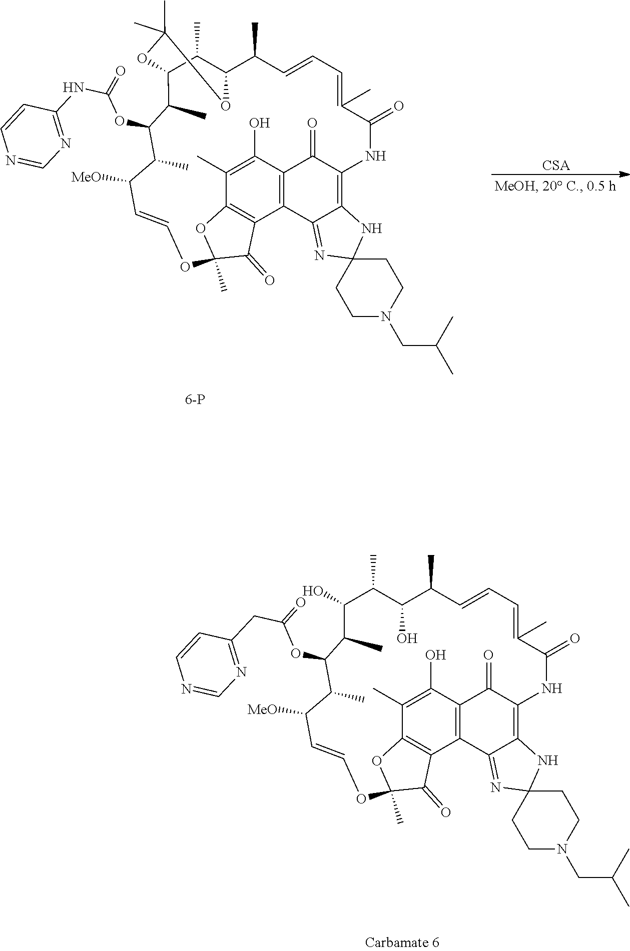 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@@H]2OC(C)(C)O[C@@H]([C@@H](C)[C@H](OC(=O)Nc3ccncn3)[C@@H]1C)[C@@H]2C)C4=O ; "CSA" ; CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)Cc2ccncn2)[C@@H]1C)C4=O]

To a solution of 6-P (70 mg, 72.45 umol) in MeOH (1 mL) was added CSA (36.27 mg, 144.91 umol). The mixture was stirred at 20° C. for 0.5 hr. LC-MS showed 6-P was consumed completely and desired mass was detected. The NaHCO₃ (30 mg) was added after which the mixture was partitioned between DCM (3 mL) and water (3 mL). The aqueous phase was back extracted with DCM (3*5 mL). The organic layers were combined, dried (Na₂SO₄) and concentrated to give a residue. The residue was purified by prep-HPLC (neutral condition column: Waters Xbridge BEH C18 100*30 mm*10 um; mobile phase: [Water (NH₄HCO₃)-ACN]; B %: 50%-70%, 8 min to afford Carbamate 6 (34.2 ng, 50.97% yield, 100% purity) as a purple solid. ¹H NMR CHLOROFORM-d 400 MHz δ=14.71 (br s, 1H), 9.36 (br s, 1H), 9.15 (br s, 1H), 8.86 (s, 1H), 8.59 (d, J=5.9 Hz, 1H), 8.23 (s, 1H), 8.01 (d, J=5.5 Hz, 1H), 6.39-6.21 (m, 3H), 5.96 (dd, J=6.6, 15.4 Hz, 1H), 5.27 (dd, J=8.5, 12.6 Hz, 1H), 4.76 (br d, J=10.6 Hz, 1H), 3.70-3.62 (m, 2H), 3.32 (br dd, J=3.0, 8.5 Hz, 1H), 3.27 (s, 1H), 3.18-3.12 (m, 1H), 2.96 (s, 5H), 2.66 (br s, 2H), 2.39 (s, 4H), 2.31 (br d, J=7.3 Hz, 2H), 2.12 (br d, J=8.8 Hz, 1H), 2.06 (s, 3H), 2.01 (dt, J=3.5, 6.9 Hz, 3H), 1.86 (br dd, J=6.7, 13.4 Hz, 2H), 1.76 (s, 4H), 1.45-1.36 (m, 1H), 1.04 (br d, J=7.0 Hz, 3H), 0.96 (d, J=6.5 Hz, 6H), 0.84 (br d, J=6.9 Hz, 3H), 0.65 (br d, J=6.9 Hz, 3H), −0.01 (br d, J=7.0 Hz, 3H)

### Synthesis of Carbamate 7

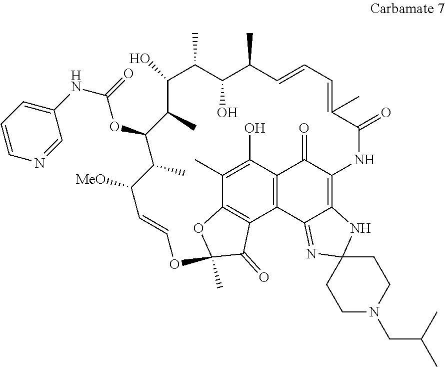 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)Nc2cccnc2)[C@@H]1C)C4=O]

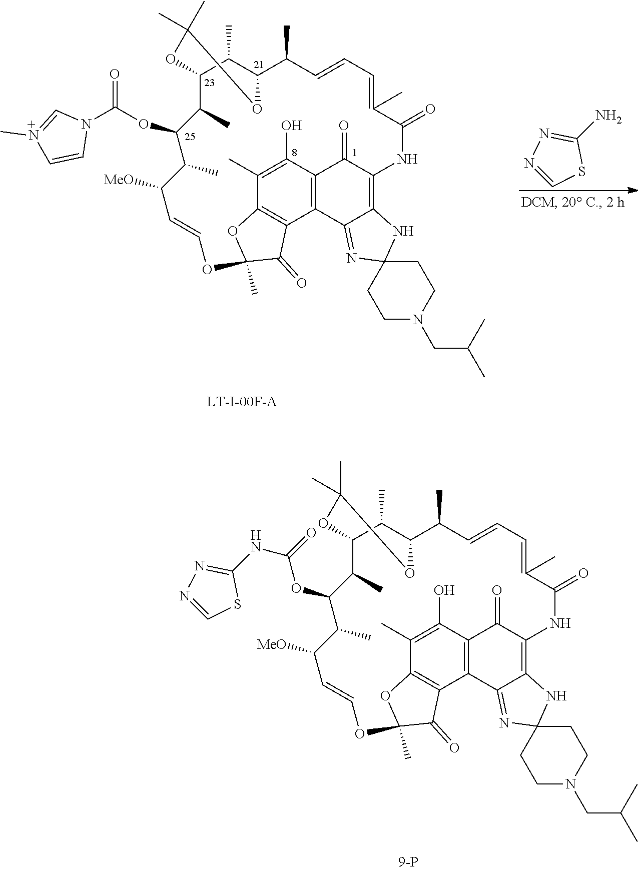

To a solution of LT-I-00F-A (250 mg, 262.02 umol) in DCM (2 mL) was added pyridin-3-amine (73.98 mg, 786.06 umol). The mixture was stirred at 20° C. for 3 hr. LC-MS showed LT-I-00F-A was consumed completely and desired mass was detected. The reaction mixture was concentrated under reduced pressure to remove solvent. The residue was purified by prep-TLC (SiO₂, Petroleum ether/Ethyl acetate/Methanol=7/2/1, P1 Rf=0.31) to afford 7-P (130 mg, 51.41% yield) as a purple solid.

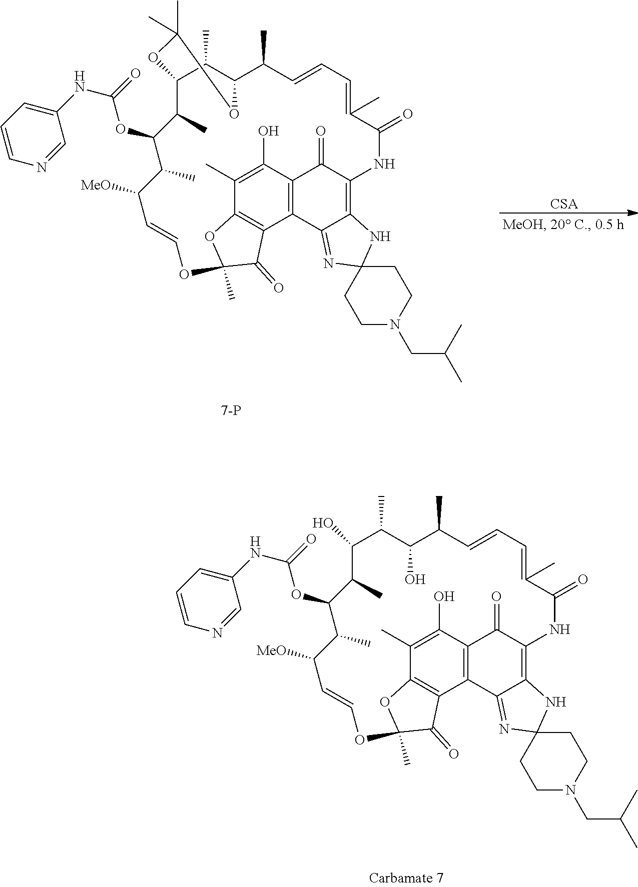 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@@H]2OC(C)(C)O[C@@H]([C@@H](C)[C@H](OC(=O)Nc3cccnc3)[C@@H]1C)[C@@H]2C)C4=O ; "CSA" ; CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)Nc2cccnc2)[C@@H]1C)C4=O]

To a solution of 7-P (120 mg, 124.33 umol) in MeOH (1 mL) was added CSA (62.24 mg, 248.67 umol). The mixture was stirred at 20° C. for 0.5 hr. LC-MS showed 7-P was consumed completely and desired mass was detected. The NaHCO₃ (30 mg) was added after which the mixture was partitioned between DCM (3 mL) and water (3 mL). The aqueous phase was back extracted with DCM (3*5 mL). The organic layers were combined, dried (Na₂SO₄) and concentrated to give a residue. The residue was purified by prep-HPLC (neutral condition column: Waters Xbridge BEH C18 100*30 mm*10 um; mobile phase: [water (NH₄HCO₃-ACN]; B %: 60%-80%, 8 min to afford Carbamate 7 (30.91 mg, 33.41 umol, 26.87% yield) as a purple solid. ¹H NMR CHLOROFORM-d 400 MHz δ=14.77 (s, 1H), 9.13 (br s, 1H), 8.42 (br d, J=1.5 Hz, 1H), 8.31 (br d, J=4.3 Hz, 1H), 8.19 (s, 1H), 8.00 (br s, 1H), 7.26-7.22 (m, 1H), 6.90 (br s, 1H), 6.42-6.33 (m, 1H) 6.28-6.13 (m, 2H), 5.99 (dd, J=6.8, 15.8 Hz, 1H), 5.14 (dd, J=7.6, 12.5 Hz, 1H), 4.69 (br d, J=10.5 Hz, 1H), 3.94 (br d, J=5.3 Hz, 1H), 3.65 (br d, J=9.9 Hz, 1H), 3.48 (s, 1H), 3.40 (br d, J=5.8 Hz, 1H), 3.16 (br dd, J=5.3, 9.0 Hz, 1H), 3.05 (s, 3H), 2.96 (br s, 2H), 2.62 (br s, 2H), 2.41-2.31 (m, 4H), 2.27 (br d, J=6.6 Hz, 2H), 2.11 (br d, J=5.9 Hz, 1H), 2.04 (s, 3H), 1.95 (br s, 2H), 1.86-1.72 (m, 71H), 1.52-1.41 (m, 1H), 1.03 (d, J=6.9 Hz, 31H), 0.93 (br d, J=6.4 Hz, 6H), 0.83 (d, J=6.9 Hz, 3H), 0.62 (d, J=6.9 Hz, 3H), −0.01 (d, J=7.0 Hz, 3H)

### Synthesis of Carbamate 8

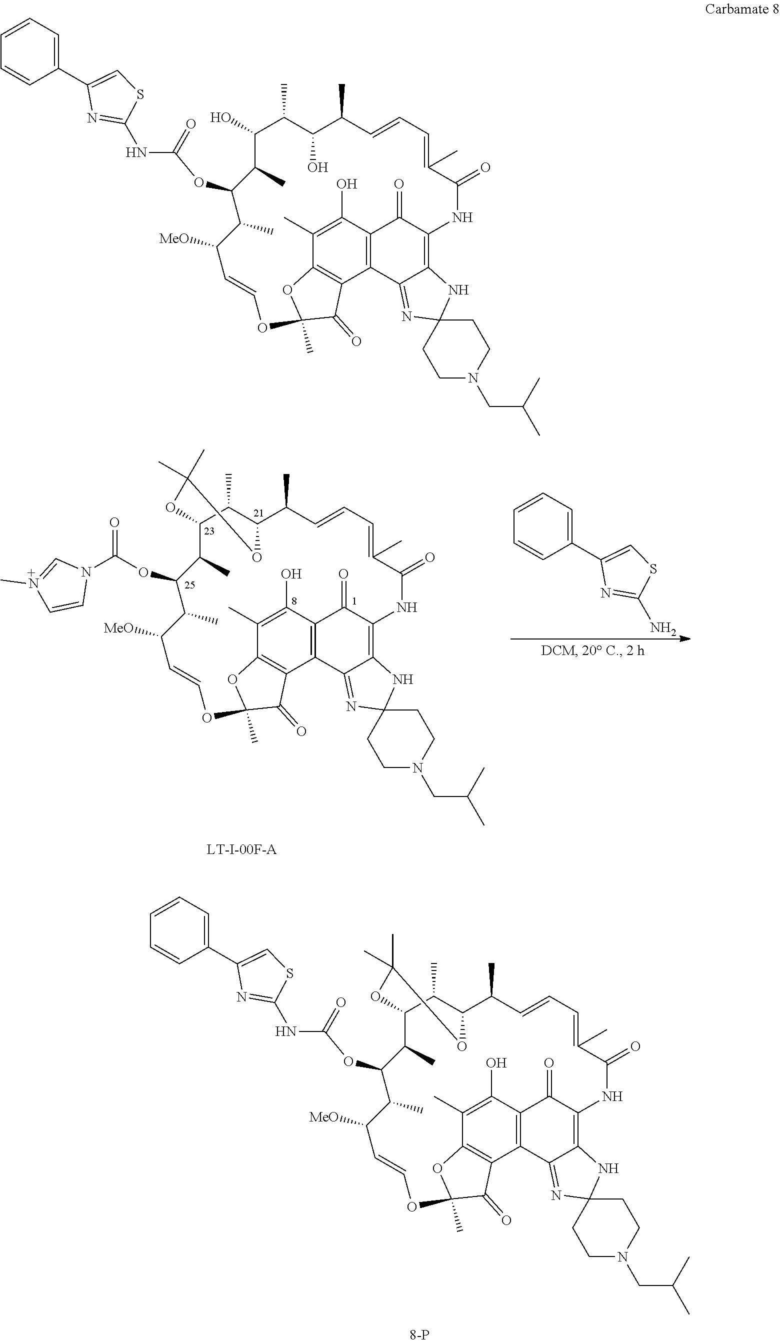

To a solution of LT-I-00F-A (250 mg, 262.02 umol) in DCM (2 mL) was added 4-phenylthiazol-2-amine (138.53 mg, 786.06 umol) The mixture was stirred at 20° C. for 2 hr. LC-MS showed LT-I-00F-A was consumed completely and desired mass was detected. The reaction mixture was concentrated under reduced pressure to remove solvent. The residue was purified by prep-TLC (SiO₂, Petroleum ether/Ethyl acetate/Methanol=7/2/1, P1 Rf=0.31) to afford 8-P (100 mg, 30.98% yield, 85% purity) as a purple solid.

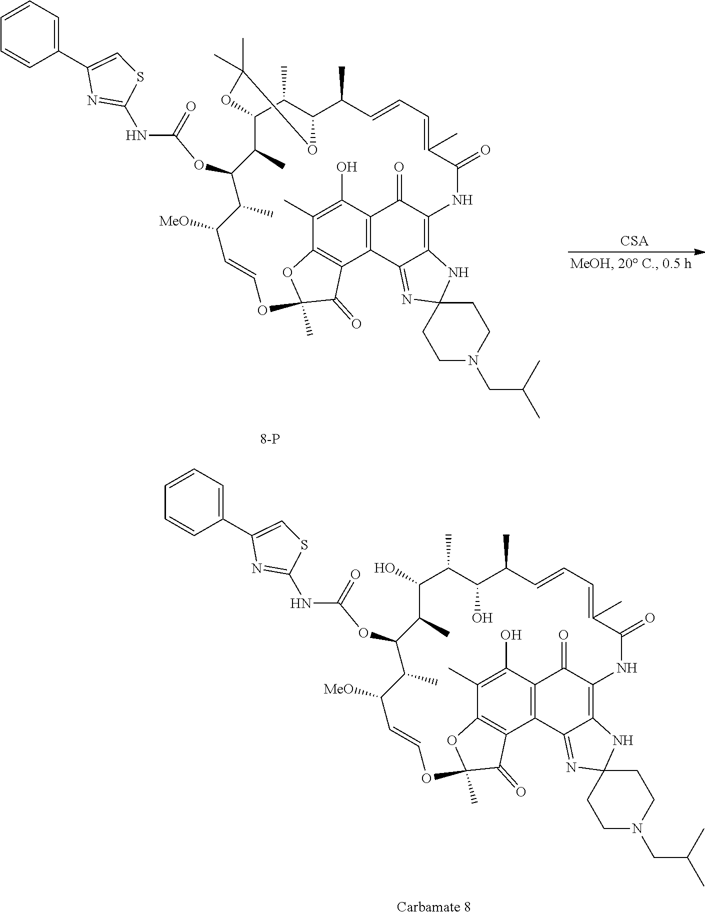 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@@H]2OC(C)(C)O[C@@H]([C@@H](C)[C@H](OC(=O)Nc3nc(-c5ccccc5)cs3)[C@@H]1C)[C@@H]2C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)Nc2nc(-c3ccccc3)cs2)[C@@H]1C)C4=O]

To a solution of 8-P (100 mg, 95.9 umol) in MeOH (1 mL) was added CSA (47.80 mg, 190.97 umol). The mixture was stirred at 20° C. for 0.5 hr. LC-MS showed 8-P was consumed completely and ˜52% of desired mass was detected. The NaHCO₃ (30 mg) was added after which the mixture was partitioned between DCM (3 mL) and water (3 mL). The aqueous phase was back extracted with DCM (3*5 mL). The organic layers were combined, dried (Na₄SO₄) and concentrated to give a residue. The residue was purified by prep-H PLC (neutral condition column: Waters, Xbridge BEH C18 100*30 mm*10 um; mobile phase: [water(NH₄HCO₃)-ACN]; B %: 60%-80%, 8 min to afford Carbamate 8 (15.6 mg, 15.57% yield) was obtained as a purple solid. ¹H NMR CHLOROFORM-d 400 MHz δ=: 14.79 (s, 1H), 9.15 (br s, 1H), 8.82-8.66 (m, 1H), 8.19 (s, 1H), 7.82 (d, J=7.4 Hz, 2H), 7.44-7.39 (m, 2H), 7.36-7.31 (m, 1H), 7.14 (s, 1H), 6.46-6.36 (m, 1H), 6.33-6.26 (m, 1H), 6.20 (d, J=12.5 Hz, 1H), 6.04 (br dd, J=6.7, 15.7 Hz, 1H), 5.20 (dd, J=7.4, 12.5 Hz, 1H), 4.82 (br d, J=9.0 Hz, 1H), 3.77-3.66 (m, 2H), 3.45 (s, 1H), 3.39 (br d, J=5.4 Hz, 1H), 3.21-3.14 (m, 1H), 3.05 (s, 3H), 3.03-2.96 (m, 2H), 2.70 (br s, 2H), 2.41 (s, 4H), 2.35 (br dd, J=2.9, 7.1 Hz, 2H), 2.18-2.14 (m, 1H), 2.09 (s, 3H), 1.89 (td, J==6.3, 13.1 Hz, 2H), 1.84-1.71 (m, 7H), 1.57-1.45 (m, 1H), 1.07 (d, J=7.0 Hz, 3H), 0.98 (d, J=6.4 Hz, 6H), 0.87 (d, J=6.9 Hz, 3H), 0.59 (br d, J=4.8 Hz, 3H), −0.01 (d, J=7.01 Hz, 3H)

### Synthesis of Carbamate 9

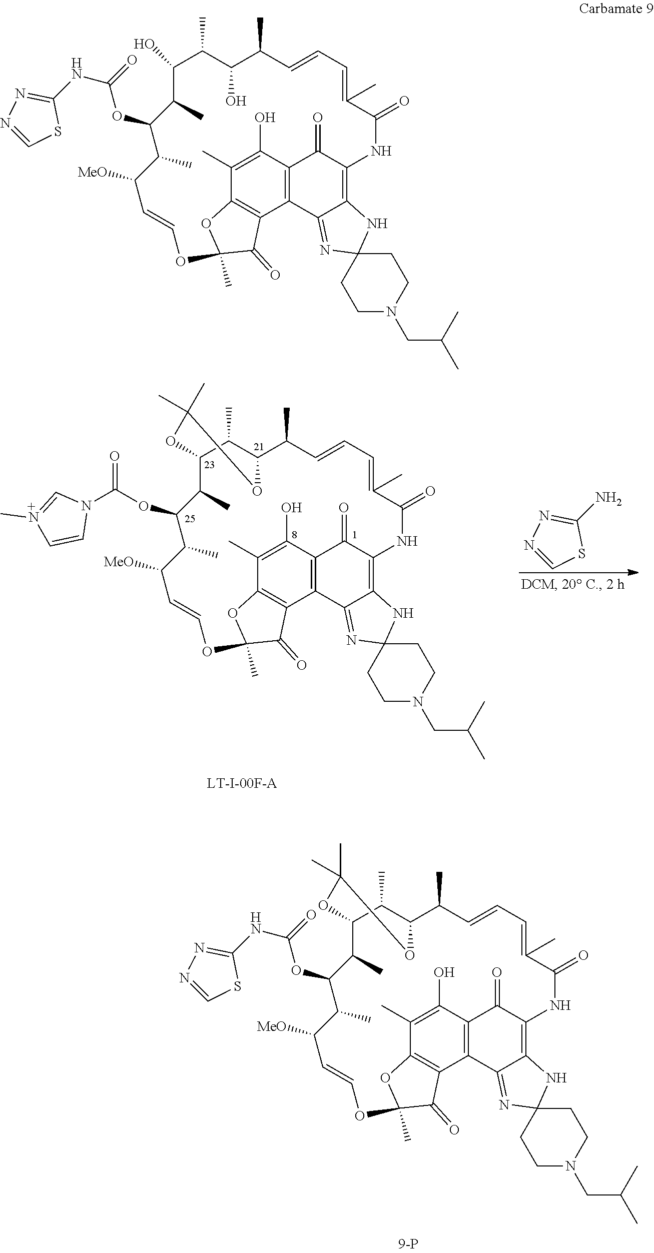

To a solution of LT-1-OOF-A (250 mg, 262.02 umol) in DCM (2 mL) was added 1,3,4-thiadiazol-2-amine (79.49 mg, 786.06 umol). The mixture was stirred at 20° C. for 3 hr. LC-MS showed LT-I-00F-A was consumed completely and desired mass was detected. The reaction mixture was concentrated under reduced pressure to give residue. The residue was purified by prep-TLC (SiO₂, Petroleum ether/Ethyl acetate/Methanol=7/2/1, P1 Rf=0.31) to afford 9-P (70 mg, 27.48% yield) as a purple solid.

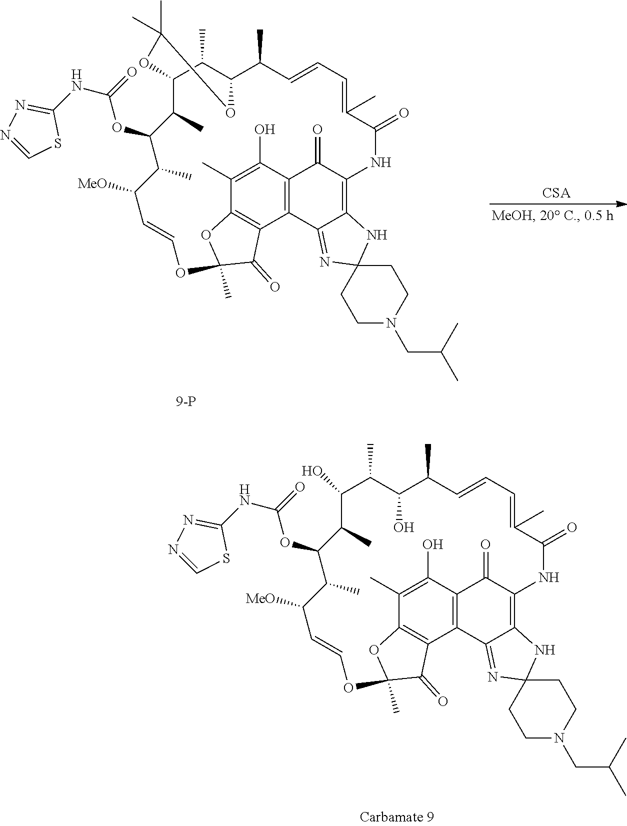 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@@H]2OC(C)(C)O[C@@H]([C@@H](C)[C@H](OC(=O)Nc3nncs3)[C@@H]1C)[C@@H]2C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)Nc2nncs2)[C@@H]1C)C4=O]

To a solution of 9-P (60 mg, 61.72 umol) in MeOH (1 mL) was added CSA (30.90 mg, 123.44 umol). The mixture was stirred at 20° C. for 0.5 hr. LC-MS showed 9-P was consumed completely and desired mass was detected. The NaHCO₃ (30 mg) was added after which the mixture was partitioned between DCM (3 mL) and water (3 mL). The aqueous phase was back extracted with DCM (3*5 mL). The organic layers were combined, dried (Na₂SO₄) and concentrated under vacuum to give residue. The residue was purified by prep-HPLC (neutral condition column: Waters Xbridge Prep OBD C18 150*40 mm*10 um; mobile phase: [water (NH₄HCO₃)-ACN]; B %: 45%-75%, 8 min to afford Carbamate 9 (14.92 mg, 15.53 umol, 25.16% yield, 97% purity) as a purple solid. ¹H NMR CHLOROFORM-d 400 MHz δ=14.74 (s, 1H), 9.31 (br s, 1H), 8.79 (s, 1H), 8.23 (s, 1H), 6.42-6.18 (m, 3H), 6.02 (br dd, J=6.6, 15.6 Hz, 1H), 5.33 (dd, J=8.0, 12.5 Hz, 1H), 4.90 (br d, J=10.5 Hz, 1H), 3.72 (br d, J=9.4 Hz, 2H), 3.46 (br d, J=5.9 Hz, 1H), 3.32 (br s, 1H), 3.27-3.17 (m, 1H), 3.00 (s, 5H), 2.72 (br s, 2H), 2.49-2.30 (m, 7H), 2.09 (s, 5H), 1.98-1.84 (m, 4H), 1.79 (s, 4H), 1.53-1.42 (m, 1H), 1.07 (br d, J=6.9 Hz, 3H), 0.97 (br dd, J=2.9, 5.7 Hz, 6H), 0.88 (br d, J=6.9 Hz, 3H), 0.68 (br d, J=6.6 Hz, 3H), −0.01 (br d, J=7.0 Hz, 3H)

### Synthesis of Carbamate 10

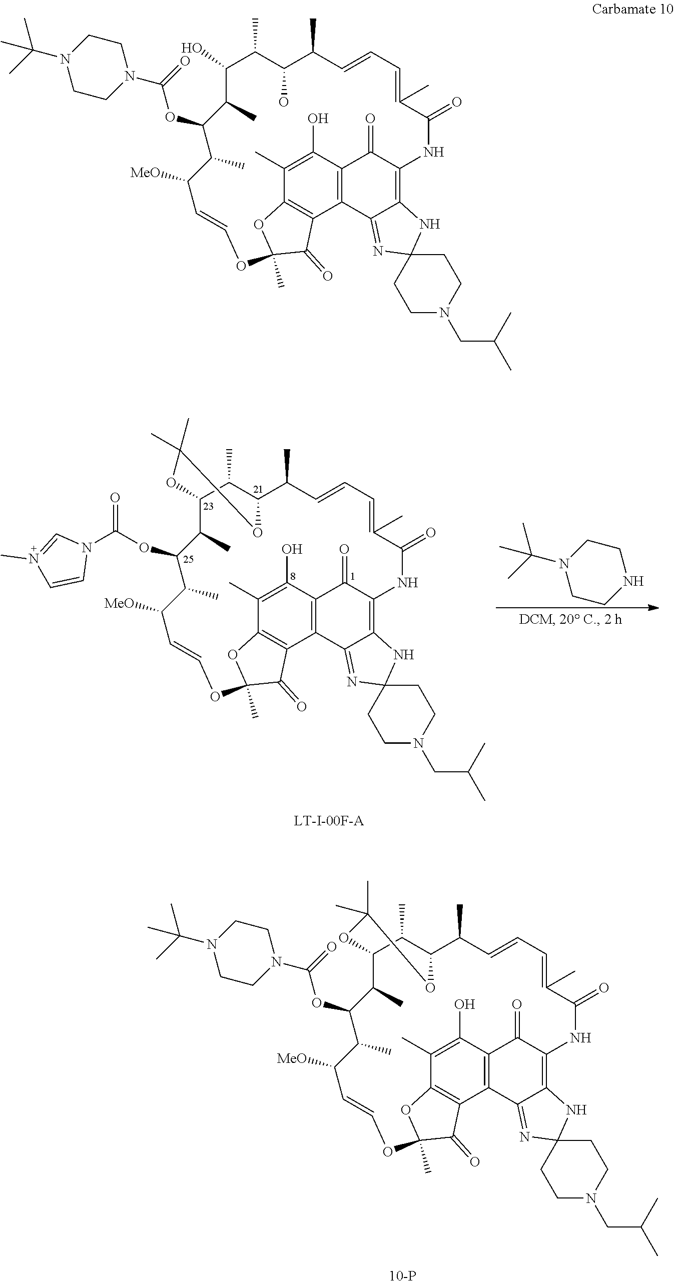

To a solution of LT-I-00F-A (200 mg, 209.61 umol) in DCM (2 mL) was added 1-tert-butylpiperazine (89.45 mg, 628.84 umol). The mixture was stirred at 20° C. for 3 hr. LC-MS showed LT-I-00F-A was consumed completely and desired mass was detected. The reaction mixture was concentrated under reduced pressure to give reside. The residue was purified by prep-TLC (SiO₂, Petroleum ether/Ethyl acetate/Methanol=7/2/1, P1 Rf=0.31) to afford 10-P (100 mg,

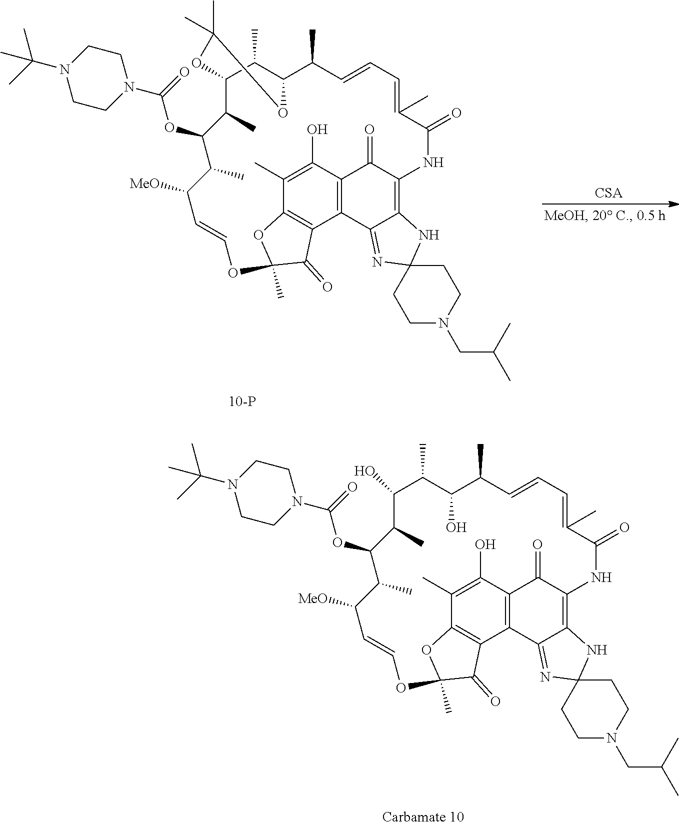

To a solution of 10-P (100 mg, 98.68 umol) in MeOH (1 mL) was added CSA (49.41 mg, 197.38 umol). The mixture was stirred at 20° C. for 0.5 hr. LC-MS showed 10-P was consumed completely and desired mass was detected. The NaHCO₃ (30 mg) was added after which the mixture was partitioned between DCM (3 mL) and water (3 mL). The aqueous phase was back extracted with DCM (3*5 mL). The organic layers were combined, dried (Na₂SO₄) and concentrated under vacuum to give reside. The residue was purified by prep-HPLC (neutral condition column: Phenomenex C18 75*30 mm*3 um; mobile phase: [water (NH₄HCO₃)-ACN]; B %: 60%-95%, 10 min to afford Carbamate 10 (46.7 mg, 45.71% yield, 94% purity) as a purple solid. ¹H NMR CHLOROFORM-d 400 MHz δ=14.87 (s, 1H), 8.95 (br s, 1H), 8.16 (s, 1H), 6.50-6.41 (m, 1H), 6.27 (d, J=10.5 Hz, 1H), 6.18-6.07 (m, 2H), 5.06 (dd, J=6.3, 12.4 Hz, 1H), 4.77 (d, J=4.0 Hz, 1H), 4.64 (d, J=10.4 Hz, 1H), 3.84 (s, 1H), 3.70 (d, J=9.8 Hz, 1H), 3.52-3.37 (m, 5H), 3.10 (s, 3H), 3.07 (br dd, J=2.8, 10.3 Hz, 1H), 2.99 (br dd, J=3.5, 7.4 Hz, 2H), 2.71-2.59 (m, 2H), 2.50 (br d, J=5.0 Hz, 4H), 2.39 (br dd, J=70, 16.4 Hz, 1H), 2.35-2.29 (m, 5H), 2.24-2.14 (m, 1H), 2.08 (s, 3H), 2.03-1.93 (m, 2H), 1.89-1.82 (m, 2H), 1.80 (s, 4H), 1.67 (ddd, J=2.4, 7.3, 10.2 Hz, 1H), 1.59-1.53 (m, 1H), 1.09-1.04 (m, 1H), 0.97 (d, J=6.5 Hz, 6H), 0.87 (d, J=6.9 Hz, 3H), 0.61 (d, J=6.9 Hz, 3H), −0.01 (d, J=7.1 Hz, 3H)

### Synthesis of Carbamate 11

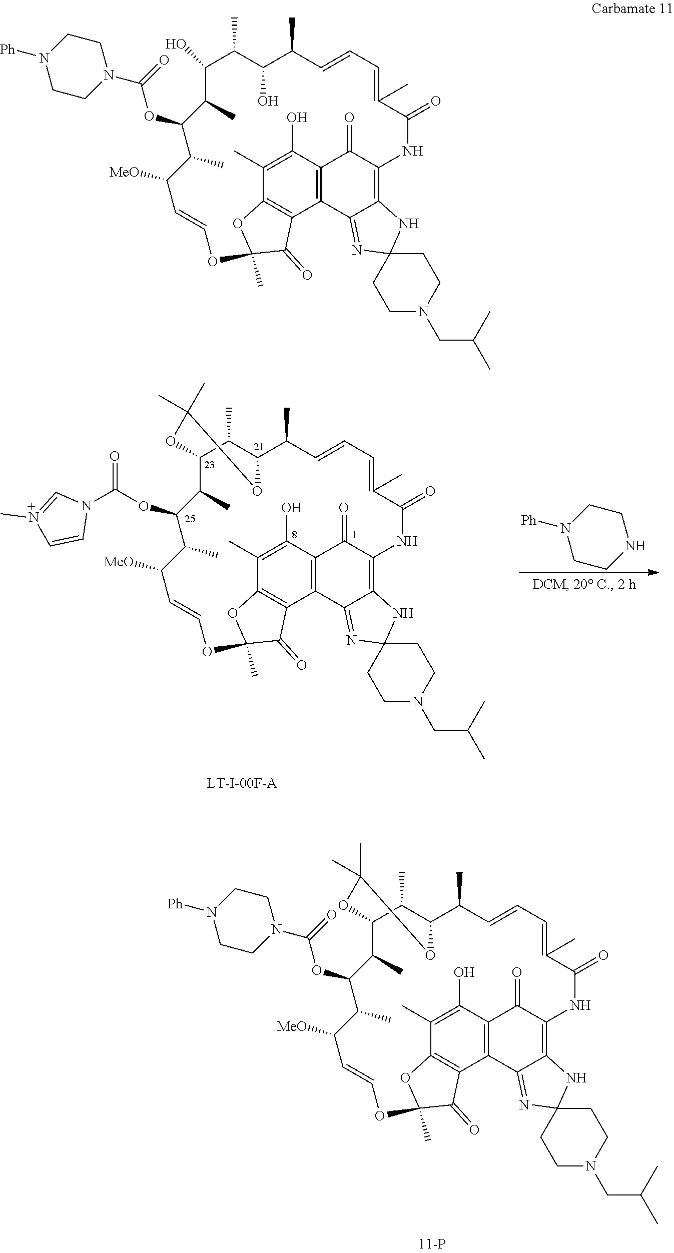

To a solution of LT-I-00F-A (250 mg, 262.02 umol) in DCM (2 mL) was added 1-phenylpiperazine (127.52 mg, 786.06 umol). The mixture was stirred at 20° C. for 3 hr. LC-MS showed LT-I-00F-A was consumed completely and desired mass was detected. The reaction mixture was concentrated under reduced pressure to remove solvent. The residue was purified by prep-TLC (SiO₂, Petroleum ether/Ethyl acetate/Methanol=7/2/1, P1 Rf=0.31) to afford 11-P (100 mg, 32.50% yield, 88% purity) as a purple solid.

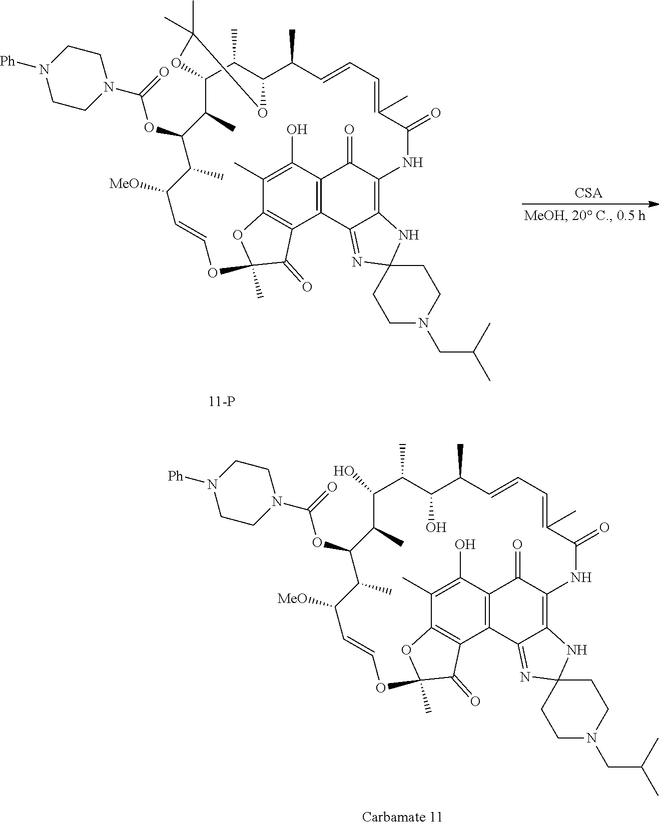 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@@H]2OC(C)(C)O[C@@H]([C@@H](C)[C@H](OC(=O)N3CCN(c5ccccc5)CC3)[C@@H]1C)[C@@H]2C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)N2CCN(c3ccccc3)CC2)[C@@H]1C)C4=O]

To a solution of 11-P (100 mg, 96.78 umol) in MeOH (1 mL) was added CSA (48.45 mg, 193.56 umol). The mixture was stirred at 20° C. for 0.5 hr. LC-MS showed 11-P was consumed completely and desired mass was detected. The NaHCO₃ (30 mg) was added after which the mixture was partitioned between DCM (3 mL) and water (3 mL). The aqueous phase was back extracted with DCM (3*5 mL). The organic layers were combined, dried (Na₂SO₄) and concentrated to give a residue. The residue was purified by prep-HPLC (neutral condition column: colunm: Waters Xbridge BEH C18 100*30 mm*10 um; mobile phase: [water (NH₄HCO₃)-ACN]; B %: 50%-80%, 8 min to afford Carbamate 11 (46.9 mg, 45.33 umol, 46.84% yield, 96% purity) as a purple solid. ¹H NMR CHLOROFORM-d 400 MHz δ=14.83 (s, 1H), 9.00 (br s, 1H), 8.14 (s, 1H), 7.30-7.26 (m, 2H), 6.93-6.87 (m, 3H), 6.47-6.35 (m, 1H), 6.25 (br d, J=10.1 Hz, 1H), 6.16 (d, J=12.3 Hz, 1H), 6.03 (dd, J=6.9, 15.8 Hz, 1H), 5.04 (dd, J=6.9, 12.4 Hz, 1H). 4.62-4.55 (m, 2H), 3.75 (s, 1H), 3.68-3.51 (m, 5H), 3.36 (dd, J=1.4, 6.8 Hz, 1H), 3.16-3.01 (m, 8H), 2.96 (br d, J=4.0 Hz, 2H), 2.74-2.53 (m, 2H), 2.40-2.26 (m, 6H), 2.19-2.09 (m, 1H), 2.05 (s, 3H), 1.98 (br d, J=15.4 Hz, 2H), 1.88-1.80 (m, 2H), 1.75 (s, 5H), 1.56-1.44 (m, 1H), 1.03 (d, J=7.0 Hz, 3H), 0.94 (d, J=6.5 Hz, 6H), 0.83 (d, J=6.9 Hz, 3H), 0.60 (d, J=6.8 Hz, 3H), −0.01 (d, J=7.1 Hz, 3H)

### Synthesis of Carbamate 12

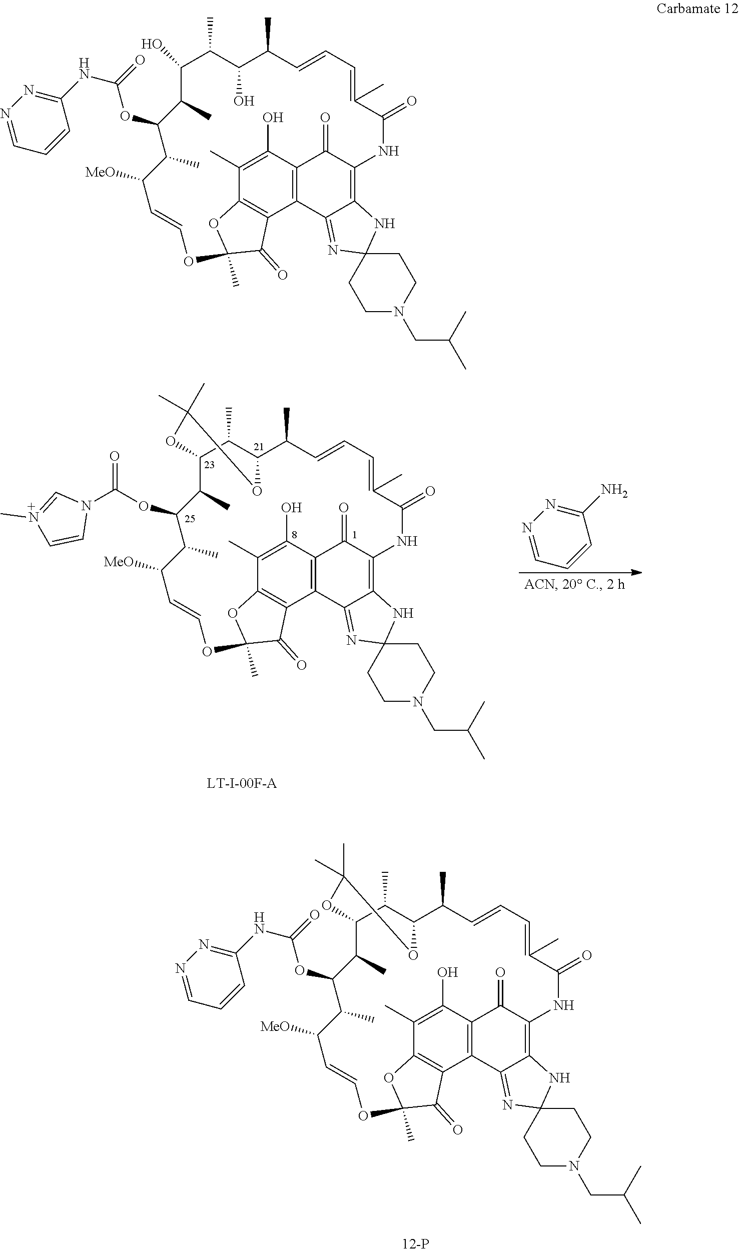

To a solution of LT-I-00F-A (250 mg, 262.02 umol) in DCM (2 mL) was added pyridazin-3-amine (74.76 mg, 786.06 umol). The mixture was stirred at 20° C. for 3 hr. LC-MS showed LT-I-00F-A was consumed completely and desired mass was detected. The reaction mixture was concentrated and purified by prep-TLC (SiO₂, Petroleum ether/Ethyl acetate/Methanol=7/2/1, P1 Rf=0.31) to afford 12-P (80 mg, 23.07% yield, 73% purity) as a purple solid.

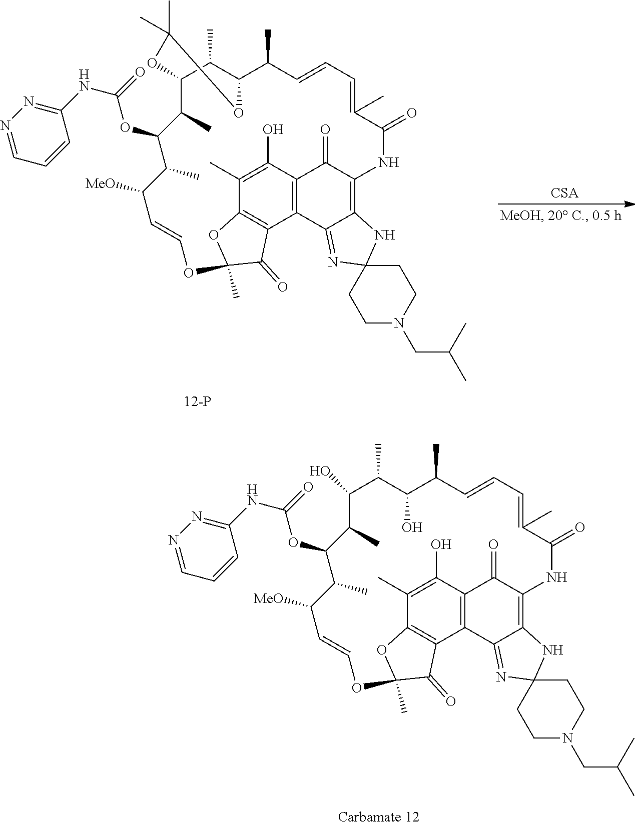 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@@H]2OC(C)(C)O[C@@H]([C@@H](C)[C@H](OC(=O)Nc3cccnn3)[C@@H]1C)[C@@H]2C)C4=O ; CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)Nc2cccnn2)[C@@H]1C)C4=O]

To a solution of 12-P (80 mg. 82.80 umol) in MeOH (1 mL) was added CSA 41.45 mg, 165.61 umol). The mixture was stirred at 20° C. for 0.5 hr. LC-MS showed 12-P was consumed completely and desired mass was detected. The NaHCO₃ (30 mg) was added after which the mixture was partitioned between DCM (3 mL) and water (3 mL). The aqueous phase was back extracted with DCM (3*5 mL). The organic layers were combined, dried (Na₂SO₄) and concentrated to give a residue. The residue was purified by prep-HPLC (neutral condition column: Waters Xbridge BEH C18 100*30 mm*10 um: mobile phase: [water (NH₄HCO₃)-ACN]; B %: 60%-80%, 8 min to afford Carbamate 12 (26.9 mg, 35.08% yield, 100% purity) as a purple solid. ¹H NMR CHLOROFORM-d 400 MHz δ=14.74 (s, 1H), 9.28 (br s, 1H), 8.90 (br d, J=3.9 Hz, 1H), 8.29-8.21 (m, 2H), 7.99 (s, 1H), 7.48 (dd, J=4.6, 9.0 Hz, 1H), 6.41-6.33 (m, 1H), 6.30-6.20 (m, 2H), 6.02 (dd, J=6.5, 15.6 Hz, 1H), 5.28 (dd, J=8.1, 12.6 Hz, 1H), 4.80 (br d, J=10.3 Hz, 1H), 3.79-3.69 (m, 2H), 3.45-3.38 (m, 1H), 3.34 (s, 1H), 3.26-3.17 (m, 1H), 3.10-2.95 (m, 5H), 2.67 (br s, 2H), 2.46-2.37 (m, 4H), 2.32 (br d, J=7.3 Hz, 2H), 2.20-2.12 (m, 1H), 2.08 (s, 3H), 2.04-1.96 (m, 2H), 1.93 (br d, J=2.9 Hz, 4H), 1.78 (s, 3H), 1.53-1.42 (m, 1H), 1.07 (d, J=7.0 Hz, 3H), 0.97 (d, J=6.5 Hz, 6H), 0.87 (br d, J=6.9 Hz, 3H), 0.68 (d, J=6.9 Hz, 3H), −0.01 (d, J=7.1 Hz, 3H).

### 25-O-(1-methylpiperidin-4-aminoacyl)-25-O-desacetyrifabutin

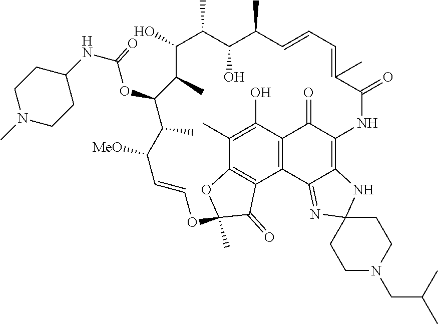 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)NC2CCN(C)CC2)[C@@H]1C)C4=O]

Prepared from LT-I-00F (200.0 mg) using carbamate formation procedure A and the general deprotection procedure to afford the title compound (21.6 mg, 10% over three steps) as a purple solid. ¹H NMR (400 MHz, CHLOROFORM-d) δ ppm −0.03 (d, J=7.13 Hz, 3H) 0.57 (d, J=6.88 Hz, 3H) 0.84 (d, J=7.00 Hz, 3H) 0.94 (d, J=6.50 Hz, 6H) 1.03 (d, J=7.00 Hz, 3H) 1.39-1.52 (m, 3H) 1.58-1.67 (m, 1H) 1.74-1.79 (m, 4H) 1.81-1.99 (m, 7H) 2.03-2.13 (m, 6H) 2.26-2.43 (m, 9H) 2.54-2.70 (m, 2H) 2.79 (br d, J=10.13 Hz, 2H) 2.98 (br d, J=5.25 Hz, 2H) 3.09 (s, 4H) 3.32-3.84 (m, 4H) 4.46-4.57 (m, 2H) 4.66 (br d, J=8.13 Hz, 1H) 5.03 (dd, J=12.44, 6.57 Hz, 1H) 6.04 (dd, J=15.70, 6.94 Hz, 1H) 6.15 (d, J=12.51 Hz, 1H) 6.25 (br d, J=10.13 Hz, 1H) 6.32-6.45 (m, 1H) 8.19 (s, 1H) 8.91 (br s, 1H) 14.85 (s, 1H); HRMS (ESI-TOF) m/z [M+H]⁺ calcd for C₅₁H₇₃N₆O₁₁ 945.5332, found 945.5368 (error 3.8 ppm).

### 25-O-(1-ethylpiperidin-4-aminoacyl)-25-O-desacetyrifabutin

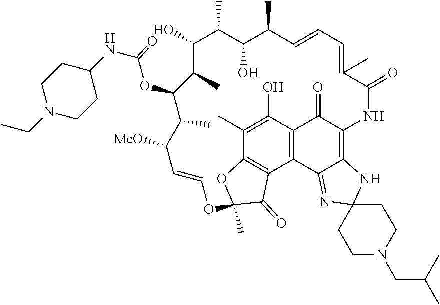 [SMILES: CCN1CCC(NC(=O)O[C@H]2[C@H](C)[C@H](O)[C@H](C)[C@@H](O)[C@@H](C)/C=C/C=C(\C)C(=O)NC3=C4NC5(CCN(CC(C)C)CC5)N=C4c4c(c(O)c(C)c5c4C(=O)[C@@](C)(O/C=C/[C@H](OC)[C@H]2C)O5)C3=O)CC1]

Prepared from LT-I-00F (300.0 mg) using carbamate formation procedure A and the general deprotection procedure to afford the title compound (35.0 mg, 10% over three steps) as a purple solid. ¹H NMR (400 MHz, CHLOROFORM-d) δ ppm −0.05 (br d, J=7.00 Hz, 3H) 0.56 (br d, J=6.75 Hz, 3H) 0.84 (d, J=6.88 Hz, 3H) 0.95 (d, J=6.50 Hz, 6H) 1.03 (d, J=7.00 Hz, 3H) 1.14 (br s, 3H) 1.40-1.67 (m, 4H) 1.83-1.99 (m, 5H) 2.00-2.12 (m, 5H) 2.19 (br d, J=9.51 Hz, 2H) 2.29-2.38 (m, 6H) 2.47-2.78 (m, 5H) 2.90-3.15 (m, 11H) 3.37-3.73 (m, 4H) 4.55 (br d, J=10.51 Hz, 2H) 4.83 (br d, J=7.88 Hz, 1H) 5.03 (dd, J=12.32, 6.44 Hz, 1H) 5.89-6.49 (m, 4H) 8.28 (s, 1H) 8.70-9.12 (m, 1H) 14.84 (br s, 1H); HRMS (ESI-TOF) m/z [M+H]⁺ calcd for C₅₂H₇₅N₆O₁₁ 959.5488, found 959.5521 (error 3.4 ppm).

### 25-O-(1-isopropylpiperidin-4-aminoacyl)-25-O-desacetyrifabutin

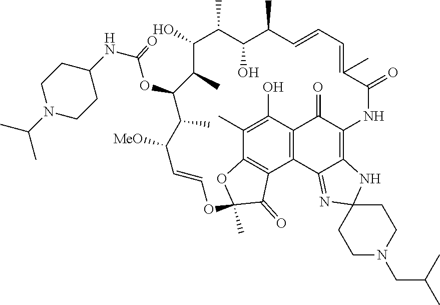 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)NC2CCN(C(C)C)CC2)[C@@H]1C)C4=O]

Prepared from LT-I-00F (200.0 mg) using carbamate formation procedure A and the general deprotection procedure to afford the title compound (19.8 mg, 10% over three steps) as a purple solid. 1H NMR (400 MHz, CHLOROFORM-d) δ ppm −0.03 (d, J=7.0 Hz, 3H) 0.57 (br d, J=6.88 Hz, 3H) 0.84 (d, J=6.88 Hz, 3H) 0.94 (d, J=6.50 Hz, 6H) 1.00-1.07 (m, 9H) 1.26-1.55 (m, 4H) 1.62-1.79 (m, 7H) 1.81-2.00 (m, 7H) 2.06 (s, 5H) 2.22-2.40 (m, 8H) 2.55-2.78 (m, 3H) 2.84 (br d, J=10.88 Hz, 2H) 2.97 (br d, J=4.25 Hz, 2H) 3.36-3.52 (m, 2H) 3.64 (br d, J=9.76 Hz, 1H) 3.77 (s, 1H) 4.46-4.59 (m, 2H) 4.65 (br d, J=8.25 Hz, 1H) 5.03 (dd, J=12.44, 6.57 Hz, 1H) 6.04 (br dd, J=15.57, 6.94 Hz, 1H) 6.15 (d, J=12.38 Hz, 1H) 6.25 (br d, J=9.88 Hz, 1H) 6.40 (br dd, J=15.63, 10.26 Hz, 1H) 8.17 (s, 1H) 8.91 (br s, 1H) 14.86 (s, 1H); HRMS (ESI-TOF) m/z [M+H]⁺ calcd for C₅₃H₇₇N₆O₁₁ 973.5645, found 973.5668 (error 2.4 ppm).

### 25-O-(1-isobutylpiperidin-4-aminoacyl)-25-O-desacetyrifabutin

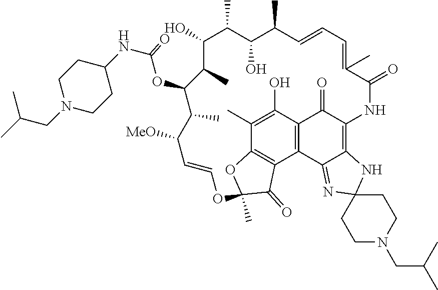 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)NC2CCN(CC(C)C)CC2)[C@@H]1C)C4=O]

Prepared from LT-I-00F (200.0 mg) using carbamate formation procedure A and the general deprotection procedure to afford the title compound (35.2 mg, 15% over three steps) as a purple solid. 1H NMR (400 MHz, CHLOROFORM-d) δ ppm −0.03 (br d, J=7.00 Hz, 3H) 0.57 (br d, J=6.75 Hz, 3H) 0.84 (d, J=6.88 Hz, 3H) 0.89 (br d, J=6.25 Hz, 6H) 0.95 (d, J=6.50 Hz, 6H) 1.03 (d, J=7.00 Hz, 3H) 1.37-1.53 (m, 3H) 1.55-1.68 (m, 1H) 1.70-1.80 (m, 5H) 1.81-1.98 (m, 5H) 2.00-2.13 (m, 8H) 2.26-2.43 (m, 6H) 2.63-2.89 (m, 4H) 2.95-3.15 (m, 6H) 3.31-3.54 (m, 3H) 3.58-3.96 (m, 2H) 4.38-4.76 (m, 3H) 5.02 (dd, J=12.51, 6.38 Hz, 1H) 5.90-6.72 (m, 4H) 8.15 (s, 1H) 8.87 (br s, 1H) 14.84 (s, 1H); HRMS (ESI-TOF) m/z [M+H]⁺ calcd for C₅₄H₇₉N₆O₁₁ 987.5801, found 987.5798 (error 0.3 ppm).

### 25-O-(pyrimidin-4-aminoacyl)-25-O-desacetyrifabutin

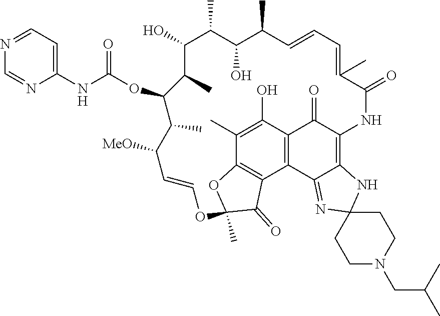 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)Nc2ccncn2)[C@@H]1C)C4=O]

Prepared from 3 (29.9 mg) using carbamate formation procedure B and the general deprotection procedure to afford the title compound (16.0 mg. 49% over two steps) as a purple solid. ¹H NMR (601 MHz, CDCl₃) δ 14.67 (s, 1H), 9.44-9.20 (br, 1H), 8.82 (d, J=1.3 Hz, 1H), 8.57 (d, J=5.8 Hz, 1H), 8.48 (s, 1H), 8.18 (s, 1H), 7.97 (dd, J=5.9, 1.3 Hz, 1H), 6.33 (dd, J=15.7, 10.1 Hz, 1H), 6.26 (dd, J=10.1, 1.7 Hz, 1H), 6.22 (d, J=12.6 Hz, 1H), 5.95 (dd, J=15.7, 6.6 Hz, 1H), 5.25 (dd, J=12.6, 8.7 Hz, 1H), 4.75 (dd, J=10.7, 1.9 Hz, 1H), 3.65 (d, J=10.0 Hz, 1H), 3.59 (d, J=6.7 Hz, 1H), 3.31 (dd, J=8.5, 3.2 Hz, 1H), 3.21 (s, 1H), 3.13 (ddd, J=9.7, 6.7, 2.2 Hz, 1H), 3.05-2.89 (m, 1H), 2.97 (s, 3H), 2.73-2.55 (m, 2H), 2.38 is, 3H), 2.43-2.22 (m, 4H), 2.05 (d, J=1.5 Hz, 3H), 2.00-1.91 (m, 2H), 1.90-1.79 (m, 2H), 1.74 (s, 3H), 1.78-1.70 (m, 1H), 1.44-1.36 (m, 1H), 1.03 (d, J=7.0 Hz, 3H), 1.00-0.89 (br, 6H), 0.83 (d, J=6.9 Hz, 3H), 0.64 (d, J=6.9 Hz, 3H), −0.03 (d, J=7.1 Hz, 3H); ¹³C NMR (151 MHz, CDCl₃) δ 192.5, 181.0, 171.5, 168.4, 168.2, 158.3, 158.2, 157.9, 153.5, 145.6, 141.9, 140.8, 133.0, 131.9, 125.1, 124.0, 115.4, 114.7, 111.8, 109.3, 109.2, 107.7, 104.7, 94.9, 82.1, 76.8, 75.2, 72.4, 66.4, 56.5, 51.6, 51.6, 48.5, 43.6, 38.8, 38.3, 37.8, 36.4, 35.6, 33.1, 26.0, 24.1, 22.4, 21.0, 20.4, 17.4, 11.8, 11.3, 9.0, 7.8; MS (ESI) m/z [M+H]⁺ 926.4, [M−H]⁻ 924.4.

### 25-O-(pyrazin-2-aminoacyl)-25-O-desacetyrifabutin

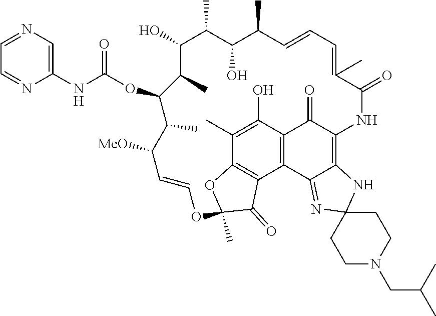 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)Nc2cnccn2)[C@@H]1C)C4=O]

Prepared from 3 (40.0 mg) using carbamate formation procedure B and the general deprotection procedure to afford the title compound (28.2 mg. 64% over two steps) as a purple solid. ¹H NMR (601 MHz, CDCl₃) δ 14.70 (s, 1H), 9.30 (s, 1H), 8.29 (d, J=2.6 Hz, 1H), 8.21-8.18 (m, 2H), 7.86 (s, 1H), 6.36 (dd, J=15.8, 10.1 Hz, 1H), 6.25 (dd, J=10.1, 1.8 Hz, 1H), 6.21 (d, J=12.6 Hz, 1H), 5.96 (dd, J=15.8, 6.7 Hz, 1H), 5.20 (dd, J=12.6, 8.1 Hz, 1H), 4.73 (dd, J=10.6, 1.8 Hz, 1H), 3.73 (d, J=6.1 Hz, 1H), 3.65 (d, J=9.8 Hz, 1H), 3.36 (s, 1H), 3.35 (d, J=3.0 Hz, 1H), 3.05-2.90 (br, 2H), 3.01 (s, 3H), 2.75-2.51 (br, 2H), 2.41-2.36 (m, 1H), 2.36 (s, 3H), 2.34-2.20 (m, 2H), 2.04 (s, 3H), 2.15-1.94 (m, 3H), 1.95-1.87 (m, 2H), 1.87-1.80 (m, 1H), 1.78˜1.74 (m, 3H), 1.74 (s, 3H), 1.48-1.40 (m, 1H), 1.03 (d, J=7.2 Hz, 3H), 0.95 (s, 6H), 0.83 (d, J=6.9 Hz, 3H), 0.63 (d, J=6.9 Hz, 3H), −0.00 (d, J=7.1 Hz, 3H); ¹³C NMR (151 MHz, CDCl₃) δ 192.5, 181.1, 171.5, 168.5, 168.3, 155.3, 153.7, 148.3, 145.3, 142.0, 141.8, 140.9, 139.5, 136.2, 133.1, 131.6, 125.1, 124.2, 115.3, 114.7, 111.9, 109.1, 107.6, 104.8, 94.7, 81.7, 75.4, 72.6, 66.4, 56.6, 51.6, 38.6, 38.1, 38.0, 36.3, 35.5, 33.2, 25.9, 22.3, 21.0, 20.4, 17.4, 11.6, 11.4, 9.0, 7.8; MS (ESI) m/z [M+H]⁺ 926.5, [M−H]⁻ 924.4.

### 25-O-(thiazol-5-aminoacyl)-25-O-desacetyrifabutin

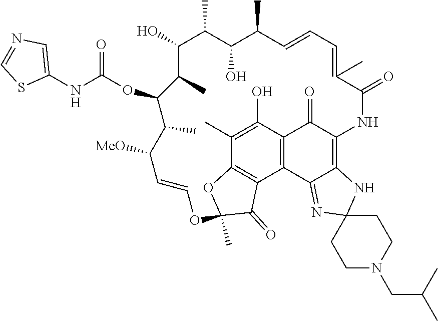 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)Nc2cncs2)[C@@H]1C)C4=O]

Prepared from 3 (40.0 mg) using carbamate formation procedure B and the general deprotection procedure to afford the title compound (36.0 mg, 82% over two steps) as a purple solid. ¹H NMR (601 MHz, CD₂Cl₂) δ 14.83 (s, 1H), 9.13 (s, 1H), 8.39 (s, 1H), 8.16 (s, 1H), 7.45 (s, 1H), 6.35 (dd, J=15.8, 10.3 Hz, 1H), 6.24 (dd, J=10.4, 1.9 Hz, 1H), 6.15 (d, J=12.5 Hz, 1H), 6.01 (dd, J=15.8, 6.8 Hz, 1H), 5.11 (dd, J=12.6, 7.3 Hz, 1H), 4.77 (d, J=10.5 Hz, 1H), 3.86 (d, J=6.0 Hz, 1H), 3.68 (d, =9.8 Hz, 1H), 3.45 (s, 1H), 3.23 (s, 1H), 3.15 (br, 1H), 3.02 (s, 3H), 2.97-2.89 (m, 2H), 2.70-2.50 (br, 28), 2.38-2.32 (m, 1H), 2.31 (s, 3H), 2.26 (d, J=7.4 Hz, 2H), 2.10-1.88 (m, 4H), 2.03 (s, 3H), 1.88-1.80 (m, 1H), 1.80-1.73 (m, 1H), 1.73-1.70 (m, 1H), 1.69 (s, 3H), 1.53-1.44 (m, 1H), 1.01 (d, J=7.0 Hz, 3H), 0.94 (d, J=6.3 Hz, 5H), 0.84 (d, J=7.0 Hz, 3H), 0.60 (d, J=6.8 Hz, 3H), −0.06 (d, J=7.0 Hz, 3H); ¹³C NMR (151 MHz, CD₂Cl₂) δ 192.9, 181.5, 171.9, 168.6, 168.6, 155.6, 154.9, 146.2, 144.5, 142.6, 141.4, 136.8, 133.0, 132.0, 128.6, 125.6, 124.4, 116.2, 114.6, 112.3, 109.3, 107.6, 105.1, 95.1, 80.5, 77.2, 76.3, 73.1, 66.8, 57.0, 51.9, 51.7, 38.7, 38.7, 38.2, 36.5, 35.7, 33.5, 26.2, 21.9, 20.99, 20.98, 20.5, 17.6, 11.4, 11.0, 9.0, 7.8; MS (ESI) m/z [M+H]⁺ 931.4, [M−H]⁻ 929.4.

### 25-O-(5,7-dimethylpyrazolo[1,5-a]pyrimidine-3-aminoacyl)-25-O-desacetyrifabutin

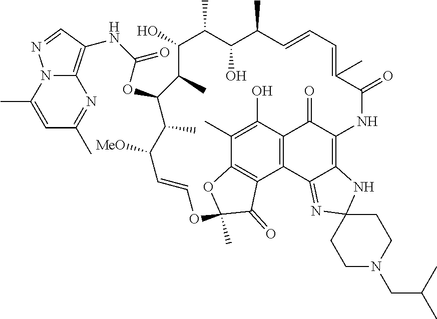 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)Nc2cnn3c(C)cc(C)nc23)[C@@H]1C)C4=O]

Prepared from 3 (135.0 ng) using carbamate formation procedure B and the general deprotection procedure to afford the title compound (25.3 mg, 15% over two steps) as a purple solid. ¹H NMR (400 MHz, CHLOROFORM-d) δ=14.85 (s, 1H), 8.94 (br s, 1H), 8.42 (s, 1-1), 8.12 (s, 1H), 6.92 (s, 1H), 6.59-6.51 (m, 1H), 6.48-6.37 (m, 1H), 6.25 (br d, J=10.3 Hz, 1H), 6.18-6.02 (m, 2H), 5.09 (dd, J=6.3, 12.4 Hz, 1H), 4.76 (br d, J=10.6 Hz, 1H), 4.40 (br d, J=4.4 Hz, 1H), 3.78-3.65 (m, 2H), 3.53 (br d, J=4.8 Hz, 1H, 3.23 (br dd, J=3.8, 9.9 Hz, 1H), 3.10 (s, 3H), 2.97 (br s, 2H), 2.71 (s, 3H), 2.66-2.49 (m, 5H), 2.40-2.25 (m, 5H), 2.21-2.10 (m, 1H), 2.06 (s, 3H), 1.96 (br s, 1H), 1.89-1.67 (m, 7H), 1.64-1.52 (m, 1H), 1.03 (br d, J=6.9 Hz, 3H), 0.95 (br d, J=6.4 Hz, 6H), 0.86 (br d, J=6.9 Hz, 3H), 0.64 (br d, J=6.8 Hz, 3H), 0.00 (br d, J=7.1 Hz, 3H); HRMS (ESI-TOF) m/z [M+H]⁺ calcd for C₅₃H₆₉N8O₁₁ 993.5080, found 993.5084 (error 3.7 ppm).

### 25-O-(5,7-dimethyl-[1,2,4]triazolol1,5-alpyrimidine-2-aminoacyl)-25-O-desacetyrifabutin

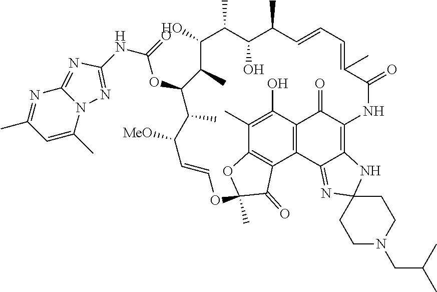 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)Nc2nc3nc(C)cc(C)n3n2)[C@@H]1C)C4=O]

Prepared from 3 (90.0 mg) using carbamate formation procedure B and the general deprotection procedure to afford the title compound (29.5 mg, 14% over two steps) as a purple solid. ¹H NMR (400 MHz, CHLOROFORM-d) δ=14.83 (s, 1H), 8.96 (s, 1H), 8.11 (s, 2H), 6.76 (s, 11H), 6.45-6.36 (m, 1H), 6.25 (br d, J=10.3 Hz, 1H), 6.15-6.03 (m, 2H), 5.07 (dd, J=6.6, 12.4 Hz, 1H), 4.85 (d, J=10.5 Hz, 1H), 3.97 (d, J=5.1 Hz, 1H), 3.71-3.64 (m, 2H), 3.54 (dd, J=1.7, 6.3 Hz, 1H), 3.25 (br dd, J=4.6, 9.4 Hz, 1H), 3.08 (s, 3H), 3.04-2.91 (m, 2H), 2.78 (s, 3H), 2.70-2.53 (m, 5H), 2.41-2.33 (m, 4H), 2.29 (d, J=7.3 Hz, 2H), 2.20-1.93 (m, 6H), 1.88-1.77 (m, 4H), 1.75 (s, 3H), 1.58 (br dd, J=7.4, 9.0 Hz, 1H), 1.02 (d, J=7.0 Hz, 3H), 0.95 (d, J=6.5 Hz, 6H), 0.85 (d, J=6.9 Hz, 3H), 0.64 (d, J=6.8 Hz, 3H), −0.02 (d, J=7.1 Hz, 3H); HRMS (ESI-TOF) m/z [M+H]⁺ calcd for C₅₂H₆₈N₉O₁₁ 994.5033, found 994.4996 (error 3.7 ppm).

### C3, C4—Modification Chemistry

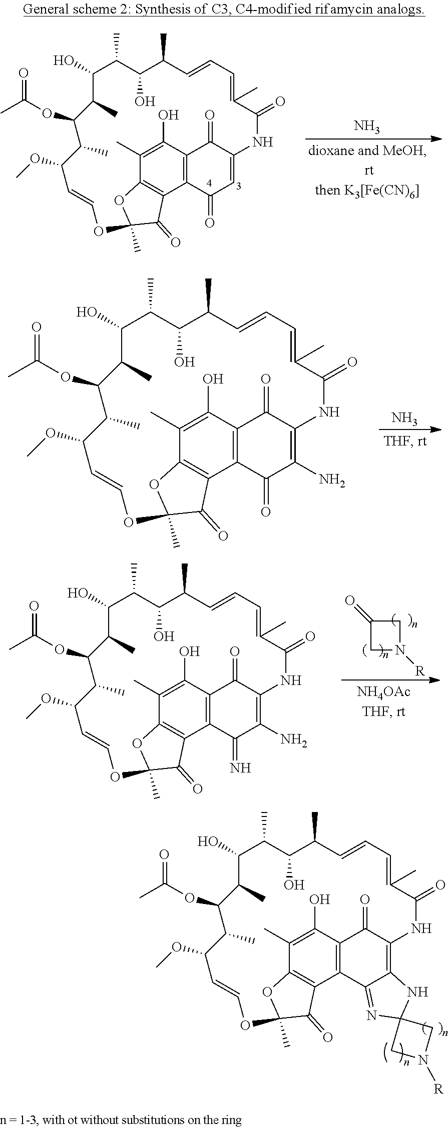

For reported procedures for C3 and C4 modification, see (1) 10.1002/hlca.19730560720; (2) 10.7164/antibiotics.34.1033.

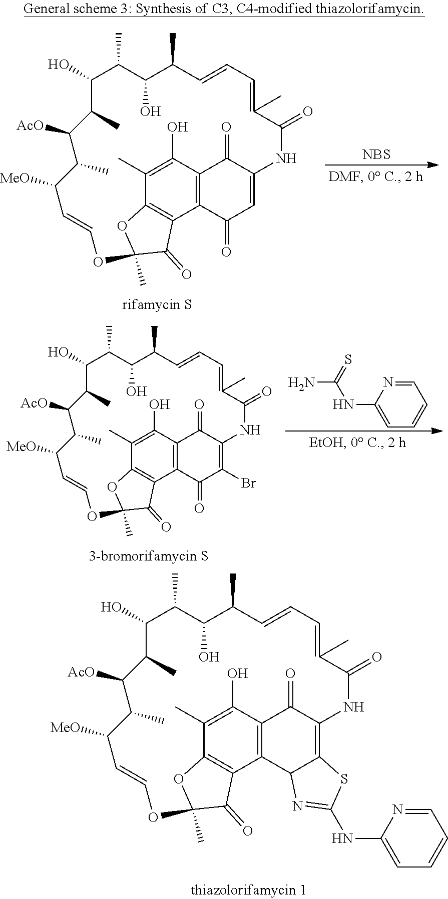

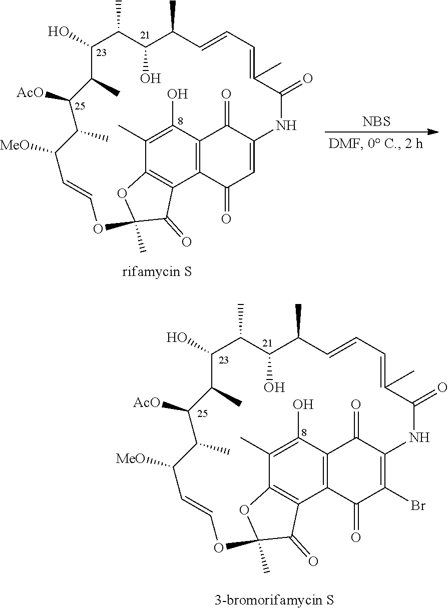

To a solution of rifamycin S (5.00 g, 7.17 mmol) in DMF (50 mL) was added NBS (1.50 g, 8.43 mmol). The mixture was stirred at 0° C. for 2 h. LC-MS showed rifamycin S was consumed completely and desired mass was detected. The reaction mixture was diluted with 100 mL H₂O and extracted with DCM (100 mL×3). The combined organic phase was dried with anhydrous Na₂SO₄, the mixture was filtered and the filtrate was concentrated under vacuum. The residue was purified by flash silica gel chromatography (ISCO®; 40 g SepaFlash® Silica Flash Column, Eluent of 0˜44% Ethyl acetate/Petroleum ethergradient @100 mL/min)(Petroleum ether/Ethyl acetate=1/1, P1 Rf=23) to afford 3-bromorifamycin S (2 g, 2.49 mmol, 34.79% yield, 96.55% purity) as a yellow solid.

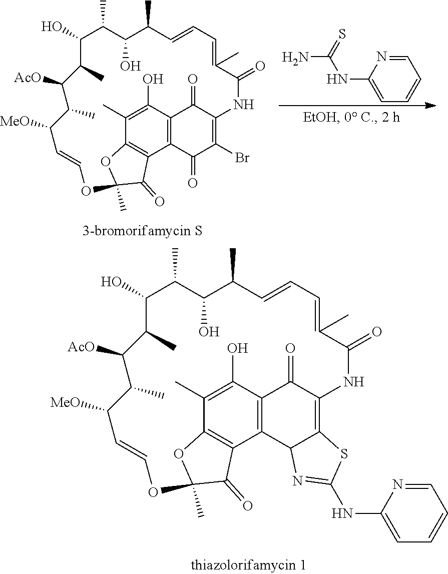

To a solution of 3-bromorifamycin S (50.0 mg, 64.55 μmol) in EtOH (2 mL) was added 2-pyridylthiourea (10.88 mg, 71.00 mol). The mixture was stirred at 0° C. for two hours. LC-MS showed the desired compound. The reaction mixture was concentrated under reduced pressure to remove solvent. The residue was purified by Prep-HPLC FA condition column: Phenomenex tuna C18 100*40 mm*5 um; mobile phase: [H₂O (0.2% FA)-ACN]; gradient: 40%-70% B over 8.0 min to afford thiazolorifamycin 1 (40.6 mg, 44.68 μmol, 69.21% yield, 96.39% purity, FA salt) as an orange solid. ¹H NMR (400 MHz, CHLOROFORM-d) δ ppm −0.71 (br d, J=6.75 Hz, 3H) 0.13 (br d, J=6.75 Hz, 3H) 0.82-1.00 (m, 7H) 1.20 (br dd, J=9.57, 7.19 Hz, 1H) 1.56 (br d, J=6.50 Hz, 1H) 1.87-2.09 (m, 9H) 2.26 (s, 3H) 2.35 (br dd, J=15.76, 7.13 Hz, 1H) 2.45-3.10 (m, 6H) 3.32 (br d, J=6.00 Hz, 1H) 3.49-3.77 (m, 3H) 4.64-4.75 (m, 1H) 4.76-4.94 (m, 1H) 5.90-6.05 (m, 1H) 6.20-6.42 (m, 2H) 6.51-6.75 (m, 2H) 7.03-7.12 (m, 1H) 7.59 (br t, J=7.25 Hz, 1H) 7.66-7.79 (m, 1H) 8.46-8.63 (m, 1H) 15.95-16.18 (m, 1H); HRMS (ESI-TOF) m/z [M+H]⁺ calcd for C₄₃H₅₁N₄O₁₁S 831.3270, found 831.3279 (error 1.1 ppm).

### Example 3

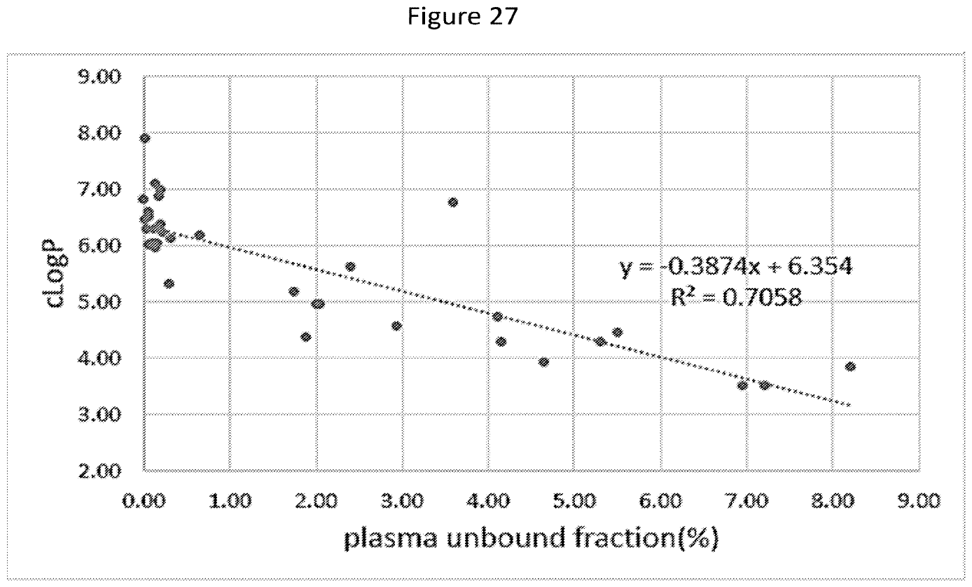

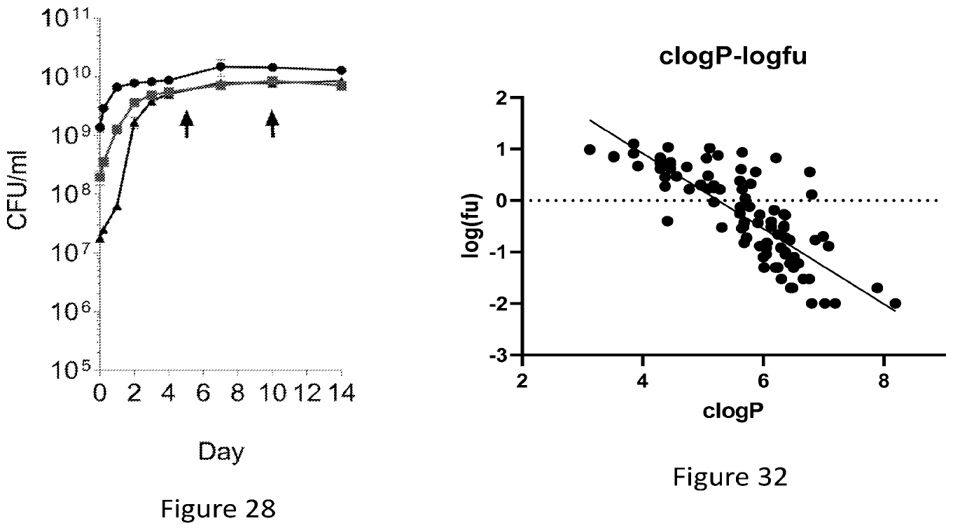

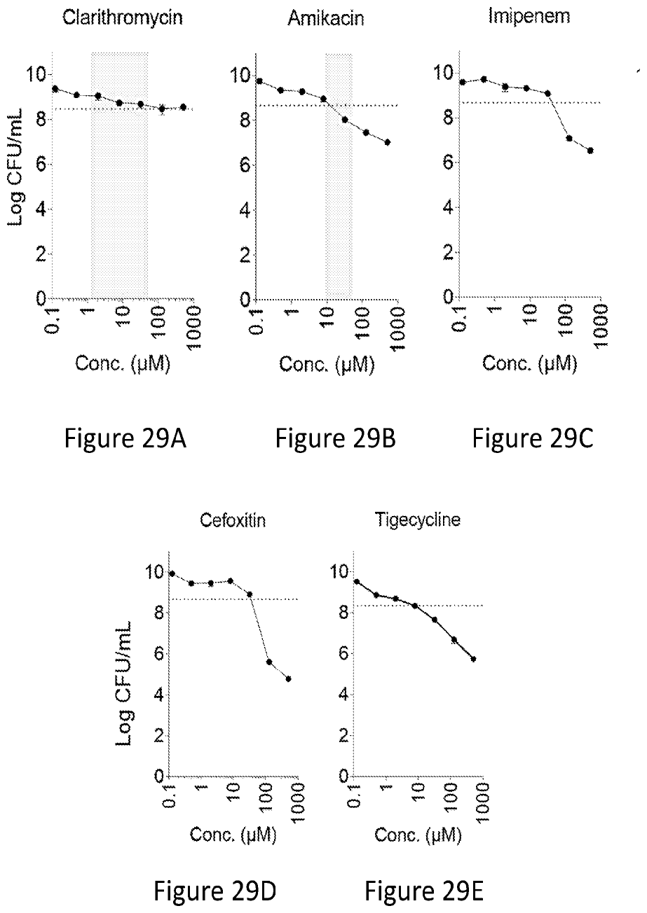

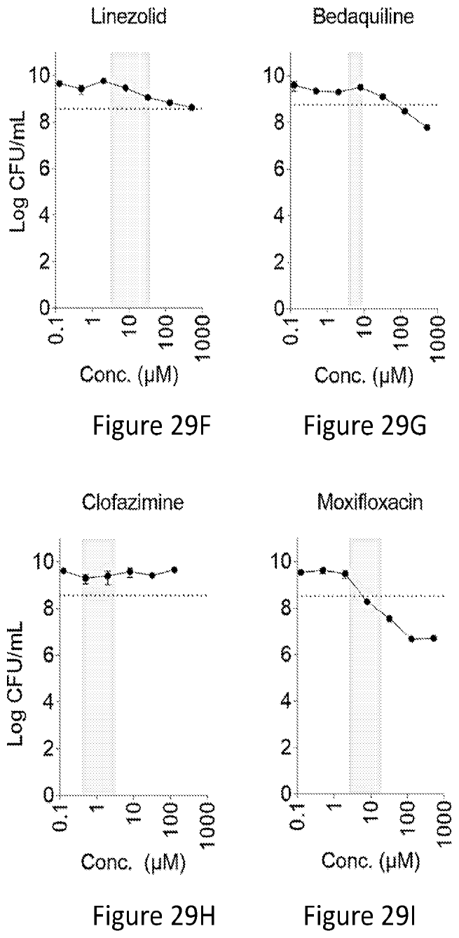

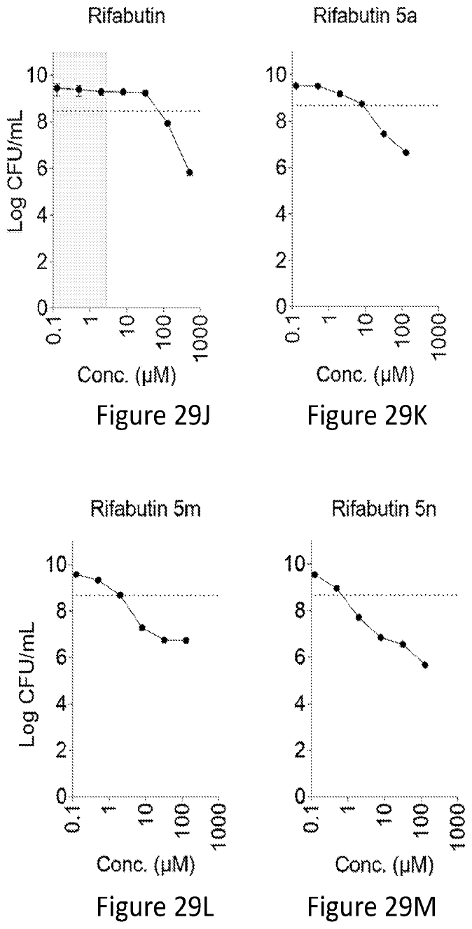

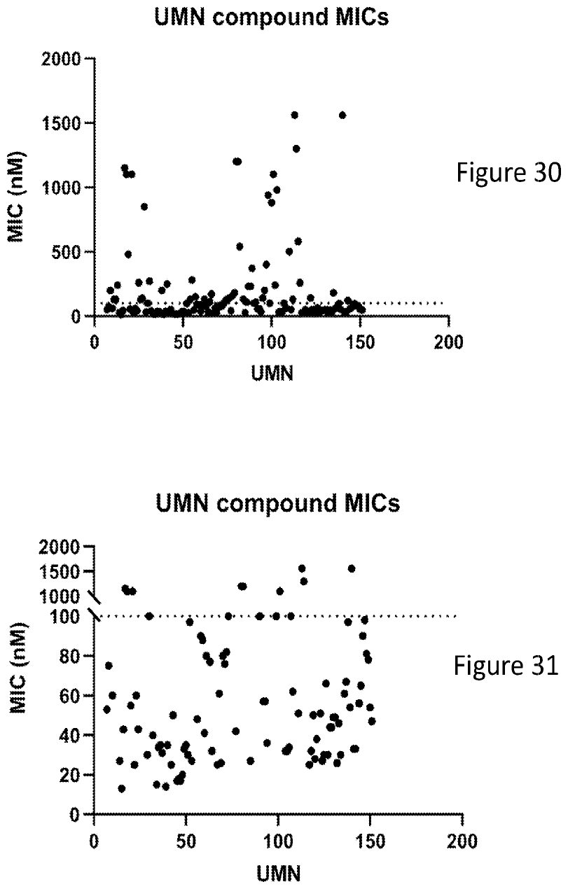

The biological data for certain exemplified compounds are shown in Table 10 and Table 11. For the cLogP-plasma unbound fraction plot, see FIG. 27. For the pharmacokinetic (PK) parameters of certain exemplified compounds, see Table 12.

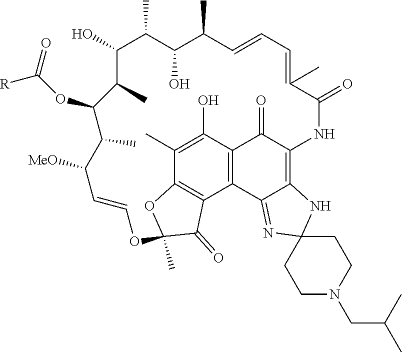

TABLE 10
Biological data of specific compounds; n.d.: not determined.

| | | | | | plasma |
|---|---|---|---|---|---|
| | | MIC_{WT} | MIC_{Δorr} | | unbound |
| cmpd | R | (μM) | (μM) | cLogP | fraction (%) |
| UMN7 | 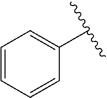 [SMILES: *c1ccccc1] | 0.053 | 0.059 | 6.30 | 0.03 |
| UMN16 | 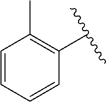 [SMILES: *c1ccccc1C] | 0.043 | 0.064 | 6.80 | 0.007 |
| UMN8 | 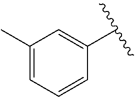 [SMILES: *c1cccc(C)c1] | 0.075 | 0.065 | 6.80 | n.d. |
| UMN9 | 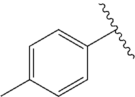 [SMILES: *c1ccc(C)cc1] | 0.2 | 0.11 | 6.80 | n.d. |
| UMN29 | 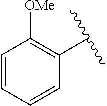 [SMILES: *c1ccccc1OC] | 0.03 | 0.03 | 6.24 | 0.22 |
| UMN23 | 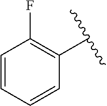 [SMILES: *c1ccccc1F] | 0.06 | 0.058 | 6.01 | 0.05 |
| UMN24 | 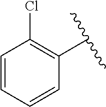 [SMILES: *c1ccccc1Cl] | 0.043 | 0.049 | 6.58 | 0.06 |
| UMN30 | 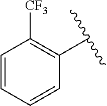 [SMILES: *c1ccccc1C(F)(F)F] | 0.1 | 0.12 | 7.19 | n.d. |
| UMN25 | 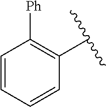 [SMILES: *c1ccccc1-c1ccccc1] | 0.26 | 0.14 | 8.19 | n.d. |
| UMN26 | 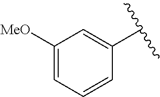 [SMILES: *c1cccc(OC)c1] | 0.13 | 0.13 | 6.48 | 0.02 |
| UMN20 | 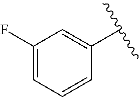 [SMILES: *c1cccc(F)c1] | 0.055 | 0.09 | 6.45 | 0.02 |
| UMN27 | 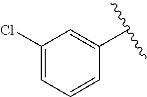 [SMILES: *c1cccc(Cl)c1] | 0.14 | 0.13 | 7.02 | 0 |
| UMN19 | 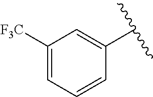 [SMILES: *c1cccc(C(F)(F)F)c1] | 0.48 | 0.36 | 7.19 | 0 |
| UMN18 | 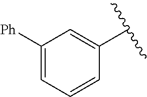 [SMILES: *c1cccc(-c2ccccc2)c1] | 1.10 | 1.05 | 8.19 | 0 |
| UMN10 | 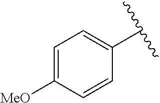 [SMILES: *c1ccc(OC)cc1] | 0.06 | 0.065 | 6.48 | 0 |
| UMN11 | 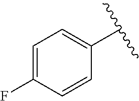 [SMILES: *c1ccc(F)cc1] | 0.13 | 0.069 | 6.45 | 0.02 |
| UMN12 | 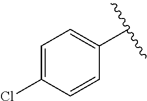 [SMILES: *c1ccc(Cl)cc1] | 0.13 | 0.22 | 7.02 | 0.01 |
| UMN13 | 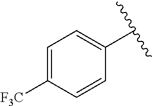 [SMILES: *c1ccc(C(F)(F)F)cc1] | 0.24 | 0.66 | 7.19 | 0 |
| UMN28 | 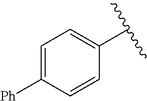 [SMILES: *c1ccc(-c2ccccc2)cc1] | 0.85 | 1.00 | 8.19 | 0 |
| UMN21 | 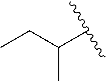 [SMILES: *C(C)CC] | 1.1 | 0.028 | 6.10 | n.d. |
| UMN17 | 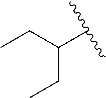 [SMILES: *C(CC)CC] | 1.15 | 0.049 | 6.63 | n.d. |
| UMN80 | 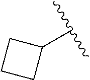 [SMILES: *C1CCC1] | 1.2 | 0.026 | 5.64 | 0.29 |
| UMN81 | 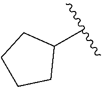 [SMILES: *C1CCCC1] | 1.2 | 0.03 | 6.20 | 0.05 |
| UMN82 | 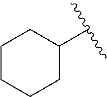 [SMILES: *C1CCCCC1] | 0.54 | 0.05 | 6.76 | 0.03 |
| UMN22 | 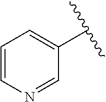 [SMILES: *c1cccnc1] | 0.025 | 0.027 | 4.96 | 2.05 |
| UMN45 | 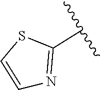 [SMILES: *c1nccs1] | 0.017 | 0.017 | 4.37 | 1.89 |
| UMN46 | 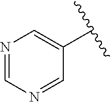 [SMILES: *c1cncnc1] | 0.018 | 0.025 | 3.92 | 4.66 |
| UMN47 | 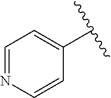 [SMILES: *c1ccncc1] | 0.017 | 0.017 | 4.96 | 2.00 |
| UMN48 | 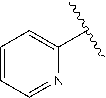 [SMILES: *c1ccccn1] | 0.02 | 0.026 | 4.56 | 2.95 |
| UMN49 | 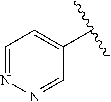 [SMILES: *c1ccnnc1] | 0.033 | 0.034 | 3.85 | 8.22 |
| UMN50 | 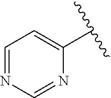 [SMILES: *c1ccncn1] | 0.035 | 0.036 | 3.52 | 7.22 |
| UMN51 | 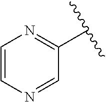 [SMILES: *c1cnccn1] | 0.03 | 0.027 | 3.52 | 6.96 |
| UMN14 | 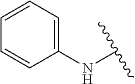 [SMILES: *Nc1ccccc1] | 0.027 | 0.042 | 6.00 | 0.08 |
| UMN31 | 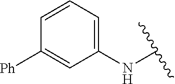 [SMILES: *Nc1cccc(-c2ccccc2)c1] | 0.27 | 0.43 | 7.89 | n.d. |
| UMN34 | 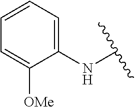 [SMILES: *Nc1ccccc1OC] | 0.015 | 0.014 | 6.05 | 0.12 |
| UMN35 | 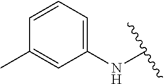 [SMILES: *Nc1cccc(C)c1] | 0.034 | 0.036 | 6.50 | 0.05 |
| UMN36 | 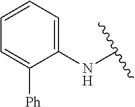 [SMILES: *Nc1ccccc1-c1ccccc1] | 0.035 | 0.05 | 7.89 | 0.02 |
| UMN37 | 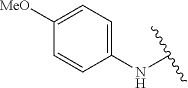 [SMILES: *Nc1ccc(OC)cc1] | 0.031 | 0.034 | 6.05 | 0.15 |
| UMN38 | 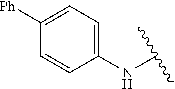 [SMILES: *Nc1ccc(-c2ccccc2)cc1] | 0.2 | 0.43 | 7.89 | n.d. |
| UMN39 | 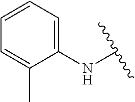 [SMILES: *Nc1ccccc1C] | 0.014 | 0.017 | 5.94 | 0.13 |
| UMN42 | 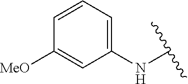 [SMILES: *Nc1cccc(OC)c1] | 0.025 | 0.03 | 6.05 | 0.09 |
| UMN43 | 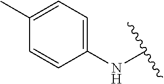 [SMILES: *Nc1ccc(C)cc1] | 0.05 | 0.03 | 6.50 | 0.05 |
| UMN15 | 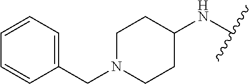 [SMILES: *NC1CCN(Cc2ccccc2)CC1] | 0.013 | 0.028 | 6.37 | 0.19 |
| UMN32 | 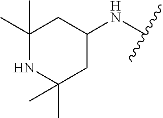 [SMILES: *NC1CC(C)(C)NC(C)(C)C1] | 0.04 | 0.09 | 6.21 | 6.70 |
| UMN40 | 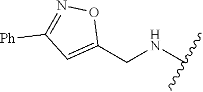 [SMILES: *NCc1cc(-c2ccccc2)no1] | 0.035 | 0.03 | 6.29 | 0.12 |
| UMN41 | 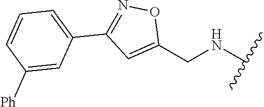 [SMILES: *NCc1cc(-c2cccc(-c3ccccc3)c2)no1] | 0.25 | 0.34 | 8.18 | n.d. |
| UMN53 | 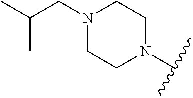 [SMILES: *N1CCN(CC(C)C)CC1] | 0.027 | 0.02 | 6.99 | 0.20 |
| UMN54 | 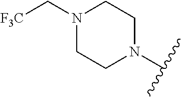 [SMILES: *N1CCN(CC(F)(F)F)CC1] | 0.13 | 0.029 | 6.63 | n.d. |
| UMN55 | 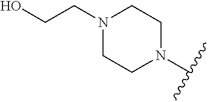 [SMILES: *N1CCN(CCO)CC1] | 0.28 | 0.13 | 5.00 | n.d. |
| UMN56 | 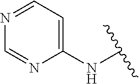 [SMILES: *Nc1ccncn1] | 0.048 | 0.05 | 4.46 | 5.50 |
| UMN57 | 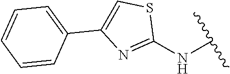 [SMILES: *Nc1nc(-c2ccccc2)cs1] | 0.15 | 0.25 | 7.17 | n.d. |
| UMN58 | 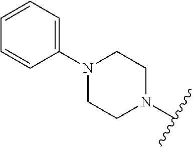 [SMILES: *N1CCN(c2ccccc2)CC1] | 0.09 | 0.049 | 7.08 | 0.13 |
| UMN61 | 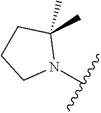 | 0.08 | 0.052 | 6.86 | 0.17 |
| UMN63 | 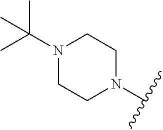 [SMILES: *N1CCN(C(C)(C)C)CC1] | 0.077 | 0.043 | 6.77 | 3.60 |
| UMN64 | 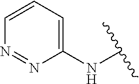 [SMILES: *Nc1cccnn1] | 0.032 | 0.034 | 4.29 | 4.15 |
| UMN65 | 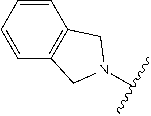 [SMILES: *N1Cc2ccccc2C1] | 0.11 | 0.052 | 6.66 | n.d. |
| UMN66 | 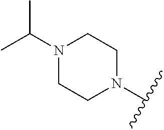 [SMILES: *N1CCN(C(C)C)CC1] | 0.17 | 0.056 | 6.37 | n.d. |
| UMN67 | 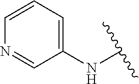 [SMILES: *Nc1cccnc1] | 0.025 | 0.025 | 5.18 | 1.75 |
| UMN68 | 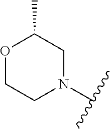 [SMILES: *N1CCO[C@H](C)C1] | 0.061 | 0.033 | 5.61 | 2.40 |
| UMN69 | 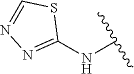 [SMILES: *Nc1nncs1] | 0.026 | 0.027 | 4.29 | 5.30 |
| UMN70 | 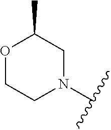 [SMILES: *N1CCO[C@@H](C)C1] | 0.08 | 0.026 | 5.61 | 0.56 |
| UMN71 | 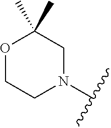 | 0.076 | 0.032 | 6.13 | 0.39 |
| UMN74 | 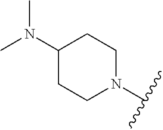 [SMILES: *N1CCC(N(C)C)CC1] | 0.12 | 0.13 | 5.08 | n.d. |
| UMN73 | 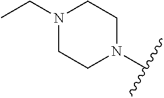 [SMILES: *N1CCN(CC)CC1] | 0.1 | 0.052 | 6.18 | 0.65 |
| UMN72 | 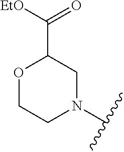 [SMILES: *N1CCOC(C(=O)OCC)C1] | 0.082 | 0.040 | 5.62 | 0.75 |
| UMN75 | 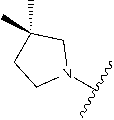 | 0.13 | 0.035 | 6.86 | n.d. |
| UMN78 | 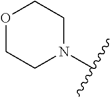 [SMILES: *N1CCOCC1] | 0.16 | 0.040 | 5.09 | n.d. |
| UMN79 | 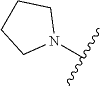 [SMILES: *N1CCCC1] | 0.18 | 0.030 | 5.82 | n.d. |
| UMN84 | 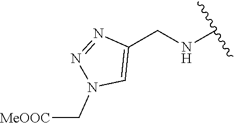 [SMILES: *NCc1cn(CC(=O)OC)nn1] | 0.13 | 0.15 | 3.64 | n.d. |
| UMN85 | 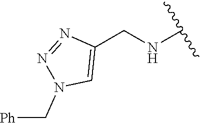 [SMILES: *NCc1cn(Cc2ccccc2)nn1] | 0.027 | 0.025 | 5.31 | 0.30 |
| UMN86 | 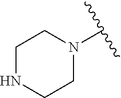 [SMILES: *N1CCNCC1] | 0.11 | 0.10 | 5.08 | n.d. |
| UMN87 | 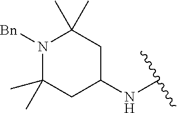 [SMILES: *NC1CC(C)(C)N(Cc2ccccc2)C(C)(C)C1] | 0.23 | 0.30 | 8.45 | n.d. |
| UMN91 | 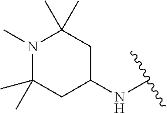 [SMILES: *NC1CC(C)(C)N(C)C(C)(C)C1] | 0.11 | 0.10 | 6.49 | n.d. |

n.d.

TABLE 11
MIC data for selected compounds against Mycobacterium abscessus
ATCC 19977

| cmpd | R | MIC (nM) |
|---|---|---|
| UMN52 | 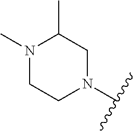 [SMILES: *N1CCN(C)C(C)C1] | 97 |
| UMN99 | 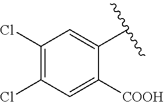 [SMILES: *c1cc(Cl)c(Cl)cc1C(=O)O] | 100 |
| UMN104 | 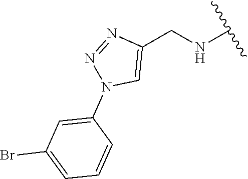 [SMILES: *NCc1cn(-c2cccc(Br)c2)nn1] | 32 |
| UMN106 | 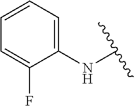 [SMILES: *Nc1ccccc1F] | 34 |
| UMN107 | 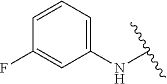 [SMILES: *Nc1cccc(F)c1] | 100 |
| UMN108 | 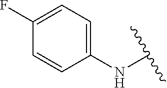 [SMILES: *Nc1ccc(F)cc1] | 62 |
| UMN121 | 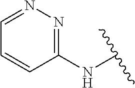 [SMILES: *Nc1cccnn1] | 38 |
| UMN122 | 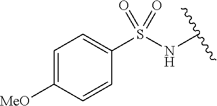 [SMILES: *NS(=O)(=O)c1ccc(OC)cc1] | 140 |
| UMN124 | 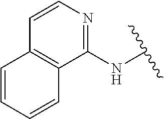 [SMILES: *Nc1nccc2ccccc12] | 27 |
| UMN127 | 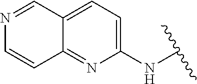 [SMILES: *Nc1ccc2cnccc2n1] | 30 |
| UMN129 | 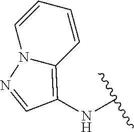 [SMILES: *Nc1cnn2ccccc12] | 44 |
| UMN130 | 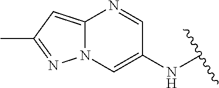 [SMILES: *Nc1cnc2cc(C)nn2c1] | 49 |
| UMN131 | 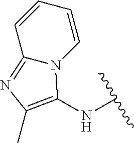 [SMILES: *Nc1c(C)nc2ccccn12] | 49 |
| UMN134 | 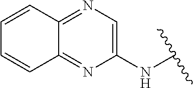 [SMILES: *Nc1cnc2ccccc2n1] | 30 |
| UMN136 | 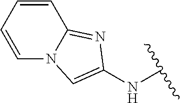 [SMILES: *Nc1cn2ccccc2n1] | 61 |
| UMN142 | 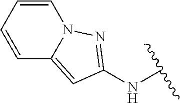 [SMILES: *Nc1cc2ccccn2n1] | 33 |
| UMN145 | 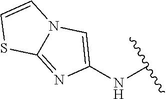 [SMILES: *Nc1cn2ccsc2n1] | 65 |

TABLE 12
Candidate pharmacokinetic (PK) parameters.

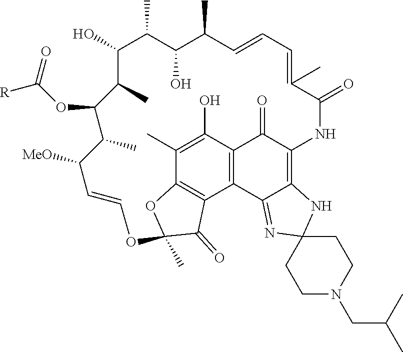

| R | 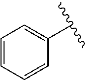 [SMILES: *c1ccccc1] | 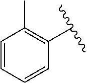 [SMILES: *c1ccccc1C] | 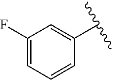 [SMILES: *c1cccc(F)c1] | 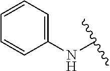 [SMILES: *Nc1ccccc1] | 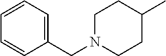 | 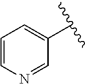 [SMILES: *c1cccnc1] |
|---|---|---|---|---|---|---|
| V_{d} (L/kg) | 2.9 | 1.1 | 1.9 | 1.9 | 4.8 | 1.6 |
| t_{1/2} (h) | 13.8 | 10.3 | 11.7 | 9.3 | 10.7 | 6.8 |
| CL | 2.4 | 1.5 | 2.7 | 2.4 | 6.4 | 3.0 |
| [mL/(kg × min)] | | | | | | |
| C-25 | 0.03 | n.d. | n.d. | 0.08 | n.d. | 0.2 |
| deacylation _{i.v.} | | | | | | |
| (%) | | | | | | |
| C-25 | 0.008 | 0.2 | n.d. | 0.001 | n.d. | 0.01 |
| deacylation _{p.o.} | | | | | | |
| (%)^{[a]} | | | | | | |
| plasma | 0.03 | 0.01 | 0.02 | 0.0008 | 0.002 | 2.05 |
| unbound | | | | | | |
| fraction (%) | | | | | | |
| ƒAUC/MIC^{[b]} | 0.08 | 0.04 | 0.06 | 0.43 | 0.62 | 28.2 |

^{[a]}For both administrative routes, deacylation % = AUC_{25-O-desacetyl RFB}/AUC_{compound} × 100%,
n.d.: undetectable.
^{[b]}fAUC/MIC was measured based on p.o. doses of 10 mg/kg

### Example 4

The in-vitro activity, calculated physicochemical properties, and fᵤ of rifabutin ester analogs are determined using methods disclosed in Example 1 and shown in Table 13.

TABLE 13
In vitro activity, calculated physicochemical properties, and fᵤ of rifabutin ester.analogs.

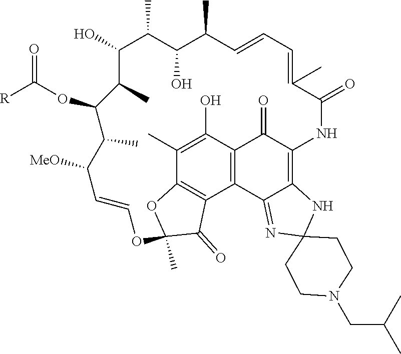

| | R |
|---|---|
| UMN26 | 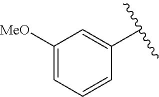 [SMILES: *c1cccc(OC)c1] |
| UMN10 | 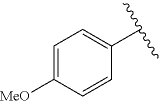 [SMILES: *c1ccc(OC)cc1] |
| UMN11 | 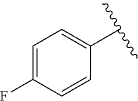 [SMILES: *c1ccc(F)cc1] |
| UMN27 | 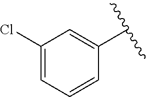 [SMILES: *c1cccc(Cl)c1] |
| UMN12 | 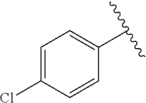 [SMILES: *c1ccc(Cl)cc1] |
| UMN19 | 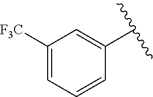 [SMILES: *c1cccc(C(F)(F)F)c1] |
| UMN13 | 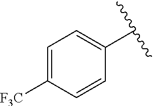 [SMILES: *c1ccc(C(F)(F)F)cc1] |
| UMN18 | 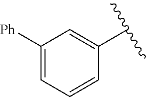 [SMILES: *c1cccc(-c2ccccc2)c1] |
| UMN28 | 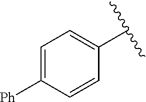 [SMILES: *c1ccc(-c2ccccc2)cc1] |
| UMN93 | 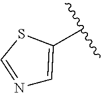 [SMILES: *c1cncs1] |
| UMN94 | 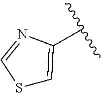 [SMILES: *c1cscn1] |
| UMN48 | 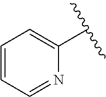 [SMILES: *c1ccccn1] |
| UMN47 | 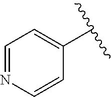 [SMILES: *c1ccncc1] |
| UMN80 | 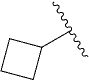 [SMILES: *C1CCC1] |
| UMN81 | 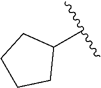 [SMILES: *C1CCCC1] |
| UMN82 | 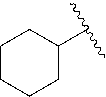 [SMILES: *C1CCCCC1] |
| UMN49 | 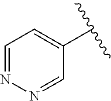 [SMILES: *c1ccnnc1] |
| UMN50 | 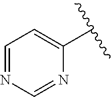 [SMILES: *c1ccncn1] |
| UMN51 | 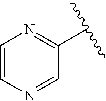 [SMILES: *c1cnccn1] |
| UMN92 | 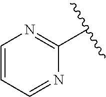 [SMILES: *c1ncccn1] |
| UMN111 | 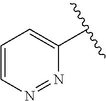 [SMILES: *c1cccnn1] |

| | | Calculated physicochemical properties | | | | | | |
|---|---|---|---|---|---|---|---|---|
| cmpd | Mab MIC (nM) | C-sp³ fraction | tPSA (Å ²) | nHD | nHA | MR | clogP | ƒᵤ (%) |
| rifabutin | 1100 | 0.59 | 209 | 5 | 14 | 245 | 4.8 | 4.5 |
| UMN26 | 130 | 0.52 | 215 | 5 | 14 | 271 | 6.5 | 0.02 |
| UMN10 | 60 | 0.52 | 215 | 5 | 14 | 271 | 6.5 | 0.02 |
| UMN11 | 130 | 0.51 | 206 | 5 | 14 | 265 | 6.4 | 0.02 |
| UMN27 | 140 | 0.51 | 206 | 5 | 13 | 270 | 7.0 | 0 |
| UMN12 | 130 | 0.51 | 206 | 5 | 13 | 270 | 7.0 | 0.01 |
| UMN19 | 480 | 0.52 | 206 | 5 | 16 | 270 | 7.2 | 0 |
| UMN13 | 240 | 0.52 | 206 | 5 | 16 | 270 | 7.2 | 0 |
| UMN18 | 1100 | 0.46 | 206 | 5 | 13 | 290 | 8.2 | 0.01 |
| UMN28 | 850 | 0.46 | 206 | 5 | 13 | 290 | 8.2 | 0 |
| UMN93 | 57 | 0.54 | 247 | 5 | 14 | 260 | 4.8 | 1.6 |
| UMN94 | 36 | 0.54 | 247 | 5 | 14 | 260 | 4.4 | 2.9 |
| UMN48 | 20 | 0.52 | 218 | 5 | 14 | 262 | 4.6 | 3.0 |
| UMN47 | 17 | 0.52 | 218 | 5 | 14 | 262 | 5.0 | 2.0 |
| UMN80 | 1200 | 0.61 | 206 | 5 | 13 | 257 | 5.6 | 0.3 |
| UMN81 | 1200 | 0.62 | 206 | 5 | 13 | 262 | 6.2 | 0.05 |
| UMN82 | 1100 | 0.63 | 206 | 5 | 13 | 267 | 6.8 | 0.03 |
| UMN49 | 33 | 0.53 | 231 | 5 | 15 | 260 | 3.8 | 8.2 |
| UMN50 | 35 | 0.53 | 231 | 5 | 15 | 260 | 3.5 | 7.2 |
| UMN51 | 30 | 0.53 | 231 | 5 | 15 | 260 | 3.5 | 7.0 |
| UMN92 | 57 | 0.53 | 231 | 5 | 15 | 260 | 3.1 | 9.8 |
| UMN111 | 51 | 0.53 | 231 | 5 | 15 | 260 | 3.8 | 12.7 |

The in vitro activity, cLogP, and fᵤ of rifabutin carbamate analogs are shown in Table 14.

TABLE 14
In vitro activity, cLogP, and ƒu of rifabutin carbamate analogs.

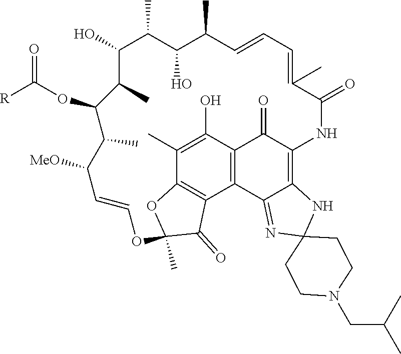

| cmpd | R | WT^{[a]} Mab MIC (nM) | clogP | ƒᵤ (%) |
|---|---|---|---|---|
| UMN15 | 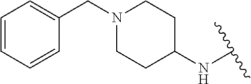 [SMILES: *NC1CCN(Cc2ccccc2)CC1] | 13 | 6.4 | 0.2 |
| UMN150 | 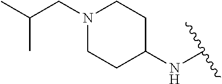 [SMILES: *NC1CCN(CC(C)C)CC1] | 54 | 5.9 | 3.6 |
| UMN147 | 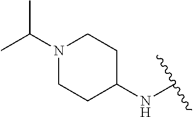 [SMILES: *NC1CCN(C(C)C)CC1] | 98 | 5.2 | 7.5 |
| UMN149 | 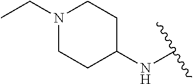 [SMILES: *NC1CCN(CC)CC1] | 78 | 5.1 | 10.4 |
| UMN146 | 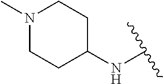 [SMILES: *NC1CCN(C)CC1] | 90 | 4.4 | 10.8 |
| UMN32 | 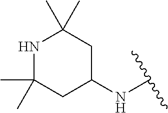 [SMILES: *NC1CC(C)(C)NC(C)(C)C1] | 40 | 6.5 | 6.7 |

The in vitro activity, cLogP, and fᵤ of heterocyclic aromatic carbamate analogs of rifabutin are shown in Table 15.

TABLE 15
In vitro activity, cLogP, and ƒu of heterocyclic aromatic carbamate
analogs of rifabutin.

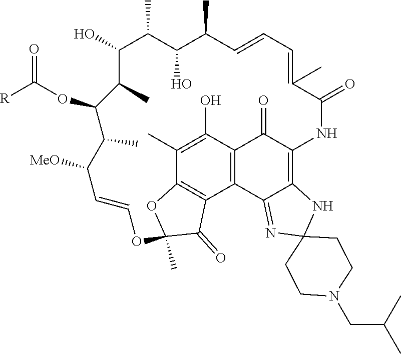

| cmpd | R | WT Mab MIC (nM) | clogP | ƒᵤ (%) |
|---|---|---|---|---|
| UMN117 | 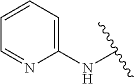 [SMILES: *Nc1ccccn1] | 25 | 5.2 | 0.9 |
| UMN105 | 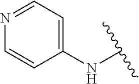 [SMILES: *Nc1ccncc1] | 25 | 5.2 | 2.0 |
| UMN118 | 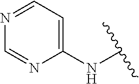 [SMILES: *Nc1ccncn1] | 32 | 4.5 | 5.1 |
| UMN119 | 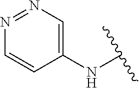 [SMILES: *Nc1ccnnc1] | 50 | 4.3 | 6.1 |
| UMN120 | 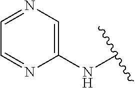 [SMILES: *Nc1cnccn1] | 28 | 4.5 | 4.2 |
| UMN133 | 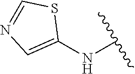 [SMILES: *Nc1cncs1] | 46 | 5.1 | 3.0 |
| UMN132 | 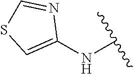 [SMILES: *Nc1cscn1] | 26 | 5.1 | 1.7 |
| UMN69 | 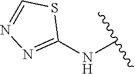 [SMILES: *Nc1nncs1] | 26 | 4.3 | 5.3 |
| UMN14 | 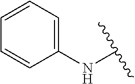 [SMILES: *Nc1ccccc1] | 27 | 6.0 | 0.1 |
| UMN39 | 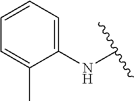 [SMILES: *Nc1ccccc1C] | 14 | 5.9 | 0.1 |
| UMN36 | 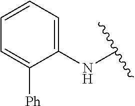 [SMILES: *Nc1ccccc1-c1ccccc1] | 35 | 7.9 | 0.02 |

### Example 5

Broth MIC, broth MBC₉₀ (bMBC₉₀), and caseum surrogate MBC₉₀ (cMBC₅₀) of clinically used antibiotics and of novel ADP-ribosylation-resistant rifabutin analogs against M. abscessus Bamboo have been evaluated. Rifamycin analogs 5a, 5m, and 5n showed improved bactericidal activity against M. abscessus in surrogate caseum, see Table 16.

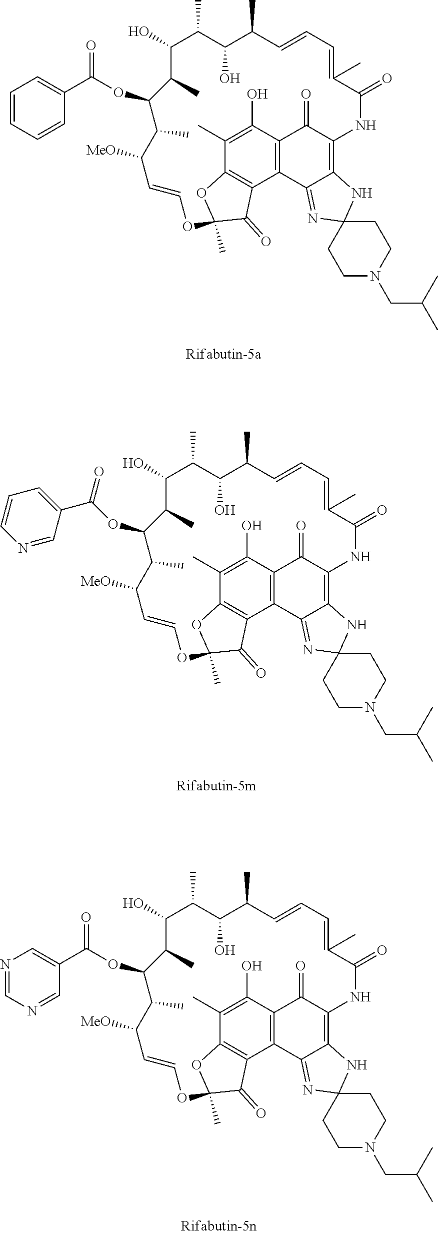

TABLE 16
Broth MIC, broth MBC₉₀ (bMBC₉₀), and caseum surrogate MBC₉₀
(cMBC₉₀) of clinically used antibiotics and of novel ADP-ribosylation-
resistant rifabutin analogs against M. abscessus Bamboo

| | | MIC | bMBC₉₀ | cMBC₉₀ | |
|---|---|---|---|---|---|
| Drug class | Drug | (μM) | (μM) | (μM) | cMBC₉₀/bMBC₉₀ |
| Macrolide | Clarithromycin | 0.4 | 2 | 128 | 64 |
| Aminoglycoside | Amikacin | 25 | 40 | 12 | 0.3 |
| β-Lactam | Imipenem | 10 | 18 | 42 | 2.3 |
| (carbapenem) | | | | | |
| β-Lactam | Cefoxitin | 29 | 45 | 35 | 0.78 |
| (cephalosporin) | | | | | |
| Glycylcycline | Tigecycline | 12.5 | 8 | 8 | 1 |
| Oxazolidinone | Linezolid | 25 | 32 | 512 | 16 |
| Diarylquinoline | Bedaquiline | 0.4 | 2^{a} | 67^{a} | 33.5 |
| Riminophenazine | Clofazimine | 12.5 | >128 | >128 | NA^{b} |
| Fluoroquinolone | Moxifloxacin | 6 | 2 | 8 | 4 |
| Rifamycin | Rifabutin | 3 | 5 | 72 | 16 |
| Rifamycin | Rifabutin-5a | 0.07 | 0.30 | 8 | 26.7 |
| Rifamycin | Rifabutin-5m | 0.02 | 0.16 | 2 | 12.5 |
| Rifamycin | Rifabutin-5n | 0.03 | 0.13 | 0.65 | 5 |

^{a}Assay extended to 10 days (instead of 5 days used for all other drugs) due to slow onset of bactericidal activity. (Tuberculosis (Edinb), 2020, 90, 301-305; J. Biol. Chem., 2008, 283, 25273-25280.)
^{b}NA: not applicable as not active.

### Biological Procedures

The surrogate matrix was generated as described previously from cultured THP-1 cells (ATCC TIB-202) (doi: 10.1128/mbio.00598-23). M. abscessus exponential cultures grown in Middlebrook 7H9 broth (Sigma Aldrich) (OD₆₀₀ 0.6-0.9) were spun down and resuspended in water to an OD₆₀₀ of 7, 0.7, and 0.07. As described for the M. tuberculosis caseum surrogate assay (doi: 10.1128/mbio.00598-23), the bacterial suspensions (at three different dilutions resulting in ˜10⁹, 10⁸, and 10⁷ starting CFU/mL) were added to the caseum surrogate in the ratio 2:1 (vol/wt), briefly homogenized with 1.4-mm zirconia beads, divided evenly into nine 1.5-mL microcentrifuge tubes, and incubated as standing cultures at 37° C. At the indicated time points, tubes were removed and used for CFU enumeration by plating on Middlebrook 7H11 agar (Sigma Aldrich). Separate tubes were used at each time point. To determine the kill curves for the drugs, cultures with a starting CFU/mL of 10⁸ were used. The experiment was repeated three times independently, yielding similar results.

At day 5 from the starting culture, after the cultures entered stationary phase, 50 μL mixtures (cultures in caseum surrogate) were exposed to drugs (1 μL in DMSO, amikacin, clarithromycin, clofazimine, imipenem, rifabutin and tigecycline were purchased from Sigma Aldrich, moxifloxacin and linezolid from Sequoia Research Products, and cefoxitin and bedaquiline from MedChemExpress. The rifabutin analogs were synthesized as described (doi: 10.1002/anie.202211498).) in the range of 0.125-512 μM (128 μM for clofazimine and rifabutin analogs 5a, 5m, 5n) for 5 days (or 10 days for bedaquiline), after which CFU was enumerated and caseum surrogate MBC90 (cMBC90) was calculated. Addition of 2% of the vehicle DMSO did not affect viable counts. The experiment was repeated twice independently, yielding similar results. A representative example is shown. The cMBC90 values shown in Table 16 are the drug concentrations that reduce CFU by 90% relative to the CFU of drug-free controls at day 10. Since the cultures were in the stationary phase on day 5 when drug treatment started, the CFUs of the drug-free cultures at day 10 were similar to the CFUs of the drug-free cultures at day 5.

### Example 6

The broad-spectrum anti-mycobacterial profiling of compound 5m is shown in Table 17. The MIC data was determined using methods disclosed in Example 1. Data suggested that the rifamycim analogs have expanded anti-mycobacterial spectrum

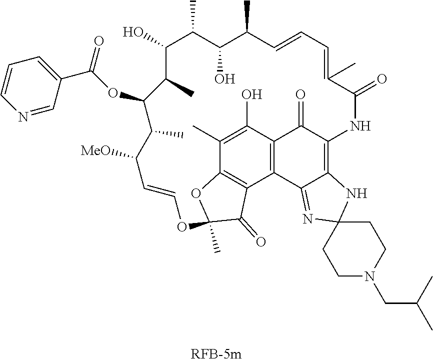 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C/C=C/[C@H](C)[C@H](O)[C@@H](C)[C@@H](O)[C@@H](C)[C@H](OC(=O)c2cccnc2)[C@@H]1C)C4=O]

TABLE 17
Broad spectrum anti-mycobacterial profiling of compound RFB-5m.

| | Rifamycins | Arr | | | | | Fold |
|---|---|---|---|---|---|---|---|
| | used | homolog | MIC (μM)^{a} | | | | improvement |
| | clinically? | present? | | | | RFB- | (RFB-5m vs |
| Strain | [23-25] | [26] | CLR^{b} | RIF | RFB | 5m | RFB) |

Rapidly growing NTM

| | | | | | | | |
|---|---|---|---|---|---|---|---|
| M. abscessus | | | | | | | |
| complex | | | | | | | |
| M. abscessus | No | Yes | 1.5 | 9.5 | 1.1 | 0.02 | 55 |
| subsp. abscessus | | | | | | | |
| ATCC 19977 | | | | | | | |
| M. abscessus | No | Yes | 4.3 | 28 | 2.2 | 0.07 | 31 |
| subsp. bolletii | | | | | | | |
| CCUG 50184T | | | | | | | |
| M. abscessus | No | Yes | 1.1 | 13 | 0.6 | 0.05 | 12 |
| subsp. | | | | | | | |
| massiliense | | | | | | | |
| CCUG 48898T | | | | | | | |
| M. chelonae | No | Yes | 0.1 | 1.4 | 0.6 | 0.02 | 30 |
| ATCC 35752 | | | | | | | |
| M. fortuitum | No | Yes | 3.2 | 6.7 | 1.2 | 0.02 | 60 |
| ATCC 6841 | | | | | | | |

Slowly growing NTM

| | | | | | | | |
|---|---|---|---|---|---|---|---|
| M. avium | | | | | | | |
| complex | | | | | | | |
| M. avium subsp. | Yes | No | 0.5 | 0.04 | 0.03 | 0.04 | 0.8 |
| hominisuis | | | | | | | |
| MAC109 | | | | | | | |
| M. intracellulare | Yes | No | 0.4 | 0.06 | 0.05 | 0.06 | 0.8 |
| ATCC 13950 | | | | | | | |
| M. chimaera | Yes | No | 0.8 | 0.07 | 0.05 | 0.06 | 0.8 |
| CCUG 50989T | | | | | | | |
| M. kansasii | Yes | No | 0.3 | 0.3 | 0.008 | 0.008 | 1.0 |
| ATCC 12478 | | | | | | | |
| M. szulgai | Yes | No | 0.3 | 0.06 | 0.01 | 0.006 | 1.7 |
| ATCC 35799 | | | | | | | |
| M. xenopi ATCC | Yes | Yes | 0.06 | 0.04 | 0.02 | 0.005 | 4.0 |
| 19250 | | | | | | | |
| M. simiae ATCC | No | Yes | 12 | >100 | 19 | 0.2 | 95 |
| 25275 | | | | | | | |

^{a}MIC values are the mean of two independent experiments.
^{b}CLR, clarithromycin; RIF, rifampicin; and RFB, rifabutin.

### Example 7

From the analog collection, it was found that plasma unbound fraction (fᵤ), an important pharmacokinetic parameter, is correlated with molecule lipophilicity, characterized by clogP. See FIG. 32.

Additional Pharmacokinetic data of some selected Rifabutin analogs are shown in Table 18. Pharmacokinetic optimization has also been carried out. See Tables 19 and 20. The optimized parameters are shown in italic.

TABLE 18
In vivo PK Evaluations of the Selected Rifabutin Analogs.

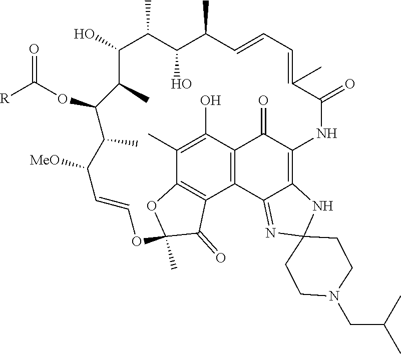

| | R |
|---|---|
| UMN15 | 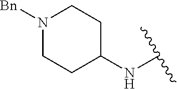 [SMILES: *NC1CCN(Cc2ccccc2)CC1] |
| UMN14 | 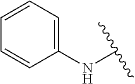 [SMILES: *Nc1ccccc1] |
| UMN94 | 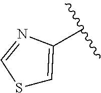 [SMILES: *c1cscn1] |
| UMN105 | 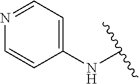 [SMILES: *Nc1ccncc1] |
| UMN120 | 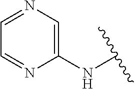 [SMILES: *Nc1cnccn1] |
| UMN69 | 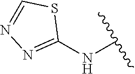 [SMILES: *Nc1nncs1] |

| cmpd | clogP | ƒᵤ (%) | V_{d} [L/kg] | CL [mL/(kg × min) | F [%] | AUC_{p.o.} [h × ng/mL] | ƒAUC/MIC |
|---|---|---|---|---|---|---|---|
| RFB | 4.8 | 4.5 | 3.51 | 10.3 | 70 | 11508 | 0.22 |
| UMN15 | 6.4 | 0.2 | 5.92 | 6.40 | 35 | 5658 | 0.8 |
| UMN14 | 6.0 | 0.1 | 1.93 | 2.40 | 38 | 25625 | 0.8 |
| UMN94 | 4.4 | 2.9 | 1.22 | 5.0 | 77 | 25765 | 22.4 |
| UMN105 | 5.2 | 2.0 | 3.78 | 7.57 | 63 | 14210 | 9.4 |
| UMN120 | 4.5 | 4.2 | 0.75 | 2.19 | 73 | 55072 | 88.8 |
| UMN69 | 4.3 | 5.3 | 1.14 | 6.19 | 45 | 12103 | 26.5 |

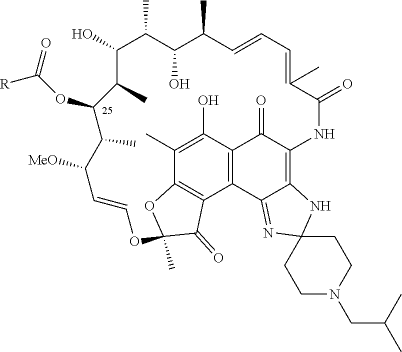

TABLE 19
Pharmacokinetic optimization of selected Rifabutin analogs.

| cmpd | rifabutin | UMN22 | UMN46 | UMN120 |
|---|---|---|---|---|
| R | 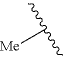 | 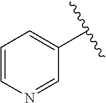 [SMILES: *c1cccnc1] | 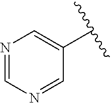 [SMILES: *c1cncnc1] | 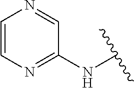 [SMILES: *Nc1cnccn1] |
| cLogP | 4.47 | 4.96 | 3.92 | 4.46 |
| MIC (nM)* | 1100 | 25 | 18 | 28 |
| V_{d} (L/kg) | 3.5 | 1.6 | 2.1 | 0.7 |
| CL [mL/(kg*min)] | 10.3 | 3.0 | 4.0 | 2.2 |
| F (%) | 70 | 78 | 86 | 73 |
| plasma unbound (ƒᵤ, %) | 3.8 | 2.0 | 4.7 | 4.2 |

*The MIC data is determined against Mycobacterium abscessus.

TABLE 20
Pharmacokinetic optimization of selected Rifabutin analogs.

| cmpd | rifabutin | UMN22 | UMN34 | UMN46 |
|---|---|---|---|---|
| R | 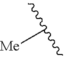 | 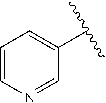 [SMILES: *c1cccnc1] | 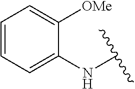 [SMILES: *Nc1ccccc1OC] | 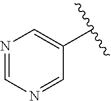 [SMILES: *c1cncnc1] |
| cLogP | 4.47 | 4.96 | 6.05 | 3.92 |
| MIC (nM)** | 1100 | 25 | 15 | 18 |
| plasma unbound (ƒᵤ, %) | 3.8 | 2.0 | 0.12 | 4.7 |
| ƒAUC/MIC | 0.22 | 45.0 | 0.9 | 87.8 |
| THP-IC/EC | 26.4 | 44.2 | 140.3 | n.d. |
| Fold-killing in THP-1* | 2.3 | 3.8 | 4.9 | 4.4 |

*Fold-killing is expressed in log-10 scale
**The MIC data is determined against Mycobacterium abscessus.

### Example 8

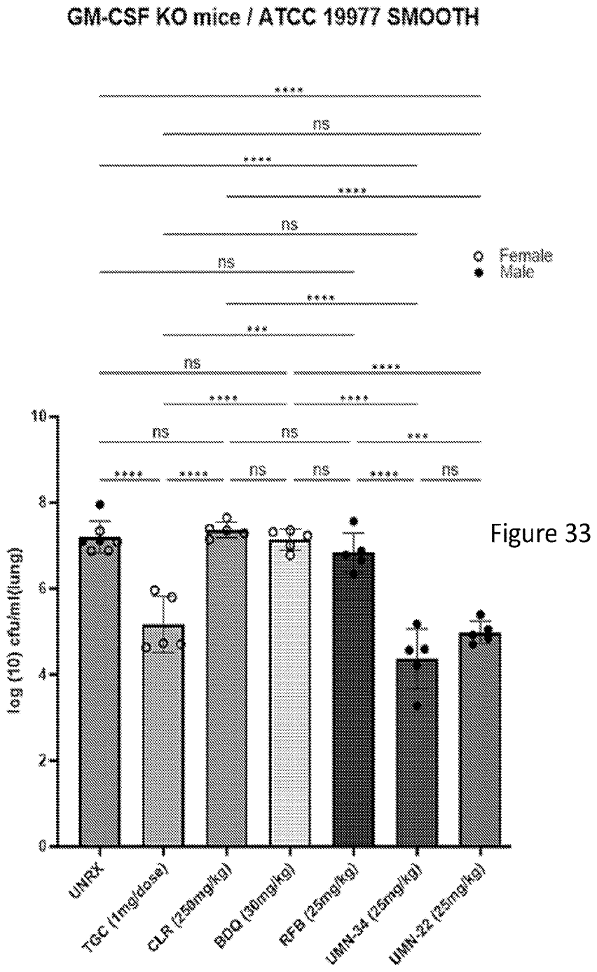

In vivo efficacy of compounds UMN22 and UMN34 were evaluated in acute Mab mouse infection model. See Table 21. Both compounds showed ˜2 log₁₀ reduction in lung CFU burden vs RFB and untreated (UNRX) groups. See FIG. 33.

TABLE 21
In vivo efficacy evaluation of UMN22 and UMN
34 using acute Mab mouse infection model.

| | |
|---|---|
| Mice | 8 weeks old, GM-CSF knockout |
| Mab Strain | ATCC 19977 (Smooth) |
| Infection | 9-day acute infection: Mab replicates from 3.5 log₁₀ |
| | cfu/mL (lung) to 7 log₁₀ cfu/mL (lung) |
| Treatment | On day 2 post-infection, 5 groups of mice are treated |
| | every day for 7 days |
| Dosing | rifabutin (RFB), UMN22, UMN34: 25 mg/kg |

### Example 9

Rifamycins induce human CYP and accelerate metabolism of co-administered drugs. CYP induction can decrease the efficacy of other drugs and is a particularly problematic for patients on multidrug therapies. The new rifabutin analogs can reduce or fully eliminate CYP induction. See Table 22.

TABLE 22
Reduction of CYP induction by the rifabutin analogs.

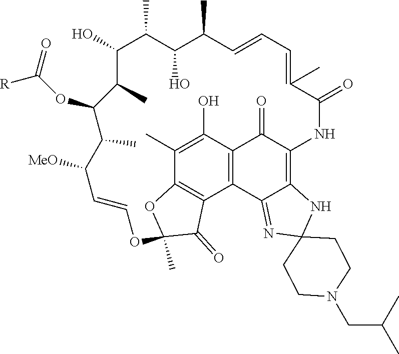

| | | | CYP3A4 |
|---|---|---|---|
| | | | gene fold- |
| | | MIC_{Mab} | induction at |
| compound | R | (nM) | 10 μM |
| rifampicin | — | 5300 | 109.3 |
| rifabutin | Me | 1150 | 40.0 |
| UMN7 | 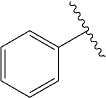 [SMILES: *c1ccccc1] | 53 | 8.6 |
| UMN15 | 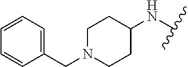 [SMILES: *NC1CCN(Cc2ccccc2)CC1] | 13 | 1.2 |
| UMN16 | 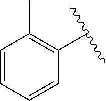 [SMILES: *c1ccccc1C] | 43 | 5.2 |
| UMN20 | 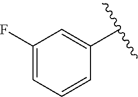 [SMILES: *c1cccc(F)c1] | 55 | 6.2 |
| UMN22 | 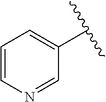 [SMILES: *c1cccnc1] | 25 | 14.1 |
| UMN32 | 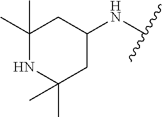 [SMILES: *NC1CC(C)(C)NC(C)(C)C1] | 40 | 0.6 |
| UMN46 | 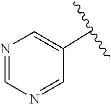 [SMILES: *c1cncnc1] | 18 | 7.9 |
| UMN123 | 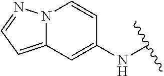 [SMILES: *Nc1ccn2nccc2c1] | 51 | 1.2 |
| UMN125 | 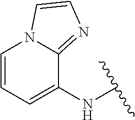 [SMILES: *Nc1cccn2ccnc12] | 30 | 3.6 |
| UMN120 | 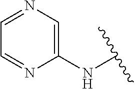 [SMILES: *Nc1cnccn1] | 28 | 0.7 |
| UMN131 | 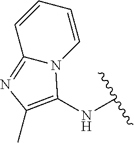 [SMILES: *Nc1c(C)nc2ccccn12] | 49 | 3.0 |
| UMN133 | 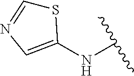 [SMILES: *Nc1cncs1] | 46 | 1.7 |
| UMN121 | 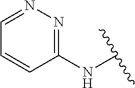 [SMILES: *Nc1cccnn1] | 38 | 2.7 |
| UMN118 | 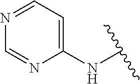 [SMILES: *Nc1ccncn1] | 32 | 2.0 |
| UMN132 | 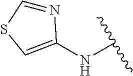 [SMILES: *Nc1cscn1] | 26 | 1.4 |

### Fold Induction Determination

The threshold cycle (CT) was measured for each isoform cDNA by real time qRT-PCR. The mRNA level of each isoform was assessed by the relative quantifiation with reference gene (GAPDH) as the normalizer method. The normalized fold induction was calculated by 2^{-ΔΔCT} method, in which 0.1% DMSO was used as the reference (vehicle control). The induction potential of each test compound was analyzed by comparing its fold induction to a pre-determined cutoff value for each lot of hepatocytes. The hepatocytes lots used in this study were validated using 10 known inducers and 5 known non-inducers for each isozyme. A cutoff value is determined based on the validation study for each hepatocytes lot. According to the the FDA Guidance for Industry —In Vitro Drug Interaction Studies —Cytochrone P450 Enzyme- and Transporter-Mediated Drug Interactions (January 2020), in the testing concentration range, if fold induction at any testing concentration of the test compound is greater than the cutoff value of a CYP isoform in at least one donor, the compound is considered an in vitro inducer for this CYP isoform.

### REFERENCES

[1] V. Novohradsky, L. Zerzankova, J. Stepankova, O. Vrana, R Raveendran, D. Gibson, J. Kasparkova, V. Brabec, J. Inorg. Biochem. 2014, 140, 72-79.
[2] M. C. McCoy, D. J. Faulkner, J. Nat. Prod. 2001, 64, 1087-1089.
[3] H. Booth, Chem. Commun (London) 1968, 802b-803.
[4] F. A. Anet, I. J. Yavari, I. J. Ferguson, A. R Katritzky. V. Moreno-Mañas, M. J. Robinson, J. Chem. Soc. Chem. Commun. 1976, 399-400.
[5] E. L. Eliel, D. Kandasamy, C. Yen, K. D. Hargrave, J. Am. Chem. Soc. 1980, 102, 3698-3707.
[6] S. Bastian, N. Veziris. A. L Roux, F. Brossier, J. L Gaillard, V. Jarlier, E. Cambau, Antimicrob. Agents Chemother. 2011, 55, 775-781
[7] M. Yee, D. Klinzing, J. R. Wei, M. Gengenbacher, E. J. Rubin, T. Dick, Genome Announc 2017, 5, e00388-17.
[8] F. Ripoll, S. Pasek, C. Schenowitz, C. Dossat, V. Barbe, M. Rottman, E. Macheras, B. Heym, J. L. Herrmann, M. Daffe, R. Brosch, J. L. Risler, J. L. Gaillard, PLoS One 2009, 4, e5660.
[9] T. Dick, S. J. Shin, W. J. Koh, V. Dartois, M. Gengenbacher, Antimicrob. Agents Chemother. 2020, 64, e01943-19.
[10] U. S. Ganapathy, T. Lan, P. Krastel, M. Lindman, M. D. Zimmerman, H. Ho, J. P. Sarathy, J. C. Evans, V. Dartois, C. C. Aldrich, T. Dick, Antimicrob. Agents Chemother. 2021, 65, e00978-21.
[11] A. Rominski, A. Roditscheff, P. Selchow, E. C. Böttger, P. Sander, J. Antimicrob. Chemother. 2017, 72, 376-384.
[12] S. Ramaswamy, J. M. Musser, Tubercle and lung disease 1998, 79, 3-29.
[13] D. B. Aziz, J. L. Low, M. L. Wu, M. Gengenbacher, J. W. P. Teo, V. Dartois, T. Dick, Antimicrob. Agents Chemother. 2017, 61, e00155-17.
[14] J. L. Low, M. L. Wu, D. B. Aziz, B. Laleu, T. Dick, Front. Microbiol. 2017, 8, 1539.
[15] W. Lin, S. Mandal, D. Degen, Y. Liu, Y. W. Ebright, S. Li, Y. Feng, Y. Zhang, S. Mandal, Y. Jiang, S. Liu, M. Gigliotti, M. Talaue, N. Connell, K. Das, E. Arnold, R. H. Ebright, Mol. Cell 2017, 66, 169-179.e168.
[16] Z. Otwinowski, W. Minor, Methods Enzymol. 1997, 276, 307-326.
[17] P. Emsley, K. Cowtan, Acta Crystallogr. Sect. D 2004, 60, 2126-2132.
[18] P. V. Afonine, M. Mustyakimov, R. W. Grosse-Kunstleve, N. W. Moriarty, P. Langan, P. D. Adams, Acta Crystallogr. Sect. D 2010, 66, 1153-1163.
[19] S. F. Altschul, W. Gish, W. Miller, E. W. Myers, D. J. Lipman, J. Mol. Biol. 1990, 215, 403-410.
[20] F. Sievers, A. Wilm, D. Dineen, T. J. Gibson, K. Karplus, W. Li, R. Lopez, H. McWilliam, M. Remmert, J. Soding, J. D. Thompson, D. G. Higgins, Mol. Syst. Biol. 2011, 7, 539.
[21] A. Fiser, R. K. Do, A. Sali, Protein Sci. 2000, 9, 1753-1773.
[22] J. Baysarowich, K. Koteva, D. W. Hughes, L. Ejim, E. Griffiths, K. Zhang, M. Junop, G. D. Wright, Proc. Natl. Acad. Sci. U S. A. 2008, 105, 4886-4891.
[23] Clin. Infect. Dis., 2020, 71, e1-e36.
[24] Am. J. Respir. Crit. Care Med., 2007, 175, 367-416.
[25] Clin. Microbiol. Rev., 2012, 25, 545-582.
[26] Sci. Rep., 2021, 11, 19775.

We claim:

1. A compound of formula I, or a pharmaceutically acceptable salt thereof: 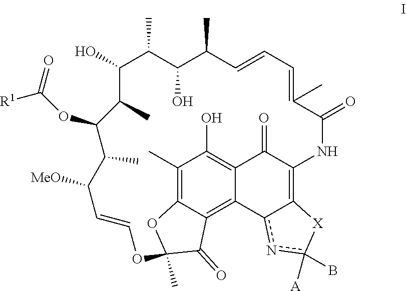
wherein:
R¹ is selected from the group consisting of optionally substituted alkyl, optionally substituted alkenyl, optionally substituted alkynyl, optionally substituted cycloalkyl, optionally substituted aryl, optionally substituted heteroaryl, optionally substituted heterocycloalkyl, —NR⁴R⁵, and —NHSO₂R⁶;
R⁴ and R⁵ are independently selected from the group consisting of hydrogen, optionally substituted alkyl, optionally substituted haloalkyl, optionally substituted alkenyl, optionally substituted alkynyl, optionally substituted cycloalkyl, optionally substituted aryl, optionally substituted heteroaryl, optionally substituted heterocycloalkyl, or
R⁴ and R⁵ together with the nitrogen atom to which they are attached, form an optionally substituted 4-8 membered heterocycloalkyl containing one or more heteroatoms selected from the group consisting of N O, and S(O)₀₋₂;
R⁶ is selected from the group consisting of optionally substituted alkyl, optionally substituted aryl, and optionally substituted heteroaryl; 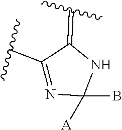
is formula (a), formula (b), or formula (c), 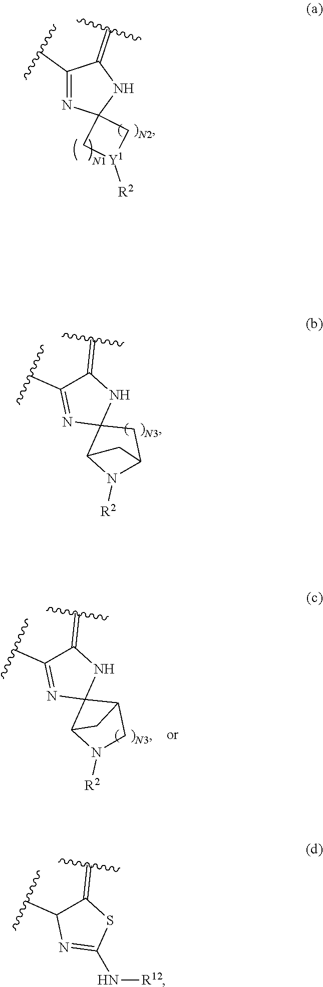
wherein
N1 and N2 are independently an integer of 1-3;
Y¹ is N, O, or CRⁱ;
R² is selected from the group consisting of hydrogen, optionally substituted alkyl, optionally substituted haloalkyl, optionally substituted alkenyl, optionally substituted alkyny, optionally substituted cycloalkyl, optionally substituted aryl, optionally substituted heteroaryl, optionally substituted hetercycloalkyl and —(CH₂)ₙR³;
Rⁱ is hydrogen, halo, optionally substituted alkyl, optionally substituted aryl, optionally substituted heteroaryl, or Rⁱ and R², together with the carbon atom to which they are attached, form an optionally substituted 4-8 membered heterocycloalkyl;
n is an integer of 1-6;
R³ is selected from the group consisting of —OR⁷, —NR⁸R⁹, —C(O)OR¹⁰, S(O)₃OR¹¹, and heteroaryl;
R⁷, R⁸, R⁹, R¹⁰, and R¹¹ are independently selected from the group consisting of hydrogen, alkyl, and haloalkyl;
R¹² is heteroaryl; and
N3 is an integer of 1-2, and
wherein R¹ is not methyl.

2. The compound of claim 1, wherein R¹ is
(i) aryl optionally substituted with one or more alkyl, alkoxy optionally substituted with halo or hydroxyl, hydroxyl, hydroxyalkyl, halo, haloalkyl, haloalkoxy, cyano, aryl, —C(O)OZ¹, —C(O)NZ²Z³, —S(O)Z⁴, —S(O)₂Z⁵, —S(O)₂NZ⁶Z⁷ or amido, wherein Z¹, Z², Z³, Z⁴, Z⁵, Z⁶ and Z⁷ are independently hydrogen, haloalkyl, alkyl, or —C(O)-alkyl;
(ii) a heteroaryl optionally substituted with one or more hydroxyl, halo, optionally substituted amino, amido, haloalkyl, or carboxylic ester;
(iii) —NR⁴R⁵ and R⁴ is hydrogen and R⁵ is
(a) an alkyl optionally substituted with one or more alkynyl or heteroaryl, said heteroaryl optionally substituted with—
one or more aryl optionally substituted with one or more aryl, optionally wherein the substituted aryl is a halo-substituted aryl; or
one or more alkyl optionally substituted with one or more aryl or carboxylic ester;
(b) a heterocycloalkyl optionally substituted with one or more alkylaryl, alkoxy, amino, aryl, —C(O)OZ⁸, or alkyl optionally substituted with one or more haloalkyl or hydroxyl;
(c) an aryl optionally substituted with one or more aryl, alkoxy, alkyl, halo, —C(O)OZ⁸, —C(O)NZ⁹Z¹⁰, —S(O)₂Z¹¹, or —S(O)₂NZ¹²Z¹³; or
(d) a heteroaryl optionally substituted with one or more alkyl, halo, haloalkyl, amino, hydroxyl, —OC(O)Z⁸, or aryl optionally substituted with aryl;
wherein Z⁸, Z⁹, Z¹⁰, Z¹¹, Z¹², and Z¹³ are independently hydrogen, alkyl, or —C(O)-alkyl;
(iv) —NR⁴R⁵ and R⁴ and R⁵, together with the nitrogen atom to which they are attached, form a 4-8 membered heterocycloalkyl containing one or more heteroatoms selected from the group consisting of N, O, and S(O)₀₋₂, said heterocycloalkyl is optionally substituted with one or more aryl, amino, haloalkyl, hydroxyalkyl, alkoxy, carboxylic ester, or alkyl; or
(v) —NHSO₂R⁶ and R⁶ is heteroaryl or aryl optionally substituted with one or more halo, alkoxy, or haloalkyl;
(vi) alkyl;
(vii) alkynyl; or
(viii) cycloalkyl.

3. The compound of claim 1, wherein R¹ is an optionally substituted aryl or an optionally substituted heteroaryl.

4. The compound of claim 1, having a formula of I(a) 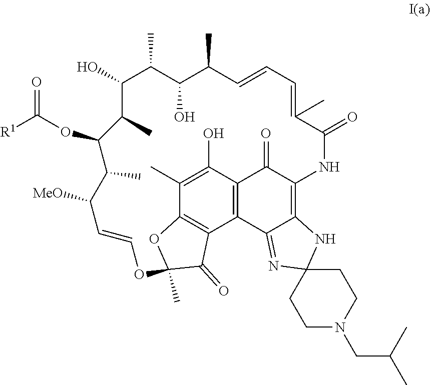
wherein R¹ is selected from the group consisting of alkyl, —NR⁴R⁵, —NHSO₂R⁶, cycloalkyl, heteroaryl optionally substituted with one or more hydroxyl, halo, optionally substituted amino, amido, haloalkyl, or carboxylic ester, and aryl optionally substituted with one or more alkyl, alkoxy optionally substituted with halo or hydroxyl, hydroxyl, hydroxyalkyl, halo, haloalkyl, haloalkoxy, cyano, aryl, —C(O)OZ¹, —C(O)NZ²Z³, S(O)Z⁴, S(O)₂Z⁵, —S(O)₂NZ⁶Z⁷ or amido, wherein Z¹, Z², Z³, Z⁴, Z⁵, Z⁶, and Z⁷ are independently hydrogen, alkyl, or —C(O)-alkyl.

5. The compound of claim 4, wherein R¹ is

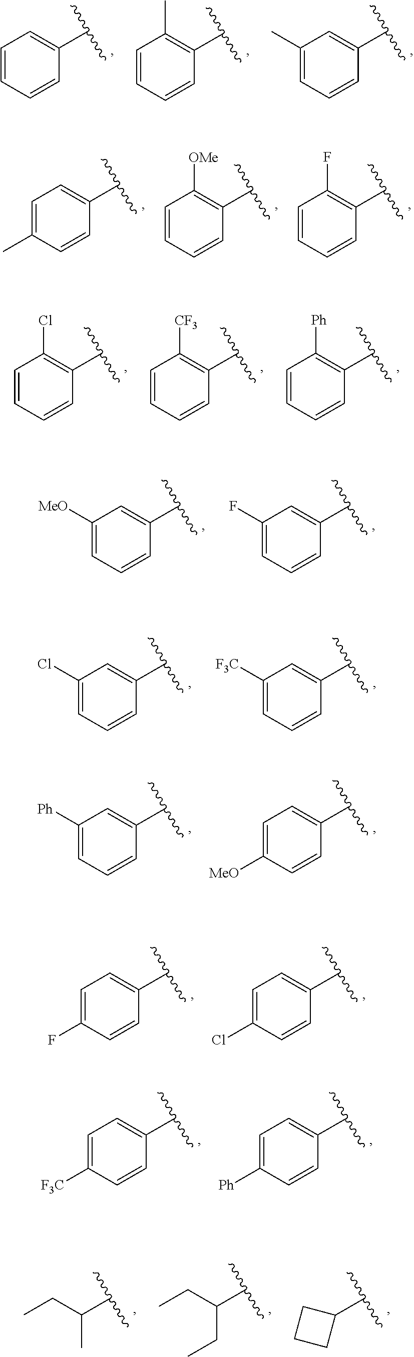

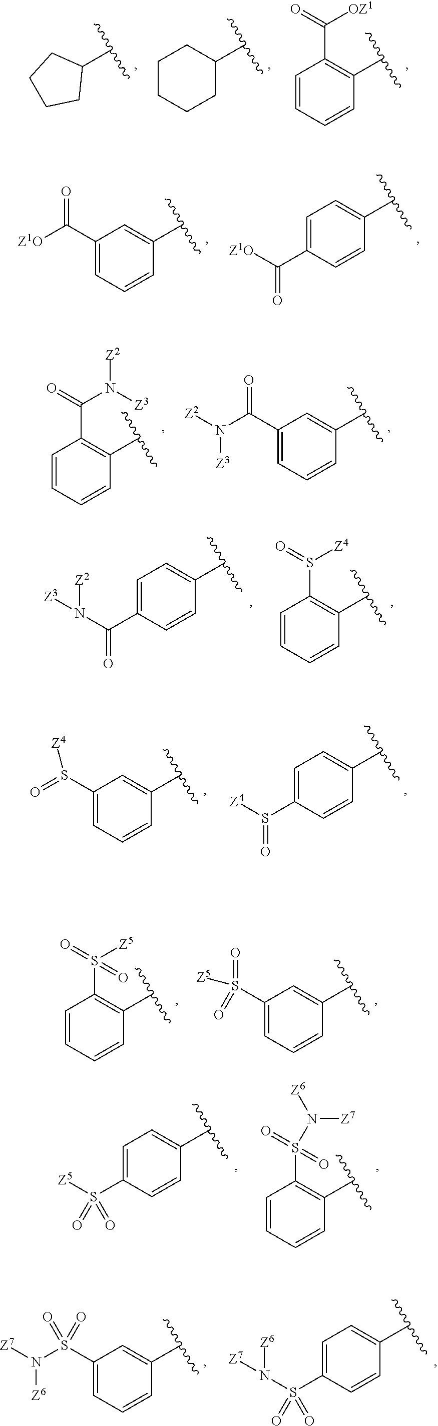

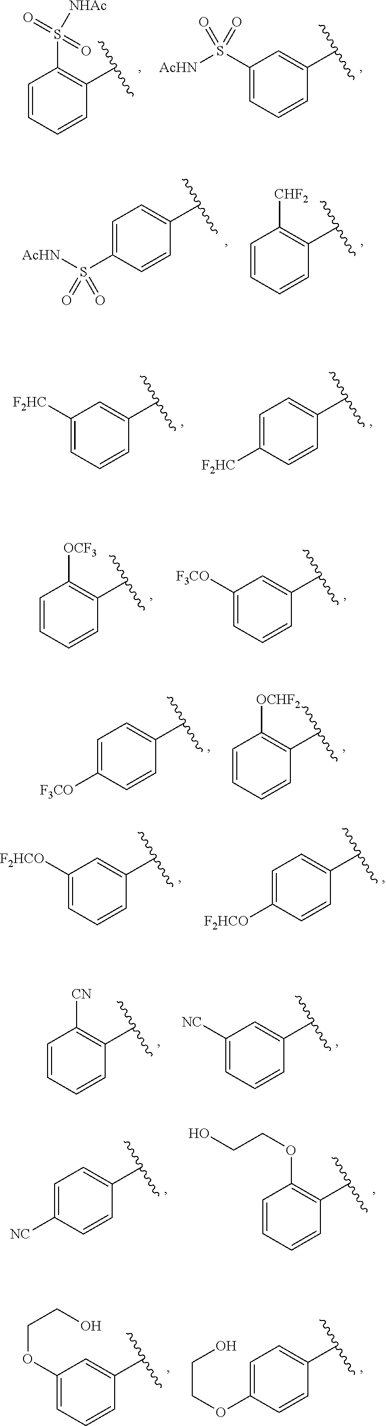

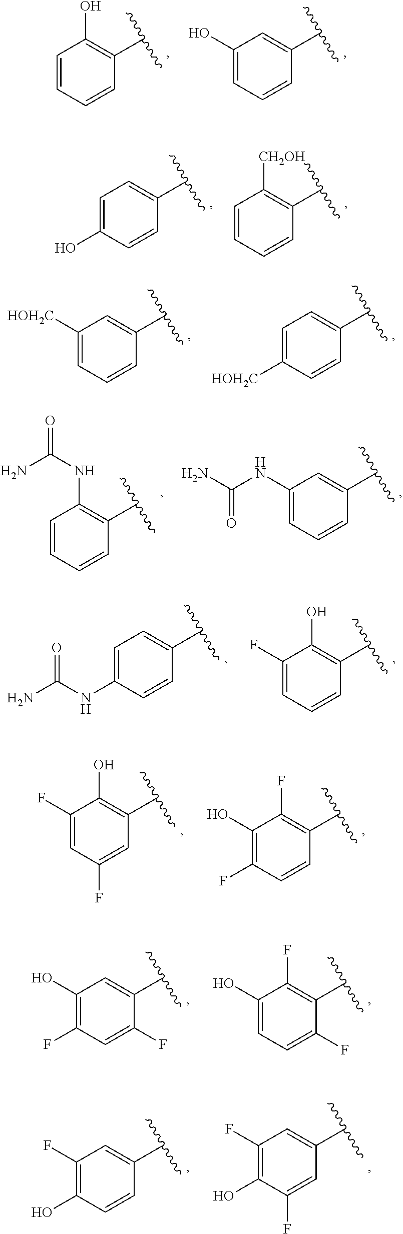

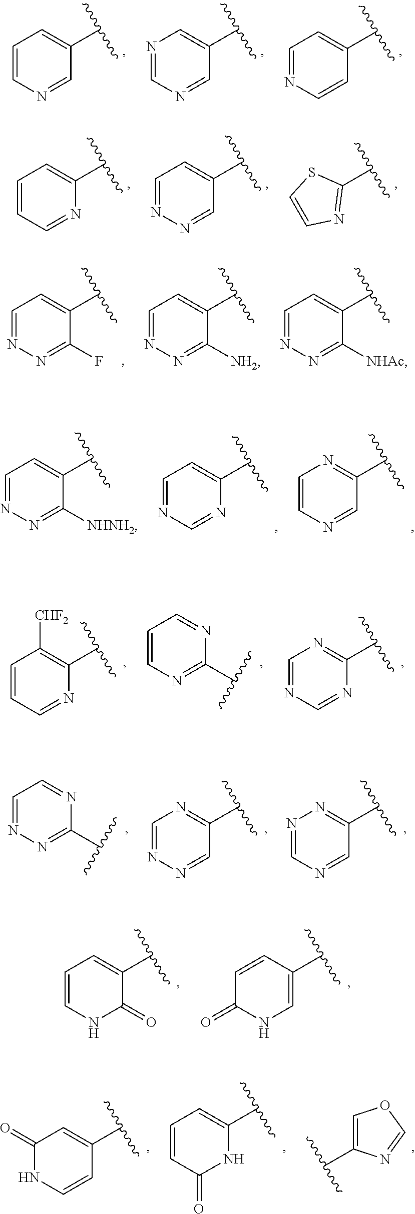

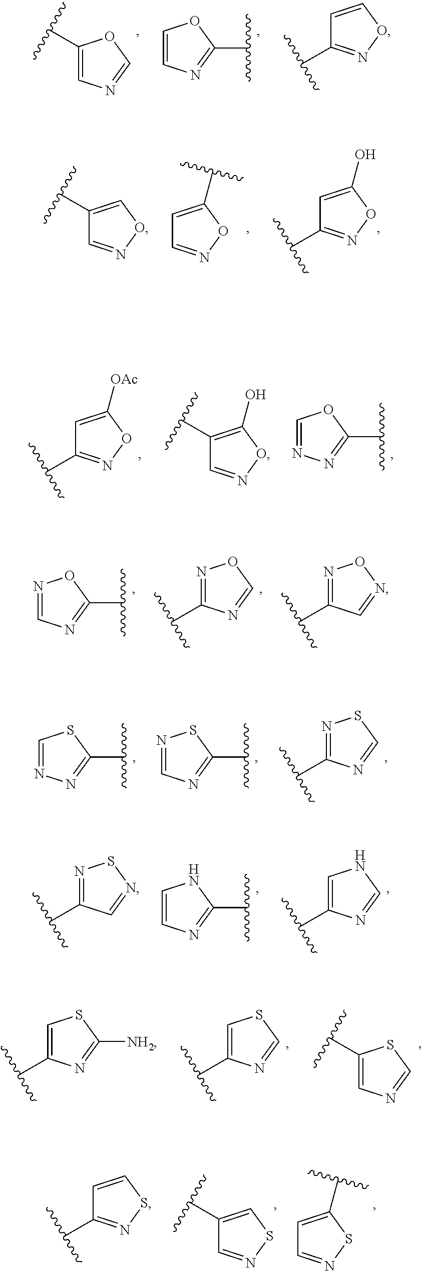

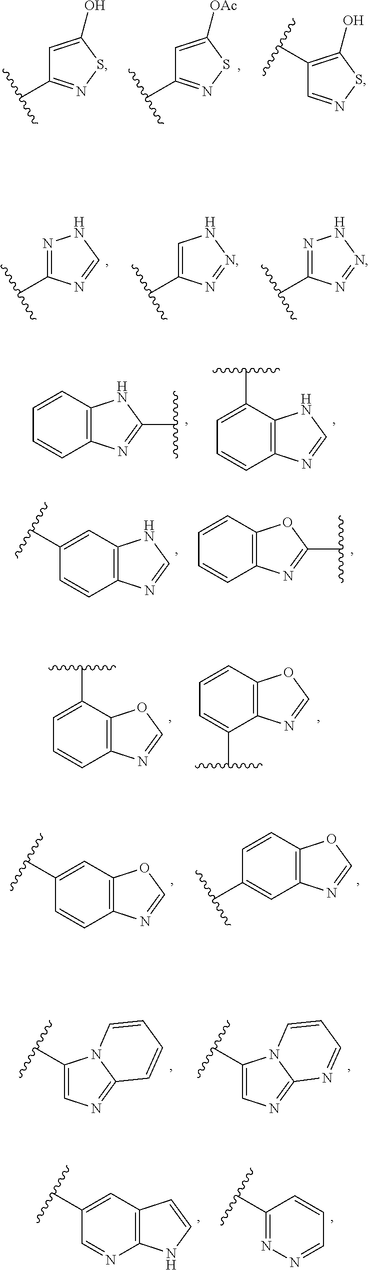

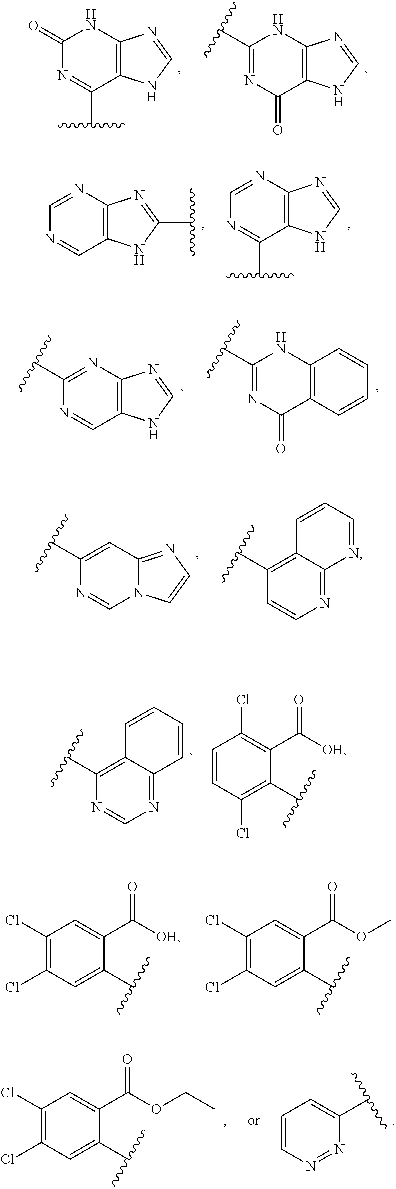

6. The compound of claim 4, wherein the compound is

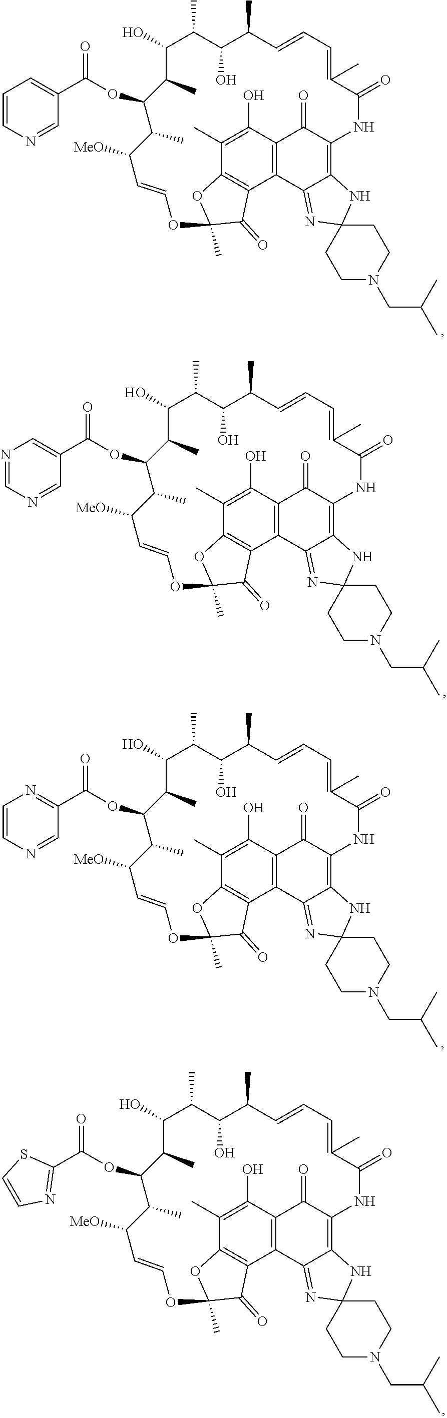

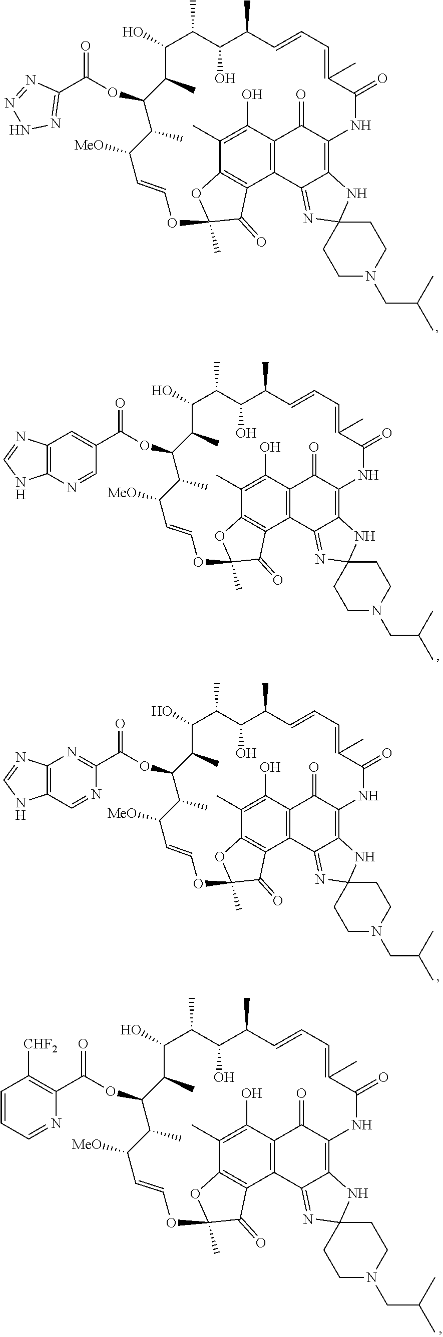

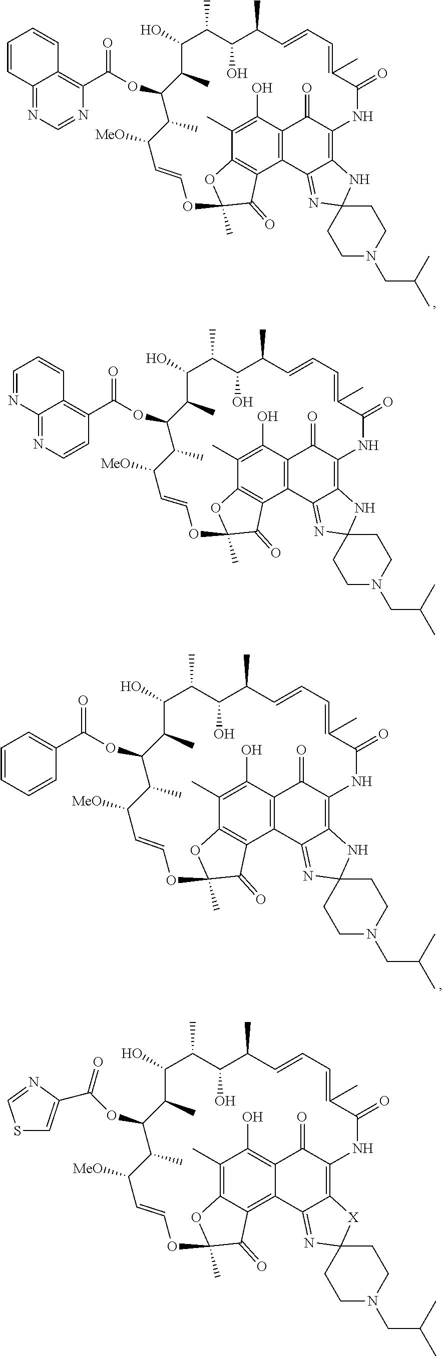

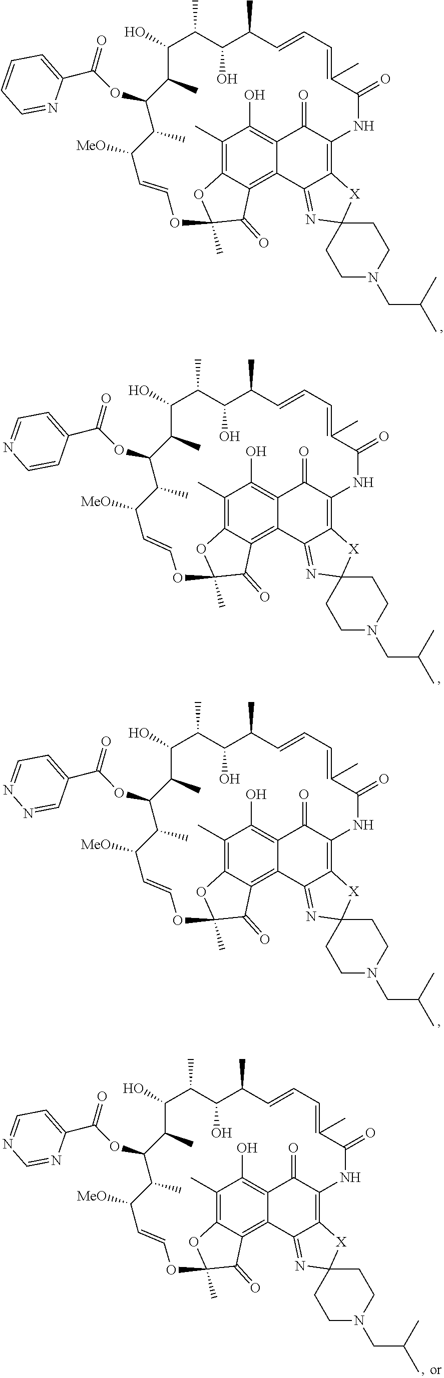

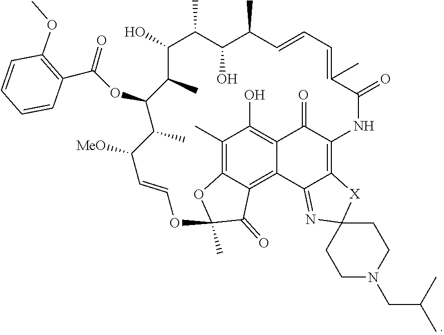

7. The compound of claim 4, wherein R¹ is —NR⁴R⁵;
wherein R⁴ and R⁵ are independently selected from the group consisting of hydrogen, alkyl, heteroaryl optionally substituted with aryl that is optionally substituted with aryl, heterocycloalkyl optionally substituted with one or more alkylaryl, alkoxy, amino, aryl, alkoxy, amino, aryl, —C(O)OZ⁸, or alkyl optionally substituted with one or more haloalkyl or hydroxyl, aryl optionally substituted with one or more aryl, alkoxy, alkyl, —C(O)OZ⁸, —C(O)NZ⁹Z¹⁰, —S(O)₂Z¹¹, or —S(O)₂NZ¹²Z¹³, and heteroaryl optionally substituted with one or more aryl, halo, haloalkyl, —OC(O)Z⁸, amino, or hydroxyl; or
R⁴ and R⁵, together with the nitrogen atom to which they are attached, form a 4-8 membered heterocycloalkyl containing one or more heteroatoms selected from the group consisting of N, O, and S(O)₀₋₂, wherein the heterocycloalkyl is optionally substituted with one or more alkyl, haloalkyl, hydroxyalkyl, aryl, alkoxy, carboxylic ester, or amino; and
wherein Z⁸, Z⁹, Z¹⁰, Z¹¹, Z¹², and Z¹³ are independently hydrogen, alkyl or —C(O)-alkyl.

8. The compound of claim 7, wherein R¹ is

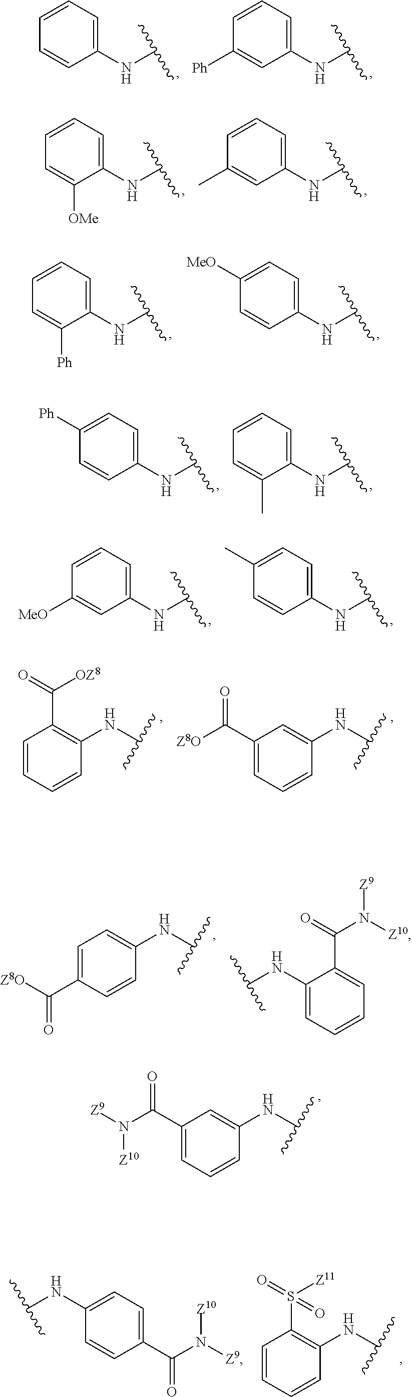

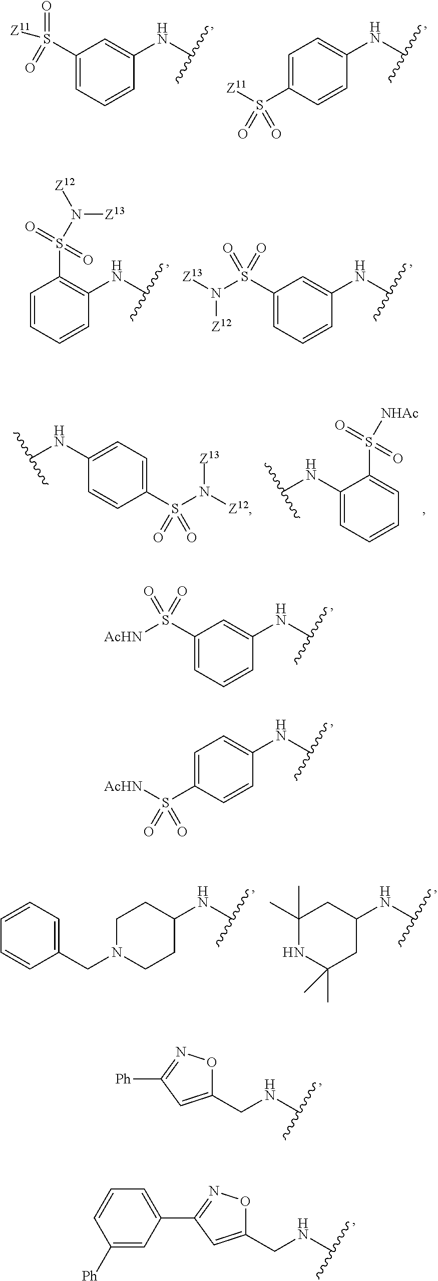

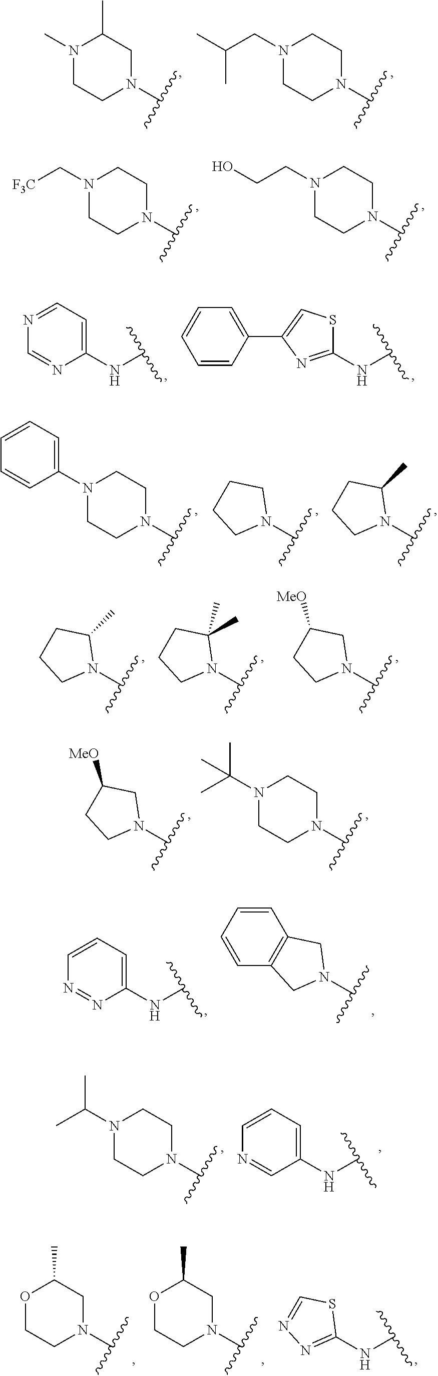

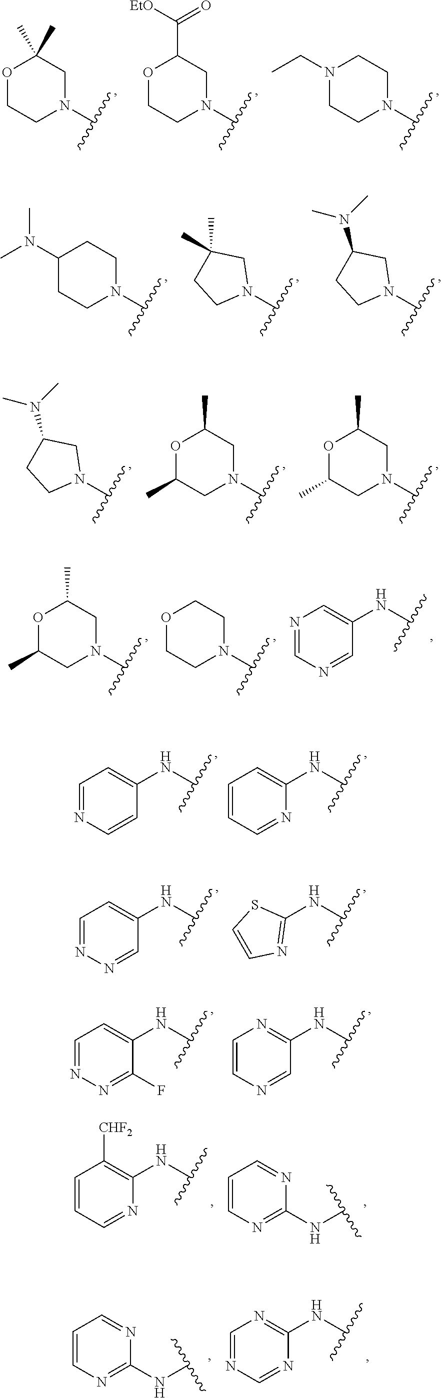

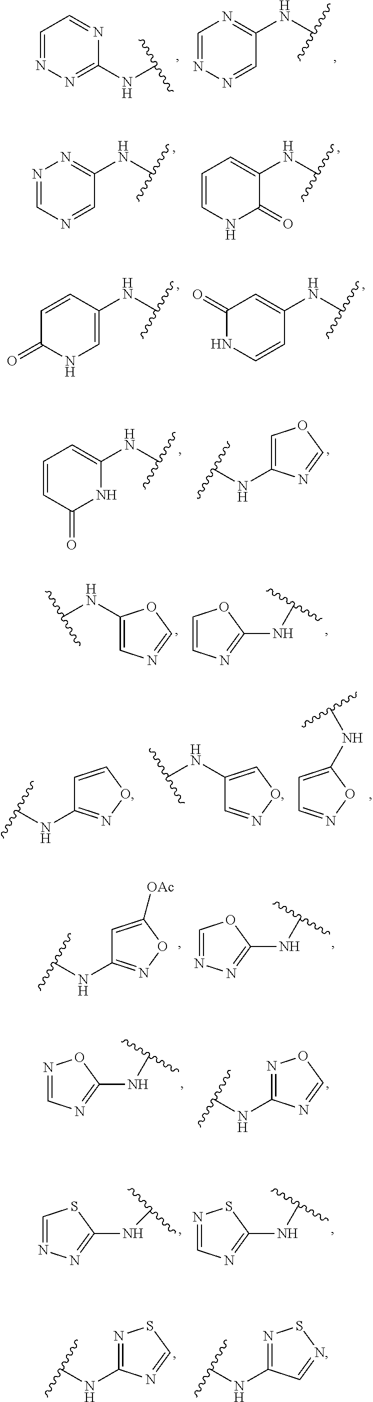

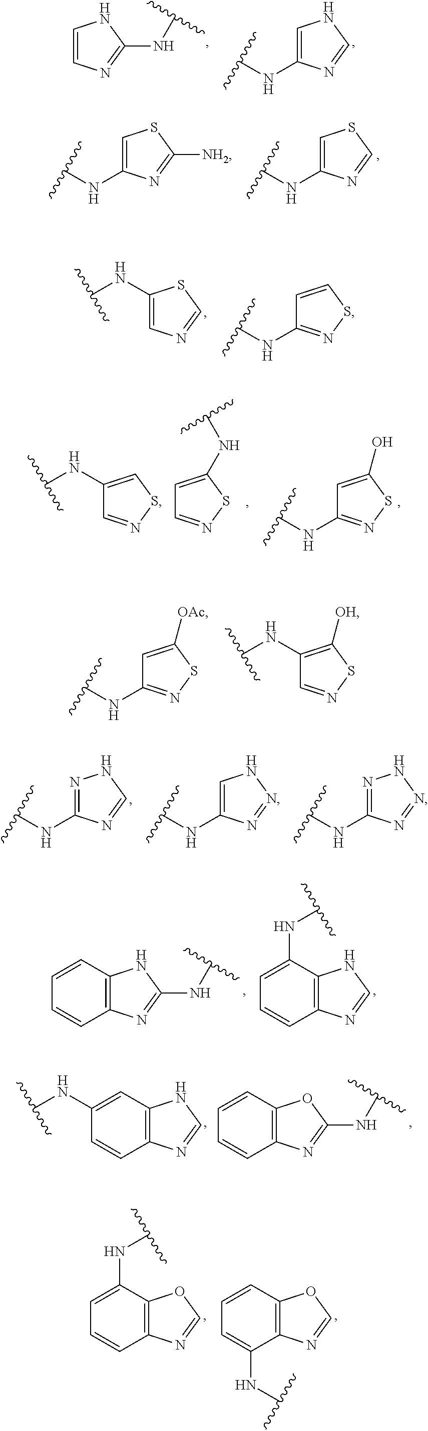

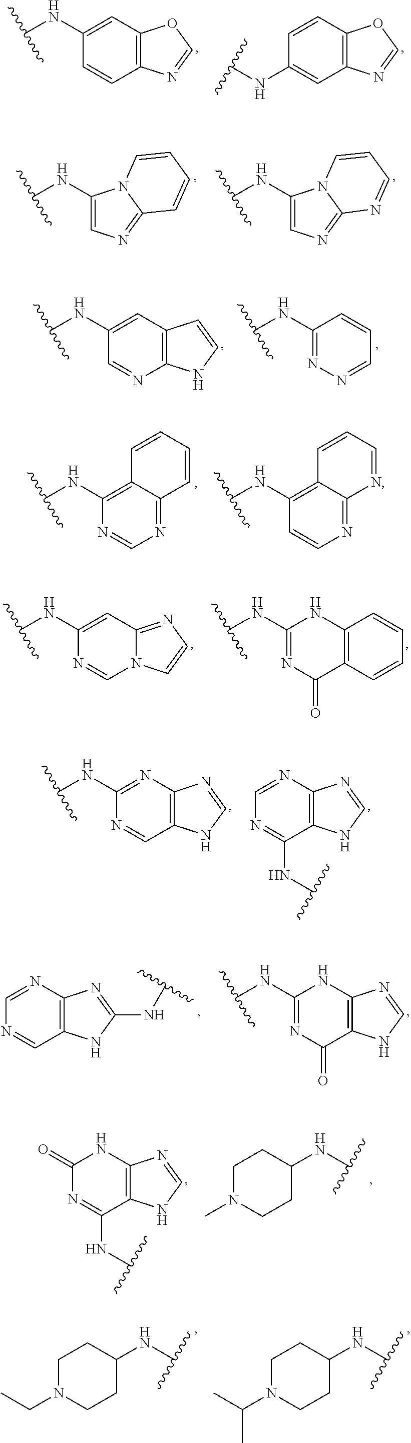

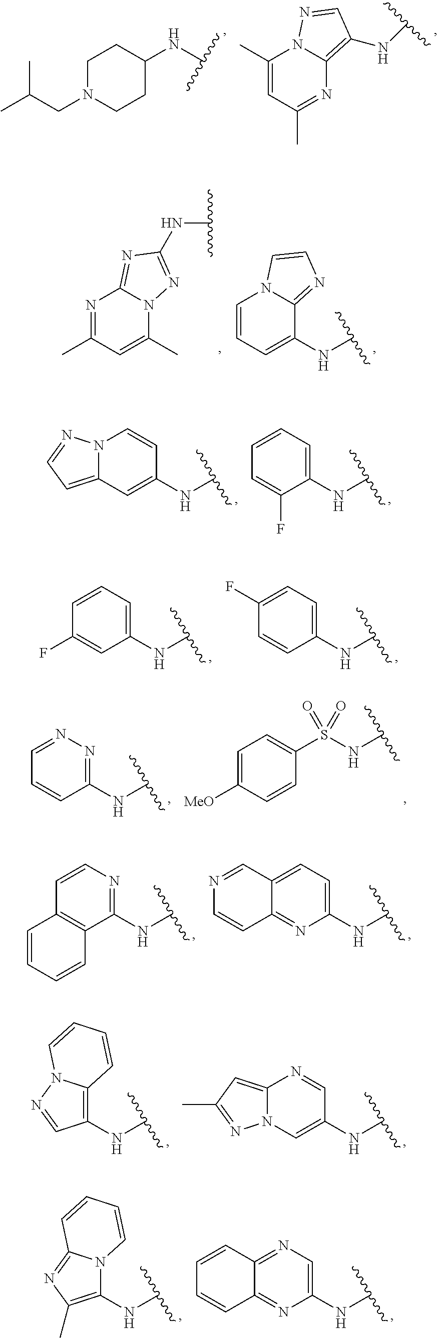

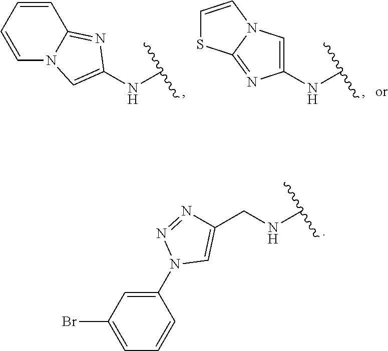 [SMILES: *Nc1cn2ccccc2n1 ; *Nc1cn2ccsc2n1 ; *NCc1cn(-c2cccc(Br)c2)nn1]

9. The compound of claim 4, wherein the compound is

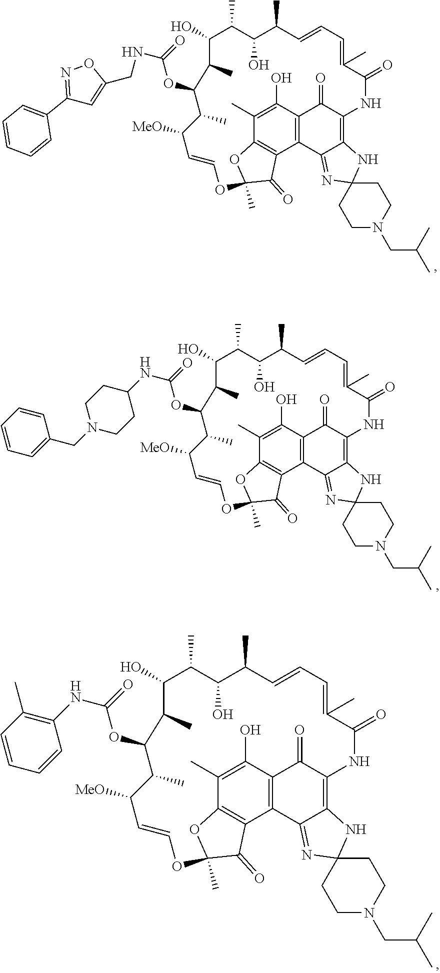

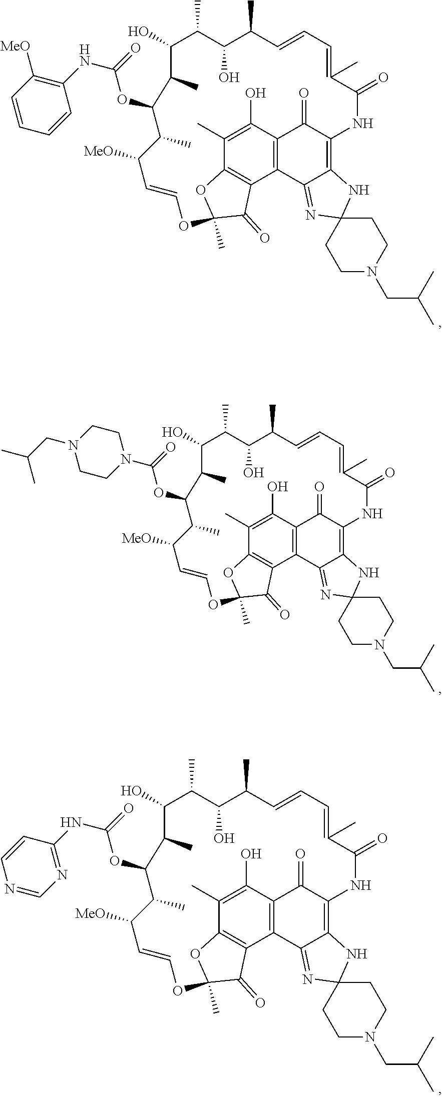

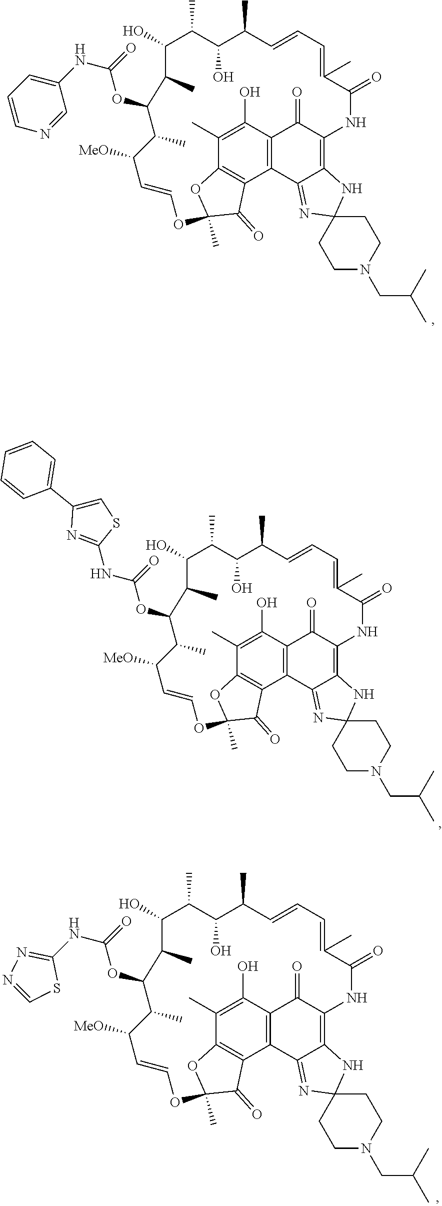

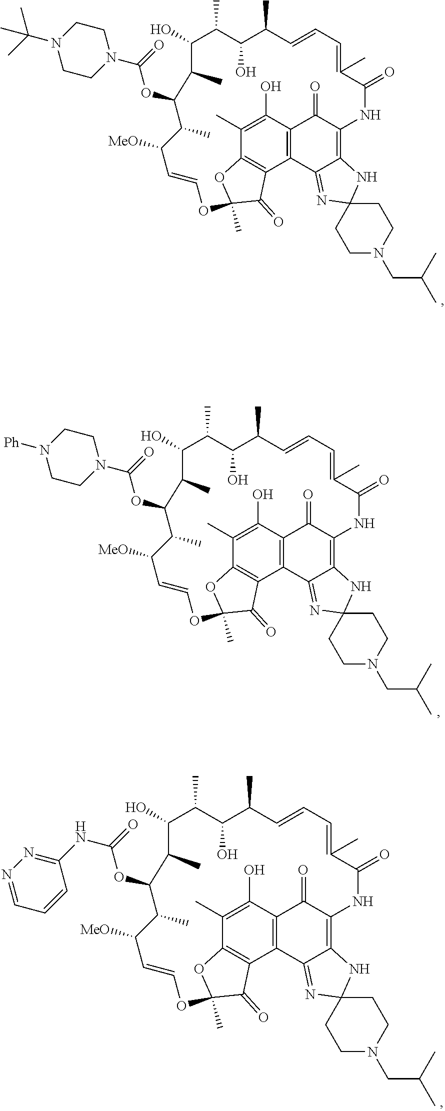

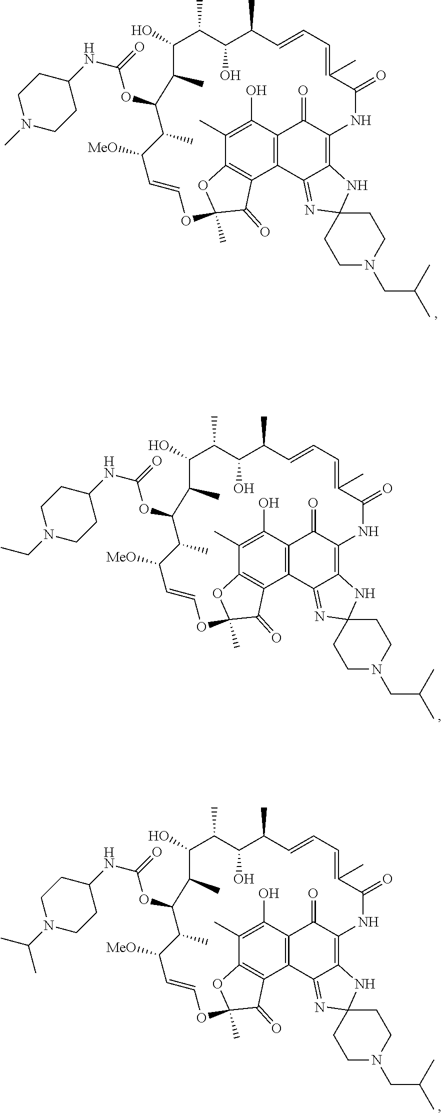

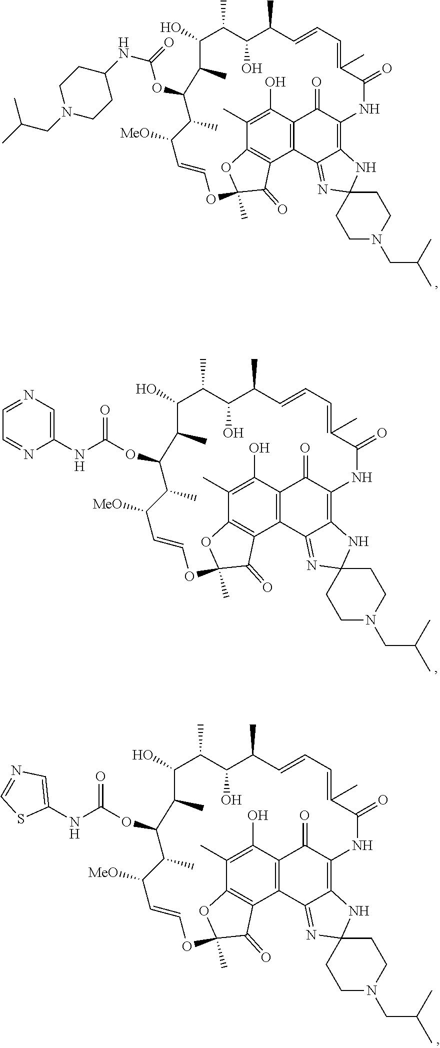

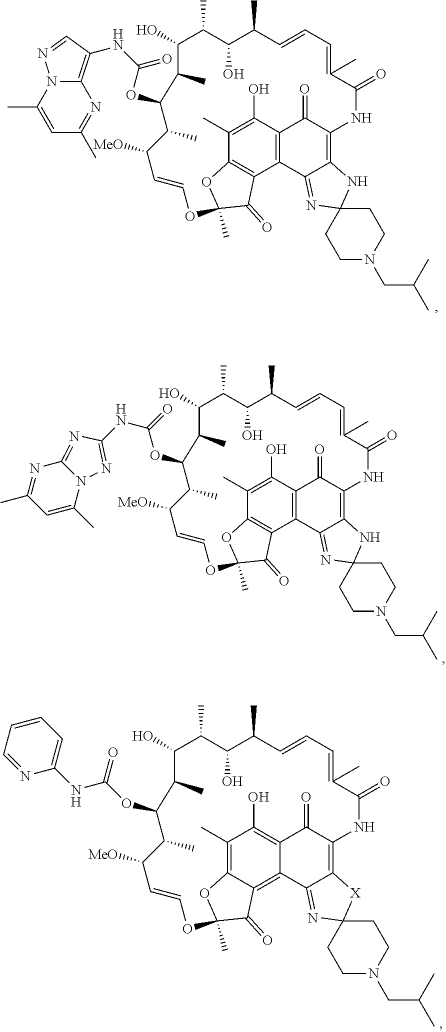

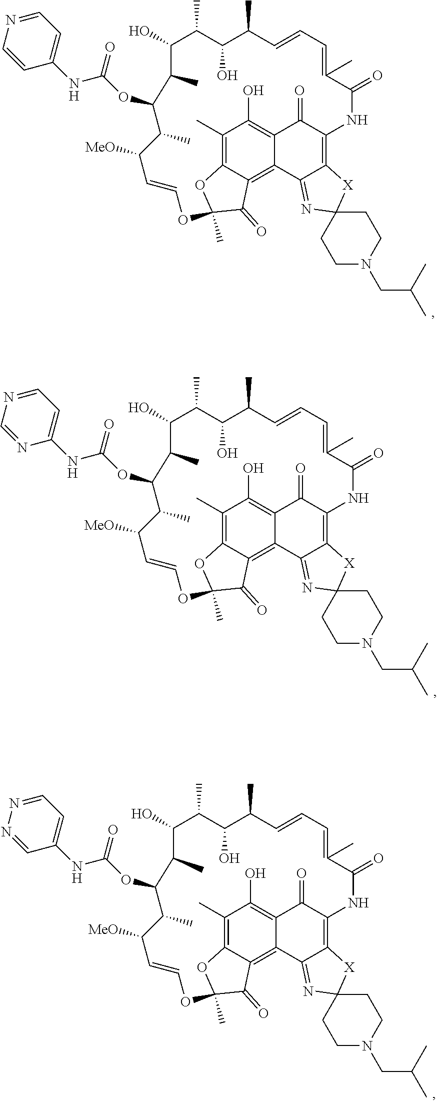

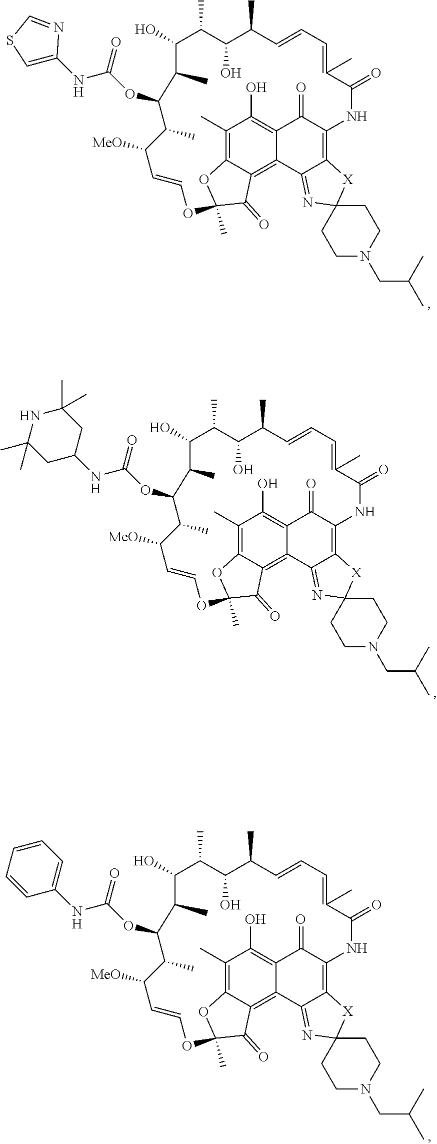

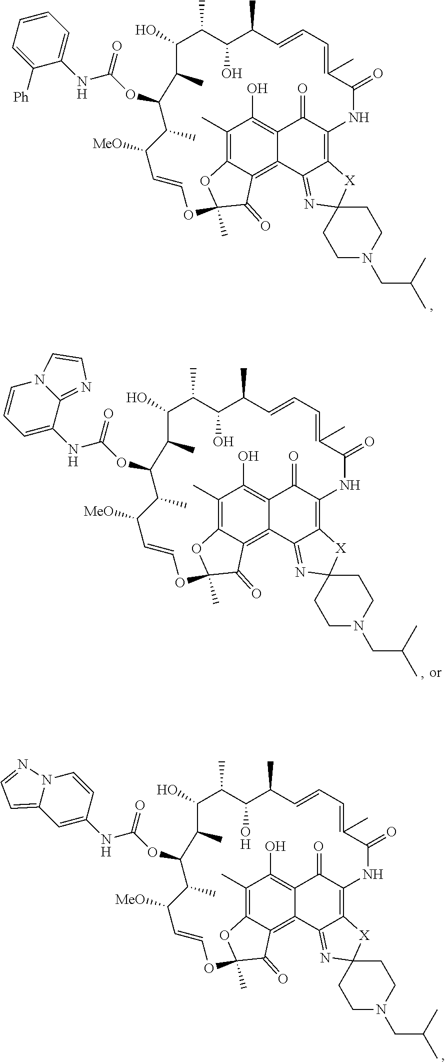

10. The compound of claim 4, wherein R¹ is NHS(S)₂R⁶; and R⁶ is heteroaryl or aryl optionally substituted with one or more halo, alkoxy, or haloalkyl.

11. The compound of claim 10, wherein the compound is

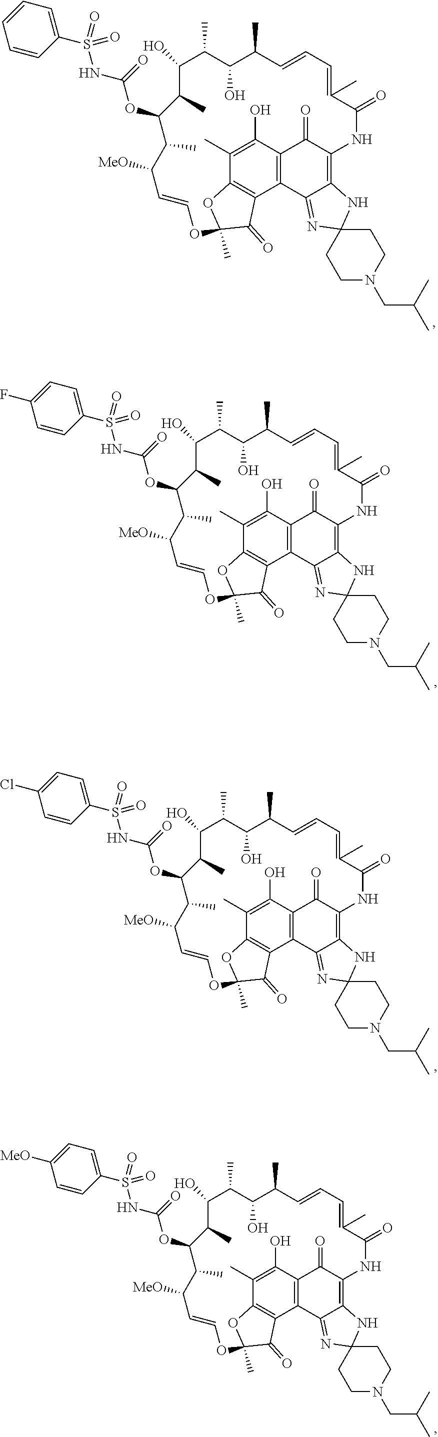

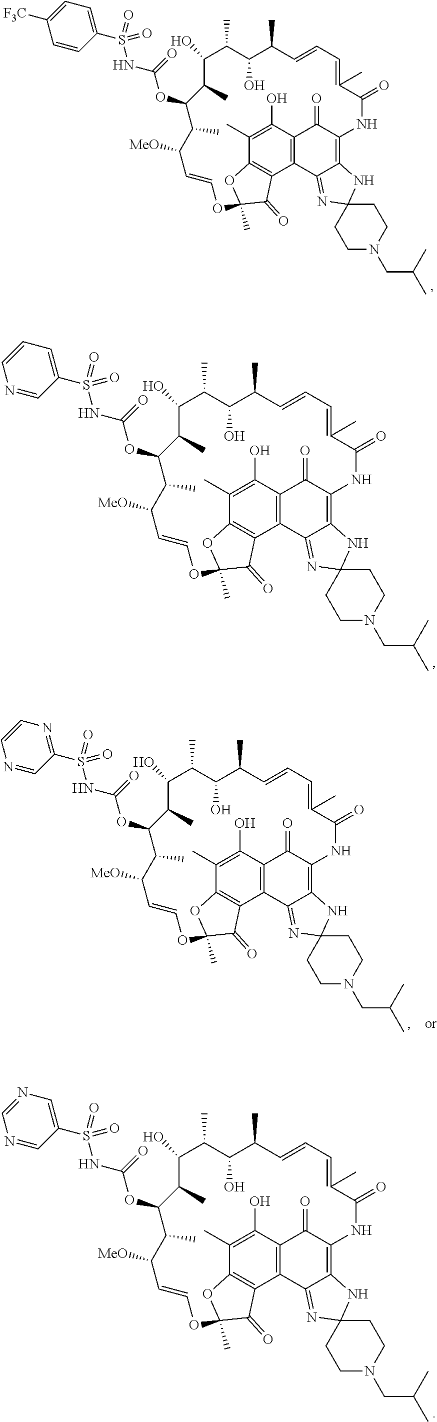

12. The compound of claim 1, wherein formula (b) is

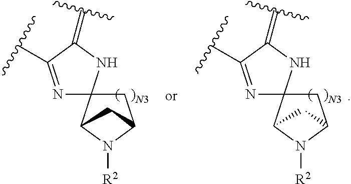

13. The compound of claim 1, wherein formula (c) is

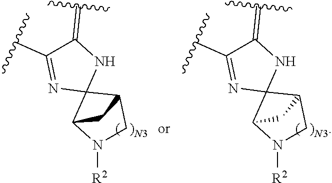

14. The compound of claim 1, wherein
R¹ is an optionally substituted aryl or an optionally substituted heteroaryl; 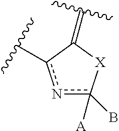
is formula (a) and N1 and N2 are 2;
Y¹ is N; and
R² is selected from the group consisting of hydrogen and alkyl optionally substituted with one or more hydroxyl, amino, carboxyl, or heteroaryl.

15. The compound of claim 14, wherein R² is selected from the group consisting of

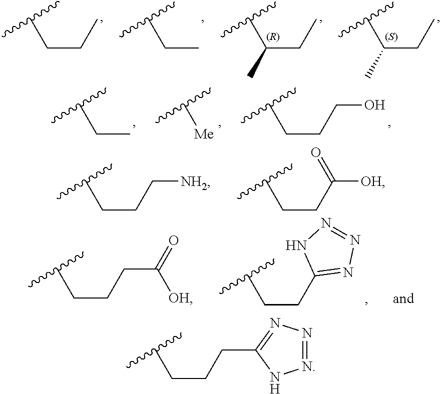

16. The compound of claim 1, wherein
R¹ is an optionally substituted aryl or an optionally substituted heteroaryl;
N1 and N2 are independently 1 or 3;
N3 is 1;
Y¹ is N; and
R² is alkyl.

17. The compound of claim 16, wherein 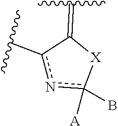
is selected from the group consisting of

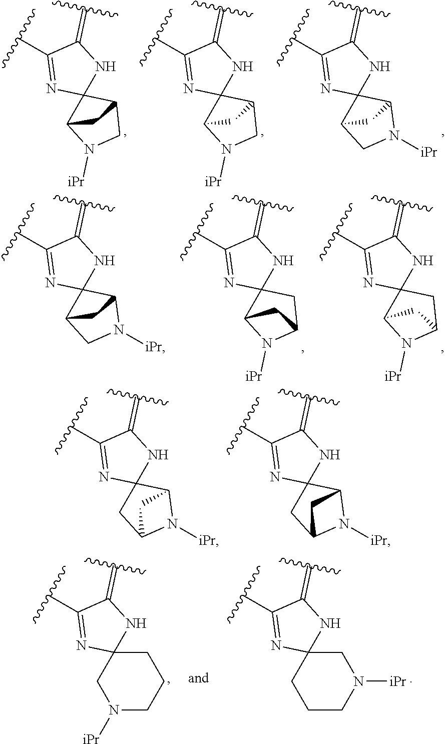

18. The compound of claim 1, wherein R¹ is selected from the group consisting of 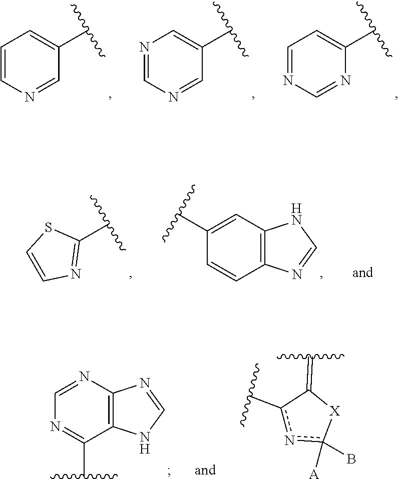
is selected from the group consisting of

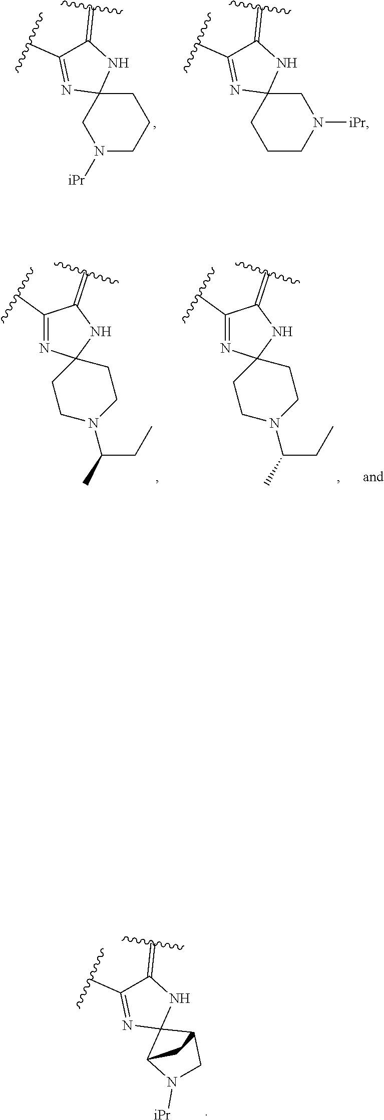

19. The compound of claim 1, wherein the compound is

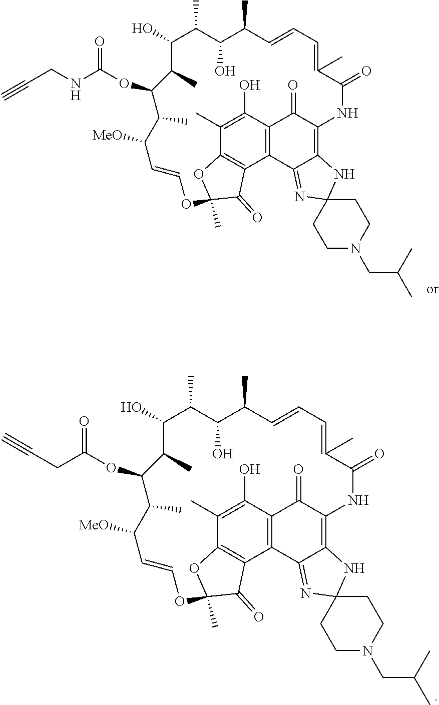

20. The compound of any one of claims 1 to 19, wherein the compound is antibacterial.

21. The compound of any one of claims 1-20, wherein the compound has less inductive effect on a cytochrome P450 enzyme than rifampicin, rifabutin or rifapentine.

22. The compound of claim 21, wherein the cytochrome P450 enzyme is cytochrome P450 3A4.

23. A pharmaceutical composition comprising a therapeutically effective amount of the compound of any one of claims 1 to 22, or a pharmaceutically acceptable salt thereof, and a pharmaceutically acceptable carrier, excipient, or diluent.

24. A method for treating or preventing an infection caused by a Mycobacterium in a subject in need thereof, the method comprising administering to the subject the compound of claim 1 or a pharmaceutically acceptable salt thereof.

25. The method of claim 24, wherein the Mycobacterium is selected from the group consisting of Mycobacterium abscessus, Mycobacterium simiae, Mycobacterium chelonae, Mycobacterium fortuitum, Mycobacterium avium complex, Mycobacterium avium subspecies hominisuis, Mycobacterium intracellulare, Mycobacterium chimaera, Mycobacterium kansasii, Mycobacterium szulgai, Mycobacterium xenopi, and combinations thereof.

26. The method of claim 24, wherein the Mycobacterium is Mycobacterium abscessus.

27. The method of claim 26, wherein the Mycobacterium abscessus is selected from the group consisting of Mycobacterium abscessus subspecies abscessus, Mycobacterium abscessus subspecies bolletii, Mycobacterium abscessus subspecies massiliense, and combinations thereof.

28. The method of any one of claims 24-27, wherein the subject has caseous necrosis.

29. The method of any one of claims 24-27, wherein the Mycobacterium is within caseum in the subject.

30. A method for treating or preventing an infection caused by a bacterium in a subject in need thereof, the method comprising administering to the subject the compound of claim 1 or a pharmaceutically acceptable salt thereof.

31. The method of claim 30, wherein the bacterium is resistant to rifampicin, rifabutin, or rifapentine.

32. The method of claim 30, wherein the bacterium expresses an enzyme that catalyzes ADP-ribosylation of rifampicin, rifabutin, or rifapentine.

33. The method of claim 32, wherein the enzyme that catalyzes ADP-ribosylation of rifampicin, rifabutin, or rifapentine is a rifamycin ADP-ribosyltransferase.

34. The method of any one of claims 30-33, wherein the bacterium is selected from the group consisting of Mycobacterium abscessus, Mycobacterium simiae, Mycobacterium chelonae, Mycobacterium fortuitum, Mycobacterium avium complex, Mycobacterium avium subspecies hominisuis, Mycobacterium intracellulare, Mycobacterium chimaera, Mycobacterium kansasii, Mycobacterium szulgai, Mycobacterium xenopi, and combinations thereof.

35. The method of claim 34, wherein the Mycobacterium is Mycobacterium abscessus.

36. The method of claim 35, wherein the Mycobacterium abscessus is selected from the group consisting of Mycobacterium abscessus subspecies abscessus, Mycobacterium abscessus subspecies bolletii, Mycobacterium abscessus subspecies massiliense, and combinations thereof.

37. The method of any one of claims 30-36, wherein the subject has caseous necrosis.

38. The method of any one of claims 30-36, wherein the bacterium is within caseum in the subject.

39. A method for treating or preventing an infection in a subject in need of an antibacterial compound, the method comprising administering the subject the compound of claim 1 or a pharmaceutically acceptable salt thereof.

40. The method of claim 39, wherein the administered compound has less inductive effect on a cytochrome P450 enzyme than rifampicin, rifabutin, or rifapentine.

41. The method of claim 40, wherein the cytochrome P450 enzyme is cytochrome P450 3A4.

42. The method of any one of claims 39-41, wherein the subject is undergoing treatment with a compound metabolized by the cytochrome P450 enzyme.

43. The method of any one of claims 39-41, wherein the subject is undergoing treatment with a compound that is sensitive to the co-administration of an inducer of the cytochrome P450 enzyme.

44. The method of any one of claims 39-41, wherein the subject is infected with a Mycobacterium selected from the group consisting of Mycobacterium abscessus, Mycobacterium simiae, Mycobacterium chelonae, Mycobacterium fortuitum, Mycobacterium avium complex, Mycobacterium avium subspecies hominisuis, Mycobacterium intracellulare, Mycobacterium chimaera, Mycobacterium kansasii, Mycobacterium szulgai, Mycobacterium xenopi, and combinations thereof.

45. The method of claim 44, wherein the Mycobacterium is Mycobacterium abscessus.

46. The method of claim 45, wherein the Mycobacterium abscessus is selected from the group consisting of Mycobacterium abscessus subspecies abscessus, Mycobacterium abscessus subspecies bolletii, Mycobacterium abscessus subspecies massiliense, and combinations thereof.

47. The method of any one of claims 39-46, wherein the subject has caseous necrosis.

48. The method of any one of claims 39-46, wherein the bacterium is within caseum in the subject.

49. The method of any one of claims 24-48, wherein the compound, or the pharmaceutically acceptable salt thereof is administered orally or intravenously.

50. The method of any one of claims 24-49, wherein the compound is the compound according to any one of claims 2-22.
